(19) United States
(12) Patent Application Publication
(10) Pub. No.: US 2026/0357210 A1
(43) Pub. Date: Nov. 12, 2026

(54) SELECTION OF CRYOPRESERVED CORD BLOOD UNITS FOR THE MANUFACTURE OF NATURAL KILLER CELLS WITH ENHANCED POTENCY

(71) Applicant: BOARD OF REGENTS, THE UNIVERSITY OF TEXAS SYSTEM, Austin, TX (US)

(72) Inventors: Katy REZVANI, Houston, TX (US); David MARIN COSTA, Houston, TX (US)

(73) Assignee: BOARD OF REGENTS, THE UNIVERSITY OF TEXAS SYSTEM, Austin, TX (US)

(21) Appl. No.: 19/485,777

(22) PCT Filed: May 31, 2024
(86) PCT No.: PCT/US2024/031821

§ 371 Date: Nov. 19, 2025

Related U.S. Application Data
(60) Provisional application No. 63/470,634, filed on Jun. 2, 2023.
(60) Provisional application No. 63/604,132, filed on Nov. 29, 2023.

(51) Int. Cl. C12N 5/0783; A61K 35/17; A61K 40/15; A61K 40/31; A61K 40/42; A61P 35/00; C07K 14/54; C07K 14/705; C12N 5/00

(52) U.S. Cl. CPC C12N 5/0646; A61K 35/17; A61K 40/15; A61K 40/31; A61K 40/4211; A61K 40/4232; A61K 40/428; A61P 35/00; C07K 14/54; C07K 14/5443; C07K 14/7051; C12N 5/525; A61K 2239/15; C12N 2510/00

(57) ABSTRACT

Embodiments of the disclosure concern methods and compositions related to optimization and selection of cord blood units for production of immune cells, such as NK cells, for adoptive cell therapy use. In specific embodiments, particular characteristics of the cord blood units and/or characteristics of cells derived therefrom are analyzed. When a threshold measurement for one or more characteristics is met for the cord blood unit(s) and/or characteristics of cells derived therefrom, the cord blood unit(s) are utilized as a source for production of immune cells. Specific characteristics for measurement include, for example, cord blood cell viability, time from birth to cord blood unit cryopreservation, total nuclear cell recovery, baby weight, baby gender, mothers age, gestational age, CD34 positive cell percentage, and/or nucleated red blood cell content. Characteristics may be determined prior to and/or post-cryopreservation.

### CROSS REFERENCE TO RELATED APPLICATIONS

This application claims priority to U.S. Provisional Patent Application Ser. No. 63/470,634 filed Jun. 2, 2023, and U.S. Provisional Patent Application Ser. No. 63/604,132 filed Nov. 29, 2023, the contents of each of which are hereby incorporated by reference in their entirety.

### SEQUENCE LISTING

The instant application contains a Sequence listing which has been submitted in ST26 format and is hereby incorporated by reference in its entirety. Said ST26 copy, created on May 30, 2024, is named MDAC_1365_Sequence_Listing.xml and is 4,812 bytes in size.

### TECHNICAL FIELD

Embodiments of the disclosure concern at least the technical fields of cell biology, molecular biology, immunology, and medicine.

### BACKGROUND

Umbilical cord blood derived natural killer (NK) cells modified to express a CAR are an effective therapy against cancer. Indeed, umbilical cord derived NK cells can be modified (either through genetic or non-genetic methods) to treat multiple malignancies and infections. Cryopreserved cord blood units are readily available in biobanks (as they are used as a source of cells for stem cell transplantation) and can provide sufficient numbers of NK cells to manufacture multiple cell therapy products for clinical use. The alternative to the use of cord blood units as a source of NK cells is to obtain cells from healthy donors by the means of leukapheresis. This procedure is complex and it is not exempt of risk to the donor. The clinical efficacy of an NK cell product is heavily influenced by the characteristics of the cryopreserved cord units. The present disclosure satisfies a long-felt need in the art of procuring suitable cells for cell therapy.

### BRIEF SUMMARY

The present disclosure is directed to methods and compositions related to cell therapy for an individual. The cell therapy may be of any kind, but in specific embodiments the cell therapy comprises adoptive cell therapy with immune cells, including at least immune cells that eventually may be modified prior to administration to an individual in need of the cells. In particular embodiments, the disclosure concerns identification of cord blood units particularly suited to produce effective immune cells for adoptive cell therapy for an individual, including that is more effective than selection of cord blood in the absence of the identification.

The present disclosure concerns what can be multi-part strategies to identify cord blood units that are most likely to produce highly efficacious immune cell therapy products for the treatment of patients, including treatment for any kind of medical condition, at least such as cancer or infection of any kind. The disclosure provides a set of selection criteria including criteria that is: (i) prior to the cryopreservation of the cord blood unit, (ii) post thaw and at the start of immune cell manufacture, such as in a GMP facility, and/or (iii) immune cell characteristics during and at the end of manufacture.

In some embodiments, provided herein are methods of selecting cryopreserved cord blood units (CBUs) for manufacture of improved immune cells comprising, selecting CBUs based on the time from birth of a baby from which the CBU was derived and cryopreservation of the CBU. In some embodiments, selection further comprises selecting CBUs that were cryopreserved within about or exactly 32, 30, 28, 26, 24, 22, or 20 hours following birth of a baby from which the CBU was derived. In some embodiments, selection further comprises selecting CBUs were cryopreserved within exactly or about 24 hours following birth of the baby. In some embodiments, selection further comprises selecting CBUs that comprise a nucleated red blood cell (NRBC) content that is: a) less than or equal to, exactly or about 8.5×10⁷, 8.0×10⁷, or 7.5×10⁷ cells (e.g., total cell numbers) when measured post-reduction, b) less than or equal to, exactly or about 9.9×10⁷, 9.4×10⁷, or 8.9×10⁷ cells when measured pre-reduction (e.g., total cell numbers), and/or c) less than or equal to, exactly or about 5%, 4%, or 3% of the total nucleated cells (TNC) when measured post-reduction. In some embodiments, the NRBC content is less than or equal to, exactly or about 8.0×10⁷ cells when measured post-reduction, less than or equal to, exactly or about 9.4×10⁷ cells when measured pre-reduction, and/or less than or equal to, exactly or about 4% of the total nucleated cells (TNC) when measured post-reduction.

In some embodiments, methods of selecting cryopreserved cord blood units (CBUs) for manufacture of improved immune cells does not comprise selection based on the relative levels of one or more immune cells. In some embodiments, CBUs are not selected based upon their percentage of NK cells, CD8+ T cells, CD4+ T cells, T regulatory cells, B cells, Monocyte-derived dendritic cells (Mo-DCS), and/or plasmacytoid dendritic cells (pDCs). In some embodiments, selected CBUs do not have significant differences in the percentages of NK cells, CD8+ T cells, CD4+ T cells, T regulatory cells, B cells, Monocyte-derived dendritic cells (Mo-DCS), and/or plasmacytoid dendritic cells (pDCs).

In some embodiments, methods of selecting cryopreserved cord blood units (CBUs) for manufacture of improved immune cells comprises selecting CBUs based on: a) total cell viability pre-cryopreservation, b) total CD34 positive cell percentage, c) weight of the baby, d) race of the baby's parents, e) baby's mothers age, f) gestational age of the baby, g) collection method of the cord blood, h) sex of the baby, and/or i) pre-process volume of the cord blood collected. In some embodiments, selection further comprises selecting CBUs based on: a) the total cell viability pre-cryopreservation is greater than or equal to, exactly or about 95%, b) the total CD34 positive cell percentage is greater than or equal to, exactly or about 0.2%, c) the weight of baby is greater than or equal to, exactly or about 3,000 grams, d) the baby has at least one Caucasian parents, e) the mothers age is less than or equal to, exactly or about 34 years of age, f) the gestational age of the baby is less than or equal to, exactly or about 40 weeks, g) the cord blood was collected intra-utero and/or ex-utero, h) the baby is male, and/or i) the pre-process volume of the cord blood collected was less than or equal to, exactly or about 150 ml. In some embodiments, selection further comprises selecting CBUs based on: a) the total cell viability pre-cryopreservation is equal to or greater than or equal to, exactly or about 98.5%, b) the total CD34 positive cell percentage is greater than or equal to, exactly or about 0.245%, c) the weight of baby is greater than or equal to, exactly or about 3,650 grams, d) the baby has at least one Caucasian parents, e) the mothers age is less than or equal to, exactly or about 32 years of age, f) the gestational age of the baby is less than or equal to, exactly or about 38 weeks, g) the cord blood was collected intra-utero, h) the baby is male, and/or i) the pre-process volume of the cord blood collected was less than or equal to, exactly or about 120 ml. In some embodiments, at least 3 of the selection factors are utilized.

In some embodiments, immune cells derived from selected CBUs comprise phenotypic, transcriptional, and/or epigenetic signatures that are distinct from immune cells not selected based on the time from birth of a baby from which the CBU was derived and cryopreservation of the CBU. In some embodiments, the immune cells have an increased polyfunctional strength index (PSI). In some embodiments, the increased PSI comprises an increased effector PSI, increased stimulatory PSI, and/or increased chemoattractive PSI. In some embodiments, the immune cells have increased chromatin accessibility and/or transcriptional levels of genes encoding ZIC2, GLI3, TBX21, IRF2, IRF3, IRF4, IRF7, IRF8, IRF9, NKX2-3, NKX2-8, GLI2, EOMES, GZMA, CXCR6, CMKLR1, NKG2D, CD16, 2B4, T-BET, PFN, GZMA, and/or PRF1. In some embodiments, the immune cells have an increased population doubling rate and/or increased protein secretion rate. In some embodiments, the immune cells have an increased basal respiration and/or maximal respiration rate. In some embodiments, the immune cells have decreased chromatin accessibility and/or transcriptional levels of genes encoding ATF1, ATF2, ATF3, ATF7, CREB1, CREB5, NFAT2, NFATC2, FOX, JUN, JUNB, SMAD2, SMAD3, HIF1A, MAFF, JMJD6, DDIT3, SIAH2, NR4A1, DNAJA1, BAK1, NFKB1, IL-10, LAG3, HASP90AB1, HSPA5, and/or HSPA13. In some embodiments, the immune cells have a decreased rate of trogocytosis and/or decreased transcriptional levels of hallmarks of TNFa signaling via NF-κβ, UV response, hypoxia, IL2 STAT5 signaling, Heme metabolism, apoptosis, inflammatory response, estrogen response early, G2M checkpoint, TGFβ signaling, p53 pathway, cholesterol homeostasis, KRAS signaling, and/or Myc targets V1. In some embodiments, the immune cells have decreased NR4A1, JUND, BCL3, MEF2D, HOXA5, FOXB, JUN, MAFF, ZNF281, KLF6, REL, CEBPG, KLF16, HIF1A, FOS, BCLAF1, GATA3, FOSL2, RARG, EGR2, and/or MAF regulon activity.

In some embodiments, the immune cells are natural killer (NK) cells. In some embodiments, methods further comprise the step of expanding the NK cells. In some embodiments, the CBUs are re-selected based on: a) the NK cell expansion between days 0 and 15 of culture, and/or b) the NK cell expansion between days 6 and 15 of culture. In some embodiments, the CBUs are re-selected based on: a) the NK cell expansion between days 0 and 15 of culture is greater than or equal to, exactly or about 350 fold, and/or b) the NK cell expansion between days 6 and 15 of culture is greater than or equal to, exactly or about 50 fold. In some embodiments, the CBUs are re-selected based on: a) the NK cell expansion between days 0 and 15 of culture is greater than or equal to, exactly or about 450 fold, and/or b) the NK cell expansion between days 6 and 15 of culture is greater than or equal to, exactly or about 70 fold.

In some embodiments, methods provided herein further comprise the step of modifying the NK cells. In some embodiments, the NK cells are modified to express one or more non-endogenous gene products. In some embodiments, the non-endogenous gene product comprises an antigen receptor, a cytokine, a homing receptor, a chemokine receptor, and combinations thereof. In some embodiments, the non-endogenous receptor is a chimeric receptor. In some embodiments, the chimeric receptor is a chimeric antigen receptor (CAR). In some embodiments, the CAR targets CD19, CD70, and/or TROP2. In some embodiments, the non-endogenous receptor is a non-natural T-cell receptor . In some embodiments, methods further comprise expression of one or more non-endogenous cytokines. In some embodiments, the cytokine is IL-15 and/or IL-21. In some embodiments, the NK cells are pre-activated with one or more cytokines. In some embodiments, the cytokines are IL-2, IL-7, IL-12, IL-15, IL-18, and/or IL-21. In some embodiments, the NK cell comprises one or more engineered mutations in an endogenous gene. In some embodiments, the endogenous gene is GR, TGFBR2, CISH, and/or CD38.

Also provided herein are compositions comprising a CBU identified by any one or more of the methods described herein. In some embodiments, the composition is comprised in a pharmaceutically acceptable carrier. In some embodiments, the composition is formulated with one or more cryoprotectants. In some embodiments, the composition comprises a population of immune cells derived from CBUs selected using any one or more of the methods described herein.

In specific embodiments, the immune cells are natural killer (NK) cells. In specific embodiments, the cells are not cells other than NK cells. Methods may further comprise the step of expanding the NK cells and/or modifying the NK cells. In some cases, the NK cells are modified to express one or more non-endogenous gene products, such as one or more non-endogenous receptors (such as one or more chimeric receptors, including one or more chimeric antigen receptors and/or one or more non-natural T-cell receptors). In some cases, the non-endogenous gene product comprises one or more non-endogenous receptors, one or more cytokines, one or more chemokines, one or more enzymes, or a combination thereof. The NK cells may be modified to have disruption of expression of one or more endogenous genes in the NK cells.

In some embodiments, the time from birth to CBU cryopreservation is less than or equal to, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any range derivable therein. In some embodiments, the time from birth to CBU cryopreservation is less than or equal to, exactly or about 25, 24, 23, 22, 21, or 20 hours.

In specific cases, the cord blood cell viability in is greater than or equal to, exactly or about 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9, or 100%.

In specific cases, the TNC recovery in (b) is greater than or equal to, exactly or about 77, 78, 79, 80, 81, 82, 83, 84, 85, 86, 87, 88, 89, 90, 91, 92, 93, 94, 95, 96, 97, 98, 99, or 100%.

In specific cases, the NRBC content is less than or equal to, exactly or about 9.9×10⁷, 9.8×10⁷, 9.7×10⁷, 9.6×10⁷, 9.5×10⁷, 9.4×10⁷, 9.3×10⁷, 9.2×10⁷, 9.1×10⁷, 9.0×10⁷, 8.9×10⁷, 8.8×10⁷, 8.7×10⁷, 8.6×10⁷, 8.5×10⁷, 8.4×10⁷, 8.3×10⁷, 8.2×10⁷, 8.1×10⁷, 8.0×10⁷, 7.9×10⁷, 7.8×10⁷, 7.7×10⁷, 7.6×10⁷, 7.5×10⁷, 7.0×10⁷, 6.0×10⁷, 5.0×10⁷, 4.0×10⁷, 3.0×10⁷, 2.0×10⁷, 1.0×10⁷, 9.0×10⁶, 8.0×10⁶, 7.0×10⁶, 6.0×10⁶, 5.0×10⁶, 4.0×10⁶, 3.0×10⁶, 2.0×10⁶, 1.0×10⁶, 9.0×10⁵, 8.0×10⁵, 7.0×10⁵, 6.0×10⁵, 5.0×10⁵, 4.0×10⁵, 3.0×10⁵, 2.0×10⁵, 1.0×10⁵, 9.0×10⁴, 8.0×10⁴, 7.0×10⁴, 6.0×10⁴, 5.0×10⁴, 4.0×10⁴, 3.0×10⁴, 2.0×10⁴, 1.0×10⁴, 9.0×10³, 8.0×10³, 7.0×10³, 6.0×10³, 5.0×10³, 4.0×10³, 3.0×10³, 2.0×10³, 1.0×10³, 9.0×10², 8.0×10², 7.0×10², 6.0×10², 5.0×10², 4.0×10², 3.0×10², 2.0×10², 1.0×10², and so forth.

In specific cases, the weight of the baby from which the cord blood is derived is greater than 3650 grams.

In specific cases, the race of the biological mother from which the cord blood is derived is Caucasian and/or biological father of the baby from which the cord blood is derived is Caucasian.

In specific embodiments, the gestational age of the baby from which the cord blood is derived is less than or equal to, exactly or about 38 weeks.

In certain embodiments, the cord blood may be obtained by any suitable method, but in specific embodiments it is obtained in utero, extra utero, or both, although in particular cases it is obtained in utero only.

In certain embodiments, the volume of the extracted cord blood in addition to a volume of about 35 mL of anticoagulant is ≤120 mL, such that the volume of the extracted cord blood is no greater than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL or less in volume.

Any method encompassed herein may further comprise the step of deriving immune cells from the thawed cord blood composition. In certain embodiments the immune cells may be NK cells, invariant NK cells, NK T cells, T cells B cells, monocytes, granulocytes, myeloid cells neutrophils, eosinophils, basophils, mast cells, monocytes, macrophages, dendritic cells, stem cells, or a mixture thereof. In specific cases, the immune cells derived from the cord blood composition following thawing are NK cells. In specific embodiments, the immune cells have cytotoxicity greater than or equal to, exactly or about 66.7%. In specific embodiments, cytotoxicity may be greater than or equal to, exactly or about 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In some embodiments, cytotoxicity is measured in any suitable manner. In some embodiments, cytotoxicity is measured utilizing a chromium release assay and/or a tumor lysis assay measured by Incucyte®.

In some embodiments, the cord blood is derived from a fetus or infant at less than or equal to, exactly or about 39, 38, or 37 weeks of gestational age. The cord blood may be derived from a fetus or infant at less than or equal to, exactly or about 39, 38, 37, 36, 35, 34, 33, 32, 31, 30, 29, 28, 27, 26, 25, or 24 weeks or less of gestational age.

In some cases, the method further comprises determining viability of cord blood cells following thawing. In specific aspects, the viability of cord blood cells following thawing is greater than or equal to, exactly or about 86.5%, such as greater than or equal to, exactly or about 87, 88, 89, 90, 91, 92, 93, 94, 95, 96, 97, 98, 99, or 100%.

When the immune cells derived from the thawed cord blood composition are NK cells, they may be expanded. The expansion parameters may or may not be determined on a case-by-case basis. The expansion may be quantified after a particular number of days in culture, such as between day 0 and day 15 and any range therebetween. The fold of expansion by the cells may be of any suitable quantity, such as at least, or greater than about, 3-fold, 5-fold, 7-fold, 10-fold, 20-fold, 25-fold, 50-fold, 75-fold, 10⁰-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, and so forth. In some cases, the expansion of the NK cells between days 0 and 6 in culture is greater than or equal to, exactly or about 7-fold. In some cases, the expansion of the NK cells between days 6 and 15 in culture is greater than or equal to, exactly or about 10-fold. In specific cases, the expansion is between 0 and 15 days or 6 and 15 days or 0 and 6 days (and any range therebetween) and has a greater than 70-fold expansion. In specific cases, the expansion is between 0 and 15 days (and any range therebetween) and has a greater than 450-fold expansion. Ranges of days of expansion with any fold level may include 0-15, 0-14, 0-13, 0-12, 0-11, 0-10, 0-9, 0-8, 0-7, 0-6, 0-5, 0-4, 0-3, 0-2, 0-1, 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, 14-15, and so forth.

The NK cells may be modified, such as modified to express one or more non-endogenous gene products, such as a non-endogenous receptor, including a chimeric receptor, such as a chimeric antigen receptor or non-endogenous receptor is a non-natural T-cell receptor . In some cases, the non-endogenous gene product comprises one or more non-endogenous receptors, one or more cytokines, one or more chemokines, one or more enzymes, or a combination thereof. In specific cases, immune cells derived from the thawed cord blood composition are modified to have disruption of expression of one or more endogenous genes in the cells.

In a specific case, the cord blood cell viability is greater than 98% or 99%, and the NRBC content is lower than 7.5×10⁷ or 8.0×10⁷ or any range therebetween, including 7.5×10⁷-8.0×10⁷, 7.5×10⁷-7.9×10⁷, 7.5×10⁷-7.8×10⁷; 7.5×10⁷-7.7×10⁷; 7.5×10⁷-7.6×10⁷; 7.6×10⁷-8.0×10⁷; 7.6×10⁷-7.9×10⁷; 7.6×10⁷-7.8×10⁷, 7.6×10⁷-7.7×10⁷, 7.7×10⁷-8.0×10⁷; 7.7×10⁷-7.9×10⁷; 7.7×10⁷-7.8×10⁷; 7.8×10⁷-8.0×10⁷; 7.8×10⁷-7.9×10⁷; 7.9×10⁷-8.0×10⁷. In specific embodiments, the cord blood is derived from a fetus or infant at less than or equal to, exactly or about 39, 38, or 37 weeks of gestational age, the viability of cord blood cells following thawing is greater than or equal to, exactly or about 86.5% (and this is optional), the expansion of the NK cells between days 0 and 6 in culture is greater than or equal to, exactly or about 3-fold, and the expansion of the NK cells between days 6 and 15 in culture is greater than or equal to, exactly or about 100-fold, and the expansion of the NK cells between days 0 and 15 is greater than or equal to, exactly or about 900-fold. In specific cases, the expansion is between 6 and 15 days and has a greater than 70-fold expansion. In specific cases, the expansion is between 0 and 15 days and has a greater than 450-fold expansion.

Embodiments of the disclosure comprise cord blood compositions identified by any one or more of the methods encompassed herein. The composition may be comprised in a pharmaceutically acceptable carrier. The composition may be formulated with one or more cryoprotectants. Embodiments of the disclosure comprise compositions comprising a population of immune cells derived from any method encompassed herein.

Also provided herein are methods of treating a subject comprising administering a population of immune cells derived from selected CBUs to an individual. In some embodiments, the method of treating a subject comprising administering a population of immune cells derived from selected CBUs provides an increased rate of overall response (OR), complete response (CR), progression-free survival (PFS), and/or overall survival (OS) relative to a subject not treated with the population of immune cells.

Also provided herein, in some embodiments, are methods for the manufacture of engineered immune cells, comprising: engineering an immune cell population to express one or more non-endogenous gene products, wherein the immune cells are derived from a population of cord blood cells from the birth of a baby, and wherein prior to cryopreservation the population of cord blood cells has the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml. In some embodiments, (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction, and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml. In some embodiments, 1, 2, 3, 4, 5, 6, 7, 8, or 9 of characteristics (c)-(k) are present and/or considered in the method.

Also provided herein, in some embodiments, are methods for the manufacture of a source material for the manufacture of a composition comprising immune cells, the method comprising, cryopreserving a cell population comprising immune cells derived from a population of cord blood cells from the birth of a baby, wherein prior to cryopreservation such population of cord blood cells has the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml. In some embodiments, (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction, and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml. In some embodiments, 1, 2, 3, 4, 5, 6, 7, 8, or 9 of characteristics (c)-(k) are present and/or considered in the method.

Also provided herein, in some embodiments, are compositions comprising an isolated population of cord-blood derived immune cells, wherein the immune cells are derived from one or more cord blood units from the birth of a baby that have the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml. In some embodiments, (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml. In some embodiments, 1, 2, 3, 4, 5, 6, 7, 8, or 9 of characteristics (c)-(k) are present and/or considered in the method.

Certain embodiments of the present disclosure are characterized through the following enumerated aspects.

Aspect 1 is a method of selecting cryopreserved cord blood units (CBUs) for manufacture of improved immune cells comprising, selecting CBUs based on the time from birth of a baby from which the CBU was derived and cryopreservation of the CBU.

Aspect 2 is the method of aspect 1, further comprising selecting CBUs that were cryopreserved within about or exactly 32, 30, 28, 26, 24, 22, or 20 hours following birth of a baby from which the CBU was derived.

Aspect 3 is the method of aspect 1 or 2, wherein the CBUs were cryopreserved within exactly or about 24 hours following birth of the baby.

Aspect 4 is the method of any one of aspects 1 to 3, further comprising selecting CBUs that comprise a nucleated red blood cell (NRBC) content that is: a) less than or equal to, exactly or about 8.5×10⁷, 8.0×10⁷, or 7.5×10⁷ cells when measured post-reduction, b) less than or equal to, exactly or about 9.9×10⁷, 9.4×10⁷, or 8.9×10⁷ cells when measured pre-reduction, and/or c) less than or equal to, exactly or about 5%, 4%, or 3% of the total nucleated cells (TNC) when measured post-reduction.

Aspect 5 is the method of aspect 4, wherein the NRBC content is less than or equal to, exactly or about 8.0×10⁷ cells when measured post-reduction, less than or equal to, exactly or about 9.4×10⁷ cells when measured pre-reduction, and/or less than or equal to, exactly or about 4% of the total nucleated cells (TNC) when measured post-reduction.

Aspect 6 is the method of any one of the preceding aspects, wherein the CBUs are not selected based on the relative levels of one or more immune cells.

Aspect 7 is the method of aspect 6, wherein the CBUs are not selected based upon their percentage of NK cells, CD8+ T cells, CD4+ T cells, T regulatory cells, B cells, Monocyte-derived dendritic cells (Mo-DCS), and/or plasmacytoid dendritic cells (pDCs).

Aspect 8 is the method of aspect 6 or 7, wherein the CBUs do not have significant differences in the percentages of NK cells, CD8+ T cells, CD4+ T cells, T regulatory cells, B cells, Monocyte-derived dendritic cells (Mo-DCS), and/or plasmacytoid dendritic cells (pDCs).

Aspect 9 is the method of any one of aspects 1 to 8, further comprising selecting CBUs based on: a) total cell viability pre-cryopreservation, b) total CD34 positive cell percentage, c) weight of the baby, d) race of the baby's parents, e) baby's mothers age, f) gestational age of the baby, g) collection method of the cord blood, h) sex of the baby, and/or i) pre-process volume of the cord blood collected.

Aspect 10 is the method of aspect 9, further comprising selecting CBUs based on a) the total cell viability pre-cryopreservation is greater than or equal to, exactly or about 95%, b) the total CD34 positive cell percentage is greater than or equal to, exactly or about 0.2%, c) the weight of baby is greater than or equal to, exactly or about 3,000 grams, d) the baby has at least one Caucasian parents, e) the mothers age is less than or equal to, exactly or about 34 years of age, f) the gestational age of the baby is less than or equal to, exactly or about 40 weeks, g) the cord blood was collected intra-utero and/or ex-utero, h) the baby is male, and/or i) the pre-process volume of the cord blood collected was less than or equal to, exactly or about 150 ml.

Aspect 11 is the method of aspect 9 or 10, further comprising selecting CBUs based on: a) the total cell viability pre-cryopreservation is equal to or greater than or equal to, exactly or about 98.5%, b) the total CD34 positive cell percentage is greater than or equal to, exactly or about 0.245%, c) the weight of baby is greater than or equal to, exactly or about 3,650 grams, d) the baby has at least one Caucasian parents, e) the mothers age is less than or equal to, exactly or about 32 years of age, f) the gestational age of the baby is less than or equal to, exactly or about 38 weeks, g) the cord blood was collected intra-utero, h) the baby is male, and/or i) the pre-process volume of the cord blood collected was less than or equal to, exactly or about 120 ml.

Aspect 12 is the method of aspect 11, wherein at least 3 of the selection factors are utilized.

Aspect 13 is the method of any one of aspects 1 to 12, wherein the immune cells phenotypic, transcriptional, and/or epigenetic signatures are distinct from immune cells not selected based on the time from birth of a baby from which the CBU was derived and cryopreservation of the CBU.

Aspect 14 is the method of aspect 13, wherein the immune cells have an increased polyfunctional strength index (PSI).

Aspect 15 is the method of aspect 14, wherein the increased PSI comprises an increased effector PSI, increased stimulatory PSI, and/or increased chemoattractive PSI.

Aspect 16 is the method of any one of aspects 13-15, wherein the immune cells have increased chromatin accessibility and/or transcriptional levels of genes encoding ZIC2, GLI3, TBX21, IRF2, IRF3, IRF4, IRF7, IRF8, IRF9, NKX2-3, NKX2-8, GLI2, EOMES, GZMA, CXCR6, CMKLR1, NKG2D, CD16, 2B4, T-BET, PFN, GZMA, and/or PRF1.

Aspect 17 is the method of any one of aspects 13-16, wherein the immune cells have an increased population doubling rate and/or increased protein secretion rate.

Aspect 18 is the method of any one of aspects 13-17, wherein the immune cells have an increased basal respiration and/or maximal respiration rate.

Aspect 19 is the method of any one of aspects 13-18, wherein the immune cells have decreased chromatin accessibility and/or transcriptional levels of genes encoding ATF1, ATF2, ATF3, ATF7, CREB1, CREB5, NFAT2, NFATC2, FOX, JUN, JUNB, SMAD2, SMAD3, HIF1A, MAFF, JMJD6, DDIT3, SIAH2, NR4A1, DNAJA1, BAK1, NFKB1, IL-10, LAG3, HASP90AB1, HSPA5, and/or HSPA13.

Aspect 20 is the method of any one of aspects 13-19, wherein the immune cells have a decreased rate of trogocytosis and/or decreased transcriptional levels of hallmarks of TNFα signaling via NF-κβ, UV response, hypoxia, IL2 STAT5 signaling, Heme metabolism, apoptosis, inflammatory response, estrogen response early, G2M checkpoint, TGFβ signaling, p53 pathway, cholesterol homeostasis, KRAS signaling, and/or Myc targets V1.

Aspect 21 is the method of any one of aspects 13-21, wherein the immune cells have decreased NR4A1, JUND, BCL3, MEF2D, HOXA5, FOXB, JUN, MAFF, ZNF281, KLF6, REL, CEBPG, KLF16, HIF1A, FOS, BCLAF1, GATA3, FOSL2, RARG, EGR2, and/or MAF regulon activity.

Aspect 22 is the method of any one of aspects 1 to 21, wherein the immune cells are natural killer (NK) cells.

Aspect 23 is the method of aspect 22, further comprising the step of expanding the NK cells.

Aspect 24 is the method of aspect 23, wherein the CBUs are re-selected based on: a) the NK cell expansion between days 0 and 15 of culture, and/or b) the NK cell expansion between days 6 and 15 of culture.

Aspect 25 is the method of aspect 24, wherein: a) the NK cell expansion between days 0 and 15 of culture is greater than or equal to, exactly or about 350 fold, and/or b) the NK cell expansion between days 6 and 15 of culture is greater than or equal to, exactly or about 50 fold.

Aspect 26 is the method of aspect 24 or 25, wherein: a) the NK cell expansion between days 0 and 15 of culture is greater than or equal to, exactly or about 450 fold, and/or b) the NK cell expansion between days 6 and 15 of culture is greater than or equal to, exactly or about 70 fold.

Aspect 27 is the method of any one of aspects 22 to 26, further comprising the step of modifying the NK cells.

Aspect 28 is the method of aspect 27, wherein the NK cells are modified to express one or more non-endogenous gene products.

Aspect 29 is the method of aspect 28, wherein the non-endogenous gene product comprises an antigen receptor, a cytokine, a homing receptor, a chemokine receptor, and combinations thereof.

Aspect 30 is the method of aspect 29, wherein the non-endogenous receptor is a chimeric receptor .

Aspect 31 is the method of aspect 30, wherein the chimeric receptor is a chimeric antigen receptor (CAR).

Aspect 32 is the method of aspect 31, wherein the CAR targets CD19, CD70, and/or TROP2.

Aspect 33 is the method of aspect 29, wherein the non-endogenous receptor is a T-cell receptor (TCR).

Aspect 34 is the method of aspect any one of aspects 28 to 33, further comprising expression of one or more non-endogenous cytokines.

Aspect 35 is the method of aspect 34, wherein the cytokine is IL-15 and/or IL-21.

Aspect 36 is the method of any one of aspects 22 to 35, wherein the NK cells are pre-activated with one or more cytokines.

Aspect 37 is the method of aspect 36, wherein the cytokines are IL-2, IL-7, IL-12, IL-15, IL-18, IL-21, or a combination thereof.

Aspect 38 is the method of any one of aspects 22 to 37, wherein the NK cell comprises one or more engineered mutations in an endogenous gene.

Aspect 39 is the method of aspect 38, wherein the endogenous gene is GR, TGFBR2, CISH, and/or CD38.

Aspect 40 is a composition comprising a CBU identified by any one of the methods of aspects 1 to 39.

Aspect 41 is the composition of aspect 40, comprised in a pharmaceutically acceptable carrier.

Aspect 42 is the composition of aspect 40, formulated with one or more cryoprotectants.

Aspect 43 is a composition comprising a population of immune cells derived from CBUs selected using the method of any one of aspects 1 to 39.

Aspect 44 is a method of treating a subject with cancer comprising, administering the population of immune cells according to aspect 43.

Aspect 45 is the method of aspect 44, wherein the subject has increased rates of overall response (OR), complete response (CR), progression-free survival (PFS), and/or overall survival (OS) relative to a subject not treated with the population of immune cells.

Aspect 46 is a method for the manufacture of engineered immune cells, comprising: engineering an immune cell population to express one or more non-endogenous gene products, wherein the immune cells are derived from a population of cord blood cells from the birth of a baby, and wherein prior to cryopreservation the population of cord blood cells has the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml.

Aspect 47 is the method of aspect 46, wherein: (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction; and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml.

Aspect 48 is a method for the manufacture of a source material for the manufacture of a composition comprising immune cells, the method comprising, cryopreserving a cell population comprising immune cells derived from a population of cord blood cells from the birth of a baby, wherein prior to cryopreservation such population of cord blood cells has the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml.

Aspect 49 is the method of aspect 48, wherein: (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction, and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml.

Aspect 50 is a composition comprising an isolated population of cord-blood derived immune cells, wherein the immune cells are derived from one or more cord blood units from the birth of a baby that have the following characteristics: (a) cord blood cells were cryopreserved within about or exactly 32, 30, 28, 26, 24, 22, or 20 hours following birth of a baby from which the cord blood cells were obtained; and (b) comprise a nucleated red blood cell (NRBC) content that is: i) less than or equal to, exactly or about 8.5×10⁷, 8.0×10⁷, or 7.5×10⁷ cells when measured post-reduction, ii) less than or equal to, exactly or about 9.9×10⁷, 9.4×10⁷, or 8.9×10⁷ cells when measured pre-reduction, and/or iii) less than or equal to, exactly or about 5%, 4%, or 3% of the total nucleated cells (TNC) when measured post-reduction; and optionally, (c) the total cell viability pre-cryopreservation is greater than or equal to, exactly or about 95%, (d) the total CD34 positive cell percentage is greater than or equal to, exactly or about 0.2%, (e) the weight of baby is greater than or equal to, exactly or about 3,000 grams, (f) the baby has at least one Caucasian parents, (g) the mothers age is less than or equal to, exactly or about 34 years of age, (h) the gestational age of the baby is less than or equal to, exactly or about 40 weeks, (i) the cord blood was collected intra-utero and/or ex-utero, (j) the baby is male, and/or (k) the pre-process volume of the cord blood collected was less than or equal to, exactly or about 150 ml.

Aspect 51 is the composition of aspect 50, wherein: (a) cord blood cells were cryopreserved within exactly or about 24 hours following birth of the baby; and (b) comprise a nucleated red blood cell (NRBC) content that is: i) less than exactly or about 8.0×10⁷ cells when measured post-reduction, ii) less than or equal to, exactly or about 9.4×10⁷ cells when measured pre-reduction, and/or iii) less than or equal to, exactly or about 4% of the total nucleated cells (TNC) when measured post-reduction, and optionally, (c) the total cell viability pre-cryopreservation is equal to or greater than or equal to, exactly or about 98.5%, (d) the total CD34 positive cell percentage is greater than or equal to, exactly or about 0.245%, (e) the weight of baby is greater than or equal to, exactly or about 3,650 grams, (f) the baby has at least one Caucasian parents, (g) the mothers age is less than or equal to, exactly or about 32 years of age, (h) the gestational age of the baby is less than or equal to, exactly or about 38 weeks, (i) the cord blood was collected intra-utero, (j) the baby is male, and/or (k) the pre-process volume of the cord blood collected was less than or equal to, exactly or about 120 ml.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1, depict the clinical responses for the 37 patients treated in the clinical study and describes CAR-NK cell persistence in the peripheral blood (PB) of patients after CAR19/IL-15 NK cell infusion. Provided is a bar graph showing the diagnosis and best response for the 37 patients treated in the study; CR: complete response; PR: partial response; SD: stable disease; PD: progressive disease; NHL: non-Hodgkin's lymphoma; low-grade NHL: follicular lymphoma and marginal zone lymphoma; CLL: chronic lymphocytic leukemia; CLL-RT: CLL with Richter's transformation; DLBCL: diffuse large B cell lymphoma; Other: mantle cell lymphoma (n=1), acute lymphoblastic leukemia (n=1), and lymphoplasmacytic lymphoma (n=1).

FIGS. 2A-2B, depict a land mark analysis of the clinical study described in FIG. 1. The Overall Survival (OS) (FIG. 2A) and Progress Free Survival (PFS) (FIG. 2B) for the 30 patients who remained in the study after the day +30 evaluation. The 18 patients who had achieved an OR (green line) at day 30 had a statistically significantly superior OS (p=0.011) and PFS (p=0.016) than the 12 patients who had failed to respond (blue line). Tick marks indicate the times at which data were censored for a given patient. The responders number, non-responders number, responders OS or PFS percentage, and non-responders OS or PFS percentages are noted below the graphs for months 1, 3, 6, 9, and 12 post infusion.

FIGS. 3A-3B, depict box plots comparing the peak copy number of CAR-NK cells according to the day 30 patient response. The peak copy numbers of CAR-NK during the first month after treatment with iC9/CD19-CAR/IL15 (“CAR19/IL15”; also referred to herein as “CD19-CAR NK”) CB-NK cells was statistically significantly higher in patients who achieved OR (FIG. 3A; p=0.0002) or CR (FIG. 3B; p=0.02) compared to those patients who failed to respond.

FIGS. 4A-4G, depict box plots comparing inflammatory cytokines in patients peripheral blood at baseline levels of to the highest levels observed within the 42 days of post infusion toxicity monitoring period. There was not a statistically significant (P=0.12) increase in the plasma levels of IL-15 (FIG. 4A) or IL-1B1 (FIG. 4C; p=0.35) when comparing baseline to max value post infusion at day+42. There was a modest increase in the plasma levels of IL-6 (FIG. 4B; p=0.005), TNFα (FIG. 4D; p=0.02), and INF-γ (FIG. 4E; p=0.005) when comparing baseline to max value post infusion at day+42, however the increase did not reach levels that would be expected in cases of Cytokine release syndrome (CRS). Circles and stars represent outliers and extreme outliers, respectively. FIG. 4F depicts bar graphs showing levels of peripheral blood markers of cytokine release syndrome (IL-1B, left; and IL-6, right) at baseline and maximal levels in the first 6 weeks (up to 42 days) and maximal levels after 3 months (after 90 days) after CAR19/IL-15 NK-cell infusion. FIG. 4G depicts bar graphs showing levels of peripheral blood levels of effector cytokines (IL-15, left; IFN-γ, middle; and TNF-α, right) at baseline and maximal levels in the first 6 weeks (up to 42 days) and maximal levels after 3 months (after 90 days) after CAR19/IL-15 NK-cell infusion. P values were determined by Kruskal-Wallis test. Each symbol represents an individual patient, the outlier is identified by the black dot.

FIGS. 5A-5C, depict peripheral blood B cell and T cell counts. FIG. 5A is a box plot showing peripheral blood B-cell counts measured by flow cytometry in responding patients. The figure shows the B-cell count at 90 days post infusion and at the last follow up in the 10 patients who had achieved CR by day 30. CAR-NK cell infusion induced peripheral blood b-cell aplasia (B-cell count<100 cell/μL) in all responding patients. Most patients still had B-cell aplasia at last follow up although there was a modest, but significant (p=0.028) recovery in the peripheral blood B-cell count. FIG. 5B is a spider plot showing B cell counts calculated based on CD19+B-cell frequencies by flow cytometry in PB samples collected from patients at baseline and at multiple timepoints post CAR-NK cell infusion (n=37 patients). The dotted line represents the threshold for B-cell lymphopenia (<100 B-cells/μL). The shadowed area represents B-cell aplasia (<1 B-cell/μL). The solid blue line represents the mean. FIG. 5D Spider plot showing CD3+ T cell counts calculated based on flow cytometry in PB samples from patients at baseline and at multiple timepoints post CAR-NK cell infusion (n=37 patients). The solid green line represents the mean.

FIGS. 6A-6C, depict quantification of CAR-NK copy numbers as measured by qPCR. The CAR-NK cell copy numbers was found to be independent of the dose level received by the patient when measured at one week after the infusion. The CAR-NK copy numbers in peripheral blood during the first 7 days after the infusion was proportional to the dose received (FIG. 6A). Beyond this time point the CAR-NK level in peripheral blood was found to be independent of the dose level, indicating that copy number after the first week was mostly driven by cell expansion and persistence. FIG. 6B shows the highest CAR-NK copy numbers observed between day 8 and day 28 post-infusion according to the dose level received by the patient. FIG. 6C shows measurements of CAR-NK cells in PB samples (CAR copy number) by quantitative polymerase-chain-reaction (qPCR) in overall responders (ORs, blue; n=18 patients) vs. non-responders (NRs, black; n=19 patients) after treatment with CAR19/IL-15 NK cells. Each dot represents a measurement for one patient at one time point. Measurements for individual patients are connected using dashed lines. The solid lines represent the mean values for each group. Data are shown as median+95% CI. P-values were determined by mixed-effects model with Geisser-Greenhouse correction.

FIGS. 7A-7C, depict quantification of CAR-NK copy number as measured by qPCR was independent of the degree of HLA matching between donor and patient. Shown are CAR-NK copy number in peripheral blood at day+7 after the infusion (FIG. 7A) and the highest CAR-NK copy numbers in peripheral blood between days+8 and +28 after the infusion (FIG. 7B) according to the degree of HLA matching between CBU and the patient. No association was observed between the CAR-NK levels and the degree of HLA matching (p=0.55, and p=0.26 respectively). FIG. 7C are measurements of CAR copy number by qPCR in PB samples of patients according to the degree of match at HLA-A, HLA-B and HLA-DR loci between the cord donor and the patient; HLA match 0/6 (n=5 patients), HLA match 1-3/6 (n=13 patients), HLA match 4/6 (n=19 patients). Data are shown as median+95% CI. P-values were determined by mixed-effects model.

FIGS. 8A-8E, depict Kaplan-Meier curves showing OS and PFS for the 37 patients enrolled in the clinical trial and a schematic overview of the clinical trial. FIG. 8A depicts OS, while FIG. 8B depicts PFS for all 37 patients. When patients were stratified as being non-responders (black line) or responders (blue line), statistically significant differences in OS (FIG. 8C; p=0.0111), and PFS (FIG. 8D; p=0.0156) were observed. Tick marks indicated the times at which data were censored for a given patient. Numbers above each line represent the number of patients at risk. Numbers in parentheses represent the probabilities of OS or PFS at a given time point; mo=months. P-values were determined by log-rank test, and the shaded areas represent 95% confidence interval (CI) of survival probability. FIG. 8E depicts a schematic overview of the CAR19/IL-15 NK cell therapy trial.

FIGS. 9A-9B, depict exemplary Receiver Operating Characteristic (ROC) curves that were utilized to study predictive values of various CBU characteristics of interest, and identify the appropriate cut-off value that will allowed classification of each individual CBU as likely (“good”) or unlikely (“bad”) to induce clinical response in patients. FIG. 9A depicts an ROC for CBU characteristic post reduction nucleated red blood cell content (NRBC). The blue arrow on the ROC curve indicated the value on the NRBC content that can be used to classify the CBU as “good or bad” with the best sensitivity and specificity (this is determined by the closets point to 100% sensitivity and 100% [1-specificity]). In this case the value was 8.0×10⁷ cells. Similar ROC curves were created for each of the CBU characteristics of interest disclosed herein. FIG. 9B depicts an ROC for pre-frozen CBU cell viability. The blue arrow on the ROC curve indicated the value on the CBU cell viability that can be used to classify the CBU as “good or bad” with the best sensitivity and specificity (this is determined by the closets point to 100% sensitivity and 100% [1-specificity]). In this case the value was 98.5%.

FIGS. 10A-10D, depict Kaplan-Meier curves showing OS and PFS for the 37 patients enrolled in the clinical trial when the patients were categorized based on the Number of Favorable Characteristics (NFC) found in the CBUs utilized to generate the patient's CD19 CAR-NK treatment (e.g., CAR19/IL15 NK cells). FIG. 10A shows how PFS improved with the NFC in the CBU from which the CD19 CAR-NK infused to the patient were derived (1, 2, 3, 4, or 5 NFCs respectively; p=0.00002). FIG. 10B shows how PFS was found to be improved when the patients were grouped into those receiving CBU derived NK cells with 0-3 NFC, or 4-5 NFC (p=0.00004). FIG. 10C shows how OS improved with the NFC in the CBU from which the CD19 CAR-NK infused to the patient were derived (1, 2, 3, 4, or 5 NFCs respectively; p=0.04). FIG. 10D shows how OS was found to be improved when the patients were grouped into those receiving CBU derived NK cells with 0-3 NFC, or 4-5 NFC (p=0.003).

FIGS. 11A-11B, depict exemplary ROC curves using multiple CBU characteristics as described herein. FIG. 11A shows an ROC curve when 5 CBU characteristics (e.g., viability≥98.5%, NRBC content <8, Caucasian ethnicity, time from birth to cryopreservation≤24 h, and baby's weight >3650 grams) were utilized together, the predictive value of the five criteria set on clinical response was 90.3%. FIG. 11B shows an ROC curve when 10 CBU characteristics (e.g., viability≥98.5%, NRBC content <8, Caucasian ethnicity, time from birth to cryopreservation≤24 h, baby's weight >3650 grams, gestational age≤38 weeks, male gender, CD34%>0.245%, pre-process CBU volume≤120 ml, and NK cell expansion between days 0 and 15 in culture ≥450 fold) were utilized together, the predictive value of the ten criteria set on clinical response was 97%.

FIGS. 12A-12B, depict Kaplan-Meier curves showing OS (FIG. 12A) and PFS (FIG. 12B) for the 37 patients enrolled in the clinical trial when the patients were categorized based on the quality of CBUs utilized to generate the patient's CD19 CAR-NK treatment (e.g., CAR19/IL15 NK cells). The CBUs were considered optimal (n=16; “Opt-Cs”) if the time from birth to cryopreservation was ≤24 h and the post-reduction NRBC content was ≤8×10⁷. The CBUs were considered sub-optimal (n=21; “Sub-Cs”) when CBUs had a time to freezing >24 h and/or an NRBC content (e.g., a pre- or post-reduction NRBC content) greater than a certain level, such as a post-reduction NRBC content of >8×10⁷. CBU quality was the most significant predictor of response to CB-derived CD19 CAR-NK cell treatment, with both OS and PFS rates being significantly improved when NK cells were derived from CBUs that had both a time to freezing≤24 h and a post-reduction NRBC content of ≤8×10⁷ (p=0.0031, and p<0.0001 respectively; referred to in these Figures and the associated Examples as “Opt-Cs” or “Opt-NK”). This classification method provided an easy, abbreviated, and robust manner with which to classify CBUs as potentially efficacious or not. Tick marks indicated the times at which data were censored for a given patient. Numbers above each line represent the number of patients at risk. Numbers in parentheses represent the probabilities of OS or PFS at a given time point; mo=months. P-values were determined by log-rank test, and the shaded areas represent 95% confidence interval (CI) of survival probability.

FIGS. 13A-13G, depicts phenotypic analyses of NK cell traits from suboptimal (“Sub-Cs”) CBUs when compared to more optimal (“Opt-Cs”) CBUs. Opt-Cs CBUs had a time to freezing≤24 h and a post-reduction NRBC content of ≤8×10⁷, and Sub-Cs CBUs had a time to freezing >24 h and/or a pre-reduction NRBC content of >8×10⁷. Unmanipulated NK cells derived from cord blood mononuclear cells (CBMCs) of Opt-Cs or Sub-Cs displayed distinct phenotypic, transcriptional, and epigenetic signatures. Cryopreserved CBMCs that were stored in MD Anderson Cancer Center's (MDACC's) cord bank from each of the cords used to manufacture the clinical CAR19/IL15 CB-NK cell products were utilized to characterize differences in the phenotype of unmanipulated NK cells between Opt-Cs and Sub-Cs. FIG. 13A, dot plots (left panels) represented SPADE analysis of CyTOF data showing the phenotype of unmanipulated live hCD45+CD56+CD3-NK cells in CBMCs of Sub-Cs (n=18 donors) or Opt-Cs (n=13 donors) that were used to generate the clinical CAR-NK cell products. Samples were pooled and separated into two categories: Opt-Cs vs. Sub-Cs. Clustering by SPADE analysis revealed 4 main clusters (the clusters are called “S^{val}1”, “S^{val}2”, “S^{val}3”, and “S^{val}4”). Frequencies of each cluster were indicated; with size and color of nodes within each cluster representing numbers of clustered cells. FIG. 13A, heatmap (right panel) representing the expression levels of NK cell markers within the main sub-clusters of S^{val}1, “S^{val}2, S^{val}3, and S^{val}4, the expression level for each marker is represented on a color scale ranging from the color blue (low) to the color red (high). To validate the results, CBMCs obtained from an independent cohort of CBs from MDACC's Cord Blood bank were analyzed. FIG. 13B depicts SPADE analysis of CyTOF data showing the phenotype of unmanipulated live hCD45+CD56 CD3-NK cells in CBMCs of Sub-Cs (n=6 donors) or Opt-Cs (n=6 donors). The phenotypic signatures of collected NK cells were evaluated by CyTOF, down-sampled to 10,000 cells per sample, pooled and separated into two categories: Sub-Cs vs. Opt-Cs. Clustering by SPADE revealed 4 main clusters (Clusters 1v-4v). Frequencies of each cluster were indicated; size and color of nodes represent numbers of clustered cells. P-values were determined by two-tailed student's t test. FIG. 13C depicts a bar graph showing the percentage (%) of NK cells within Cluster 1v in CBMCs from Sub-Cs vs. Opt-Cs from FIG. 13B. FIG. 13D depicts a heatmap representing the expression levels of NK cell markers within the main sub-clusters of cluster 1v-4v from FIG. 13B. Each column represents a major node within the SPADE tree clusters. The major nodes are those that are representative of the majority of cells across all conditions. The expression level for each marker is represented on a color scale ranging from the color blue (low) to the color red (high). FIG. 13E depicts another SPADE analysis of CyTOF data showing the phenotype of unmanipulated live hCD45+CD56+CD3-NK cells in CBMCs of Sub-Cs (n=18 donors) or Opt-Cs (n=13 donors) that were used to generate the clinical CAR NK-cell products and were available for the analysis. Only samples with viable CBMCs>1,500 cells were analyzed. Samples were pooled and separated into two categories: Opt-Cs vs. Sub-Cs. Clustering by SPADE analysis revealed 4 main clusters (Clusters 1-4). Frequencies of each cluster were indicated; size and color of nodes within each cluster represent numbers of clustered cells. FIG. 13F depicts a bar graph shows the percentage (%) of NK cells within Cluster 1 of FIG. 13E for each Sub-Cs vs. Opt-Cs used to generate the clinical CAR19/IL-15 NK-cell products. FIG. 13G depicts a heatmap representing the expression levels of NK cell markers within the main sub-clusters of Clusters 1~4 of FIG. 13E. Each column represents a major node within the SPADE tree clusters. The major nodes are those that are representative of the majority of cells across all conditions. The expression level for each marker was represented on a color scale ranging from the color blue (low) to the color red (high). P-values were determined by two-tailed Student's t test and data shown as mean±s.e.m. Each symbol represents an individual sample.

FIGS. 14A-14E, shows how NK cells from optimal cords (“Opt-Cs”) vs. suboptimal cords (“Sub-Cs”) as described in FIGS. 12A-12B and FIG. 13, have distinct transcriptional profiles. FIG. 14A depicts a transcriptional heatmap (split into two levels to aid display) for differentially expressed genes (DEGs; adjusted p-value <0.1 and absolute log 2 fold-change (FC)>1.5) in unmanipulated NK cells derived from Opt-Cs (n=18 samples) and Sub-Cs (n=14 samples) Selected genes of interest were highlighted. Hierarchical clustering at the sample level was applied; TPM=transcripts per million. FIG. 14B displays a box plot showing that Opt-Cs NK cells have higher Activation Scores relative to Sub-Cs NK cells. Activity of NK function signature (e.g., GZMA, PRF1, GZMB, and CD247) was estimated in each sample using ssGSEA implemented in the R package GSVA. Difference between Opt-Cs and Sub-Cs was computed using two tailed Student's t-test. FIGS. 14C-14E show enrichment plots for selected pathways identified to be differentially regulated using gene set enrichment analysis (GSEA) of NK cells from CBMCs of Opt-Cs relative to Sub-Cs. FIG. 14C shows how Sub-Cs cells displayed reduced enrichment scores for hallmarks of hypoxia signaling relative to Opt-Cs cells (p=0.0006). FIG. 14D shows how Sub-Cs cells displayed reduced enrichment scores for hallmarks of apoptosis signaling relative to Opt-Cs cells (p=0.0016). FIG. 14E shows how Sub-Cs cells displayed reduced enrichment scores for hallmarks of inflammatory response relative to Opt-Cs cells (p=0.0019).

FIGS. 15A-15D, shows how NK cells from optimal cords (“Opt-NK″) displayed an improved epigenetic state of activation and fitness when compared to NK cells derived from suboptimal cords (“Sub-NK”). FIG. 15A displays a volcano plot showing the difference in motif-based transcription factor (TF) levels between Sub-Cs (n=9 samples) and Opt-Cs (n=8 samples). X-axis denotes the logarithmic scale of fold-change (FC). Positive values represented TFs up-regulated in Opt-Cs (yellow) while negative values represent those upregulated in Sub-Cs (black). Motif sequences of differentially enriched TFs of interest were identified, for Sub-Cs these included at least Smad2/Smad3, CREB1, and/or FOSB/JUN; while for Opt-Cs these included at least TBX21, EOMES, and/or IRF2 as provided in the figure. P values were determined by Wilcox test, each symbol represents an individual donor, data are shown as mean±s.e.m. FIG. 15B displays ATAC-seq tracks for selected genes (PRF1 top, GZMA middle, and EOMES bottom). Each panel compared signal tracks for 8 Sub-Cs with 8 Opt-Cs. The higher peaks indicated more abundant reads of the gene. The right plot showed the comparison of gene-level accessibility score between NK cells from CBMCs of Sub-Cs vs. Opt-Cs. FIG. 15C depicts motif sequences of differentially enriched TFs of interest as described in FIG. 15A, for Sub-Cs these included at least Smad2/Smad3, CREB1, and/or FOSB/JUN (SEQ ID NOs: 1-3, respectively); while for Opt-Cs these included at least TBX21, EOMES, and/or IRF2 (SEQ ID NOs: 4-6, respectively). FIG. 15D depicts ATAC-seq tracks for selected genes (from top to bottom are EOMES, TBX21, GZMA, PRF1). The y-axis of each panel denoted the number of reads covering peak regions for Sub-Cs (n=8 samples; top, black) with Opt-Cs (n=8 samples; bottom, yellow). Higher peaks indicated more reads covering the regions. The right boxplot shows the comparison of gene-level accessibility score between NK cells from Sub-Cs vs. those from Opt-Cs. P values were determined by two-tailed Student's t test, each symbol represents an individual donor, data are shown as mean±s.e.m.

FIGS. 16A-16I, displays how CAR19/IL-15 NK cells derived from Opt-Cs demonstrated superior effector function compared to those derived from Sub-Cs. The clinical CAR19/IL-15 cord blood (CB)-NK cell products were utilized to characterize differences in the phenotypes/functions of expanded CAR-NK cells between Opt-Cs and Sub-Cs in FIGS. 16A-16D. For the experiments described in FIGS. 16E-16I, CAR19/IL-15 NK cells were generated from an independent cohort of CB units obtained from the MDACC bank. FIG. 16A shows tumor rechallenge assay results where clinical CAR19/IL-15 NK cell products derived from either Sub-Cs or Opt-Cs were rechallenged with Raji^{mCherry} at an effector-to-target (E: T) ratio of 5:1. Tumor cells (100,000 cells) were added every 2-3 days for two weeks and tumor cell killing was measured by the tumor cell index representing tumor cell counts with mCherry detection. The bar graph compares the area under curve (AUC) of tumor cell index between the two groups (n=3 donors per each group). FIG. 16B is a bar graph showing the polyfunctional strength index (PSI) of clinical CAR19/IL-15 NK-cell products derived from Opt-Cs (n=3 donors) vs. Sub-Cs (n=3 donors) following CD19 antigen stimulation. FIG. 16C shows oxygen consumption rate (OCR) as a surrogate for oxidative phosphorylation (OXPHOS) by mito stress test of clinical CAR19/IL-15 NK-cell products derived from Opt-Cs (n=3 donors) vs. Sub-Cs (n=3 donors) (left); bar graphs of basal respiration (middle), and maximal respiration (right); Oligo: Oligomycin, Rot/AA: Rotenone/Antimycin A. FIG. 16D is a bar graph of the glycolytic capacity measured by glyco stress test of clinical CAR19/IL-15 NK cell products derived from Opt-Cs (n=3 donors) vs. Sub-Cs (n=3 donors); ECAR: extracellular acidification rate. FIG. 16E Bar graph showing the percentage (%) of CAR expression on transduced NK cells derived from an independent cohort of Sub-Cs vs. Opt-Cs (n=5 donors per group). FIG. 16F Cumulative population doublings (PDs) of CAR19/IL-15 NK cells derived from Sub-Cs vs. Opt-Cs over 21 days of expansion (n=5 per each group).

FIG. 16G shows results of tumor rechallenge assays where CAR19/IL-15 NK cells derived from either Sub-Cs or Opt-Cs were rechallenged with Raji^{mCherry} at an E:T ratio of 2:1. Tumor cells (100,000 cells) were added every 2-3 days for two weeks and tumor cell killing was measured by the tumor cell index representing tumor cell counts with mCherry detection. The bar plots compare the AUC of tumor cell index between the two groups (n=5 donors per each group). FIG. 16H provides a bar graph showing the PSI of CAR19/IL-15 NK cells secreting different cytokines after CD19 antigen stimulation. FIG. 16I shows OCR as a surrogate for OXPHOS of CAR19/IL-15 NK cells derived from Opt-Cs (n=3 donors) vs. Sub-Cs (n=3 donors) by mito stress test (left); bar graphs of basal OCR (middle), and maximal OCR (right). P-values were determined by two-tailed Student's t test in FIGS. 16A, 16C, 16D, 16E, 16G, and 16I, or two-tailed one-way ANOVA in panels FIGS. 16B, 16F, and 16H. Each symbol represents an individual sample, data are shown as mean±s.e.m.

FIGS. 17A-17G, show how Opt-Cs derived NK cells (as described in FIGS. 12-16) displayed superior tumor control and engraftment in a lymphoma (Raji cells) mouse model relative to Sub-Cs derived NK cells. FIG. 17A displays a schematic of one of two experimental procedures, where mice were irradiated on day-1 and inoculated with 0.2×10⁵ Raji cells and 1×10⁷ CAR19/IL-15 NK cells derived from Opt-Cs or Sub-Cs respectively, 14 days after cell injection, blood and tissues were harvested for phenotypic analysis. A second independent experiment was performed using the same parameters, but animals were not sacrificed on day 14 and were instead imaged over time (n=5 mice per group). FIG. 17B displays the results of the second experiment described in FIG. 17A, where Bioluminescence imaging (BLI) photographs were taken on day 0, day 14, day 21, and day 28, the results showed that mice receiving Opt-Cs derived CAR19/IL15 NK cells had superior tumor cell control relative to mice that received tumor cells only, or to mice that received tumor cells and Sub-Cs derived CAR19/IL15 NK cells. FIG. 17C displays absolute CAR-NK cell counts in peripheral blood (PB) 10 days after CAR19/IL15 NK cell injection (p=0.0138; two-tailed two-way ANOVA). FIG. 17D the animals were sacrificed at day 14 after CAR19/IL-15 NK-cell injection. Shown are the absolute numbers of CAR19/IL15 NK cells (left) and Raji cells (right) in the hCD45+ cell populations as harvested from the bone marrow (BM). The results showed that Opt-Cs derived CAR19/IL15 NK cells engrafted at a significantly greater rate than Sub-Cs derived CAR19/IL15 NK cells (left panel; p=0.0126), and that while both Sub-Cs and Opt-Cs derived CAR19/IL15 NK cells significantly improved tumor cell control (right panel; P=0.0379), Opt-Cs derived CAR19/IL15 NK cells provided significantly improved tumor cell control relative to Sub-Cs derived CAR19/IL15 NK cells (right panel; p=0.0483) (log-rank test for each analysis). FIG. 17E depicts SPADE analysis showing the phenotype of live hCD45+CD56+CD3-CAR19/IL-15 NK cells derived from Sub-Cs (n=5 mice) or Opt-Cs (n=3 mice). BM samples were collected from Raji-engrafted mice 14 days after CAR19/IL-15 NK cell treatment. The CyTOF data were down-sampled to 10,000 cells per sample, pooled and divided into two categories: Sub-Cs vs. Opt-Cs. SPADE analysis revealed 6 main clusters (Cluster 1-6). Frequencies of each cluster are indicated in each condition, statistical analysis by two-tailed Student's t test. FIG. 17F depicts a heatmap representing the expression levels of NK cell markers within the main sub-clusters of clusters 1-6. The expression level for each marker is represented on a color scale ranging from the color blue (low) to the color red (high), statistical analysis by two-tailed Student's t test and two-tailed one-way ANOVA. FIG. 17G displays quantification of the results provided in FIG. 17B, left panel showed tumor burden in the mice was assessed by weekly bioluminescence imaging (BLI). Each line refers to the luciferase (Luc) radiance for each mouse (black: no treatment; grey: treated with CAR19/IL-15 NK cells from Sub-Cs; yellow: treated with CAR19/IL-15 NK cells from Opt-Cs; n=5 mice per group). FIG. 17H shows Kaplan-Meier survival curves of Raji mouse model animals after treatment with CAR19/IL-15 NK cells from Opt-Cs vs. Sub-Cs; data were pooled from two independent experiments (e.g., mice presented in the left panel and FIG. 17B, and a second independent experiment).

FIGS. 18A-18H, show how Opt-Cs derived NK cells (as described in FIGS. 12-17) displayed superior tumor control and engraftment in a CD70+ myeloma (MM.1S cells) mouse model relative to Sub-Cs derived NK cells. FIG. 18A displays a schematic of the experimental procedure, where mice were irradiated, inoculated with 5×10⁵ MM.1S cells on day-3, and sham injected or injected (IV) with a single infusion of 1×10⁷ CD70 targeted CAR and IL-15 expressing NK cells (“CAR70/IL15 NK”) derived from Opt-Cs or Sub-Cs, respectively. Animals were imaged weekly, and blood samples were taken at different time points (e.g., day 10 and day 20). FIG. 18B displays the results of the experiment described in FIG. 18A, where Bioluminescence imaging (BLI) photographs taken on days-3, 22, 26, 43, and 50 were displayed. The results showed that mice receiving Opt-Cs derived CD70 CAR-NK cells had superior tumor cell control relative to mice that received tumor cells only, or to mice that received tumor cells and Sub-Cs derived CD70 CAR-NK cells. FIG. 18C left panel displays quantification of the results provided in FIG. 18B, where animals receiving Opt-Cs derived CD70 CAR-NK cells displayed reduced radiance relative to animals receiving tumor cells alone, or tumor cells and Sub-Cs derived CD70 CAR-NK cells (p<0.0001; two-tailed two-way ANOVA). FIG. 18C right panel displays a Kaplan-Meier survival curve for the animals described in FIG. 18A, where animals receiving Opt-Cs derived CD70 CAR-NK cells displayed significantly longer survival than those receiving tumor cells alone, or tumor cells and Sub-Cs derived CD70 CAR-NK cells (p=0.0329; log-rank test). Animals receiving tumor cells and Sub-Cs derived CD70 CAR-NK cells displayed significantly longer survival than those receiving tumor cells alone (p=0.0020). FIG. 18D displays flow cytometry data with mean +/−s.e.m., with each symbol representing an individual mouse; the left panel displays the CAR-NK cell counts in blood samples taken at day 10 and day 20 post NK cell injection as described in FIG. 18A. CD70 CAR-NK cells derived from Opt-Cs displayed significantly higher counts per ml relative to CD70 CAR-NK cells derived from Sub-Cs (p=0.0001; two-tailed two-way ANOVA). FIG. 18D right panel displays the percentage of CD138+MM.1S cells found in the bone marrow of moribund animals described in FIG. 18A. BM samples were collected at the end-time points of sacrifice. CD70 CAR-NK cells derived from Opt-Cs displayed significantly lower percentages of MM.1S cells relative to CD70 CAR-NK cells derived from Sub-Cs (p=0.0389). Animals receiving Opt-Cs or Sub-Cs CD70 CAR-NK cells displayed significantly lower percentages of MM.1S cells relative to tumor only controls (p=0.0005; two-tailed one-way ANOVA). FIG. 18E shows a schematic diagram representing the timelines of the in vivo experiment using a mouse model of TROP2+ SKOV3 tumor . Mice received a single intraperitoneal (i.p.) injection of CAR-TROP2/IL-15 NK cells derived from Sub-Cs or Opt-Cs. FIG. 18F is a plot showing the tumor burden in the different mouse groups from the experiment described in FIG. 18E. Tumor burden was assessed by weekly BLI. Each line refers to an individual mouse (grey: tumor alone, no treatment; black: treated with CAR-TROP2/IL-15 NK cells from Sub-Cs; yellow: treated with CAR-TROP2/IL-15 NK cells from Opt-Cs; n=4 mice per group). FIG. 18G are Kaplan-Meier curves showing survival of mice from the experiment depicted in FIG. 18E (n=4 mice per group). FIG. 18H are BLI corresponding to the SKOV3 mouse model treated with CAR-TROP2/IL-15 NK cells described in FIG. 18E. The results showed that mice receiving Opt-Cs derived CAR-TROP2/IL-15 NK cells had superior tumor cell control relative to mice that received tumor cells only, or to mice that received tumor cells and Sub-Cs derived CAR-TROP2/IL-15 NK cells. P-values were determined by two-tailed two-way ANOVA, log-rank test, two-tailed Student's t test, or two-tailed one-way ANOVA. Data analyzed by flow cytometry are shown as mean±s.e.m. Each symbol represents an individual mouse sample.

FIGS. 19A-19C, show that expression of trogocytosis (TROG) antigen on CAR19+NK cells was predictive of patient outcomes after CAR19/IL-15 NK-cell treatment. FIG. 19A depicts a comparison of normalized CD19-MFI expression on B cells in the PB of patients before and 14 days after CAR19/IL-15 NK cell therapy. The data shown were for TROG^{low} group (n=23 patients; left) and TROG^{high} group (n=13 patients; right). FIGS. 19B and 19C are Kaplan-Meier curves showing (19B) overall survival (OS) and (19C) progression-free survival (PFS) for patients categorized as TROG^{low} ((“i”) n=23 patients) vs. TROG^{high} ((“ii”) n=13 patients); mo=months. Numbers above each line represent the number of patients at risk. Numbers in parentheses represent the probabilities of OS or PFS at a given time point. The shaded areas represent 95% confidence interval (CI) of survival probability. Trogocytosis data were not available for one patient. P-values were determined by two-tailed paired student t test in FIG. 19A, or by log-rank test in FIG. 19B-19C.

FIGS. 20A-20D, shows Odds Ratios and Hazard Ratios for patients who received optimal cords (Opt-Cs) vs. those who did not. The charts show posterior distributions of the, (20A) Log (Odds Ratio) of the probability of 30-day OR, (20B) Log (Odds Ratio) of the probability of 1-year CR, (20C) Log (Hazard Ratio) of 1-year PFS, and (20D) Log (Hazard Ratio) of 1-year OS, for patients who received optimal cords (Opt-Cs) vs. those who did not. Bayesian models allowed plotting of the distribution of the probabilities of possible clinical outcomes considering the influence of the variables of interest. Given the trial's data, the distribution of probabilities of the effect of receiving an Opt-C on the four clinical outcomes was computed; namely, (20A) 30-day OR, (20B) 1-year CR, (20C) 1-year PFS, and (20D) 1-year OS. Each distribution of probabilities was plotted, similar to a histogram. The area under the curve of a probability plot equaled 1 and was used to compute the probabilities of specific outcomes. For example, in panel 20A, the area to the right of Log (Odds Ratio) 2, which is about 0.50, was the probability that the Log (Odds Ratio) for the effect of Opt-Cs on the 30-day OR rate was larger than 2. This provided a basis for computing the probability of a beneficial effect (PBE) of the variable of interest on a particular outcome. PBE was defined as the area in the distribution of probability to the right of 0. This was the probability that the rate of a favorable outcome, either (20A) 30-day OR, or (20B) 1-year CR, was higher for patients who received Opt-Cs NK cells compared to patients who did not receive Opt-Cs NK cells. In general, a PBE near 1 implied that the variable was likely to have a beneficial effect, a PBE near 0 implied that the variable was unlikely to have a beneficial effect, and a PBE near 0.50 corresponded to no effect. FIGS. 20A and 20B showed that the PBE for Opt-Cs on either the rate of OR, or the rate of CR was extremely high, and numerical computations bore this out with respective PBE values of 0.991 and 0.981. For the outcomes of (20C) 1-year PFS and (20D) 1-year OS, PBE was represented graphically by the portion in the distribution of probabilities of Log (Hazard Ratio) to the left of 0, since it was better to have a smaller risk of death for OS, or of progression or death for PFS. In FIGS. 20C and 20D, the PBE was the probability that patients with Opt-Cs had (20C) a lower rate of progression or death or (20D) a lower rate of death. FIGS. 20C and 20D showed that the PBE for Opt-Cs on the rate of death, or the rate of progression, was extremely high, and the numerical computations bore this out with both PBE values equal to 1.00.

FIGS. 21A-21H, depict the characterization of expanded NK cells generated from Opt-Cs and suboptimal cords (Sub-Cs). FIGS. 21A-21C show characterization of clinical CAR19/IL-15 cord blood (CB)-NK cell products, where differences in CAR transduction (21A), proliferation (21B), and phenotypes (21C) of expanded CAR-NK cells from Opt-Cs and Sub-Cs were observed. FIGS. 21D-21H show characterization of expanded non-transduced NK cells (NT-NKs) from an independent cohort of CB units obtained from MD Anderson cell banks. FIG. 21A shows bar graphs with the percentages (%) of CAR expression in clinical CAR19/IL-15 NK-cell products derived from Sub-Cs (n=21 donors) or Opt-Cs (n=16 donors). FIG. 21B shows cumulative population doublings (PDs) for CAR-expressing NK cells derived from Sub-Cs (n=21 donors) or Opt-Cs (n=16 donors) expanded with K562-based feeder cells and IL-2 (200 U/mL) over 14 days. FIG. 21C displays SPADE analysis of CyTOF data showing the phenotype of the clinical CAR19/IL-15 NK-cell products (expanded for 14-21 days) from Sub-Cs (n=17 donors) or Opt-Cs (n=8 donors). The phenotypic signature of live hCD45+CD56+CD3 NK cells were evaluated by CyTOF, downsampled to 10,000 cells per sample, pooled and divided into two categories: Sub-Cs vs. Opt-Cs. Clustering by SPADE analysis revealed six main clusters (branches; Clusters 1-6). Frequencies of each cluster were indicated; size and color of nodes represent numbers of clustered cells. FIG. 21D shows cumulative PDs of NT-NK cells derived from an independent cohort of Sub-Cs vs. Opt-Cs (n=4 donors per each group) over 21 days of expansion. FIG. 21E are tumor rechallenge assay results where NT-NK cells expanded from the independent cohort of either Sub-Cs or Opt-Cs were rechallenged with Raji^{mCherry} at an E:T ratio of 5:1. Tumor cells (16,700 cells) were added every 2 days for 8 days and tumor cell killing was measured by the tumor cell index. The bar plots showed the area under curve (AUC) of tumor cell index representing the tumor cell count by mCherry detection (n=3 donors per each group). FIG. 21F are representative images from the serial tumor rechallenge assay from the experiment described in FIG. 21E. FIG. 21G is a bar graph showing the polyfunctionality strength index (PSI) of NT-NK cells secreting different cytokines after Fc-CD16 stimulation. FIG. 21H Oxygen consumption rate (OCR) was used as a surrogate for oxidative phosphorylation (OXPHOS), and OCR was measured on NT-NK cells that were expanded from a subset of Sub-Cs (n=3 donors) and Opt-Cs (n=3 donors) described in FIG. 21D. The results of the mito stress test (top), and the bar graphs of basal respiration (bottom left) and maximal respiration (bottom right) are presented; Oligo: Oligomycin, Rot/AA: Rotenone/Antimycin A. P-values were determined by two-tailed Student's t test in FIGS. 21A, 21E, and 21H, or two-tailed one-way ANOVA in FIGS. 21B, 21D, and 21G. Each symbol represented an individual donor, data were shown as mean±s.e.m.

FIGS. 22A-22C, show the immune composition of cord blood mononuclear cells (CBMCs) used for the generation of the clinical CAR19/IL-15 NK-cell products and validated in an independent CBMC cohort. Cryopreserved CBMCs that were stored in MD Anderson cord banks from each of the cords used to manufacture the clinical CAR19/IL-15 CB-NK-cell products were utilized to characterize differences in the composition of the immune subsets between Opt-Cs (n=9 donors) and Sub-Cs (n=4 donors) in the experiment described in FIG. 22A. For the experiment described in FIG. 22B, CBMCs from an independent cohort of CB units obtained from MD Anderson cord banks were analyzed, with Sub-Cs (n-6 donors) and Opt-Cs (n=6 donors) samples. FIGS. 22A and 22B depict bar plots showing the frequencies of NK cells (CD45+CD56+CD3−), CD8 T cells (CD45+CD56-CD3+CD8+), CD4 T cells (CD45+CD56-CD3+CD4+), gamma delta T (Tgd) cells (CD45+CD56-CD3+TCRgd+), B cells (CD45+CD56-CD3-CD19+CD20+), monocytes (Mo)+dendritic cells (DC) (CD45+CD56-CD3-CD14+CD11c+), and plasmacytoid DCs (pDC) (CD45+CD56-CD3-CD11c+CD123+). FIG. 22C shows the gating strategy for the immunophenotyping of NK cells from CBMCs by CyTOF as presented. Single live cells were determined based on natural-abundance Iridium selection, beads depletion and Live/Dead separation. Hematopoietic cells within the live population were then selected by gating on hCD45+. Differential expressions of CD56 and CD3 were used to discern NK cell populations (CD56+CD3−). P-values were determined by two-tailed Student's t test and shown as mean±s.e.m. Non-significant P-values were not added to the figures. Each symbol represents an individual sample.

FIGS. 23A-23F, show how unmanipulated NK cells from Opt-Cs and Sub-Cs were characterized by unique transcriptomic and epigenetic signatures. FIG. 23A is a PCA plot based on the top five thousand variably expressed genes, showing separation of Opt-Cs (n=18 samples) and Sub-Cs (n=14 samples). FIG. 23B are box plots showing the NK functional scores for NK cells from CBMCs of Sub-Cs (n=13 samples) vs. Opt-Cs (n=18 samples). FIG. 23C is a bar graph of pathway enrichment analysis. Significantly differentially regulated pathways were identified by GSEA (q<0.1). Positive values indicate upregulation in Opt-Cs and negative values indicate upregulation in Sub-Cs. FIG. 23D are enrichment plots for selected pathways identified to be differentially regulated using GSEA of NK cells from CBMCs of Opt-Cs relative to Sub-Cs (left are hallmarks of protein secretion; right are hallmarks of TNFα signaling via NK-κB). FIG. 23E a PCA plot based on the 128,972 variably accessible peaks, showing separation of Opt-Cs (n=8 samples) and Sub-Cs (n=9 samples). FIG. 23F is a heatmap showing regulon activity AUC scores of differentially active regulons (see Methods, adjusted p-value<0.01) between Opt-C (n=18 samples) and Sub-Cs (n=14 samples). The AUC scores were scaled and indicated by the color intensity. P-values were determined by two-tailed Student's t test in 23B, Wilcoxon test for 23E, two-tailed Student's t test with Bonferroni correction in 23F. Data are shown as median with range of minimum to maximum in 23B, and each symbol represents an individual donor .

FIGS. 24A-24D, show SPADE analysis of live hCD45+CD56+CD3-NK cells in the bone marrow (BM) of mice collected 14 days after CAR19/IL-15 NK-cell injection. The phenotypic signatures of all gated NK cells were evaluated by CyTOF, downsampled to 10,000 cells per sample, pooled and divided into two categories: Sub-Cs vs. Opt-Cs. SPADE analysis was used to visualize the phenotypic differences. The mean expression of the key NK cell markers EOMES (FIG. 24A), T-bet (FIG. 24B), GrB (FIG. 24C), and PFN (FIG. 24D) were shown for each sub-cluster. The dot plots showed the frequencies of the NK cells expressing the marker of interest (Sub-Cs; n=5 mice vs. Opt-Cs; n=3 mice).

FIGS. 25A-25C, show how the immunosuppressive properties of NRBCs and prolonged CB collection-to-cryopreservation impacted CAR-NK function. FIG. 25A Bar graphs showing levels of arginase-1 (measured by ELISA), TGF-β1, and TGF-β2 (assessed by Milliplex) released by NRBCs (500,000 cells/ml) purified from CB (n=5 CB donors). NRBCs were cultured for up to 72 hours and the maximum level for each analyte was plotted. Media alone was used as a negative control. FIG. 25B and FIG. 25C, five CB units with an NRBC count ≤8×10⁷ were each divided into two fractions (Fractions A and B) soon after collection. Fraction A was cryopreserved <12 hours from collection while fraction B was frozen 24-48 hours post-collection in the MD Anderson CB bank. Both fractions were then thawed at the same time and NK cells were isolated, expanded, and transduced with the CAR19/IL-15 retroviral vector following standard procedures in the MD Anderson GMP facility. IncuCyte® imaging system was utilized to assess the cytotoxicity of CAR19/IL-15 NK cells generated from matched Fractions A and B from the same CB unit (n=5 CB donors) against Raji^{mCherry} cells (at an ET ratio of 10:1 and adjusted for CAR transduction efficiency) in a tumor rechallenge assay. Tumor cells were added every 2-3 days for 9 days and tumor cell killing measured by the tumor cell index. FIG. 25B bar plots show the area under curve (AUC) of tumor cell index, representing the tumor cell count detected by mCherry. The AUCs for 4 tumor challenges, as a measure of the anti-tumor activity of CAR19/IL-15 from Fractions A and B, were displayed (n=5 donors per each fraction). FIG. 25C shows representative images from the serial tumor rechallenge assay from the experiment described in FIG. 25B. P-values were determined by two-tailed Student's t test. Each symbol represented an individual donor, data were shown as mean±s.e.m.

### DETAILED DESCRIPTION

Autologous anti-CD19 chimeric antigen receptor (CAR) T-cells have been reported to induce remissions in 57-71% of patients with chronic lymphocytic leukemia (CLL), 52-82% of patients with diffuse large B-cell lymphoma (DLBCL) and 78-92% of patients with low grade non-Hodgkin lymphoma (LG-NHL) ¹⁻⁴. Indeed, there are currently multiple FDA-approved autologous anti-CD 19 CAR T-cell products available for clinical use. However, CAR T-cells have recognized limitations including the cost of therapy and the time required to collect the T-cells and manufacture the product. Moreover, a proportion of patients treated with CAR T-cells develop toxicities such as cytokine release syndrome (CRS), neurotoxicity or hemophagocytic lymphohistiocytosis (HLH) which carry significant morbidity, requiring CAR-T cells to be administered by specialized teams further limiting access to these saving therapies ^{5,6}. Therefore, there is great interest to develop off-the-shelf cell therapies that are cost effective, safe, and potent.

Natural killer (NK) cells target cancer cells that downregulate HLA class-I molecules or express stress markers, thus playing a critical role in cancer immune-vigilance ⁷⁻⁹. These cells can be engineered to express a CAR and can be safely administered without the need for HLA-matching, thus, eliminating the need to produce the CAR product on an individual basis ^{10,11} Indeed, the inventors have previously reported on the phase I part of a phase I-II clinical trial where patients with CD19 expressing malignancies were treated with escalating doses of allogeneic cord blood (CB) derived NK cells that had been modified with a retroviral vector to express genes encoding for (i) anti-CD19 CAR, (ii) interleukin-15 (IL-15) to enhance the in vivo expansion and persistence of the transduced NK cells, and (iii) inducible caspase-9 (iC9) to trigger apoptosis of the CAR-NK cells in the event of unacceptable toxicity (which can be referred to herein as “CAR19/IL-15”) ¹². In that study, CAR-NK cells were safe, and responses were seen in the majority of patients at all dose levels. A subsequent study to investigate the safety and efficacy of this strategy in patients with CD19-expressing malignancies was conducted and reported on for the dose-escalation portion of the trial ¹³. While that study was designed to manufacture of each CAR-NK cell product from a different CB donor, the inventors found that from a single CB unit it was possible to manufacture hundreds of doses of CAR-NK cells ^{12.}

As inter-donor variability in immune effector cell function may dramatically impact the likelihood of response ¹⁴⁻¹⁸, the inventors determined that it was of paramount importance to define the selection criteria for CB units that were most likely to result in optimal NK cell products. Indeed, clinical responses to autologous CAR T-cell therapies have been associated with specific baseline T-cell characteristics, such as markers of T-cell fitness and exhaustion, indicating that T-cells from some patients with cancer may not be adequate for the manufacturing of a sufficiently-potent cell therapy product. As provided herein, the inventors report on the final results of the trial and identify CB characteristics that can be used to select the CB units most likely to induce a clinical response. Furthermore, the inventors investigated the underlying biological mechanisms for the observed heterogeneity in NK cell potency, and validated the discovered CB selection criteria for NK cell (e.g., CAR-NK cell) production using multiple pre-clinical tumor models and target antigens.

### I. Examples of Definitions

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or .” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more. The terms “about”, “substantially” and “approximately” mean, in general, the stated value plus or minus 5%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of′ is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “cord blood composition” or “cord blood unit” (“CBU”) as used herein refers to a volume of cord blood originally obtained from a placenta and/or in an attached umbilical cord after childbirth. The cord blood unit or cord blood composition may or may not be stored in a storage facility following its collection. In some cases, the cord blood unit or cord blood composition contains blood that is derived from a single individual, whereas in alternative cases the cord blood unit or cord blood composition is a mixture from multiple individuals.

The term “cryopreservation” as used herein refers to the process of cooling and storing cells at a temperature below the freezing point. In specific examples, the temperature for cryopreservation is at least as low as −80 °C. In some examples, CBUs are cryopreserved and kept in liquid nitrogen (e.g., approximately −196 °C) or in the gas phase of a tank with liquid nitrogen (e.g., approximately −140 °C.) The cryopreservation may or may not include addition of one or more cryoprotectants to the cells prior to freezing. Examples of cryoprotectants include Dimethyl Sulfoxide (DMSO), hetastarch, Dextran 40, or a combination thereof. In one specific example, one may utilize 6% hetastarch in 0.9% sodium chloride in 5 ml of 55% Dimethyl Sulfoxide/5% Dextran 40 in 0.9% sodium chloride.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “engineered” “or “engineering” as used herein refers to an entity that is generated by the hand of man (or the process of generating same), including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as synthetic antigen receptors and/or cytokines), in which case(s) the engineering is all performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

The term “heterologous” as used herein refers to being derived from a different cell type or a different species than the recipient. In specific cases, it refers to a gene or protein that is synthetic and/or not from an NK cell. The term also refers to synthetically derived genes or gene constructs. The term also refers to synthetically derived genes or gene constructs. For example, a cytokine may be considered heterologous with respect to a NK cell even if the cytokine is naturally produced by the NK cell because it was synthetically derived, such as by genetic recombination, including provided to the NK cell in a vector that harbors nucleic acid sequence that encodes the cytokine.

The term “immune cell” as used herein refers to a cell that is part of the immune system and helps the body fight infections and other diseases. Immune cells include natural killer cells, invariant NK cells, NK T cells, T cells of any kind (e.g., regulatory T cells, CD4.sup.+ T cells, CD8.sup.+ T cells, or gamma-delta T cells), B cells, monocytes, granulocytes, myeloid cells neutrophils, eosinophils, basophils, mast cells, monocytes, macrophages, dendritic cells, and/or stem cells (e.g., mesenchymal stem cells (MSCs) or induced pluripotent stem (iPSC) cells). Also provided herein are methods of producing and engineering the immune cells following selection of the appropriate cord blood unit, as well as methods of using and administering the cells for adoptive cell therapy, in which case the cells may be autologous or allogeneic with respect to the source of the cord blood and the recipient of the cells.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating, or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

The term “optionally” as used herein refers to an element, step, or parameter that may or may not be utilized in any method of the disclosure.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “viability” as used herein refers to the ability of a specific cell or plurality of cells to maintain a state of survival.

### II. Embodiments of the Methods

Embodiments of the disclosure include methods for identifying predictors for a response of immune cells, such as NK cells, derived from cord blood cells. In particular embodiments, cord blood units are tested for one or a variety of predictors that may produce immune cells better suited for adoptive cell therapy than cord blood units lacking in one or more of the predictors. Parameters being evaluated that can predict for an improved response of immune cells derived from cord blood cells in comparison to cells not so tested may comprise cell production, cell engineering, and/or cell activity processes. The parameters may regard the cord blood units themselves, or the parameters may regard any cells derived from the cord blood units, or manipulation or modification thereof. Such parameters include viability of cord blood units; red blood cell content of the cord blood units (pre- or post-processing); total mononuclear cell recovery from the cord blood units; time from birth to cryopreservation of the cord blood units; expansion of immune cells derived from thawed cord blood units (including at one or more ranges of time points); volumes of materials; gender, age and/or weight of the baby; race of one or more biological parents of the baby; age of the mother; one or more marker of the cells; engineering of immune cells derived from thawed cord blood units; cytotoxicity of immune cells derived from the thawed cord blood units; gestational age of a mother from which the cord blood is derived; cytotoxicity of immune cells derived from the thawed cord blood units (including cytotoxicity against cancer cells or cells infected with a pathogen); viability of cord blood units following thawing; and so forth. Methods described herein may include 1, 2, 3, 4, 5, 6, 7, 8, 9, 10, or more than 10 selection criteria described herein.

Embodiments of the disclosure include methods for selecting cryopreserved cord blood units for the manufacture of cells for adoptive cell therapy having a higher potency (such as by being measured using cytotoxicity assays and the proportion of patients who respond) for a specific purpose, including clinical applications, than cells not so selected. In specific embodiments, the methods are for selecting cryopreserved cord blood units for the manufacture of engineered immune cells with a higher potency for adoptive cell therapy than cells not so selected, including for the treatment of cancer, for example. In particular aspects, the methods are for selecting cryopreserved cord blood units for the manufacture of engineered natural killer cells with a higher potency for adoptive cell therapy than cells not so selected, including for the treatment of cancer of any kind, for example.

In particular embodiments, methods encompassed herein include those in which a risk is reduced of selecting cord blood units (which may be referred to as cord blood compositions) that would produce immune cells, such as NK cells, that are ineffective or inferior at being engineered, expanded, and/or at being utilized clinically, such as for the treatment of cancer. In specific embodiments, the methods reduce the risk of selecting cord blood units that would produce immune cells lacking high potency, such as for cancer therapy as adoptive cell therapy. In specific cases, the methods encompassed herein increase the likelihood of producing adoptive NK cell therapy that is efficacious against one or more types of cancer. In certain embodiments, methods provided herein select for immune cells that display distinct phenotypic, transcriptional, and/or epigenetic signatures.

The methods of the disclosure select for cells for adoptive cell therapy that are quantitatively and/or qualitatively better at cell therapy than cells not so selected. Qualitatively, the cells may be more cytotoxic, may expand to a greater capacity, may have greater persistence, may be more conducive to engineering, may have a greater proportion of patients who respond, or a combination thereof. Quantitatively, the selected cord blood units from the method may have cell viability levels that are at least about or exactly 10%, 15%, 20%, 25%, 30%, 35%, 40%, 45%, 50%, 55%, 65%, 70%, 75%, 80%, 85%, 90%, 95%, 96%, 97%, 98%, 99%, or greater compared to cord blood units selected without knowledge of one or more of the selection parameters encompassed herein. The selected cord blood units from the method may have cell viability levels that are at least at least 10-fold, 20-fold, 30-fold, 40-fold, 50-fold, 60-fold, 70-fold, 80-fold, 90-fold, 10⁰-fold, 250-fold, 500-fold, 750-fold, 10⁰⁰-fold, or greater compared to cord blood units selected without knowledge of one or more of the selection parameters encompassed herein. The selected cord blood units from the method may produce total mononuclear cell recovery that is greater than at least 10%, 15%, 20%, 25%, 30%, 35%, 40%, 45%, 50%, 55%, 65%, 70%, 75%, 80%, 85%, 90%, 95% or more than cord blood units selected without knowledge of one or more of the selection parameters encompassed herein. The selected cord blood units from the method may produce total mononuclear cell recovery that is greater than at least 10-fold, 20-fold, 30-fold, 40-fold, 50-fold, 60-fold, 70-fold, 80-fold, 90-fold, 10⁰-fold, 250-fold, 500-fold, 750-fold, 10⁰⁰-fold, or greater or more than cord blood units selected without knowledge of one or more of the selection parameters encompassed herein. The selected cord blood units from the method may have a nucleated red blood cell content that is at least 1×10³, 1×10⁴, 1×10⁵, 1×10⁶, 1×10⁷, 5×10⁷, 6×10⁷, 7×10⁷, 8×10⁷, 9×10⁷, or lower than cord blood units selected without knowledge of one or more of the selection parameters encompassed herein. The selected cord blood units from the method may have a nucleated red blood cell content that is at least 10%, 20%, 30%, 40%, 50%, 60%, 70%, 80%, 90%, or lower than cord blood units selected without knowledge of one or more of the selection parameters encompassed herein.

In certain embodiments, the weight of the baby at the time of collection of cord blood tissue may be considered in methods of the disclosure, whether or not in utero or ex utero. In specific embodiments, the weight of the baby is greater than 3650 grams, such as greater than 3650, 3700, 3750, 3800, 3850, 3900, 3950, 4000, 4050, 4100, 4150, 4200, 4250, 4500 grams, and so forth. In specific embodiments, this is measured prior to cryopreservation and/or use.

In specific embodiments, the race of one or more biological parents of the baby is Caucasian. In some cases, both biological parents of the baby are Caucasian, in some cases the biological mother is Caucasian, and in some cases the biological father is Caucasian.

In specific embodiments, the timing of collection of the cord blood from the baby is a factor in the method. In specific embodiments, the cord blood is obtained from the cord of the baby in utero. The collection step may be by any suitable method, and the party obtaining the cord blood may or may not be the party that manipulates, stores, and/or analyzes the cord blood for one or more parameters. In specific embodiments, upon collection or soon thereafter the cord blood is combined with one or more anticoagulants and the volume of the anticoagulant may or may not be a standard amount. In specific cases, the preprocess volume is the volume of cord blood collected plus anticoagulant, and in certain cases the preprocess volume is the volume of cord blood collected plus anticoagulant of a specific volume, such as 35 mL or about 35 mL. In particular embodiments, the volume of the extracted cord blood is no greater than about or exactly 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL, or less, in volume. In some cases, the volume of the anticoagulant is or is about 25, 26, 27, 28, 29, 30, 31, 32, 33, 34, 35, 36, 37, 38, 39, 40, 41, 42, 43, 44, 45, 46, 47, 48, 49, or 50 mL or more. In specific cases, the volume of the anticoagulant is or is about 35 mL. The anticoagulant may be of any kind, including at least CPD (and may be CDP-A (CDP+adenosine); citrate-phosphate-double dextrose (CP2D); acid citrate dextrose (ACD); Heparin, etc.). In particular embodiments, cells in the collected cord blood may express one or more particular markers. In specific cases, cells in the collected cord blood may express CD34. In certain embodiments, a particular percentage of cells express any marker, including CD34. In certain cases, >0.4% cells in the collected blood express CD34. In certain cases, >0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In certain cases, at least 0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In specific embodiments, this is measured prior to cryopreservation and/or use.

The selected cord blood cells from the method may produce immune cells that have cytotoxicity levels that are at least 10%, 15%, 20%, 25%, 30%, 35%, 40%, 45%, 50%, 55%, 65%, 70%, 75%, 80%, 85%, 90%, 95%, 96%, 97%, 98%, 99%, 100%, or greater than 100% when compared to immune cells produced from cord blood cells selected without knowledge of one or more of the selection parameters encompassed herein. In some cases, the immune cells produced from the selected cord blood cells may have cytotoxicity levels that are at least 10-fold, 20-fold, 30-fold, 40-fold, 50-fold, 60-fold, 70-fold, 80-fold, 90-fold, 10⁰-fold, 250-fold, 500-fold, 750-fold, 10⁰⁰-fold, or greater compared to immune cells selected from cord blood cells without knowledge of one or more of the selection parameters encompassed herein.

Particular aspects of the disclosure select for one or more product characteristics of cord blood units prior to freezing of any kind, and in some aspects there are one or more product characteristics selected for following thawing of the frozen cord blood units. Such action(s) allows for selecting cord blood units that are best suited (among a collection of cord blood units from which to choose) to produce cell products, including cell products for adoptive cell therapy. The characteristics of cord blood units post-thaw may or may not be directly related to production of the cell product. That is, in some cases, the production of cell therapy by engineering of the cells derived from the cord blood units is enhanced by selecting the appropriate cord blood units, and in additional or alternative cases, the activity of cell therapy following engineering of cells derived from the cord blood units is enhanced by selecting the appropriate cord blood units (e.g., activity such as cytoxicity, persistence in vivo, and so forth).

Embodiments of the disclosure include methods in which one or more parameters are characterized for one or more cord blood units from one or more storage banks of any kind of cord blood units. In specific cases, following characterization of the one or more cord blood units, one or more particular cord blood units may be rejected as being unsuitable to provide for optimal responses (e.g., activity upon therapeutic administration). In additional cases, one or more particular cord blood units may be determined to be suitable for enhanced activities, such as upon therapeutic administration. In some situations when more than one cord blood unit is determined by methods of the disclosure to be worthy of selection, they may or may not be combined prior to thawing or subsequent to thawing. Immune cells produced from selected cord blood units may be combined following derivation from the cord blood units.

In specific embodiments, the disclosure provides a novel set of criteria to identify cord blood units for the manufacture of NK cell therapy products with the highest potency for treatment of a disease. In specific embodiments, the disclosure provides a novel set of criteria to identify cord blood units for the manufacture of NK cell therapy products with the highest potency for the treatment of cancer. NK cells generated from these highly potent cord blood units are most likely to result in an optimal response in cancer patients. As such, the methods of the disclosure are used to select cord blood units with the highest potency as a material source for the manufacture of NK cell therapy products and to avoid the selection of cord blood units and/or the generation of NK cells unlikely to induce a clinical response or likely to induce an ineffective clinical response. In particular embodiments, high potency NK cells produced from cord blood units selected by methods of the disclosure have the highest probability of inducing remissions, overall survival (OS), partial response (PR), complete response (CR), and/or progression free survival (PFS) in patients with cancer following adoptive infusion. In specific cases, high potency NK cells produced from cord blood units selected by methods of the disclosure have a greater probability of inducing remissions in patients with cancer following adoptive infusion than NK cells produced from cord blood units that lack the disclosed beneficial characteristics.

Embodiments of the disclosure include methods of selecting a cord blood composition, comprising the steps of identifying a cord blood composition that, prior to cryopreservation, is determined to have one or more of the following: (a) optionally cord blood cell viability greater than or equal to, exactly or about 98%, 98.5%, or 99%; (b) optionally total mononuclear cell (TNC) recovery is greater than or equal to, exactly or about 76.3%; (c) optionally nucleated red blood cell (NRBC) content is less than or equal to, exactly or about 9.4×10⁷ (pre-processing), less than or equal to, and/or exactly or about 8.0×10⁷ (post-processing), or less than or equal to, exactly or about 4% of total TNC (post-processing); (d) optionally weight of the baby from which the cord blood is derived is greater than or equal to, exactly or about 3650 grams; (e) optionally race of the biological mother and/or biological father of the baby from which the cord blood is derived is Caucasian; (f) optionally gestational age of the baby from which the cord blood is derived is less than or equal to, exactly or about 38 weeks; (g) optionally intra utero collection of the cord blood (although extra utero or a combination of intra utero and extra utero may be used in any method of the disclosure); (h) optionally a biologically male baby from which the cord blood is derived; (i) optionally a pre-process volume (volume of the cord blood collected plus anticoagulant (35 ml CPD))≤120 mL; (j) optionally, cells of the extracted cord blood are >0.245% CD34+; (k) optionally measuring cytotoxicity of immune cells derived from the cord blood composition following thawing; (l) optionally measuring expansion of the cells in culture; (m) optionally mothers age is less than or equal to, exactly or about 32 years; and (n) optionally time from birth to cryopreservation is less than or equal to, exactly or about 24 hours. In some cases, the cord blood composition prior to cryopreservation is determined to have at least the characteristics of (a), (b), (c), (d), (e), (f), (g), (h), (i), (j), (k), (l), (m), and/or (n). In some cases, the cord blood composition prior to cryopreservation is determined to have at least the characteristics of (c) and (n). In some cases, the cord blood composition prior to cryopreservation is determined to have at least the characteristics of (c) and (n), and at least 1, 2, 3, or more than 3 additional characteristics. In some cases, the cord blood composition prior to cryopreservation is determined to have 1, 2, 3, or all of the characteristics of (a), (c), (d), (e), and (n), and they may be in any combination. In some cases, the cord blood composition prior to cryopreservation is determined to have (a), (c), (d), (e) and (n). In some cases, the cord blood composition prior to cryopreservation is determined to have 1, 2, 3, or all of the characteristics of a), (c), (d), (e) and (n) optionally in addition to one or more of any of the other characteristics.

A. Time from Birth to Cryopreservation

Embodiments of the disclosure include methods in which the time from birth to cryopreservation of CBUs is measured, and the measurement provides information whether or not the cord blood unit is suitable, such as suitable for selection for derivation of immune cells for adoptive cell therapy. In some embodiments, the time from birth to CBU cryopreservation is less than or equal to, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any range derivable therein. In some embodiments, the time from birth to CBU cryopreservation is less than or equal to, exactly or about 24 hours. In some embodiments, the time from birth to CBU cryopreservation is less than or equal to, exactly or about 23 hours. In some embodiments, the time from birth to CBU cryopreservation is less than or equal to, exactly or about 22 hours. In some embodiments, the time from birth to CBU cryopreservation is less than or equal to, exactly or about 21 hours. In some embodiments, the time from birth to CBU cryopreservation is less than or equal to, exactly or about 20 hours. In some embodiments, CBUs are characterized as non-desirable if the time from birth to CBU cryopreservation is greater than about 24 hours. In some embodiments, CBUs are characterized as non-desirable if the time from birth to CBU cryopreservation is greater than or equal to, exactly or about 25 hours. In some embodiments, CBUs are characterized as non-desirable if the time from birth to CBU cryopreservation is greater than or equal to, exactly or about 26 hours. In some embodiments, CBUs are characterized as non-desirable if the time from birth to CBU cryopreservation is greater than or equal to, exactly or about 27 hours. In some embodiments, CBUs are characterized as non-desirable if the time from birth to CBU cryopreservation is greater than or equal to, exactly or about 28 hours.

In some embodiments time from birth to cryopreservation comprises the time between CBU collection and the time a controlled rate freezing process is initiated. In some embodiments, the time between CBU collection and delivery of the baby is less than or equal to, exactly or about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 minutes. In some embodiments, the time between CBU collection and delivery of the baby is less than or equal to, exactly or about 5 minutes. In some embodiments, the time between CBU collection and delivery of the baby is less than or equal to, exactly or about 4 minutes. In some embodiments, the time between CBU collection and delivery of the baby is less than or equal to, exactly or about 3 minutes. In some embodiments, the time between CBU collection and delivery of the baby is less than or equal to, exactly or about 2 minutes. In some embodiments, the time between CBU collection and delivery of the baby is less than or equal to, exactly or about 1 minute.

### B. Measurement of Cell Viability

Embodiments of the disclosure include methods in which the viability of cells in cord blood units is measured, and the measurement provides information whether or not the cord blood unit is suitable, such as suitable for selection for derivation of immune cells for adoptive cell therapy. The cord blood cells being tested for viability may be a mixture of cells in the cord blood, such as mononuclear, stem cells (e.g., hematopoietic or mesenchymal), white cells, immune system cells (monocytes, macrophages, neutrophils, basophils, eosinophils, megakaryocytes, dendritic cells, T cells (including T helper and cytotoxic), B cells, NK cells), and so forth. The viability of cells in the cord blood can be observed through one or more physical properties of the cells and/or one or more activities of the cells. In some embodiments, the viability measurement of the cells is not total white blood cell (WBC) viability. In some embodiments, the viability measurement of the cells does not comprise, consist essentially of, or consist of measurement of culture forming units (CFUs). In some embodiments, In some embodiments, the viability measurement of the cells does not comprise, consist essentially of, or consist of measurement of Granulocyte Macrophage CFUs (CFU-GM), Granulocyte, Erythrocyte, Macrophage and Megakaryocyte CFUs (CFU-GEMM), and/or Burst Forming Unit Erythroid CFUs (BFU-E).

Although the viability may be determined by any suitable method(s), in specific cases the measurements are performed by flow cytometry, tetrazolium reduction assay, resazurin reduction assay, protease viability marker assay, ATP Assay, sodium-potassium ratio, lactate dehydrogenase assay, neutral red uptake, propidium iodide, TUNEL (terminal deoxynucleotidyl transferase-mediated dUTP nick end labeling) assay, formazan-based assay, Evans blue, Trypan blue, ethidium homodimer assay, or a combination thereof.

Cell viability for cord blood cells may be measured prior to cryopreservation and/or subsequent to cryopreservation. In some embodiments, cord blood cell viability for a desired CBU is greater than or equal to, exactly or about 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9, or 100%. In some embodiments, cord blood cell viability for a desired CBU is greater than 98.5%. In some embodiments, cord blood cell viability for a desired CBU is greater than 98.4%. In some embodiments, cord blood cell viability for a desired CBU is greater than 98.6%. In some embodiments, CBUs are characterized as non-desirable if the cord blood cell viability is equal to or less than, exactly or about 98.5%. In some embodiments, CBUs are characterized as non-desirable if the cord blood cell viability is equal to or less than, exactly or about 98.4%. In some embodiments, CBUs are characterized as non-desirable if the cord blood cell viability is equal to or less than, exactly or about 98.3%. In some embodiments, CBUs are characterized as non-desirable if the cord blood cell viability is equal to or less than, exactly or about 98.2%. In some embodiments, CBUs are characterized as non-desirable if the cord blood cell viability is equal to or less than, exactly or about 98.1%. In some embodiments, CBUs are characterized as non-desirable if the cord blood cell viability is equal to or less than, exactly or about 98.0%.

In cases wherein viability is measured in addition to one or more other characteristics, such as total nuclear cell recovery and measurement of nucleated red blood cell content, the cell viability may or may not be prior to one or more other measurements. In specific cases, viability is measured prior to TNC recovery and NRBC measurement or is measured subsequent to TNC recovery and NRBC measurement. In some cases, viability is measured after TNC but before NRBC or is measured after NRBC but before TNC recovery.

### C. Measurement of Total Nuclear Cell Recovery

In particular embodiments, the total nuclear cell (TNC) recovery is measured in which nucleated cells are measured following cord blood processing. The TNC recovery measures nucleated cells that are both live and dead. This step may or may not be optional.

Any suitable assay for measurement of TNC may be utilized, but in specific embodiments, the TNC recovery assay includes flow cytometry; Trypan blue; 3% Acetic Acid with Methylene Blue; hematology analyzer analysis; or a combination thereof. The TNC recovery assay may or may not be automated, in specific cases.

In one embodiment, TNC recovery is greater than or equal to, exactly or about 76.3, 76.4, 76.5, 76.6, 76.7, 76.8, 76.9, 77, 78, 79, 80, 81, 82, 83, 84, 85, 86, 87, 88, 89, 90, 91, 92, 93, 94, 95, 96, 97, 98, or 99%.

In particular embodiments, TNC recovery of cord blood units is measured prior to cryopreservation.

In cases wherein TNC recovery is measured in addition to one or more other characteristics, such as cell viability, time from birth to cryopreservation, and/or measurement of NRBC content, etc., the TNC recovery may or may not be prior to one or more other measurements. In specific cases, TNC recovery is measured prior to cell viability and NRBC measurement or is measured subsequent to cell viability and NRBC measurement. In some cases, TNC recovery is measured after cell viability but before NRBC or is measured after NRBC but before cell viability. In some cases, TNC recovery is utilized to determine relative NRBC content.

### D. Measurement of Nucleated Red Blood Count

In particular embodiments, cord blood units are selected based on the measurement of nucleated red blood cell (NRBC) content. The measurement may be manual or automated. In particular embodiments, cord blood units with lower NRBC content are more effective at producing efficacious immune cells than cord blood units with higher NRBC content. The level of NRBC in cord blood units can determine the response rate of individuals treated with immune cells, such as NK cells, derived from the particular cord blood unit. The NRBC content may be measured by density centrifugation, such as on a Sepax® device. The NRBC content may be measured as a total content post-processing (post-reduction), total content pre-processing, and/or as a percentage of TNC post-processing. In some embodiments, NRBC content is measured at one or more different moments during the process of CBU processing and/or cryopreservation. In some embodiments, NRBC content is measured as an absolute number. In some embodiments, NRBC content is measured as a percentage of TNCs. In some embodiments, NRBC content measurements at various time points during the process of CBU processing and/or cryopreservation are correlated. In some embodiments, one or more NRBC content measurement can be utilized. In some embodiments, post-reduction absolute number NRBC content is utilized. In some embodiments, post-reduction absolute number NRBC content is more predictive than pre-reduction absolute number and/or post-reduction percentage of TNC.

In specific embodiments, the NRBC content is less than or equal to, exactly or about 10.9×10⁷, 10.8×10⁷, 10.7×10⁷, 10.6×10⁷, 10.5×10⁷, 10.4×10⁷, 10.3×10⁷, 10.2×10⁷, 10.1×10⁷, 10.0×10⁷, 9.9×10⁷, 9.8×10⁷, 9.7×10⁷, 9.6×10⁷, 9.5×10⁷, 9.4×10⁷, 9.3×10⁷, 9.2×10⁷, 9.1×10⁷, 9.0×10⁷, 8.9×10⁷, 8.8×10⁷, 8.7×10⁷, 8.6×10⁷, 8.5×10⁷, 8.4×10⁷, 8.3 ×10⁷, 8.2×10⁷, 8.1×10⁷, 8.0×10⁷, 7.9×10⁷, 7.8×10⁷, 7.7×10⁷, 7.6×10⁷, 7.5×10⁷, 7.0×10⁷, 6.0×10⁷, 5.0×10⁷, 4.0×10⁷, 3.0×10⁷, 2.0×10⁷, 1.0×10⁷, 9.0×10⁶, 8.0×10⁶, 7.0×10⁶, 6.0×10⁶, 5.0×10⁶, 4.0×10⁶, 3.0×10⁶, 2.0×10⁶, 1.0×10⁶, 9.0×10⁵, 8.0×10⁵, 7.0×10⁵, 6.0×10⁵, 5.0×10⁵, 4.0×10⁵, 3.0×10⁵, 2.0×10⁵, 1.0×10⁵, 9.0×10⁴, 8.0×10⁴, 7.0×10⁴, 6.0×10⁴, 5.0×10⁴, 4.0×10⁴, 3.0×10⁴, 2.0×10⁴, 1.0×10⁴, 9.0×10³, 8.0×10³, 7.0×10³, 6.0×10³, 5.0×10³, 4.0×10³, 3.0×10³, 2.0×10³, 1.0×10³, 9.0×10², 8.0×10², 7.0×10², 6.0×10², 5.0×10², 4.0×10², 3.0×10², 2.0×10², 1.0×10², and so forth, including to an undetectable level.

In particular embodiments, NRBC content is measured prior to cryopreservation. In particular embodiments, NRBC content is measured pre-processing. In particular embodiments, NRBC content is measured post-processing. In particular embodiments, NRBC content is measured post-processing as a percentage of TNC. In particular embodiments, NRBC content is measured as total NRBC content per CBU.

In specific embodiments, where NRBC content is measured pre-processing, the NRBC content is less than or equal to, exactly or about 10.9×10⁷, 10.8×10⁷, 10.7×10⁷, 10.6×10⁷, 10.5×10⁷, 10.4×10⁷, 10.3×10⁷, 10.2×10⁷, 10.1×10⁷, 10.0×10⁷, 9.9×10⁷, 9.8×10⁷, 9.7×10⁷, 9.6×10⁷, 9.5×10⁷, 9.4×10⁷, 9.3×10⁷, 9.2×10⁷, 9.1×10⁷, 9.0×10⁷, 8.9×10⁷, 8.8×10⁷, 8.7×10⁷, 8.6×10⁷, 8.5×10⁷, 8.4×10⁷, 8.3×10⁷, 8.2×10⁷, 8.1×10⁷, 8.0×10⁷, or lower. In specific embodiments, where NRBC content is measured pre-processing, the NRBC content is less than or equal to, exactly or about 9.4×10⁷. In specific embodiments, where NRBC content is measured pre-processing, the NRBC content is less than or equal to, exactly or about 9.6×10⁷. In specific embodiments, where NRBC content is measured pre-processing, the NRBC content is less than or equal to, exactly or about 9.2×10⁷. In some embodiments, where NRBC content is measured pre-processing, CBUs are characterized as non-desirable if the NRBC content is greater than 9.4×10⁷. In some embodiments, where NRBC content is measured pre-processing, CBUs are characterized as non-desirable if the NRBC content is greater than 9.5×10⁷. In some embodiments, where NRBC content is measured pre-processing, CBUs are characterized as non-desirable if the NRBC content is greater than 9.6×10⁷. In some embodiments, where NRBC content is measured pre-processing, CBUs are characterized as non-desirable if the NRBC content is greater than 9.7×10⁷. In some embodiments, where NRBC content is measured pre-processing, CBUs are characterized as non-desirable if the NRBC content is greater than 9.8×10⁷. In some embodiments, where NRBC content is measured pre-processing, CBUs are characterized as non-desirable if the NRBC content is greater than 9.9×10^{7.}

In specific embodiments, where NRBC content is measured post-processing, the NRBC content is less than or equal to, exactly or about 8.9×10⁷, 8.8×10⁷, 8.7×10⁷, 8.6×10⁷, 8.5×10⁷, 8.4×10⁷, 8.3×10⁷, 8.2×10⁷, 8.1×10⁷, 8.0×10⁷, 7.9×10⁷, 7.8×10⁷, 7.7×10⁷, 7.6×10⁷, 7.5×10⁷, 7.0×10⁷, or lower. In specific embodiments, where NRBC content is measured post-processing, the NRBC content is less than or equal to, exactly or about 8.0×10⁷. In specific embodiments, where NRBC content is measured post-processing, the NRBC content is less than or equal to, exactly or about 8.2×10⁷. In specific embodiments, where NRBC content is measured post-processing, the NRBC content is less than or equal to, exactly or about 7.8×10⁷. In some embodiments, where NRBC content is measured post-processing, CBUs are characterized as non-desirable if the NRBC content is greater than 8.0×10⁷. In some embodiments, where NRBC content is measured post-processing, CBUs are characterized as non-desirable if the NRBC content is greater than 8.1×10⁷. In some embodiments, where NRBC content is measured post-processing, CBUs are characterized as non-desirable if the NRBC content is greater than 8.2×10⁷. In some embodiments, where NRBC content is measured post-processing, CBUs are characterized as non-desirable if the NRBC content is greater than 8.3×10⁷. In some embodiments, where NRBC content is measured post-processing, CBUs are characterized as non-desirable if the NRBC content is greater than 8.4×10⁷. In some embodiments, where NRBC content is measured post-processing, CBUs are characterized as non-desirable if the NRBC content is greater than 8.5×10^{7.}

In specific embodiments, where NRBC content is measured post-processing as a percentage of TNC, the NRBC content is less than or equal 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, or 3.0%, or less than 3.0% of TNC. In some embodiments, where NRBC content is measured post-processing as a percentage of TNC, the NRBC content is less than or equal to, exactly or about 4.0%. In some embodiments, where NRBC content is measured post-processing as a percentage of TNC, the NRBC content is less than or equal to, exactly or about 4.1%. In some embodiments, where NRBC content is measured post-processing as a percentage of TNC, the NRBC content is less than or equal to, exactly or about 3.9%. In some embodiments, where NRBC content is measured post-processing as a percentage of TNC, CBUs are characterized as non-desirable if the NRBC content is greater than 4%. In some embodiments, where NRBC content is measured post-processing as a percentage of TNC, CBUs are characterized as non-desirable if the NRBC content is greater than 4.1%. In some embodiments, where NRBC content is measured post-processing as a percentage of TNC, CBUs are characterized as non-desirable if the NRBC content is greater than 4.2%. In some embodiments, where NRBC content is measured post-processing as a percentage of TNC, CBUs are characterized as non-desirable if the NRBC content is greater than 4.3%. In some embodiments, where NRBC content is measured post-processing as a percentage of TNC, CBUs are characterized as non-desirable if the NRBC content is greater than 4.4%. In some embodiments, where NRBC content is measured post-processing as a percentage of TNC, CBUs are characterized as non-desirable if the NRBC content is greater than 4.5%.

In cases wherein NRBC content is measured in addition to one or more other characteristics, such as total nuclear cell recovery, time from birth to cryopreservation, and/or measurement of NRBC content, etc., the NRBC may or may not be prior to one or more other measurements. In specific cases, NRBC content is measured prior to TNC recovery and cell viability or is measured subsequent to TNC recovery and cell viability. In some cases, NRBC content is measured after TNC but before cell viability or is measured after cell viability but before TNC recovery.

### E. Weight of the Baby

In some embodiments, the weight of the baby from which the cord blood is derived is utilized as a parameter in any method encompassed by the disclosure. The weight of the baby may be taken just prior to collection of the cord blood, such as within days or hours or minutes, for example. In some cases, the weight of the baby may be determined in utero by using prenatal ultrasound. In some cases, the weight of the baby is determined ex utero, such as on a standard scale. The party measuring the weight of the baby may or may not be the party that manipulates, stores, and/or analyzes the cord blood for one or more parameters. This step may occur before and/or after any other step prior to cryopreservation. In specific embodiments, the weight of the baby is greater than a certain amount, and this may or may not generally correlated with gestational age. In some cases, the weight of the baby is greater than about 3000, 3050, 3100, 3150, 3200, 3250, 3300, 3350, 3400, 3450, 3500, 3550, 3600, 3650, 3700, 3750, 3800, or 3850 grams, or greater. In some cases, the weight of the baby is greater than about 3650 grams, such as greater than 3650, 3700, 3750, 3800, 3850, 3900, 3950, 4000, 4050, 4100, 4150, 4200, 4250, 4500 grams, and so forth. In some embodiments, CBUs are characterized as non-desirable if the weight of the baby is less than about 3650. In some embodiments, CBUs are characterized as non-desirable if the weight of the baby is less than about 3600. In some embodiments, CBUs are characterized as non-desirable if the weight of the baby is less than about 3550. In some embodiments, CBUs are characterized as non-desirable if the weight of the baby is less than about 3500. In specific embodiments, this is measured prior to cryopreservation and/or use.

### F. Race of the Biological Parents

In specific embodiments, the race of one or more of the biological parents is Caucasian. In some cases the biological mother is Caucasian and the biological father is Caucasian. In some cases, the biological mother is Caucasian but the biological father is not Caucasian. In some cases, the biological father is Caucasian but the biological mother is not Caucasian.

### G. Collection Parameters for Cord Blood

In some embodiments, the cord blood is obtained by standard methods in the art, such as via a needle from the umbilical vein after the baby is born. For ex utero extraction, this is done after the placenta has been expelled, and the cord blood is inserted into a sterile collection bag that comprises an anticoagulant, or an anticoagulant may be added. For in utero extraction, this is done through the umbilical vein while the placenta is still inside the mother, following which it is inserted into a sterile collection bag that comprises an anticoagulant, or an anticoagulant may be added. In some cases, cord blood from the same baby is combined from in utero and ex utero extractions. In a specific embodiment, in utero extraction is a method of choice over ex utero extraction.

In particular embodiments, the volume of extracted cord blood is considered in the methods of the disclosure. For example, the volume of the combination of both cord blood and anticoagulant as a pre-processing composition is considered in methods of the disclosure. In specific cases, the volume of the combination of cord blood and anticoagulant is ≤120 mL. As one example, when the volume of anticoagulant is about 35 mL, the volume of the cord blood is less than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mL in volume.

### H. Cord Blood Cell Markers

In specific embodiments, at least some of cells of any type in the cord blood may collectively express one or more particular markers. In specific embodiments, a particular percentage of cells of the cord blood express CD34. Examples of cord blood cell types include stem cells, progenitor cells, red blood cells, white blood cells, B lymphocytes, T lymphocytes, NK cells, monocytes, and platelets. In some cases, greater than 0.245% cells in the collected cord blood express CD34. In certain cases, greater than or equal to, exactly or about 0.01, 0.1, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In certain cases, at least 0.01, 0.1, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.4, 0.5, 0.6, 0.7, 0.8, 0.9, 1.0, 1.5, 2, 3, 4, 5, 6, 7, 8, 9, 10, 15, 20, 25, 30, 40, 50, 60, 70, 75, 80, 85, 90, or 95% cells in the collected blood express CD34. In some cases, greater than 0.2% cells in the collected cord blood express CD34. In some cases, greater than 0.3% cells in the collected cord blood express CD34. In some cases, greater than exactly or about 0.245% cells in the collected cord blood express CD34. In specific embodiments, CBUs are characterized as non-desirable if less than or equal to, exactly or about 0.245% cells in the collected cord blood express CD34. In specific embodiments, CBUs are characterized as non-desirable if less than or equal to, exactly or about 0.225% cells in the collected cord blood express CD34. In specific embodiments, CBUs are characterized as non-desirable if less than or equal to, exactly or about 0.2% cells in the collected cord blood express CD34. In specific embodiments, CBUs are characterized as non-desirable if less than or equal to, exactly or about 0.175% cells in the collected cord blood express CD34. In specific embodiments, CBUs are characterized as non-desirable if less than or equal to, exactly or about 0.4% cells in the collected cord blood express CD34. In some embodiments, CD34 expression is measured prior to cryopreservation and/or use.

### I. Measurement of Cytotoxicity

Embodiments of the disclosure include measurement of cytotoxicity of immune cells of any kind, including NK cells, derived from cord blood units. In specific embodiments, there is measurement of cytotoxicity of NK cells derived from the cord blood unit(s). In particular cases, cord blood cell unit(s) are characterized for viability, NRBC, and TNC recovery, and following the selection of the cord blood cell unit(s) based on this characterization, and optionally following cryopreservation and thawing, cells from the cord blood unit(s) may be measured for cytotoxicity.

Cytotoxicity assays often rely on dying cells having highly compromised cellular membranes that allow the release of cytoplasmic content or the penetration of fluorescent dyes within the cell structure. Cytotoxicity can be measured in a number of different ways, such as measuring cell viability using vital dyes (formazan dyes), protease biomarkers, or by measuring ATP content, for example. The formazan dyes are chromogenic products formed by the reduction of tetrazolium salts (INT, MTT, MTS and XTT) by dehydrogenases, such as lactate dehydrogenase (LDH) and reductases that are released at cell death. Other assays include sulforhodamine B and water-soluble tetrazolium salt assays that may be utilized for high throughput screening. One can measure cytotoxicity with an Incucyte® device.

In specific embodiments, one can utilize dyes that selectively penetrate dead cells, such as Trypan blue. In other cases, one can utilize fluorescent DNA binding dyes that penetrate dead cells, such as Hoechst 33342, YO-PRO-1, or CellTox Green.

In specific embodiments wherein the cells being tested for being cytotoxic are T cells or NK cells, one may utilize the ⁵¹Cr release assay.

### J. Measurement of NK Cell Expansion

In specific embodiments of methods of the disclosure, the extent of NK cell expansion following cryopreservation and thawing of cord blood units (including cord blood units selected based on criteria encompassed herein) is a predictor of clinical response. That is, following thawing of cord blood, the thawed blood is processed and cultured under conditions such that the quantity of NK cells in the culture is increased. Cord blood units that meet selection criteria encompassed herein may or may not be pooled prior to expansion of NK cells. The quantitative extent of the NK cell expansion, including at certain time points in some cases, in some embodiments is utilized as a selection criteria for NK cells that will have greater clinical efficacy compared to NK cells derived from randomly selected cord blood units.

In particular cases, the NK cells are expanded, and the expansion level is determined. When the NK cells at a certain time point are expanded to at least a particular level, the NK cells have a greater clinical efficacy compared to NK cells that are not able to be expanded to such a level. In at least some cases, NK cells that would have clinical efficacy at a range between days 0 and 6 is greater than or equal to, exactly or about 2-fold, 3-fold, 4-fold, 5-fold, 6-fold, 7-fold (including 8-fold, 9-fold, 10-fold, 12-fold, 15-fold, 20-fold, 50-fold, 10⁰-fold, 150-fold, 200-fold, 250-fold, 500-fold, 10⁰⁰-fold, 1500-fold, 2000-fold, and so forth). In at least some cases, NK cells may have an insufficient clinical efficacy if at a range between days 0 and 6 the expansion is less than 7-fold (including less than 6-fold, 5-fold, 4-fold, 3-fold, or 2-fold). In at least some cases, NK cells would have clinical efficacy if at a range between days 6 and 15 the expansion in culture is greater than or equal to, exactly or about 10²-fold, 10³-fold, 10⁴-fold, 10⁵-fold (including 10⁶-fold, 10⁷-fold, 10⁸-fold, 10⁹-fold, 10¹⁰-fold, 10¹¹-fold, 10¹²-fold, 10¹³-fold, and so forth). In some cases, the NK cells have an expansion between days 6 and 15 in culture of at least or equal to, exactly or about 70 fold. In at least some cases, NK cells may have an insufficient clinical efficacy if at a range between days 6 and 15 the expansion in culture is less than 10⁵-fold (including less than 10⁴-fold, 10³-fold, 10²-fold, and so forth). In at least some cases, NK cells that would have clinical efficacy at a range between days 0 and 15 is greater than or equal to, exactly or about 900-fold, 1000-fold, 1100-fold, 1200-fold, 1300-fold, 1400-fold, 1500-fold, 1600-fold, 1700-fold, 1800-fold, 1900-fold, 2000-fold, 2500-fold, 3000-fold, 4000-fold, 5000-fold, 10^{,000}-fold, or greater. In some cases, the NK cells have an expansion between days 0 and 15 in culture of at least or equal to, exactly or about 450 fold. In at least some cases, NK cells may have an insufficient clinical efficacy if at a range between days 6 and 15 the expansion in culture is less than 900-fold, such as less than 800-fold, 700-fold, 600-fold, 500-fold, 400-fold, 300-fold, 200-fold, 10⁰-fold, and so forth.

In some embodiments, the NK cell expansion utilizes a particular in vitro method for expanding NK cells. In some cases, there is pre-activation of a population of NK cells in a pre-activation culture comprising an effective concentration of IL-12, IL-15, and/or IL-18 to obtain pre-activated NK cells; and then expanding the pre-activated NK cells in an expansion culture comprising artificial antigen presenting cells (aAPCs) expressing CD137 ligand . In certain aspects, the aAPCs further express a membrane-bound cytokine. In some aspects, the membrane-bound cytokine is membrane-bound IL-21 (mIL-21) and/or membrane-bound IL-15 (mIL-15). In some aspects, the aAPCs have essentially no expression of endogenous HLA class I, II, or CD1d molecules. In certain aspects, the aAPCs express ICAM-1 (CD54) and LFA-3 (CD58). In some aspects, the aAPCs are further defined as leukemia cell-derived aAPCs. In certain aspects, the leukemia-cell derived aAPCs are further defined as K562 cells engineered to express CD137 ligand and/or mIL-21. The K562 cells may be engineered to express CD137 ligand and mIL-21. In certain aspects, engineered is further defined as retroviral transduction. In particular aspects, the aAPCs are irradiated. In particular cases, the pre-activating step is for 10-20 hours, such as 14-18 hours (e.g., about 14, 15, 16, 17, or 18 hours), particularly about 16 hours. In certain aspects, the pre-activation culture comprises IL-18 and/or IL-15 at a concentration of 10-100 ng/mL, such as 40-60 ng/mL, particularly about 50 ng/mL. In some aspects, the pre-activation culture comprises IL-12 at a concentration of 0.1-150 ng/ml, such as 1-20 ng/ml, particularly about 10 ng/ml. In additional aspects, the expansion culture further comprises IL-2. In some aspects, the IL-2 is present at a concentration of 10-500 U/mL, such as 100-300 U/mL, particularly about 200 U/mL. In some aspects, the IL-12, IL-18, IL-15, and/or IL-2 is recombinant human IL-2. In some aspects, the IL-2 is replenished in the expansion culture every 2-3 days. In some aspects, the aAPCs are added to the expansion culture at least a second time. In some aspects, the method is performed in serum-free media.

In one embodiment, the expansion step comprises culturing the NK cells in the presence of an effective amount of universal antigen presenting cells (UAPC) engineered to express (1) CD48 and/or CS1 (CD319), (2) membrane-bound interleukin-21 (mbIL-21), and (3) 41BB ligand (41BBL)). In some aspects, the immune cells and UAPCs are cultured at a ratio of 3:1 to 1:3, such as 3:1, 3:2, 1:1, 1:2, or 1:3. In particular aspects, the immune cells and UAPCs are cultured at a ratio of 1:2. In some aspects, the UAPC has essentially no expression of endogenous HLA class I, II, or CD1d molecules. In certain aspects, the UAPC expresses ICAM-1 (CD54) and LFA-3 (CD58). In certain aspects, the UAPC is further defined as a leukemia cell-derived aAPC. In some aspects, the leukemia-cell derived UAPC is further defined as a K562 cell. In certain aspects, the UAPCs are added at least a second time.

In some aspects, the expanding is in the presence of IL-2. In specific aspects, the IL-2 is present at a concentration of 10-500 U/mL, such as 10-25, 25-50, 50-75, 75-10, 100-150, 150-200, 200-250, 250-300, 300-350, 350-400, or 400-500 U/mL. In certain aspects, the IL-2 is present at a concentration of 100-300 U/mL. In particular aspects, the IL-2 is present at a concentration of 200 U/mL. In some aspects, the IL-2 is recombinant human IL-2. In specific aspects, the IL-2 is replenished every 2-3 days, such as every 2 days or 3 days.

### K. Mothers Age

In specific embodiments of methods of the disclosure, the age of the mother is a predictor of clinical response in patients receiving CBU derived immune cells. In specific embodiments, the mothers age is less than or equal to, exactly or about 39, 38, 37, 36, 35, 34, 33, 32, 31, 30, 29, 28, 27, 26, 25, 24, 23, 22, or 21, or less than 21 years of age. In specific embodiments the mothers age is less than 32 years of age. In specific embodiments, the mothers age is less than 33 years of age. In specific embodiments, the mothers age is less than 31 years of age. In specific embodiments, CBUs are characterized as non-desirable if the mothers age is greater than 29 years of age. In specific embodiments, CBUs are characterized as non-desirable if the mothers age is greater than 30 years of age. In specific embodiments, CBUs are characterized as non-desirable if the mothers age is greater than 31 years of age. In specific embodiments, CBUs are characterized as non-desirable if the mothers age is greater than 32 years of age. In specific embodiments, CBUs are characterized as non-desirable if the mothers age is greater than 33 years of age. In specific embodiments, CBUs are characterized as non-desirable if the mothers age is greater than 34 years of age. In specific embodiments, CBUs are characterized as non-desirable if the mothers age is greater than 35 years of age.

### L. Gestational Age

In specific embodiments of methods of the disclosure, the gestational age of the baby is a predictor of clinical response in patients receiving CBU derived immune cells. In specific embodiments, the gestational age is less than or equal to, exactly or about 41, 40 39, 38, 37, 36, 35, 34, 33, 32, 31, 30, 29, 28, 27, 26, 25, 24, 23, 22, or 21 weeks of gestation. In specific embodiments, the gestational age of the baby is less than or equal to, exactly or about 38 weeks. In specific embodiments, the gestational age of the baby is less than or equal to, exactly or about 39 weeks. In specific embodiments, the gestational age of the baby is less than or equal to, exactly or about 37 weeks. In specific embodiments, CBUs are characterized as non-desirable if the gestational age is greater than or equal to, exactly or about 40 weeks. In specific embodiments, CBUs are characterized as non-desirable if the gestational age is greater than or equal to, exactly or about 39 weeks. In specific embodiments, CBUs are characterized as non-desirable if the gestational age is greater than or equal to, exactly or about 38 weeks. In specific embodiments, CBUs are characterized as non-desirable if the gestational age is greater than or equal to, exactly or about 37 weeks.

### M. Gender of the Baby

In specific embodiments of methods of the disclosure, the gender of the baby is a predictor of clinical response in patients receiving CBU derived immune cells. In specific embodiments, the gender of the baby is male. In specific embodiments, the gender of the baby is not female. In specific embodiments, CBUs are characterized as non-desirable if the gender of the baby is not male.

III. Immune Cells Derived from the Cord Blood

Certain embodiments of the present disclosure concern immune cells that are derived from cord blood unit(s) that are selected for processing based upon one or more criteria encompassed herein. The immune cells may be of any kind including NK cells, invariant NK cells, NKT cells, T cells (e.g., regulatory T cells, CD4+ T cells, CD8+ T cells, or gamma-delta T cells), monocytes, granulocytes, myeloid cells, macrophages, neutrophils, dendritic cells, mast cells, eosinophils, basophils, stem cells (e.g., mesenchymal stem cells (MSCs) or induced pluripotent stem (iPSC) cells), and so forth.

In some embodiments, the immune cells are, or are not, NK cells. In some embodiments, the immune cells are, or are not, invariant NK cells. In some embodiments, the immune cells are, or are not, NKT cells. In some embodiments, the immune cells are, or are not, T cells. In some embodiments, the immune cells are, or are not, monocytes. In some embodiments, the immune cells are, or are not, granulocytes. In some embodiments, the immune cells are, or are not, myeloid cells. In some embodiments, the immune cells are, or are not, macrophages. In some embodiments, the immune cells are, or are not, neutrophils. In some embodiments, the immune cells are, or are not, dendritic cells. In some embodiments, the immune cells are, or are not, mast cells. In some embodiments, the immune cells are, or are not, eosinophils. In some embodiments, the immune cells are, or are not, basophils. In some embodiments, the immune cells are, or are not, stem cells.

Also provided herein are methods of producing and engineering the immune cells as well as methods of using and administering the cells for adoptive cell therapy, in which case the cells may be autologous or allogeneic with respect to the individual(s) from which the cord blood was obtained. Thus, the immune cells may be used as immunotherapy, such as to target cancer cells.

The immune cells may be isolated from cord blood units from human subjects. The cord blood can be obtained from a subject of interest, such as a subject suspected of having a particular disease or condition, a subject suspected of having a predisposition to a particular disease or condition, or a subject who is undergoing therapy for a particular disease or condition. The cord blood can be obtained from a subject for the purpose of banking the cord blood in case it (including immune cells derived from it) is needed later in lift. The immune cells derived from the cord blood may be used directly, or they can be stored for a period of time, such as by freezing. The cord blood may or may not be pooled, such as may be from 2 or more sources, such as 3, 4, 5, 6, 7, 8, 9, 10 or more sources (e.g., donor subjects).

The cord blood from which the immune cells are derived can be obtained from a subject in need of therapy or suffering from a disease of any kind, including associated with reduced immune cell activity. Thus, the cells will be autologous to the subject in need of therapy. Alternatively, the population of immune cells can be obtained from a donor, preferably a histocompatibility matched donor . The immune cell population can be harvested from the peripheral blood, cord blood, bone marrow, spleen, or any other organ/tissue in which immune cells reside in said subject or donor . The immune cells can be isolated from a pool of subjects and/or donors, such as from pooled cord blood.

When the population of immune cells is obtained from cord blood units from a donor distinct from the subject, the donor is preferably allogeneic, provided the cells obtained are subject-compatible in that they can be introduced into the subject. Allogeneic donor cells are may or may not be human-leukocyte-antigen (HLA)-compatible. To be rendered subject-compatible, allogeneic cells can be treated to reduce immunogenicity.

### A. NK Cells

In some embodiments, the immune cells derived from the selected cord blood unit(s) are NK cells. In some embodiments, the cells derived from the selected CBUs are not cells other than NK cells. NK cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of IL-2, strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors (Campbell, 2006). When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-.gamma., tumor necrosis factor-.alpha. and granulocyte-macrophage colony-stimulating factor (GM-CSF), which activate both innate and adaptive immune cells as well as other cytokines and chemokines (Wu et al., 2003). Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

In certain aspects, the NK cells are isolated and expanded by the previously described method of ex vivo expansion of NK cells (Shah et al., 2013). In this method, CB mononuclear cells are isolated by Ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56+/CD3-cells or NK cells. In other methods, umbilical CB is used to derive NK cells by the isolation of CD34+ cells and differentiation into CD56+/CD3-cells by culturing in medium contain SCF, IL-7, IL-15, and IL-2.

### B. T Cells

In some embodiments, the immune cells derived from the selected cord blood unit(s) are T cells. In some embodiments, the immune cells derived from the selected cord blood unit(s) are not T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor TCR; and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, one or more subsets of T cells are derived from the selected cord blood, such as CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the T cells may come from cord blood that is allogeneic or autologous, or a mixture thereof.

Certain types of T cells may be derived from the selected cord blood. Among the sub-types and subpopulations of T cells (e.g., CD4.sup.+ and/or CD8.sup.+ T cells) there are naive T (T_{N}) cells, effector T cells (T_{EFF}), memory T cells and sub-types thereof, such as stem cell memory T (TSC_{M}), central memory T (TC_{M}), effector memory T (TE_{M}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the immune cells derived from the selected cord blood unit(s) are not naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and/or delta/gamma T cells.

In some embodiments, one or more of the T cell populations derived from the cord blood is enriched for or depleted of cells that are positive for one or more specific markers, such as surface markers, or that are negative for one or more specific markers. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from the cord blood sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation.

In some embodiments, the T cells are cultured in interleukin-2 (IL-2), and in any case they may be pooled prior to expansion. Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15). The non-specific T-cell receptor stimulus can include around 30 ng/ml of OKT3, a mouse monoclonal anti-CD3 antibody (available from Ortho-McNeil.RTM., Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15. The in vitro-induced T cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+allogeneic lymphocytes and IL-2, for example.

### C. Stem Cells

In some embodiments, the immune cells derived from the selected cord blood unit(s) may be stem cells, such as induced pluripotent stem cells (PSCs), mesenchymal stem cells (MSCs), or hematopoietic stem cells (HSCs), or a mixture thereof. In some embodiments, the immune cells derived from the selected cord blood unit(s) are not stem cells. In some embodiments, the immune cells derived from the selected cord blood unit(s) are not induced PSCs. In some embodiments, the immune cells derived from the selected cord blood unit(s) are not MSCs. In some embodiments, the immune cells derived from the selected cord blood unit(s) are not HSCs.

The pluripotent stem cells encompassed herein may be induced pluripotent stem (iPS) cells, commonly abbreviated iPS cells or iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

Somatic cells, such as those in the cord blood unit, can be reprogrammed to produce iPS cells using methods known to one of skill in the art. One of skill in the art can readily produce iPS cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; PCT Publication NO. WO 2007/069666 A1, U.S. Pat. Nos. 8,268,620; 8,546,140; 9,175,268; 8,741,648; U.S. Patent Application No. 2011/0104125, and U.S. Pat. No. 8,691,574, which are incorporated herein by reference. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized or Oct3/4, Sox2, Nanog, and Lin28.

Mouse and human cDNA sequences of these nuclear reprogramming substances are available with reference to the NCBI accession numbers mentioned in WO 2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, in published U.S. Pat. Nos. 8,900,871 and 8,071,369, which are both incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is can be carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR.TM. medium or E8.TM./Essential 8.TM. medium.

Plasmids have been designed with a number of goals in mind, such as achieving regulated high copy number and avoiding potential causes of plasmid instability in bacteria, and providing means for plasmid selection that are compatible with use in mammalian cells, including human cells. Particular attention has been paid to the dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. In some embodiments, plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. In particular aspects, the plasmids do not comprise a tyrosinase enhancer or promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, and U.S. application Ser. No. 12/478,154 which are incorporated herein by reference.

An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector . A viral gene delivery system can be an RNA-based or DNA-based viral vector .

### IV. Engineering of Cells

In some embodiments, immune cells derived from the selected cord blood unit(s) are engineered by the hand of man to be utilized for a variety of purposes. The engineering may be for the purpose of clinical or research applications. The engineered immune cells may be stored, or they may be used, such as administered to an individual in need thereof, in some cases. The engineering may or may not be performed by the same individual that generated the immune cells from the selected cord blood unit(s).

In specific embodiments, the immune cells are engineered to express one or more non-natural receptors, such as antigen receptors. The antigen may be of any kind, and the engineering of the immune cell to express the antigen facilitates use of the cell for a clinical application, in at least some cases. The antigen may be a cancer antigen (including a tumor antigen or hematopoietic cell antigen), or the antigen may be with respect to a pathogen of any kind, including bacterial, viral, fungal, parasitic, and so forth.

The immune cells from the selected cord blood unit(s) (e.g., autologous or allogeneic T cells (e.g., regulatory T cells, CD4+ T cells, CD8+ T cells, or gamma-delta T cells), NK cells, invariant NK cells, NKT cells, stem cells (e.g., MSCs or iPSCs) can be genetically engineered to express antigen receptors such as engineered TCRs and/or CARs. For example, the immune cells may be modified to express a TCR having antigenic specificity for a cancer antigen. In particular embodiments, NK cells are engineered to express a TCR. The NK cells may be alternatively or further engineered to express a CAR. Multiple CARs and/or TCRs, such as to different antigens, may be added to a single cell type, such as T cells or NK cells.

Suitable methods of modification of cells or recombination reagents are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

In some embodiments, exemplary cell modifications and/or methods of use, including combination therapies, addition of receptors (including at least CARs and recombinant TCRs), as well as methods for engineering and/or utilizing the cells, include those described, for example, in international patent application publication numbers WO2022/216992A1, WO2023/056330A1, WO2021/142127A1, WO2021/055349A1, WO2022/082224A1, WO2022/159791A1, WO2022/251377A1, WO2022/221548A1, WO2022/251504A2, WO2023/069969A1, WO2023/283644A2, WO2023/004425A2, in international patent application numbers PCT/US2023/065493, and/or in U.S. provisional application Nos. 63/500,423 filed May 5, 2023, 63/481,588 filed Jan. 25, 2023, and/or 63/434,395 filed Dec. 21, 2022; each of which are incorporated herein in their entirety for the purposes described herein.

For embodiments in which TCRs are utilized, electroporation of RNA coding for the full length TCR alpha and beta (or gamma and delta) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR .alpha. and .beta. chain are only transiently expressed. When the introduced TCR alpha and beta chain expression is diminished, only normal autologous T cells are left. This is not the case when full length TCR chains are introduced by stable retroviral transduction, which will never lose the introduced TCR chains, causing a constantly present autoreactivity in the patient.

Following genetic modification the immune cells may be immediately delivered (such as infused) or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor (as an example) is expanded ex vivo. The clone selected for expansion demonstrates the capacity to specifically recognize and lyse antigen-expressing target cells. The recombinant immune cells may be expanded by stimulation, such as with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

### A. Chimeric Antigen Receptors

In some embodiments, the immune cells are engineered to express a CAR, and the CAR may comprise: a) one or more intracellular signaling domains, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor .

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3zeta, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector . Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3−. zeta., CD137/CD3-zeta, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR.sup.+immune cells (Singh et al., 2008; Singh et al., 2011).

### B. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable .alpha. and .beta. chains (also known as TCR.alpha, and TCR .beta., respectively) or a variable .gamma. and .delta. chains (also known as TCR.gamma, and TCR.delta., respectively) and that is capable of specifically binding to an antigen peptide bound to a MHC receptor, such as a classical MHC receptor or a non-classical MHC receptor . In some embodiments, the TCR is in the .alpha . . . beta. form. In some embodiments, TCRs include those from naturally occurring invariant Natural Killer T Cells (iNKT cells). In some embodiments, the TCR is an invariant TCR (iTCR). In some embodiments, the TCR comprises an iTCR alpha and iTCR beta chain.

Typically, TCRs that exist in.alpha . . . beta. and .gamma . . . delta. forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the .alpha . . . beta. form or .gamma . . . delta. form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable .alpha. chain and variable .beta. chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the .beta.-chain can contain a further hypervariability (HV4) region.

In some embodiments, the TCR chains contain a constant domain. For example, like immunoglobulins, the extracellular portion of TCR chains (e.g., .alpha.-chain, .beta.-chain) can contain two immunoglobulin domains, a variable domain (e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5.sup.th ed.) at the N-terminus, and one constant domain (e.g., a-chain constant domain or C.sub.a, typically amino acids 117 to 259 based on Kabat, beta-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the alpha and beta chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (.gamma., .delta., and .epsilon.) in mammals and the .zeta.-chain. For example, in mammals the complex can contain a CD3-gamma chain, a CD3-delta chain, two CD3-epsilon chains, and a homodimer of CD3-zeta chains. The CD3-gamma, CD3-delta, and CD3-epsilon chains are highly related cell surface proteins of the immunoglobulin superfamily containing a single immunoglobulin domain. The transmembrane regions of the CD3-gamma, CD3-delta, and CD3-epsilon chains are negatively charged, which is a characteristic that allows these chains to associate with the positively charged T cell receptor chains. The intracellular tails of the CD3-gamma, CD3-delta, and CD3-epsilon chains each contain a single conserved motif known as an immunoreceptor tyrosine-based activation motif or ITAM, whereas each CD3-zeta chain has three. Generally, ITAMs are involved in the signaling capacity of the TCR complex. These accessory molecules have negatively charged transmembrane regions and play a role in propagating the signal from the TCR into the cell.

In some embodiments, the TCR may be a heterodimer of two chains alpha and beta (or optionally gamma and delta) or it may be a single chain TCR construct. In some embodiments, the TCR is a heterodimer containing two separate chains (alpha and beta chains or gamma and delta chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### C. Antigens

In specific cases, the immune cells derived from the selected cord blood unit(s) are engineered to express a protein that targets an antigen, such as a receptor that targets an antigen. In specific cases, the receptor is genetically engineered to comprise chimeric components from different sources. Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP,-catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE.

These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP,-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch 1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include coronavirus of any kind, including SARS-COV and SARS-CoV2, human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species, including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and IE2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK.RTM., SWISS-PROT.RTM., and TREMBL.RTM. (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y. pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSAI c-term), and exported protein 1 (PfExp=1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### D. Cytokines

In some cases, immune cells derived from the selected cord blood unit(s) are engineered to express one or more cytokines, including one or more heterologous cytokines. The cytokines may be of any kind, but in specific embodiments, the heterologous cytokine(s) is selected from the group consisting of IL-4, IL-10, IL-7, IL-2, IL-15, IL-12, IL-18, IL-21, and a combination thereof.

In specific embodiments, the cytokine is IL-15. IL-15 is tissue-restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines (e.g., IL-2, IL-12, IL-18, and/or IL-21), chemokines, and other molecules that contribute to the activation and proliferation of cells used for human application. NK cells expressing IL-15 are capable of continued supportive cytokine signaling, which is useful for their survival post-infusion. In certain embodiments, NK cells expressing IL-21 are capable of continued supportive cytokine signaling, which is useful for their survival post-infusion. In certain embodiments, a cytokine is expressed as part of a multicistronic construct with one or more functional proteins and/or marker proteins.

In some embodiments, the cells express one or more exogenously provided engineered receptors, wherein the engineered receptor comprises a chemokine receptor and/or a cytokine receptor . In some embodiments, a cytokine receptor is an IL-15 receptor . In some embodiments, a cytokine receptor is a non-naturally occurring variant of a cytokine receptor . In some embodiments, a cytokine receptor is an IL-15, IL-12, IL-2, IL-18, IL-21, IL-23, or GMCSF receptor, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine.

In one embodiments, the present disclosure concerns co-modifying immune cells expressing CAR and/or TCR immune cells with one or more cytokines, including IL-15. In addition to IL-15, other cytokines are envisioned. These include, but are not limited to, cytokines, chemokines, and other molecules that contribute to the activation and proliferation of cells used for human application. In some embodiments, NK or T cells expressing IL-15 are capable of continued supportive cytokine signaling, which is critical to their survival post-infusion.

### E. Suicide Genes

The immune cells of the present disclosure derived from cord blood unit(s) may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene. Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-.alpha., .gamma.-lyase (MET), and Thymidine phosphorylase (TP).

### F. Gene Disruption

In some embodiments, the immune cells are engineered to have disruption of expression of one or more endogenous genes. The disruption may be a knockout or knockdown, in specific cases. The disruption may be produced in the cells by any suitable method, including CRISPR, antisense technology, such as RNAi, siRNA, shRNA, and/or ribozymes, which generally result in transient reduction of expression, as well as gene editing techniques that result in targeted gene inactivation or disruption, e.g., by induction of breaks and/or homologous recombination.

In certain embodiments, NK cells of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous transgenes (e.g., TCRs, CARs, cytokines, suicide genes, etc.), whereas in other cases the gene editing occurs in NK cells that do not express a heterologous transgene but that ultimately will express one or more heterologous transgenes, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CREB, CREM, ICER, and CD39. In specific embodiments, the TGFBR2 gene is knocked out or knocked down in the NK cells. In specific embodiments, the CISH gene is knocked out or knocked down in the NK cells. In specific embodiments, the CD38 gene is knocked out or knocked down in the NK cells. In specific embodiments, the Glucocorticoid receptor (GR) gene is knocked out or knocked down in the NK cells. In certain embodiments, an endogenous gene that is disrupted by CRISPR is TIGIT.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art.

### V. Methods of Use

Once the cord blood unit(s) are selected, immune cells derived therefrom may or may not be engineered and may or may not be stored. In any event, a therapeutically effective amount of the immune cells, engineered or not, may be delivered to an individual in need thereof. The immune cells are particularly effective because they have been derived from selected cord blood unit(s) for the explicit reason of having met one or more selection criteria, as described herein.

In some embodiments, the present disclosure provides methods for immunotherapy comprising administering an effective amount of the immune cells produced by methods the present disclosure. In one embodiments, a medical disease or disorder is treated by transfer of an immune cell population that elicits an immune response. In certain embodiments of the present disclosure, cancer or infection is treated by transfer of the produced immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor . Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma;
osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma;
astrocytoma; protoplasmic astrocytoma; fibrillary astrocytoma; astroblastoma; glioblastoma;

oligodendroglioma; oligodendroblastoma; primitive neuroectodermal; cerebellar sarcoma;

ganglioneuroblastoma; neuroblastoma; retinoblastoma; olfactory neurogenic tumor;

meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL;
intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor . There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m.sup.2 fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema, and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×109, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A wide variety of chemotherapeutic agents may be used in conjunction with the produced immune cells. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, azithromycin, anthramycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

In some embodiments, radiotherapy it provided to the individual in addition to the immune cells produced herein. The radiation may include gamma-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The skilled artisan will also understand that additional immunotherapies may be used in combination or in conjunction with the immune cells produced by method encompassed herein. In the context of cancer treatment, immunotherapeutics, generally, rely on the use of immune effector cells and molecules to target and destroy cancer cells. Rituximab is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS.RTM. (brentuximab vedotin) in 2011 and KADCYLA.RTM. (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand .

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons .alpha., .beta., and .gamma., IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some cases, surgery is performed for an individual that will receive the immune cells of the disclosure or that have received them. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor . In addition to tumor resection, treatment by surgery includes laser surgery, cryosurgery, electrosurgery, and microscopically-controlled surgery (Mohs' surgery). Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Articles of Manufacture or Kits

An article of manufacture or a kit is provided comprising immune cells produced from selected cord blood unit(s) is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. In some embodiments, kits may also comprise one or more adjuvant therapies, such as but not limited to antibody based therapies. Suitable containers include, for example, bottles, vials, bags, and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In specific embodiments the article of manufacture comprises cryopreserved immune cells produced by methods described herein. The cryopreserved cells may be frozen with a particular cryoprotectant suited to prevent them from damage upon freezing or thawing.

### Examples

The following examples are included to demonstrate particular embodiments of the disclosure. It should be appreciated by those of skill in the art that the techniques disclosed in the examples that follow represent techniques discovered by the inventors to function well in the practice of the embodiments of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1-Methods

Unless otherwise described, experimental methods were conducted as outlined here.

Clinical trial design—The inventors conducted a phase I-II clinical trial to assess the safety and efficacy of escalating doses of CAR19/IL-15 CB-NK cells for patients with relapsed/refractory CD19-positive malignancies. Patients were treated between June 2017 and June 2021 and then followed for 12 months after the cell infusion. The first patient was enrolled on Jun. 30, 2017 and the last patient was enrolled on May 27, 2021. Patients 7-80 years of age with relapsed/refractory CD19-positive B-cell malignancies, a Karnofsky performance status of >70% and an adequate organ function were eligible. Patients also must have been at least three weeks from the last cytotoxic chemotherapy or at least three days from tyrosine kinase inhibitors or other targeted therapies to be eligible. Exclusion criteria included: 1) pregnancy, 2) positive serology for HIV, 3) uncontrolled infections, 4) grade III or higher toxicities from prior therapies, 5) active neurological disorders, and 6) receipt of concomitant investigational therapies. Prior CD19 targeting therapy was an exclusion criteria for the second phase of the study. The study had two phases, a dose escalation phase and an expansion phase. The dose escalation phase I (n=11) was previously reported 13. Patients received lymphodepleting chemotherapy with fludarabine 30 mg/m² and cyclophosphamide 300 mg/m² daily for 3 consecutive days followed by the infusion of CAR19/IL-15 CB-NK cells at escalating doses of 10⁵ cells/kg, 10⁶ cells/kg and 10⁷ cells/kg. The dose was escalated using the EffTox design (see below). In the expansion phase (n=26), patients were treated at the 10⁷ cells/kg CAR19/IL-15 CB-NK dose level. Then the trial was amended to include a second expansion cohort where patients received a single flat dose of 8×10⁸ CAR19/IL-15 CB-NK cells (the equivalent of 10⁷ cells/kg for an 80 kg person). The first nine patients in the Phase I portion of the study received a CAR-NK product that was partially matched with the recipient (4/6 HLA molecules: HLA-A, B and DRB1); the protocol was then amended to permit selection of cords with no consideration for HLA matching. The HLA mismatches were bidirectional for all patients. Table 1 shows the patient and cell product characteristics. No formal sample size computation was performed. Instead, patients were enrolled following a Bayesian EffTox dosing that included two Bayesian adaptive rules, taking into account both efficacy and toxicity outcomes. The median follow-up time for alive patients on the study was 12 months (10-12 months). One patient was lost to follow-up at 10 months.

The study was approved by the institutional review board and conducted according to the declaration of Helsinki. The study was overseen by the External Data Safety Monitoring Board of MD Anderson Cancer Center. Written informed consent was obtained from each patient. The trial is registered on the clinicaltrials.gov website (NCT03056339). The clinical protocol and additional information on the study design are publicly available.

Safety and toxicity monitoring—The method of Thall et al. (1995) was used to determine the maximum tolerated dose (MTD) and to construct a stopping bound for toxicity 46. A dose level was considered too toxic if the maximum upper limit on probability of dose limiting toxicity (DLT) was 0.40. The following events were considered as DLT: grade 3 or 4 graft-versus-host disease (GVHD) within 8 weeks of NK cell infusion, cytokine release syndrome (CRS) within 2 weeks of NK cell infusion requiring transfer to intensive care, grade 4 NK cell infusion related toxicity, grades 3-5 allergic reactions related to study cell infusion, grade 3-5 organ toxicity (cardiac, dermatologic, gastrointestinal, hepatic, pulmonary, renal/genitourinary, or neurologic) not pre-existing or due to the underlying malignancy or due to lymphodepleting chemotherapy or treatment-related death within 8 weeks of the study cell infusion.

Clinical trial amendments and patient enrollment-Between June 2017 and June 2021, 48 consecutive patients were enrolled in the protocol (11 were screen failures and 37 received the therapy). Patients were enrolled sequentially with a staggering interval of 14 days from the day of CAR-NK infusion to the start of the preparative regimen for the next patient within each cohort, as well as a 2-week interval as the dose was escalated to the next level. In March 2019 the inventors considered the dose finding portion of the study to be complete and the protocol was amended (amendment 16) to allow for the enrollment of additional patients at the 10⁷ cells/kg dose. In April 2020, in preparation for the introduction of a frozen product (which was not done in this trial) the inventors changed the dose of 10⁷ cells/kg dose to a flat dose of 8×10⁸ cells (amendment 22). The last version of the protocol was version 25. The data cutoff for this report was July 2022, at which point all patients had completed the 1-year follow-up.

Clinical responses-Clinical responses to therapy for CLL and NHL were based on the Lugano and iWCLL 2018 criteria, respectively ^{41,42}. OR represents the combination of PR and CR. Day+30 OR was defined as the achievement of PR or CR at any time within 30 days after the infusion. One-year CR was defined as the achievement of CR at any time within 1 year after the infusion. All patients who achieved CR during follow-up were in PR at day 30.

Manufacture of iC9/CD19-CAR/IL15-transduced NK cells (CD19 NK cells) from cord blood—The clinical cord blood (CB) units for CAR-NK production were obtained from the MD Anderson Cancer Center (MDACC) CB bank. CB was collected after informed consent from mothers at several hospitals and shipped to the MDACC CB Bank for processing and cryopreservation following standard operating procedures (SOPs). The time from collection-to-cryopreservation was the time from collection of CB at mother's bedside to the time the cord was cryobanked. The CAR-NK cells were manufactured in the MDACC Good Manufacturing Practice (GMP) facility. Briefly, the cord unit was thawed in a water bath, and NK cells were purified by CD3, CD19, and CD14 negative selection (Miltenyi beads) and cultured in the presence of engineered K562 feeder cells expressing membrane-bound IL-21 and 4-1BB ligand plus exogenous IL-2 (200 U/ml). On day 6 of culture, cells were transduced with a retroviral vector encoding anti-CD19 CAR, IL15, and iC9 genes 49. The cells were expanded for an additional nine days and harvested for fresh infusion on day 15. For a subset of patients (n=17), the products were expanded for 22 days, with a second round of universal antigen presenting cells (uAPC) stimulation at day 15 of culture. The final CAR-NK transduction efficiency for the infused product was 72.4% (range 22.7-91.1). The median CD3-positive T-cell content in the infused product was 2000 cells/kg (range 30-16000 cells/kg).

Analysis of serum cytokines and qPCR for CAR NK cell monitoring—The cytokine assays were performed on serum from peripheral blood (PB) samples collected from patients at multiple timepoints after CAR-NK cell infusion using the ProcartaPlex™ kit from Thermo Fisher Scientific (Vienna, Austria), following the manufacturer's instructions. The qPCR assays were performed on serial PB samples as previously described (see e.g., Enli Liu et al., Use of CAR-Transduced Natural Killer Cells in CD19-Positive Lymphoid Tumors. NEJM, 2020; which is incorporated herein by reference for the purposes described herein).

Donor-specific antibody (DSA) measurement-Patients were screened for the presence of donor-specific anti-HLA antibodies at the MD Anderson HLA laboratory before and at multiple time points after CAR-NK infusion. If the screen was positive, the specificity of the antibody was determined using semi-quantitative solid phase fluorescent beads antibody detection assay on a Luminex platform. Results were expressed as mean fluorescent intensity (MFI) with MFI≥1,000 being considered positive.

Cell lines, primary cells, and culture conditions-Cell lines of Raji (CCL-86), MM1S (CRL-2974), SKOV3 (HTB-77), K562 (CRL-3344) and 293T (CRL-3216) were obtained from the American Type Culture Collection (ATCC). Cells of Raji, MMIS and K562 were cultured in RPMI-1640 (Invitrogen) supplemented with 10% fetal bovine serum (FBS; HyClone), 1% penicillin-streptomycin, and 1% GlutaMAX™; 293T and SKOV3 cells were cultured in DMEM (Invitrogen) supplemented with 10% FBS, 1% penicillin-streptomycin and 1% GlutaMAX™ Raji cells were transduced with mCherry to facilitate their detection in in vitro assays; Raji, MMIS and SKOV3 cells were labeled with firefly luciferase (Ffluc)-GFP for in vivo tumor analysis by the IVIS Spectrum bioluminescence imaging system (Caliper). All cells were maintained in a 37 °C incubator with 5% CO2, and regularly tested for mycoplasma contamination using the MycoAlert® Mycoplasma Detection Kit (Lonza).

Nucleated Red Blood Cell (NRBC) isolation from CB—CB units were provided by the MD Anderson CB Bank under institutional review board-approved protocols. CB mononuclear cells were isolated by a density-gradient technique (Ficoll-Histopaque®; Sigma, St Louis, MO, USA). NRBCs were isolated by positive selection using CD71 and CD235A (Glycophorin A) beads (Miltenyi Biotec) and cultured in 48-well plates at a concentration of 500,000 cells/ml in RPMI/Click's media. Supernatants were collected for Elisa assays after 24, 48 and 72 hours of culture.

Transforming Growth Factor Beta (TGFB) Milliplex assay-TGF-B1 and 2 measurements were performed using MILLIPLEX (R) MAP TGFB (Transforming Growth Factor Beta)-3 Plex (TGFBMAG-64K-03) following the manufacturer's (Sigma-Aldrich) instructions, on a Luminex 200 instrument. The levels of TGF-B1 and 2 in media alone were subtracted from the values obtained from NRBC conditions. Data were analyzed using Bio-Plex software.

Arginase-1 ELISA assay-Arginase-1 quantitation was performed using the BMS2216 ELISA kit from Invitrogen, following the manufacturer's instructions. Data were acquired on a 96-well microplate reader.

Flow cytometry-CAR expression was measured using a conjugated goat anti-human IgG (H+L; Jackson ImmunoResearch) that recognized the IgG hinge portion of the CAR construct. The inventors utilized Ghost Dye™ Violet 450 (TONBO Biosciences) to determine viability, and aqua fixable viability dye (eBioscience) when fixation protocols were applied. Human Fc receptor blocking solution (Miltenyi Biotec) was applied to minimize non-specific staining. Cell counts were measured by AccuCheck Counting Beads (ThermoFisher). Cells were acquired on LSRFortessa™ X-20 (BD Biosciences) and data analyzed using FlowJo (Version 10.8.1, BD Biosciences).

Flow cytometry antibodies for the in vivo mouse models included: Live Dead-BV510 (Invitrogen, 1:200), Human CD45-PerCP (Biolegend, HI30, 1:50), Mouse CD45-BV650 (Biolegend, 30-F11, 1:50), Human CD56-BV605 (Biolegend, 5.1H11, 1:50), Human CD16-BV605 (Biolegend, 3G8, 1:50), Human CD3-APCY7 (Biolegend, HIT3a, 1:100), Human CD19-PECY7 (BD Biosciences, SJ25C1, 1:50), Human CD20-AF700 (BD Biosciences, 2H7, 1:50), Anti Biotin-PE (Miltenyi Biotec, Bio3-18E7, 1:20), CD19 CAR Detection reagent-Unconjugated (Miltenyi Biotec, 1:50), Human CD27-PECF594 (BD Biosciences, M-T271, 1:50), Human CD70-PECY7 (Biolegend, 113-16, 1:50), Human BCMA-PE (Miltenyi Biotec, REA315, 1:50), Human CD138-AF700 (BD Biosciences, MI15, 1:50), TROP2-PE (Biolegend, NY18, 1:50), Anti-His-APC (Biolegend, J095G46, 1:50).

In the Raji mouse model, the NK cell population was identified by first gating on lymphocytes using forward and side scatters. Cells were then gated on singlets, followed by live cells defined as Live Deadlow. Human NK cells were identified by first gating on hCD45⁺mCD45⁻ followed by CD16⁺CD56⁺CD19⁻ cells. CAR19⁺NK cells were identified using conjugated goat anti-human IgG; fluorescence minus one (FMO) or NT-NK cells were used as controls. To identify Raji cells, we first gated on the hCD45⁺mCD45⁻ population, followed by CD16⁻CD56⁻CD19⁺GFP⁺cells. In the MMIS mouse model, the NK cell population was identified by first gating on lymphocytes using forward and side scatters, then on singlets, followed by Live Deadlow, then hCD45⁺CD138⁻ and finally CD16⁻CD56⁻ cells. CAR70⁺NK cells were identified as CD16⁺CD56⁺CD27⁻, with FMO or NT-NK cells used as controls. MMIS cells were gated from the Live Deadlow population and identified as hCD45-CD138⁺.

Trogocytosis was measured by surface expression of CD19 on CAR-NK cells by flow cytometry. High trogocytosis was defined as a normalized tCD19 MFI level greater than the mean while low trogocytosis was defined as a level equal or less than the mean at more than one time point as previously described 20.

Tumor rechallenge assay in IncuCyte® system-NK cells were co-cultured at different effector-to-target (E:T) ratios with Raji tumor cells labeled with mCherry, and fresh tumor cells were added to the co-culture every 2-3 days. For rechallenge assays using CAR-NK cells, 100,000 mCherry labeled Raji cells were added at each challenge. For rechallenge assays using NT-NK cells, 16,700 mCherry labeled Raji cells were added at each challenge. The tumor cell index represented the counts of tumor cells where the intensity of mCherry fluorochrome was detected. Images of each well were captured in real-time. Data were analyzed using the IncuCyte Live-Cell Imaging System that measures the number of target cells (fluorochrome labeled) in real-time.

NK population doubling (PD) assay-NK cells were subcultured every week, with or without K562-based feeder cells, after the initial transduction and expansion. Using the equation for PD=log 10[(A/B)/2], where A is the number of harvested cells and B the number of plated cells from each subculture, the weekly PD was measured, then, the sum of each PD over time was determined as the cumulative PD. Assays were terminated three weeks after the cell count from the subculture failed to achieve at least an equal amount of seeded cells. Data were obtained from three different CB-derived NK populations for each condition.

Mass cytometry (CyTOF)-Mass cytometry was performed as previously described 44. Primary antibodies were conjugated in-house with the corresponding metal tags using MaxparX8 and MCP9 polymer antibody labeling kits per manufacturer's protocol (Standard BioTools). NK cells were washed with cell staining buffer (0.5% bovine serum albumin/phosphate-buffered saline (PBS)). Cells were then incubated with 2.5 μM cisplatin (Pt198, Standard BioTools) for 3 minutes for viability assessment, followed by washing twice with cell staining buffer. Cells were then stained with freshly prepared antibody mix against cell surface markers for 30 minutes on a shaker at room temperature, then washed twice and fixed with freshly prepared 1.6% paraformaldehyde (EMD Biosciences)/PBS for 10 minutes at room temperature. The cells were then rinsed twice with cell staining buffer and incubated overnight in −80 °C with 80% methanol. The following day, the cells were stained with intracellular marker-specific antibodies for 45-60 mins in the presence of 0.2% saponin. After an additional washing step, the cells were stored overnight in 1,000 μl Maxpar fix and perm buffer (Standard BioTools, 201067) with 125 nM of Iridium nucleic acid intercalator (Standard BioTools) in 4 °C. The cells were then washed and resuspended in MilliQ dH₂O supplemented with EQTM 4-element calibration beads, and subsequently acquired at 300 events/second on a Helios instrument (Standard BioTools). The CyTOF antibodies used with the corresponding metal tag isotopes are: CD45 (Standard Biotools, HI30, ⁸⁹Y), CCR6 (Miltenyi Biotec, REA190, ¹⁴¹Pr), EOMES (Invitrogen, WD1928, ¹⁴²Nd), KIR2DL4 (Miltenyi Biotec, REA768, ¹⁴³Nd), KIR3DL1 (BD Pharmingen, DX9, ¹⁴⁴Nd), CD70 (Biolegend, 113-16, 145Nd), KIR2DL5 (Miltenyi Biotec, REA955, ¹⁴⁶Nd), NKG2C (Miltenyi Biotec, REA205, ¹⁴⁷Sm), TRAIL (Miltenyi Biotec, REA1113, ¹⁴⁸Nd), CD25 (Standard Biotools, 2A3, ¹⁴⁹Sm), CD69 (Miltenyi Biotec, REA824, ¹⁵⁰Nd), 2B4 (Miltenyi Biotec, REA112, ¹⁵¹Eu), Granzyme B (GrB; Miltenyi Biotec, REA226, ¹⁵²Sm), TIM3 (Miltenyi Biotec, REA635, ¹⁵³Eu), CX3CR1 (Miltenyi Biotec, REA385, ¹⁵⁴Sm), KIR2DL3 (Miltenyi Biotec, REA147, ¹⁵⁵Gd), CXCR3 (Standard Biotools, G025H7, ¹⁵⁶Gd), OX40 (Miltenyi Biotec, REA621, ¹⁵⁸Gd), Perforin (PFN; Miltenyi Biotec, REA1061, ¹⁵⁹Tb), T-bet (Standard Biotools, 4B10, ¹⁶⁰Gd), TIGIT (Miltenyi Biotec, REA1004, 161Dy), Ki67 (Standard Biotools, B56, 162Dy), KIR2DL1 (Miltenyi Biotec, REA284, ¹⁶³Dy), KIR2DS1 (R&D Systems, 1127B, ¹⁶⁴Dy), PD1 (Miltenyi Biotec, PD1.3.1.3, ¹⁶⁵Ho), NKG2D (Miltenyi Biotec, REA797, ¹⁶⁶Er), CD38 (Miltenyi Biotec, REA572, ¹⁶⁷Er), CD73 (Standard Biotools, AD2, ¹⁶⁸Er), CD39 (Miltenyi Biotec, MZ18-23C8, ¹⁶⁹Tm), CD161 (Miltenyi Biotec, REA631, ¹⁷⁰Er), DNAM (Miltenyi Biotec, REA1040, 171Yb), KLRG1 (Miltenyi Biotec, REA261, ¹⁷²Yb), CXCR4 (Standard Biotools, 12G5, ¹⁷³Yb), KIR2DS4 (Miltenyi Biotec, REA860, ¹⁷⁴Yb), LAG3 (Miltenyi Biotec, REA351, ¹⁷⁵Lu), ICOS (Miltenyi Biotec, REA192, ¹⁷⁶Yb), CD16 (Standard Biotools, 3G8, ²⁰⁹Bi), CD57 (Miltenyi Biotec, REA769, ¹¹⁵ In), CD3 (Miltenyi Biotec, REA613, ¹⁹⁴ Pt), NKG2A (Miltenyi Biotec, REA110, ¹⁹⁵ Pt), HLA-DR (Miltenyi Biotec, REA805, ¹⁹⁶ Pt), LD (Standard Biotools, Cisplatin, ¹⁹⁸ Pt), CD56 (Miltenyi Biotec, REA196, ¹⁰⁶Cd), CAR (Miltenyi Biotec, REA1298, ¹¹⁰Cd), CD2 (Miltenyi Biotec, REA972, ¹¹¹Cd), CD8 (Miltenyi Biotec, REA734, ¹¹²Cd), NKP30 (Miltenyi Biotec, AF29-4D12, ¹¹³Cd), NKP46 (Miltenyi Biotec, REA808, ¹¹⁴Cd), NKP44 (Miltenyi Biotec, REA1163, ¹¹⁶Cd), CD36 (Miltenyi Biotec, REA760, ¹⁴²Nd), CD127 (Standard Biotools, A019D5, ¹⁴³Nd), CD11b (Standard Biotools, ICRF44, ¹⁴⁴Nd), CD62L (Miltenyi Biotec, REA615, ¹⁴⁵Nd), CD64 (Miltenyi Biotec, REA978, ¹⁴⁸Nd), CD86 (Miltenyi Biotec, REA968, ¹⁵⁰Nd), CD123 (Miltenyi Biotec, REA918, ¹⁵¹Eu), TCRgd (Miltenyi Biotec, REA591, 152Sm), CD27 (Miltenyi Biotec, REA499, 155Gd), CCR4 (Miltenyi Biotec, REA279, 158Gd), CD11c (Standard Biotools, Bu15, ¹⁵⁹Tb), CD80 (Standard Biotools, 2D10.4, ¹⁶¹Dy), CD66B (Standard Biotools, 80H3, ¹⁶²Dy), TCR Va7.2 (Miltenyi Biotec, REA179, ¹⁶³Dy), CD45RO (Miltenyi Biotec, REA611, ¹⁶⁴Dy), CD163 (Standard Biotools, GHI/61, ¹⁶⁵Ho), CCR7 (Miltenyi Biotec, REA546, ¹⁶⁷Er), CD45RA (Miltenyi Biotec, REA562, ¹⁶⁹Tm), CXCR5 (Miltenyi Biotec, REA103, ¹⁷¹Yb), INKT (Biolegend, 6B11, ¹⁷³Yb), CD95 (Standard Biotools, DX2, ¹⁷⁵Lu), CD19 (Miltenyi Biotec, REA675, ¹¹⁰Cd), CD4 (Miltenyi Biotec, REA623, ¹¹¹Cd), CD15 (BD Pharmingen, HI98, ¹¹³Cd), CD14 (Miltenyi Biotec, REA599, ¹¹⁴Cd), CD20 (Miltenyi Biotec, REA780, ¹¹⁶Cd), GFP (Biolegend, FM264G, ¹⁴⁴Nd), CD81 (Miltenyi Biotec, REA513, ¹⁴⁵Nd), PANKIR (R&D, 180704, ¹⁵³Eu), mCD45 (Biolegend, 30-F11, ¹⁵⁴Sm), PFN (Standard Biotools, B-D48, ¹⁹⁶ Pt), GrB (Standard Biotools, GB11, ¹⁹⁸ Pt).

Mass cytometry data analysis-Mass cytometry data were analyzed using Cytobank®. The NK cell population was identified using the following gating strategy: gating singlets followed by Pt198 (cisplatin)^{low} followed by hCD45+CD56+CD3⁻. The gating strategy was applied to all files. CAR⁺NK cells were determined compared to either isotype controls or NT-NK cell controls. NK cells from each donor were downsampled in FlowJo® using the Downsample plugin. Normalized data were pooled according to Opt-Cs vs. Sub-Cs classification and analyzed together in Cytobank. SPADE analysis was performed for clustering and visualization of high-dimensional single-cell data. Cells with phenotypical similarity were hierarchically clustered together in sub-clusters (nodes) that form clusters (branches) to indicate the diverse phenotypic landscape of the data. The expression of each marker in the sub-clusters was transformed and normalized locally and plotted as a heatmap using Morpheus matrix visualization and analysis software (Broad Institute).

Seahorse metabolic assays—The extracellular acidification rate (ECAR) (surrogate for glycolysis) and oxygen consumption rate (OCR) (surrogate for mitochondrial function) were measured using the Agilent Seahorse XF Pro Analyzer® (Agilent) following the manufacturer's protocol. ECAR was measured by Seahorse glycolysis stress test using 2 g/L D-glucose, 2.5 μM Oligomycin and 100 mM 2-Deoxyglucose (2-DG) mixed with Hoechst 33342 (Invitrogen) dye. OCR was measured by Seahorse mito stress test using 2.5 μM Oligomycin, 0.5 μM FCCP, and 0.5 μM Rotenone/Antimycin A mixed with Hoechst 33342 (Invitrogen) dye. Each NK cell condition was assayed in technical triplicates. Following the assays, live cell imaging and viable cell counting were performed in Cytation 1® machine. Normalized OCR or ECAR data per 250,000 live cells were shown. The basal respiration was calculated as follows: last rate measurement before first injection-non-mitochondrial respiration rate which represents the minimum rate measurement after Rotenone/Antimycin A. The maximal respiration was calculated as follows: maximum rate measurement after FCCP injection-non-mitochondrial respiration. The baseline glycolysis was presented as the non-glycolytic acidification, which consists of the last rate measurement prior to glucose injection. The glycolytic capacity was calculated as follows: maximum rate measurement after oligomycin injection-last rate measurement before glucose injection.

Isoplexis assays—The single cell secretome analysis was performed using the IsoCode chip from IsoPlexis® using the human natural killer cytokine panel. The assay was performed using the manufacturer's kit and following instructions (IsoPlexis, Branford, CT, USA). In brief, NT-NK cells were stimulated using purified anti-human CD16 antibody (BD Pharmingen, 555404, 1 μg/ml) and CAR-NK cells were stimulated using human CD19 antigen (ACRO, CD9-H5259, 10 μg/ml) for 4 hours at 37 °C. NK cells were washed and labeled with a fluorescent dye (Isoplexis stain cell membrane 405), and 30,000 cells were loaded onto the IsoCode chips. The IsoLight device was used to scan the chips, and IsoPlexis's proprietary IsoSpeak software was used to analyze the data. The PSI, as computed by the software, was used for data representation. Stimulatory cytokines consist of GM-CSF, IL-12, IL-15, IL-2, IL-21, IL-5, IL-7, IL-8 and IL-9. Effector cytokines consist of GZMB, IFN-γ, MIP-1α, perforin, TNF-α and TNF-β. Chemokines consist of CCL-11, IP-10, MIP-1B, RANTES 45.

Bulk RNA-seq processing and differential expression-Cord units (see e.g., Table 16) were thawed, NK cells were purified using NK negative selection beads (Miltenyi beads) and sequencing was performed in the MD Anderson Cancer Center (MDACC) Genomics Core and at Avera Institute for Human Genetics. Sequencing at MDACC Genomics Core was done as follows: Stranded mRNA libraries were prepared using the KAPA Stranded mRNA-Seq Kit (Roche). Briefly, PolyA RNA was captured from 250 ng of total RNA using magnetic Oligo-dT beads. After bead elution and cleanup, the resultant PolyA RNA was fragmented using heat and magnesium. First strand synthesis was performed using random priming followed by second strand synthesis with the incorporation of deoxyuridine triphosphate (dUTP) into the second strand. The ends of the resulting double stranded cDNA fragments were repaired, 5′-phosphorylated, 3′-A tailed, and Illumina-specific indexed adapters were ligated. The products were purified and enriched for full-length library with 12 cycles of PCR. The strand marked with dUTP was not amplified, resulting in a strand-specific library. The libraries were quantified using the Qubit dsDNA HS Assay Kit (Thermo Fisher) and assessed for size distribution using the 4200 Agilent TapeStation (Agilent Technologies). Equimolar quantities of the indexed libraries were then multiplexed, 12 libraries per pool. The library pool was quantified by qPCR, then sequenced on the Illumina NextSeq500 high output 150 flow cell using the 75nt paired end format. Sequencing at Avera Institute for Human Genetics was done as follows: A total of 21 isolated total RNAs were assessed for concentration and integrity on an RNA 6000 Nano chip ran on a 2100 BioAnalyzer (Agilent; Santa Clara, CA) where the average RNA integrity score (RIN) was 8.4 and the average concentration was 16.0 ng/μl. A sample input amount of 100 ng of total RNA was utilized for each sample for library preparation using the Illumina Stranded mRNA Library Prep Kit (Illumina, Inc; San Diego, CA). Briefly, polyA mRNA was captured utilizing oligo (dT) magnetic beads, fragmented appropriately, and primed for cDNA synthesis with random hexamers. Blunt-ended cDNA was generated after first and second strand synthesis where the addition of dUTP is incorporated to achieve strand specificity. Adenylation of the 3′ blunt-ends was followed by pre-index anchor ligation prior to the enrichment of the cDNA fragment with indexed primer sequences. Final library quality control was carried out by evaluating the fragment size on a DNA1000 chip ran on a 2100 BioAnalyzer (Agilent; Santa Clara, CA). The concentration of each library was determined by qPCR using the KAPA Library Quantification Kit for Next Generation Sequencing (KAPA Biosystems; Woburn, MA) prior to sequencing. The average concentration of final library was determined to be 82.8 nM. Libraries were normalized to 2 nmol/L in RSB/Tween 20 then pooled evenly. The library pool along with a 0.5% PhiX control was loaded onto Illumina's NextSeq2000 Sequencing System where denaturation and cluster generation were performed according to the manufacturer's specifications (Illumina, Inc; San Diego, CA). Sequencing-by-synthesis (SBS) was performed on a NextSeq2000 in a 2×100 fashion utilizing v3 chemistry with a P1 flow cell which resulted in an average of 24 million paired-end reads per sample. Sequence read data were processed and converted to FASTQ format for downstream analysis by Illumina BaseSpace software, BCL Convert 3.8.4.

Fastq file quality control was performed with FATSQC (https://www.bioinformatics.babraham.ac.uk/projects/fastqc/) using the R package fastqcr (https://cran.r-project.org/web/packages/fastqcr/index.html). Gene expression was quantified with RSEM ⁴⁶ (v1.3.3) (rsem-calculate-expression-strandedness reverse-no-bam-output-paired-end-bowtie2) using the bowtie2 ⁴⁷ (v2.4.2) as the aligner and hg19 transcriptome as the reference. TPM values from RSEM were log 2 transformed (log 2 (TPM+1)) and the top five thousand variably expressed genes were used to perform PCA and visualize clustering of the samples.

Differential expression analysis comparing NK cells from Opt-Cs to Sub-Cs was performed using DESeq2 ⁴⁸ with the counts imported from the output of RSEM using tximport⁴⁹. The differential expression model controlled for batch if samples in a comparison came from multiple sequencing batches. DEGs were identified at adjusted p-value<0.1 and absolute log 2 fold-change>1.5.

Differential pathway analysis was performed using GSEA, implemented in the Bioconductor gage ⁵⁰, using ordered gene lists. The gene lists were ordered by “stat” column of Deseq2's output. Differentially activated pathways were identified at q-value<0.1, with positive mean statistic indicating upregulation in Opt-Cs and negative values indicating upregulation in Sub-Cs. Enrichment plots were generated using the GSEA tool (https://www.gsea-msigdb.org/gsea/index.jsp). The hallmark 51 pathway definitions were used for GSEA.

TABLE 16
List of samples used in bulk RNA-seq and ATAC-seq analyses

| | Sample | Donor | Replicate ID | Cord quality |
|---|---|---|---|---|
| bulk RNA-seq | C2-10 | EX47-14 | 3 | optimal |
| | C2-1 | EX47-7 | 1 | optimal |
| | C2-11 | EX47-13 | 1 | optimal |
| | C2-12 | EX47-13 | 2 | optimal |
| | C2-13 | EX47-13 | 3 | optimal |
| | C2-2 | EX47-7 | 2 | optimal |
| | C2-3 | EX47-7 | 3 | optimal |
| | C2-4 | EX47-11 | 1 | optimal |
| | C2-5 | EX47-11 | 2 | optimal |
| | C2-6 | EX47-11 | 3 | optimal |
| | C2-8 | EX47-14 | 1 | optimal |
| | C2-9 | EX47-14 | 2 | optimal |
| | D2-10 | EX47-15 | 2 | optimal |
| | D2-11 | EX47-15 | 3 | optimal |
| | D2-3 | EX47-16 | 1 | optimal |
| | D2-4 | EX47-16 | 2 | optimal |
| | D2-5 | EX47-16 | 3 | suboptimal |
| | D2-6 | EX47-17 | 1 | suboptimal |
| | D2-7 | EX47-17 | 2 | suboptimal |
| | D2-8 | EX47-17 | 3 | suboptimal |
| | D2-9 | EX47-15 | 1 | suboptimal |
| | I1 | EX29-1 | 1 | suboptimal |
| | I2 | EX29-1 | 2 | suboptimal |
| | I3 | EX29-2 | 1 | suboptimal |
| | I4 | EX29-2 | 2 | suboptimal |
| | I5 | EX29-2 | 3 | suboptimal |
| | I7 | EX29-4 | 1 | suboptimal |
| | B11 | EX28-1 | 1 | suboptimal |
| | B21 | EX28-1 | 2 | suboptimal |
| | B31 | EX28-3 | 1 | suboptimal |
| | S4 | EX27-4 | 1 | optimal |
| | S5 | EX27-4 | 2 | optimal |
| Total | 32 | 16 | | |
| Bulk ATAC-seq | Sample_O4 | EX27-2 | 1 | optimal |
| | Sample_O5 | EX27-2 | 2 | optimal |
| | Sample_Ple_O7 | EX27-4 | 1 | optimal |
| | Sample_Ple_O8 | EX27-4 | 2 | optimal |
| | Sample_Ple_O9 | EX27-4 | 3 | optimal |
| | Sample_Ple_X1 | EX27-4 | 4 | optimal |
| | Sample_Ple_X2 | EX28-1 | 1 | optimal |
| | Sample_X7 | EX29-1 | 1 | optimal |
| | Sample_O1 | EX27-1 | 1 | suboptimal |
| | Sample_O2 | EX27-1 | 2 | suboptimal |
| | Sample_O3 | EX27-1 | 3 | suboptimal |
| | Sample_O6 | EX27-3 | 1 | suboptimal |
| | Sample_X3 | EX28-3 | 1 | suboptimal |
| | Sample_X4 | EX28-3 | 2 | suboptimal |
| | Sample_X5 | EX28-4 | 1 | suboptimal |
| | Sample_X6 | EX28-4 | 2 | suboptimal |
| | Sample_X8 | EX29-4 | 1 | suboptimal |
| Total | | 17 | 7 | |

NK functional score-Activity of NK function signature (e.g., GZMA, PRF1, GZMB and CD247) was estimated in each sample using ssGSEA 52 implemented in the R package GSVA 53. Difference between Opt-Cs and Sub-Cs was computed using two tailed Student's t-test.

Bulk RNA-seq regulon analysis—To identify key TFs and measure the activity of regulons in bulk RNA-seq data, the inventors utilized the python implementation of the SCENIC (pySCENIC) workflow described previously 54. The default pySCENIC parameters were applied on a high-performance computing system to infer regulatory interactions between pre-defined lists of TFs and candidate target genes. pySCENIC utilized gradient boosting machine regression GRNBoost2 algorithm and Arboreto library 55 to calculate co-expression patterns from transcriptomics data. This resulted in an adjacencies matrix connecting each TF with its target gene(s) along with an importance score which separates high confidence interactions from the weak ones. To generate candidate modules, TF-target gene interactions were selected and assembled into modules consisting of target genes that would be regulated by a given TF, also referred to as regulons. These modules were further refined by separating the direct targets of a given regulator from the indirect ones. This was achieved by identifying target genes that have the DNA motif specific to a certain TF in their promoter region. To do this, cis-regulatory module scoring with RcisTarget was utilized, which looks for modules with cisTarget motif enrichment using pre-computed whole-genome rankings of all motifs linked to known TFs in the pySCENIC database. The area under the curve (AUC) scores were then calculated to measure the biological activity of each regulon at the sample level. The inventors identified differentially active regulons in NK cells between Opt-C and Sub-C samples at the pre-stimulation time point using a t-test, which was corrected for multiple hypothesis testing using Bonferroni correction. An adjusted p-value<0.01 was used to display statistically significant hits on the scaled regulon activity scores and compare different conditions.

Bulk ATAC-seq analysis-ATAC-seq library preparation was performed at the MD Anderson Cancer Center Epigenomics Profiling Core following the protocol previously described ^{56,57} with minor modifications. Briefly, nuclei isolated from NK cells derived from nine donors were tagmented using Tagment DNA enzyme (Illumina) and the resulting libraries were purified using SPRISelect beads (Beckman Coulter). Libraries were sequenced 2×100 bp on an Illumina NovaSeq6000 to obtain at least 50 million high quality mapping reads per sample.

For each bulk ATAC-seq sample, the pair-end reads from fastq files were aligned to the human genome (GRCh38) using bwa mem mode with duplicated reads removed ⁵⁸ The 5′ end of ATAC-seq reads were shifted to the actual cut-site of the Transposase using alignmentSieve module implemented in DeepTools ⁵⁹. The peaks were called using MACS2 ⁶⁰ using the pair-end read information. The samples have comparable total number of reads: mean=1.05×10⁶, s.d.=0.11×10⁶ in the Sub-C samples and mean=1.19×10⁶, s.d.=0.07×10⁶ in the Opt-C samples. The minimum FDR (q-value) cutoff for peak detection was set as 0.05. The MACS2 outputs from multiple samples were loaded using DiffBind ⁶¹. The peak sets from multiple samples were identified as the overlapping ones among samples using bUseSummarizeOverlaps function in DiffBind. The TF activity level was then calculated using the function RunChromVAR in Signac ⁶² and gene-level accessibility level using the geneActivity function in Seurat. The difference between Opt-Cs and Sub-Cs on peak, gene accessibility and motif-based TF activity levels was identified using function FindMarkers in Signac ⁶². The peak track profiles of candidate genes were visualized using the online IGV tool (for replicates from the same group, the peak track profiles were aggregated together).

Viral constructs and retrovirus production—The CAR targeting CD70 construct (iC9.CD27 (ECD).CD28.zeta.2A.IL-15) referred to as CAR70/IL-15 incorporated the CD27 extracellular domain (which naturally binds to CD70), linked to the CD28 costimulatory domain and CD3ζ(CD3z) signaling domain. Additionally, it included inducible caspase 9 (iC9) as a safety switch and the interleukin 15 (IL-15) transgene.

The CAR targeting TROP2 construct (iC9.TROP2scFv (clone hRS7).CD28.zeta.2A.IL-15) referred to as CAR-TROP2/IL-15 consisted of an scFv targeting TROP2 (derived from the human RS7 [hRS7] sequence of the TROP2-targeting antibody-drug conjugate Sacituzumab govitecan), coupled with the CD28 costimulatory domain and CD35 signaling domain. Similarly, the construct included iC9 as a safety switch and IL-15.

The CAR70/IL-15 and CAR-TROP2/IL-15 constructs were cloned into the SFG retroviral backbone to generate viral vectors. Transient retroviral supernatants were produced from transfected 293T cells as previously described 69.

Xenogeneic tumor-grafted mouse models-NOD/SCID IL-2Rγnull (NSG) mice engrafted with different tumor cell lines were used to examine the anti-tumor activity of the different CAR NK-cell products. Tumor models included Raji lymphoma, MMIS multiple myeloma, and SKOV3 ovarian cancer. All experiments were performed in accordance with American Veterinary Medical Association (AVMA) and NIH recommendations under protocols approved by the MD Anderson Cancer Center Institutional Animal Care and Use Committee (protocol number 00000889-RN02). Mice were maintained under specific-pathogen-free conditions, with a 12-hour night/day cycle of light, and at a stable ambient temperature with 40-70% relative humidity. The inventors utilized an aggressive NK-resistant Raji NSG (The Jackson Laboratory, Bar Harbor, ME) xenograft model. Ten-week-old male mice were irradiated on day-1 and engrafted with Ffluc-Raji cells (0.2×10⁵). CAR19/IL-15 CB-NK cells from Opt-Cs or Sub-Cs were injected via tail vein when indicated. Weekly bioluminescence imaging (Xenogen IVIS-200 Imaging System) was performed to monitor tumor growth. Flow cytometry was used to measure NK cell trafficking, persistence, and expansion. The inventors utilized a second mouse model of MMIS to validate the results. Ten-week-old female mice were irradiated on day-4 and engrafted with Ffluc-MMIS (5×10⁵) on day-3. CAR70/IL-15 transduced CB-NK cells from Opt-Cs or Sub-Cs were injected via tail vein when indicated. Mice were subjected to weekly bioluminescence imaging. Trafficking, persistence and expansion of NK cells were measured by flow cytometry. For the ovarian cancer model SKOV3, nine-week-old female mice were injected with Ffluc-SKOV3 (5×10⁵) on day-7 intraperitoneally and mice were irradiated on day-1. CAR-TROP2/IL-15 transduced CB-NK cells from Opt-Cs or Sub-Cs were injected intraperitoneally on day 0. Mice were subjected to weekly bioluminescence imaging (Xenogen IVIS-200 Imaging System).

Statistical methods—The statistical rationale for the sample size of patients enrolled on the trial was not based on a power computation, rather, the reliability of Bayesian posterior estimators of Probably (efficacy) and Probability (toxicity) were quantified by assuming a noninformative prior for each probability and computing a posterior 95% credible interval (CrI). Probabilities of 1-year OS and PFS were calculated using the Kaplan-Meier method. For the PFS analysis, death for any reason, progression of the disease or loss of a previously achieved response were considered as the events of interest. Survival times were censored at last patient follow-up. The influence of variables on the proportion of day+30 OR or 1-year CR was examined with the Fisher's exact test. OS and PFS were compared using the log-rank test. Bayesian methods were used for multiple regression ^{70,71}. For regression of binary outcomes on patient covariates, a logistic model was assumed. For regression of each outcome on patient covariates, independent non-informative normal (0, 10) priors were assumed for all covariate parameters. In each regression model, the effect of each covariate with coefficient b on the outcome was quantified by the posterior probability of a beneficial effect, PBE=Pr (b>0|data). A PBE near 0 implied a very harmful effect of the covariate on the outcome, a PBE near 1 implied a very beneficial effect of the covariate, and PBE=0.50 corresponded to no effect. For PFS or OS, PBE=Pr (HR<1| data)=the probability of a lower risk of the failure event for the covariate. The inventors used the Wilcoxon rank-sum or the Kruskal-Wallis test to study the association between the copy number by qPCR of CAR-NK cells and other variables. The Student's t-test, one-way ANOVA and two-way ANOVA were used for the in vitro and in vivo mouse studies as indicated. For comparison of survival curves in the mouse experiments, the Kaplan-Meier method and log-rank test were used. There were 10-15 death events observed in each mouse experiment and this analysis predicted at least 80% power to detect a relative hazard ratio of 4.3-6 between two groups at the significance level of 0.5. All reported p-values were two sided and p-values of less than 0.05 were considered significant. The analyses were performed using SPSS Version 26.0, R version 4.2.1, JAGS version 4.3.1, and GraphPad Prism version 7.0.

### Example 2-Clinical Trial Study Design, Patients, and Responses

The Inventors conducted a phase I-II clinical trial at MD Anderson Cancer Center that was designed to assess the safety and efficacy of escalating doses of iC9/CD19-CAR/IL15 CB-NK cells (“CAR19NK”) as treatment for relapsed/refractory CD19-positive malignancies. 37 patients with CD19-positive malignancies (see Table 1) were treated between June 2017 and June 2021 and then followed for 12 months after the CAR-NK cell infusion. The study had two phases, a dose escalation phase that was previously reported, and an expansion phase. In the dose escalation phase (n=11), patients received lymphodepleting chemotherapy with fludarabine 30 mg/m² and cyclophosphamide 300 mg/m² daily for 3 consecutive days followed by the infusion of iC9/CD19-CAR/IL15 CB-NK cells at escalating doses of 10⁵ cells/kg, 10⁶ cells/kg, and 10⁷ cells/kg. The dose was escalated using the EfftoxEfftoxT design. In the expansion phase (n=26), additional patients were treated at the 10⁷ cells/kg iC9/CD19-CAR/IL15 CB-NK dose level. A second infusion separated one week apart was allowed at the discretion of the investigator for patients with bulky or rapidly progressive disease (n=7). Then the trial was amended to include a second expansion cohort (N=15) where patients received a single flat dose of 8×10⁸ iC9/CD19-CAR/IL15 CB-NK cells (the equivalent of 10⁷ cells/kg for an 80 kg person). The first 9 patients in the Phase I portion of the study received a CAR-NK product that was partially matched with the recipient (4/6 HLA molecules: HLA-A, B and DRβ1); the protocol was then amended to permit selection of cords with no consideration for HLA matching. Table 1 shows the patient and cell product characteristics. Clinical responses to therapy for CLL and NHL were based on the Lugano and iWCLL 2018 criteria, respectively [see e.g., Cheson, B. D., et al., Recommendations for initial evaluation, staging, and response assessment of Hodgkin and Non-Hodgkin Lymphoma: The Lugano Classification. Journal of Clinical Oncology (2014); and Hallek, M., et al., iwCLL guidelines for diagnosis, indications for treatment, response assessment, and supportive management of CLL. Blood Special Report (2018); each of which are incorporated herein in their entirety by reference for the purposes described herein). Objective or overall response (OR) represents the combination of partial response (PR) and complete response (CR).

The study was approved by the institutional review board and conducted according to the declaration of Helsinki. Written informed consent was obtained from each patient. The trial was registered on the clinicaltrials.gov website (NCT03056339). The clinical protocol and additional information on the study design are available in the Supplementary appendix thereof. Safety and efficacy (defined as day 30 overall response) were the primary objective of the trial. No significant toxicities were observed, particularly no cytokine release syndrome or neurotoxicity. None of the patients developed neurotoxicity or graft-versus-host disease and only one developed CRS (grade I). Lymphodepleting chemotherapy caused reversible hematological toxicity in all patients (see e.g., Table 3), and the maximum tolerated dose was not reached.

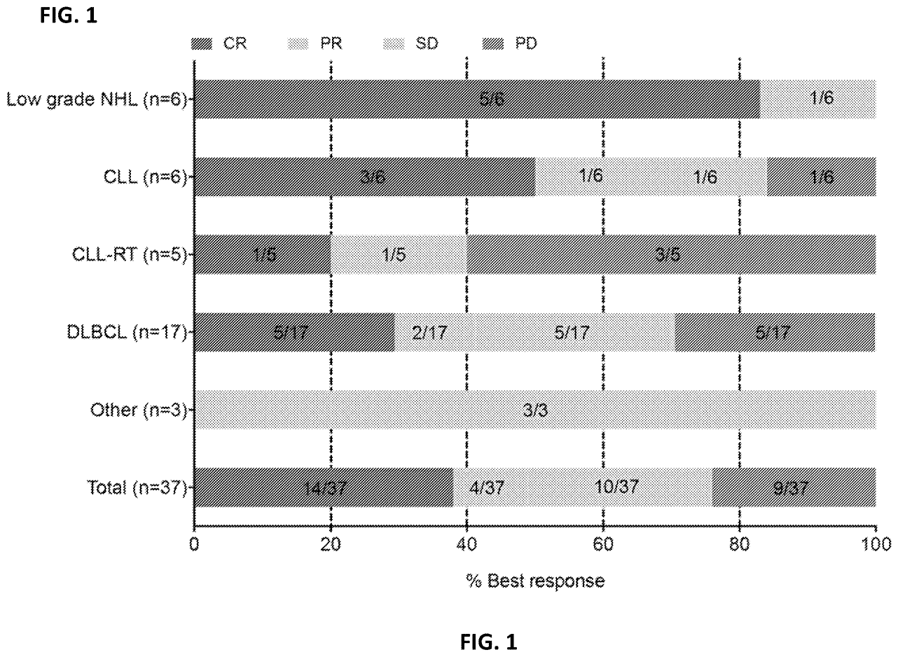

Secondary objective of this trial included progression free survival, overall survival, and CAR19/IL-15 persistence. The study enrolled 37 patients with multiple B-cell malignancies, such as CLL, CLL (including Richter's transformation), DLBCL, follicular lymphoma, marginal cell lymphoma, high grade lymphoma, acute lymphoblastic leukemia, mantle cell lymphoma, and plasmacytic lymphoma (Table 1). The day 30 OR and 1-year best complete response (CR) rates for patients with low grade NHL were 100% and 83% respectively (n=6), for CLL without transformation were 67% and 50% (n=6), for DLBCL 41% and 29% (n=17), and for CLL with Richter's transformation 20% and 20% (n=5), respectively. FIG. 1 summarizes the patient responses. Table 2 shows responses according to patient and disease characteristics.

Responses were seen at similar proportions in all four dose levels with no apparent evidence of a dose effect. Responses were rapid with a median time to first response of 30 days (range 30-55 days). The responses were durable; nine of the 10 patients who had achieved a CR by day+30 remained in CR at day+180. Furthermore, four of eight patients who achieved a PR at day+30 eventually achieved a CR. For patients who achieved CR at 30 days the probability to remain in CR at 12 months was 70.0% (CI=39.7-89.2). Post-remission therapy was permitted after the day 30 assessment at the investigator's discretion only in the phase I part of the study. Four patients received consolidation including hematopoietic stem cell transplantation (n=2) or maintenance therapy with lenalidomide or rituximab (n=2) after achieving CR. Two additional patients received targeted or immunomodulatory therapy after achieving PR. Namely, a patient with Richter's transformation of CLL achieved complete remission of the high grade lymphoma after the CAR-NK infusion but had persistent CLL and received venetoclax archiving CR; a second patient with DLBCL achieved PR after the cell infusion then received lenalidomide and achieved CR. No patients received additional therapy in the dose expansion part of the study.

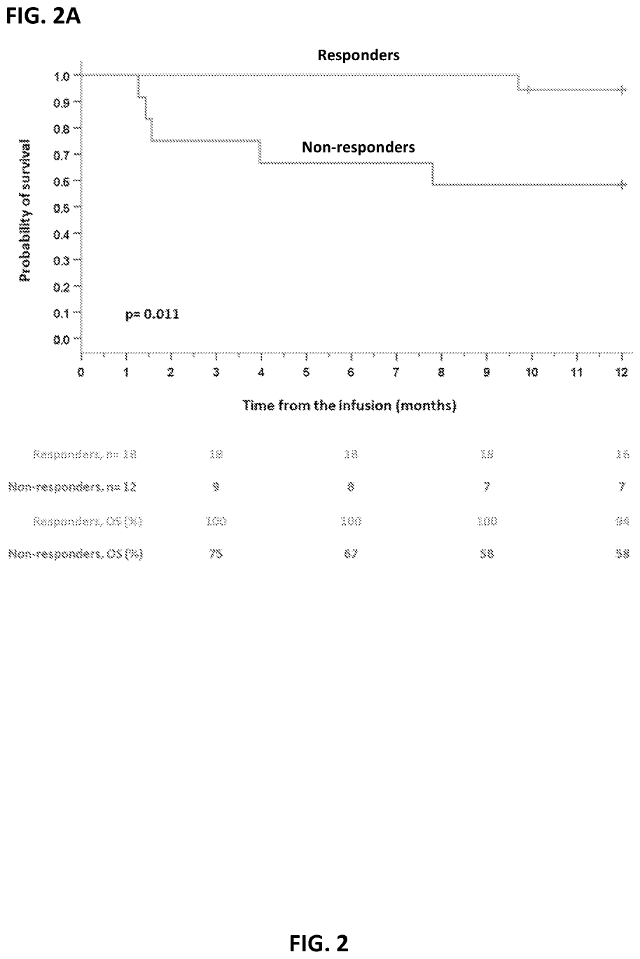

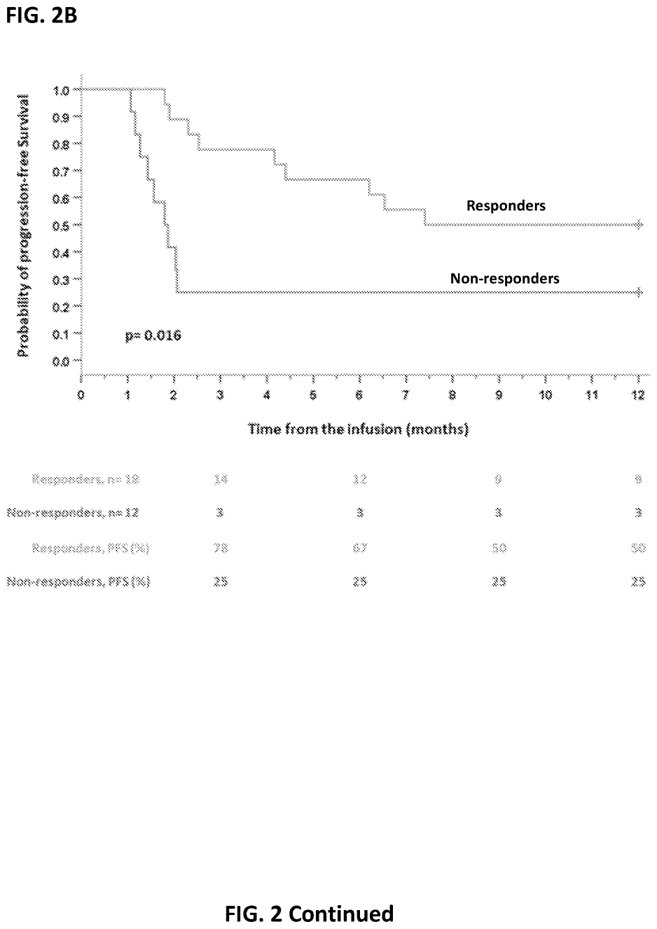

The inventors performed a landmark analysis in order to assess the effect of response on OS and PFS (excluding the 7 patients who had progressed prior to day 30). The 18 patients who had achieved an OR at day+30 after the CAR-NK infusion had significantly superior 1-year probabilities of OS (94.4% vs 58.3%, p=0.01) and PFS (50.0% vs 25.0%, p=0.016) when compared to the 12 patients who had failed to respond (FIGS. 2A-2B). When the outcome of patients with DLBCL were separately analyzed, it was confirmed that the 7 responding patients had a significantly higher 1-year PFS than the 6 non-responders (57.1% vs 16.7%, p=0.02). These results suggested that day+30 responses were associated with longer term superior survival and progression-free survival.

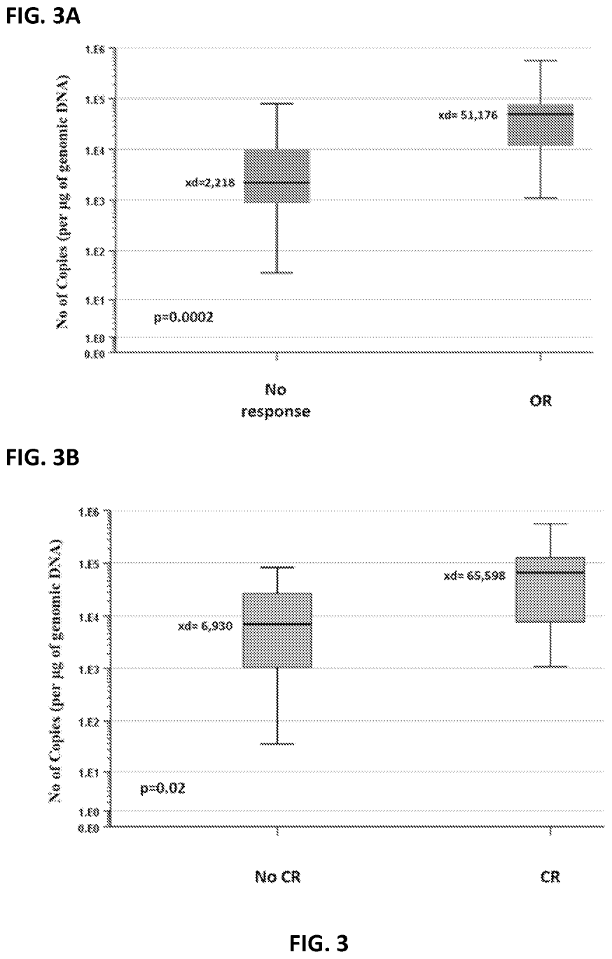

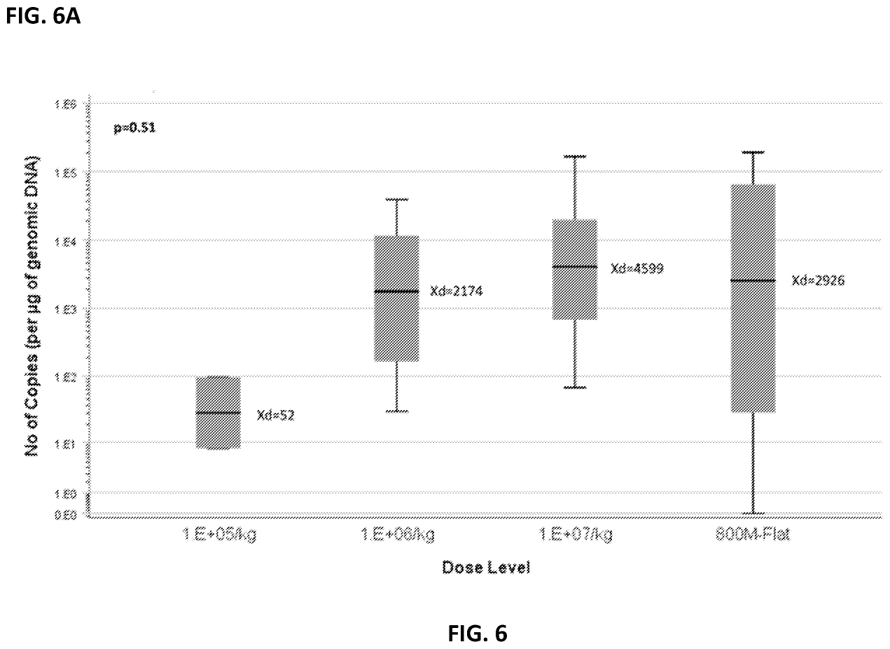

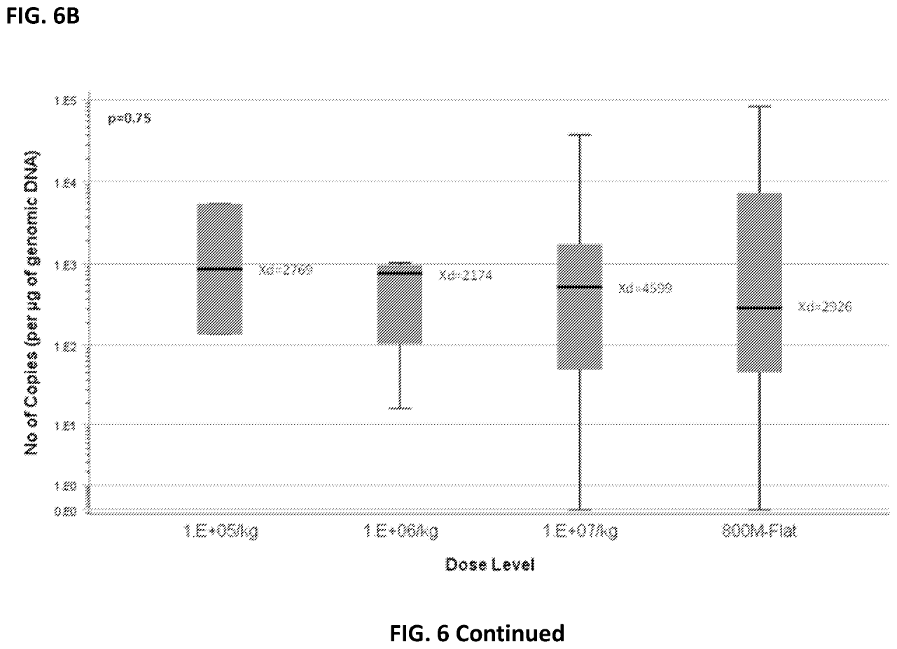

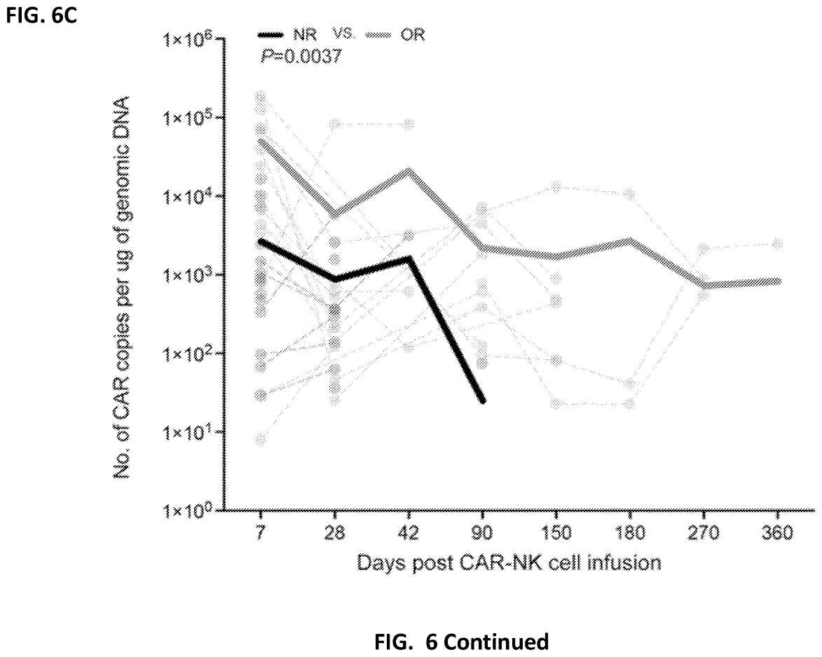

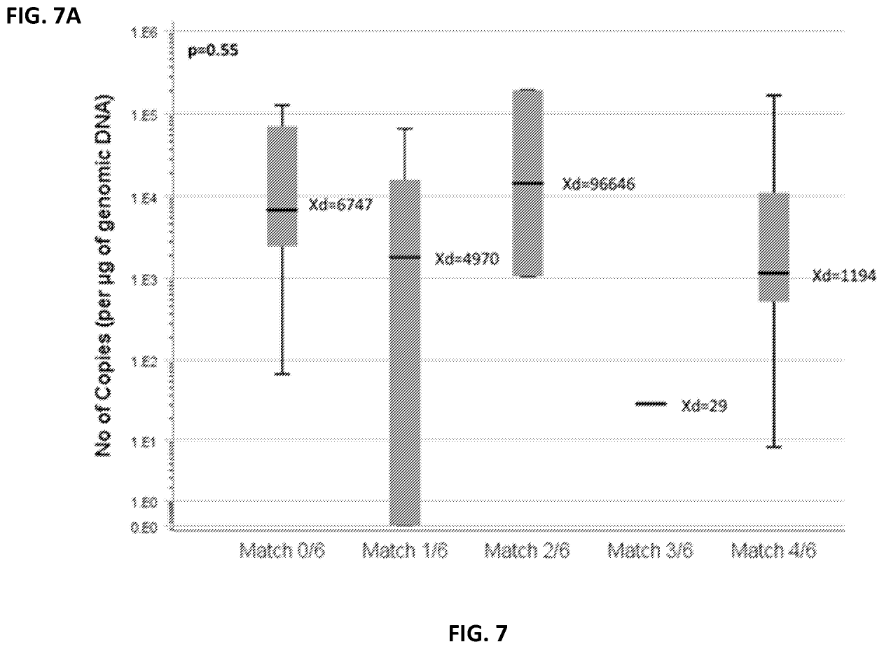

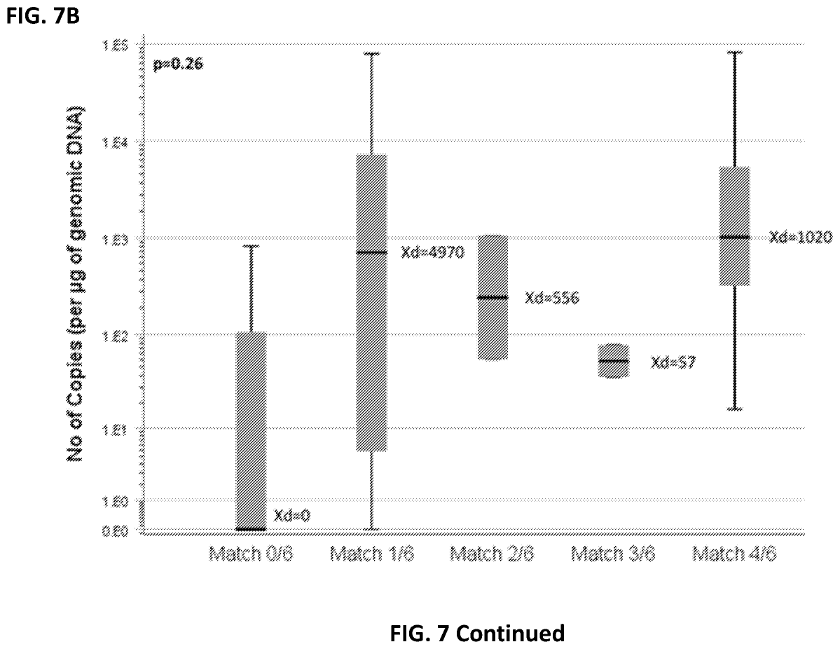

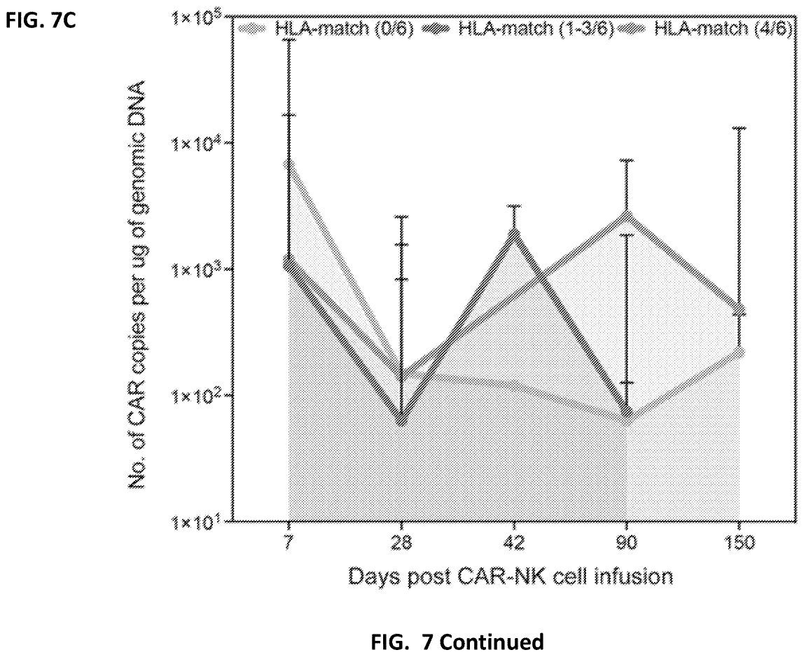

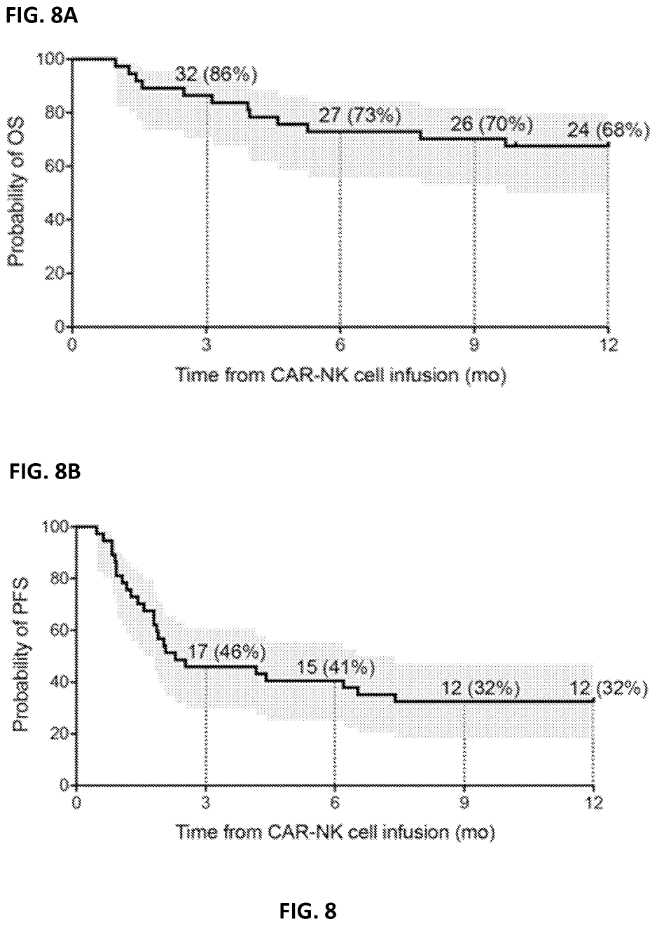

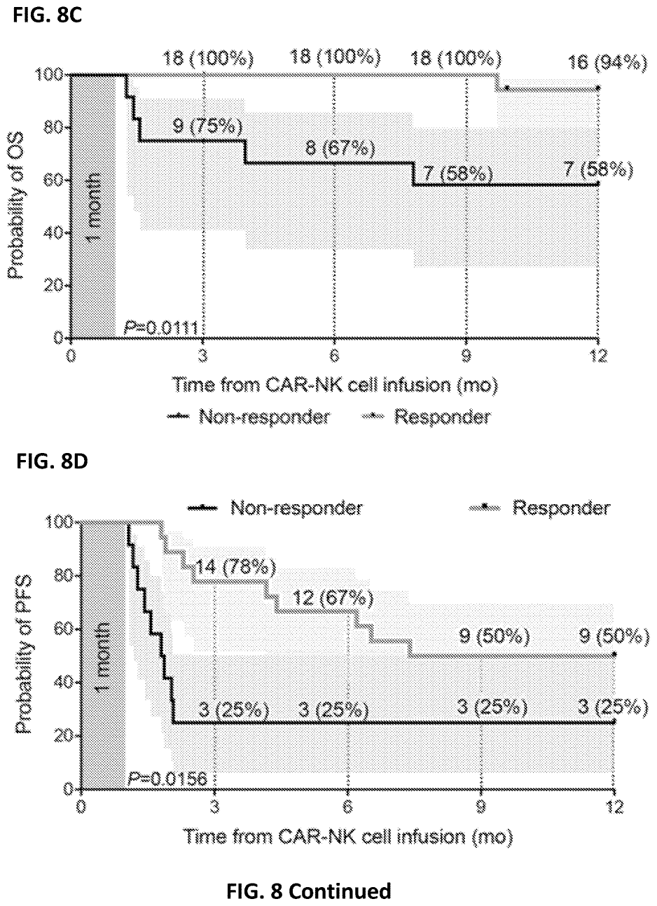

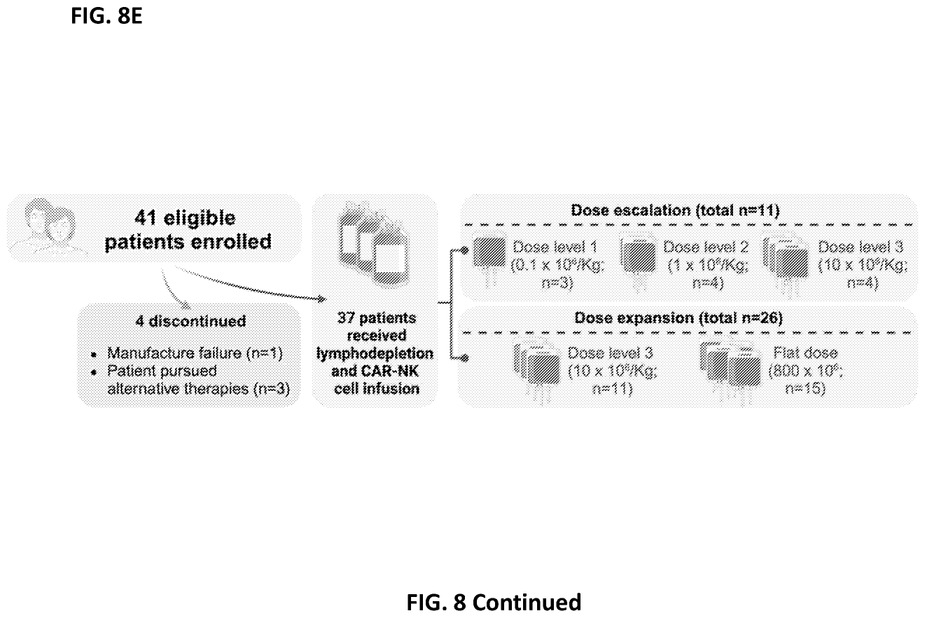

The inventors monitored the in vivo expansion of CAR-NK cells in peripheral blood samples collected at multiple timepoints post-infusion using qPCR. The median time to maximal expansion was 13.5 days (range 3-280 days). Beyond day+14, a statistically significant relationship between dose of CAR cells infused and the peak transcript number in the peripheral blood was not observed, supporting an important role for IL-15 in driving in vivo expansion of CAR-NK cells (FIGS. 6A-6B). As previously reported (see e.g., Enli Liu et al., 2020), the inventors did not observe a difference in the persistence of CAR-NK cells according to the degree of HLA mismatch with the recipient (FIGS. 7A-7B). Patients with a higher peak of CAR-NK cells in the first 28 days post-infusion had a significantly higher probability of achieving day 30 OR, or day 30 CR when compared to those with poor CAR-NK cell expansion (FIGS. 3A-3B). These results suggested that in vivo CAR-NK cell expansion was associated with clinical responses.

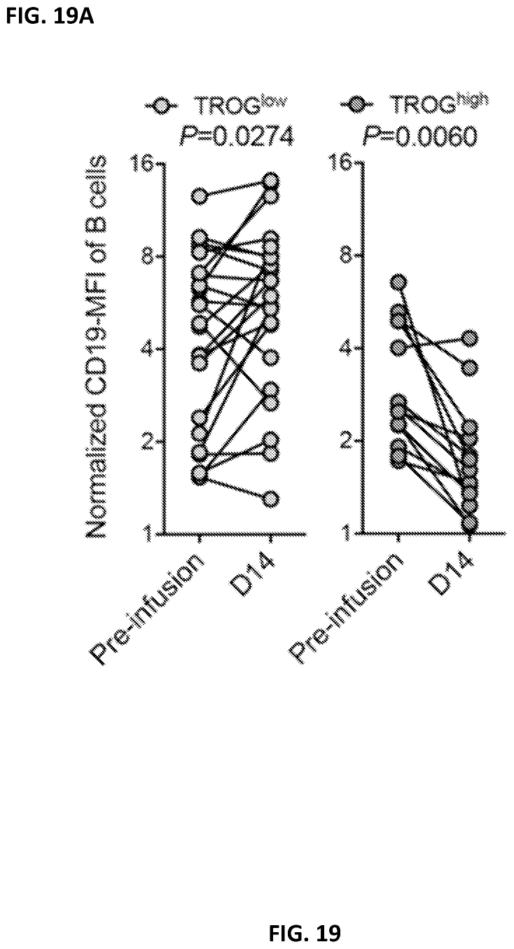

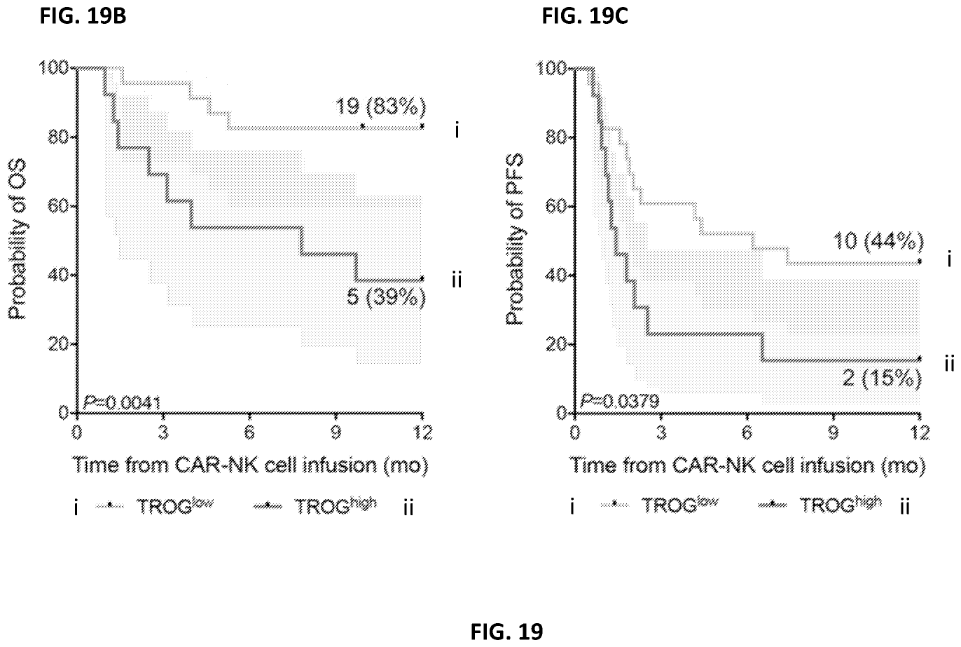

The inventors monitored trogocytosis and determined that acquisition of CD19 expression on CAR-NK cells by trogocytosis was associated with worse outcomes. Trogocytosis is a mechanism of relapse after cell therapy ^{19,20}. Thus, as a post-hoc analysis, the inventors investigated the impact of trogocytosis on outcomes by measuring CD19 levels on CAR-NK cells in PB samples from patients in the first four weeks after infusion ²⁰. Following CAR-NK cell infusion, a significant reduction in CD19 expression on B cells from patients in the trogocytosis high group (n=13) was observed when compared to those in the trogocytosis low group (n=23) (FIG. 19A). The 13 patients with high trogocytosis had a worse 1-year OS (38.5% vs. 82.6%, p=0.0041), PFS (15.4% vs. 43.5%, p=0.0379) and CR rate (7.7% vs. 56.5%, p=0.005) than the 23 patients with low levels (FIGS. 19B-19C). Additionally, trogocytosis was observed in a significantly greater proportion of patients treated with CAR-NK cells derived from Sub-Cs (TROG^{high}: 60% (12/20), p=0.001) when compared to those receiving CAR-NK cells derived from Opt-Cs (TROG^{high}: 6.3% (1/16)). Trogocytosis was not included in the multivariate analysis as it was evaluated as a post-hoc analysis.

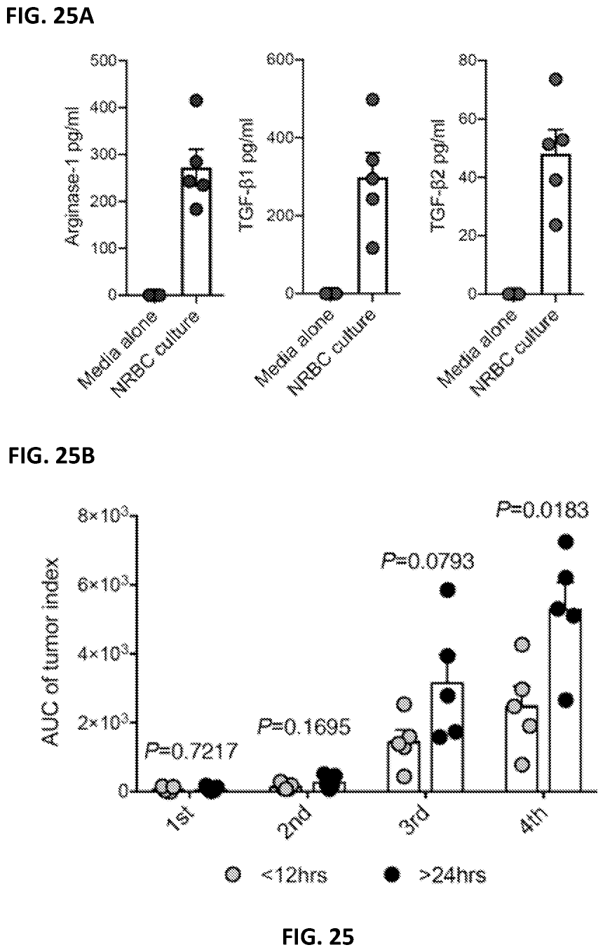

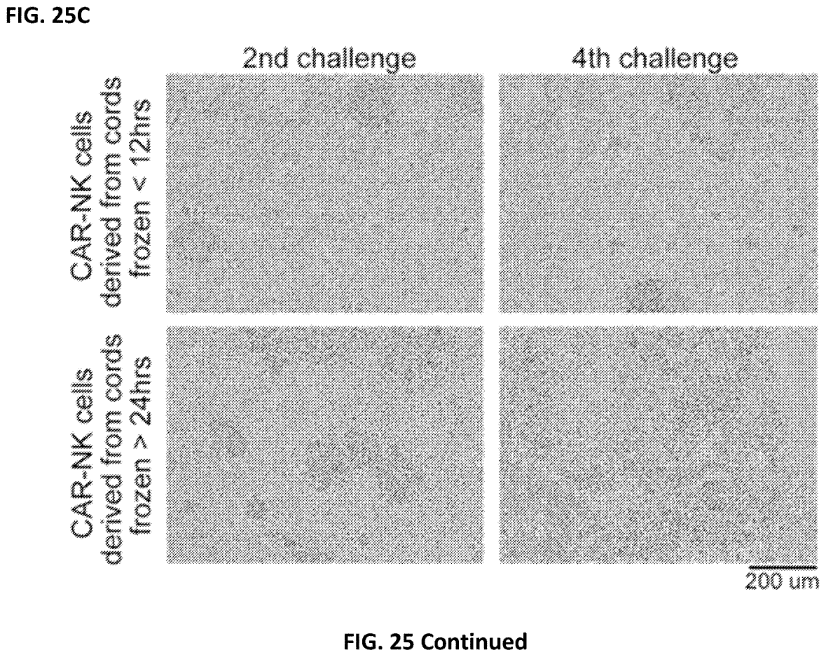

The inventors noted that NRBCs in CB units have been reported to be an indicator of fetal hypoxia and stress ^{21,22}, both of which were factors that could potentially impact NK cell functionality ^{23,24}. Additionally, NRBCs have been shown to exert immunoregulatory function by releasing immunosuppressive factors ²⁵⁻²⁷. In line with previous reports, the inventors confirmed that NRBCs isolated from CB units released significant levels of arginase-1, TGF-β1, and TGF-β2 (FIG. 25A).

To determine the influence of time from collection-to-cryopreservation on NK cell function, CB units with NRBC counts below the threshold level of ≤8×10⁷ were each divided into two equal fractions after collection. The first fraction (Fraction A) was cryopreserved within 12 hours of collection, while the second fraction (Fraction B) was cryopreserved within 24-48 hours of collection. The cord fractions were thawed and processed simultaneously. CAR19/IL-15 NK cells were generated using the inventors standard protocols and the anti-tumor efficacy of the CAR19/IL-15 NK cells was tested in a tumor rechallenge assay in vitro. CAR19/IL-15 NK cells derived from Fraction A exerted significantly better long-term cytotoxicity against Raji cells than those from the paired Fraction B (FIGS. 25B-25C). Collectively, these experimental data supported the immunosuppressive role of higher NRBCs and the negative impact of longer time from collection-to-cryopreservation on CAR-NK cell function.

After the infusion of the CAR-NK cells, none of the patients developed neurotoxicity or HLH and only one patient developed CRS (grade I). In spite of the HLA mismatch between the CAR NK cells and the recipient, there were no cases of graft-vs-host disease. After the lymphodepleting chemotherapy all patients had reversible hematological toxicity as expected. There were no cases of tumor lysis syndrome or grade IV extra-hematological toxicity. In general, the toxicity profile was mild (see Table 3) and no patient was admitted to an intensive care unit for the management of side effects related to the study. The maximal tolerated dose was not reached. Given the absence of serious toxicity, no patient received rimiducid to activate the caspase-9 safety switch to eliminate the CAR-NK cells. In addition, none of the patients required therapy with anti-IL6 or anti-IL1β blockade or corticosteroids. In view of the excellent safety profile, the treatment was moved to an outpatient setting.

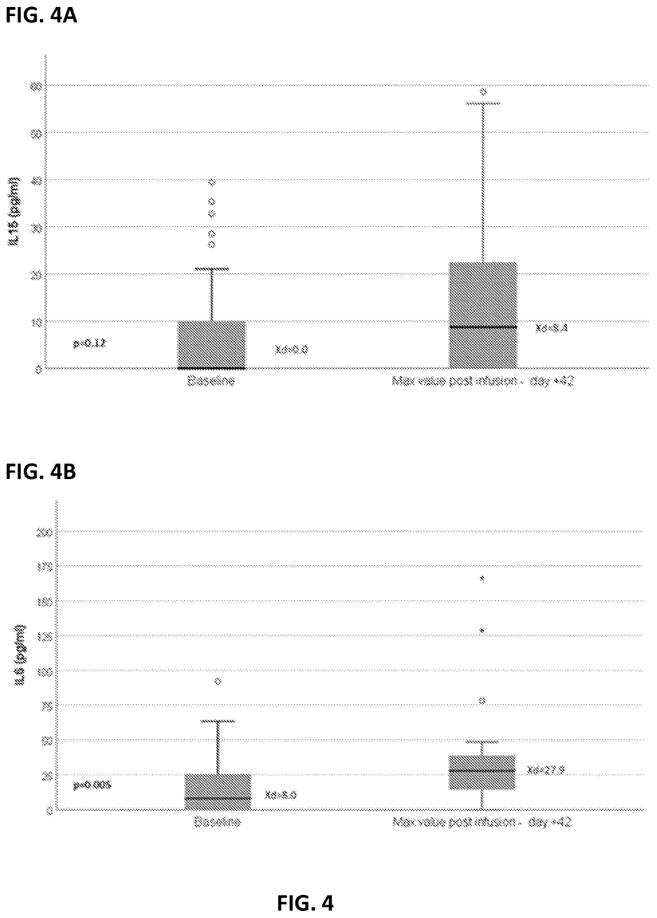

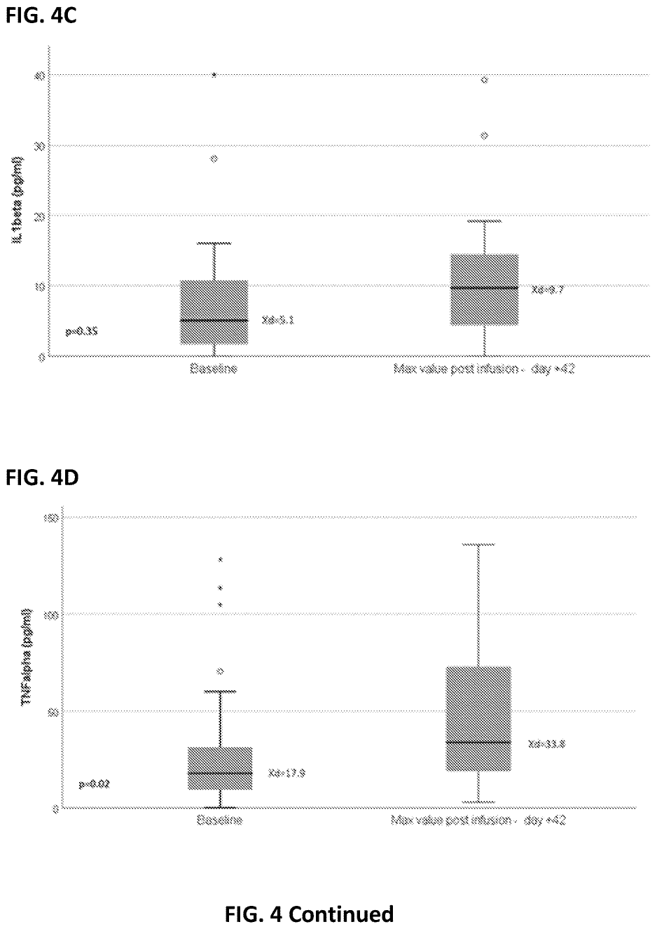

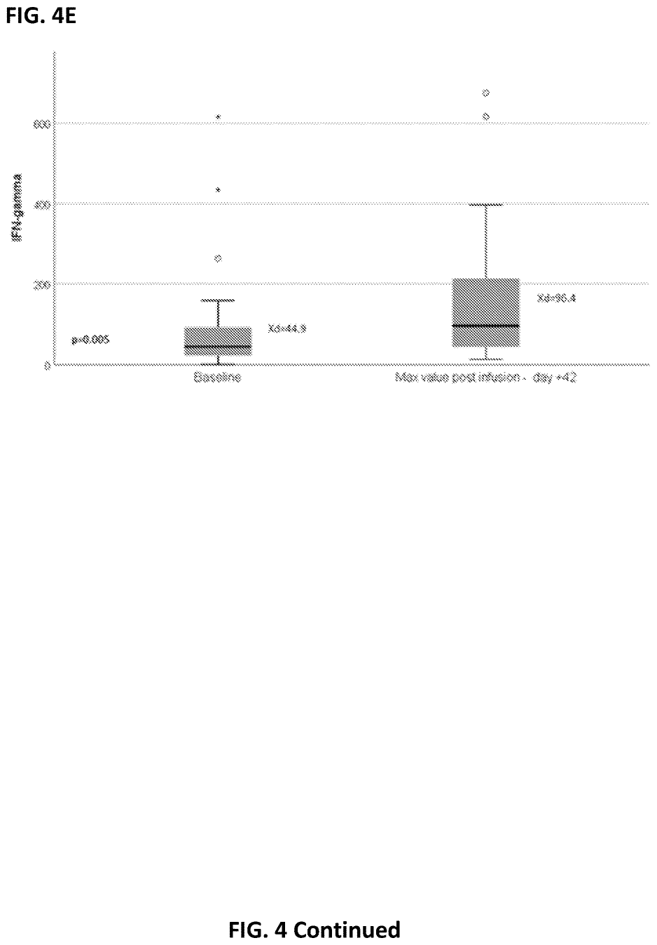

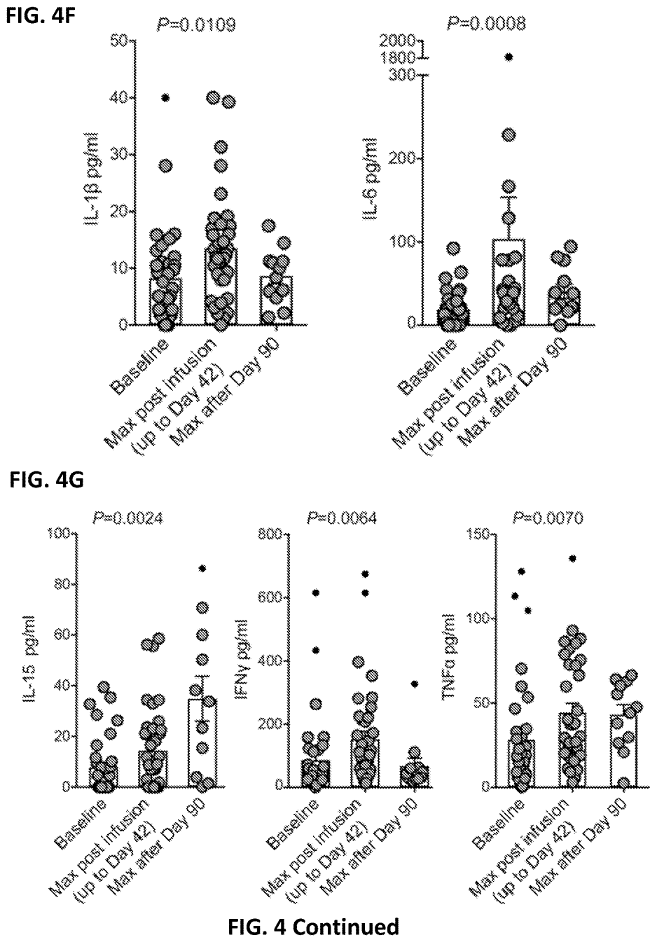

The induction of donor HLA specific antibodies (DSA) was also monitored. DSA were detected in only two of 37 patients at 27 and 124 days after the infusion. Neither patient had evidence of anti-HLA antibodies at baseline. DSAs were detected against HLA-Cw6 at a mean fluorescent intensity (MFI) of 7,132 approximately 4 weeks after infusion for one patient and against HLA-B44 at an MFI of 17,060 around 4 months following infusion for the second patient. Both patients achieved CR and had detectable CAR-NK cells by PCR after the acquisition of the antibodies. In keeping with the clinical safety profile, significant elevation (e.g., to levels associated with CRS) of inflammatory cytokines in the sera of patients collected at multiple timepoints post infusion was not observed, a modest elevation of IL-6, IL-1β and other cytokines over baseline was observed in the sera of patients post-infusion (FIGS. 4A-4E).

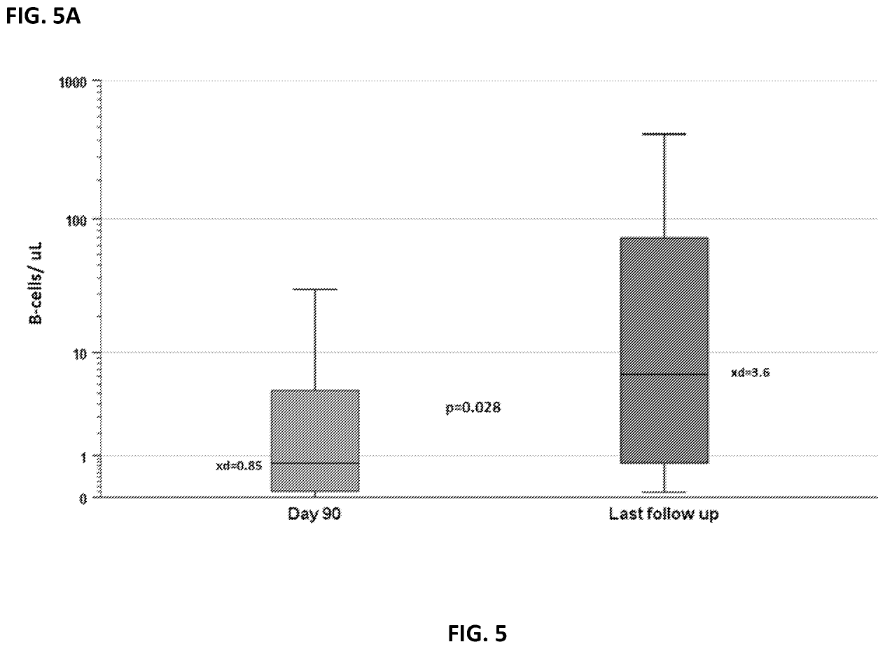

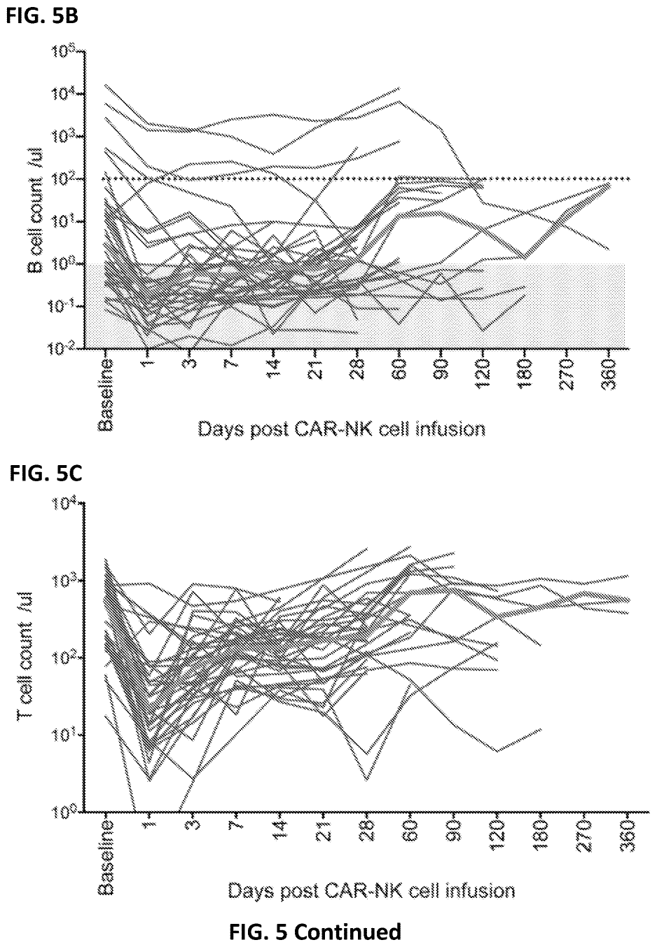

The inventors utilized flow cytometry to measure the frequencies of CD19 positive B-cells in the peripheral blood of patients after CAR-NK infusion. After lymphodepleting chemotherapy, all patients had evidence of B cell aplasia. B-cell aplasia was used as a surrogate for CAR19 T-cell activity. The inventors measured the frequencies of CD19-positive B cells in the peripheral blood of patients post CAR-NK cell treatment. At the time of enrollment, the majority of patients (31/37) had B-cell lymphopenia (B cell count<100/μl) secondary to prior B-cell targeting therapies. This number further declined, with B cells becoming nearly undetectable by flow cytometry after CAR-NK cell infusion. However, over time, there was a gradual and modest increase in the B-cell count for most patients (FIGS. 5A-5B). Among patients with available immunoglobulin G (IgG) measurements, one third had evidence of hypogammaglobulinemia (IgG<400 mg/dl) within the first 90 days following CAR-NK cell infusion. Of note, the T-cell count followed an expected trajectory, with a drop following lymphodepleting chemotherapy followed by recovery (FIG. 5C).

TABLE 1
Patient, disease, donor cord blood
and CAR-NK characteristics
Notations: 1, Four patients had follicular lymphoma
and 2 patients had marginal zone lymphoma;
2, Only for NHL patients

| Variable | n (%) | |
|---|---|---|
| Patient characteristics | | |
| Age | | |
| median (range) | 64 | (26-79) |
| <65 years | 19 | (51.4) |
| ≥65 years | 18 | (48.6) |
| Sex | | |
| Male | 24 | (64.9) |
| Female | 13 | (35.1) |
| Race | | |
| Caucasian | 31 | (83.8) |
| Hispanic | 0 | (0.0) |
| Black | 1 | (2.7) |
| Asian | 2 | (5.4) |
| Other | 3 | (8.1) |
| Weight (kg) | 78 | (47-140) |
| median (range) | | |
| Diagnosis | | |
| Indolent lymphoma *¹ | 6 | (16.2) |
| CLL | 6 | (16.2) |
| CLL-RT | 5 | (13.5) |
| DLBCL | 17 | (45.9) |
| Mantel cell lymphoma | 1 | (2.7) |
| Acute lymphoblastic leukemia | 1 | (2.7) |
| Lymphoplasmacytic lymphoma | 1 | (2.7) |
| Karnofsky | | |
| Median (range) | 90 | (80-100) |
| LDH at conditioning chemotherapy | | |
| Median (range) | 259 | (141-1765) |
| LDH>UNL | 24 | (64.9) |
| Number of prior therapies per patient | | |
| Median (range) | 4 | (2-10) |
| ≥3 prior lines of therapy | 31 | (83.8) |
| Prior Stem Cell Transplant | | |
| Autologous | 9 | (24.3) |
| Allogeneic | 1 | (2.7) |
| Disease status | | |
| Relapsed | 11 | (29.8) |
| Refractory to most recent therapy | 16 | (43.2) |
| History of primary refractory disease | 10 | (27.0) |
| Disease stage *² | | |
| I or II | 8 | (36.4) |
| III or IV | 14 | (63.6) |
| CAR-NK characteristics | | |
| Dose level | | |
| 1 × 10⁵ cells/kg | 3 | (8.1) |
| 1 × 10⁶ cells/kg | 4 | (10.8) |
| 1 × 10⁷ cells/kg | 15 | (40.5) |
| 8 × 10⁸ cells | 15 | (40.5) |
| flat dose | | |
| NK cell fold expansion days 0-15 | | |
| median (range) | 953.0 | (14.9-7630.0) |
| 25% percentile | 450.2 | |
| Mean Fluorescence Intensity | | |
| median (range) | 12975 | (4836-36328) |
| 25% percentile | 9343.5 | |
| Transduction efficiency (%) | | |
| median (range) | 72.4 | (22.7-91.1) |
| 25% percentile | 52.8 | |
| CD3+ T-cells infused/Kg | 2000 | (30-16000) |
| median (range) | | |
| CBU characteristics | | |
| HLA Allelic Match | | |
| 0/6 | 5 | (13.5) |
| 1/6 | 9 | (24.3) |
| 2/6 | 2 | (5.4) |
| 3/6 | 2 | (5.4) |
| 4/6 | 19 | (51.4) |
| KIR ligand Mismatch | | |
| No | 21 | (56.8) |
| Yes | 16 | (43.2) |
| Sex | | |
| Male | 22 | (60.0) |
| Female | 15 | (40.0) |
| CBU race | | |
| Asian | 1 | (2.7) |
| Black | 1 | (2.7) |
| Hispanic | 11 | (29.7) |
| Caucasian | 23 | (62.2) |
| Multiple | 1 | (2.7) |
| Time from collection to freezing (hours) | | |
| median (range) | 24.3 | (13.4-42.4) |
| Pre-freezing CBU viability (%) | | |
| median (range) | 99.0 | (89.0-100) |
| 25% percentile | 97.0 | |
| Days in culture | | |
| median (range) | 15 | (15-22) |
| TNC content (×10⁷ cells) | 168.6 | (87.3-248.8) |
| median (range) | | |
| 25% percentile | 128.1 | |
| NRBC content (×10⁷ cells) | | |
| median (range) | 4.8 | (0.0-23.8) |

| | |
|---|---|
| 75% percentile | 8.0 |

Abbreviations:
CLL = chronic lymphocytic leukemia; CLL-RT = chronic lymphocytic leukemia with Richter's transformation; DLBCL = diffuse large B cell lymphoma; LDH = lactate dehydrogenase; ULN = upper limit of normal; CBU = cord blood unit; TNC = total nucleated cell; NRBC = nucleated red blood cell.
Notations:
*¹ four patients had follicular lymphoma and two patients had marginal zone lymphoma; *² only for non-hodgkin's lymphoma (NHL) patients.

TABLE 2
Outcomes according to patient, disease, donor cord blood and CAR-NK characteristics

| | | | | | |
|---|---|---|---|---|---|
| | n | Day +30 | 1-year | 1-year PFS | 1-year OS |
| Variable | (%) | OR (%) | CR (%) | (95% CI) | (95% CI) |

Patient and disease characteristics

| Diagnosis | | p = 0.013 | | p = 0.08 | | p = 0.46 | | p = 0.03 | |
|---|---|---|---|---|---|---|---|---|---|
| Low grade NHL *1 | 6 | 6 | (100%) | 5 | (83.3) | 50.0 | (18.8-81.2) | 100 | |
| CLL | 6 | 4 | (66.7) | 3 | (50.0) | 33.3 | (9.7-69.9) | 83.3 | (43.7-97.0) |
| CLL-RT | 5 | 1 | (20.0) | 1 | (20.0) | 20.0 | (3.6-62.5) | 20.0 | (3.6-62.5) |
| DLBCL | 17 | 7 | (41.2) | 5 | (29.4) | 29.4 | (13.2-53.2) | 64.7 | (41.3-82.7) |
| Other | 3 | 0 | (0.0) | 0 | (0.0) | 33.3 | (6.1-79.2) | 66.7 | (20.8-93.9) |
| Age | | p = 0.75 | | p = 0.31 | | p = 0.59 | | p = 0.97 | |
| <65 years | 19 | 10 | (52.6) | 9 | (47.4) | 36.8 | (19.1-59.0) | 68.4 | (45.9-84.7) |
| ≥65 years | 18 | 8 | (44.4) | 5 | (27.8) | 27.8 | (12.5-51.0) | 66.7 | (43.8-83.7) |
| Sex | | p = 0.09 | | p = 0.17 | | p = 0.97 | | p = 0.71 | |
| Male | 24 | 9 | (37.5) | 7 | (29.2) | 37.5 | (21.1-57.3) | 66.7 | (46.8-82.0) |
| Female | 13 | 9 | (69.2) | 7 | (53.8) | 23.1 | (8.2-50.3) | 69.2 | (42.3-87.3) |
| Karnofsky | | p = 0.02 | | p = 0.17 | | p = 0.87 | | p = 0.06 | |
| ≤90 | 24 | 8 | (33.3) | 7 | (29.2) | 33.3 | (18.0-53.2) | 54.2 | (35.1-72.2) |
| 100 | 13 | 10 | (76.9) | 7 | (53.8) | 30.8 | (12.7-57.7) | 92.3 | (66.6-98.6) |
| Number of prior therapies | | p = 0.41 | | p = 0.65 | | p = 0.86 | | p = 0.44 | |
| 1-2 | 6 | 4 | (66.7) | 3 | (50.0) | 33.0 | (9.5-69.8) | 83.3 | (43.7-97.0) |
| ≥3 | 31 | 14 | (45.2) | 11 | (35.5) | 32.3 | (18.6-49.9) | 64.5 | (46.9-78.9) |
| Prior Stem Cell Transplant | | p = 1.00 | | p = 1.00 | | p = 0.45 | | p = 0.36 | |
| no | 27 | 13 | (48.1) | 10 | (37.0) | 29.6 | (15.8-48.5) | 63.0 | (44.2-78.5) |
| yes | 10 | 5 | (50.0) | 4 | (40.0) | 40.0 | (16.8-68.7) | 80.0 | (49.1-94.3) |
| Disease status | | p = 0.54 | | p = 0.30 | | p = 0.58 | | p = 0.30 | |
| Relapsed | 11 | 7 | (63.6) | 6 | (54.5) | 45.5 | (21.3-72.0) | 81.8 | (52.4-94.8) |
| Refractory to last therapy | 16 | 7 | (43.8) | 6 | (37.5) | 25.0 | (10.2-49.4) | 68.8 | (44.4-85.9) |
| Primary refractory | 10 | 4 | (40.0) | 2 | (20.0) | 30.0 | (10.8-60.3) | 50.0 | (23.7-76.3) |

CBU Characteristics

| HLA Allelic Match *² | | p = 0.85 | | p = 0.95 | | p = 0.90 | | p = 0.45 | |
|---|---|---|---|---|---|---|---|---|---|
| 0/6 | 5 | 3 | (60.0) | 2 | (40.0) | 40.0 | (11.8-76.9) | 60.0 | (23.1-88.2) |
| 1/6 | 9 | 5 | (55.6) | 4 | (44.4) | 33.3 | (12.0-64.5) | 88.9 | (56.4-98.0) |
| 2/6 | 2 | 1 | (50.0) | 1 | (50.0) | 50.0 | (9.4-90.6) 50.0 | 50.0 | (9.4-90.6) |
| 3/6 | 2 | 0 | (0.0) | 0 | (0.0) | | (9.4-90.6) | 100.0 | |
| 4/6 | 19 | 9 | (47.4) | 7 | (36.8) | 26.3 | (11.8-48.8) | 57.9 | (36.3-76.8) |
| KIR ligand Mismatch | | p = 0.51 | | p = 1.00 | | p = 0.35 | | p = 0.67 | |
| No | 21 | 9 | (42.9) | 8 | (38.1) | 28.6 | (13.8-50.1) | 71.4 | (49.9-86.2) |
| Yes | 16 | 9 | (56.3) | 6 | (37.5) | 37.5 | (18.5-61.4) | 62.5 | (38.6-81.5) |
| CBU race | | p = 0.091 | | p = 0.035 | | p = 0.40 | | p = 0.08 | |
| Other | 14 | 4 | (28.6) | 2 | (14.3) | 28.6 | (11.7-54.7) | 50.0 | (26.7-73.3) |
| Caucasian | 23 | 14 | (60.9) | 12 | (52.2) | 34.8 | (18.9-55.1) | 78.3 | (58.1-90.4) |
| CBU Gender | | p = 0.18 | | p = 0.31 | | p = 0.06 | | p = 0.12 | |
| Female | 15 | 5 | (33.3) | 4 | (26.7) | 20.0 | (7.1-45.1) | 53.3 | (30.1-75.2) |
| Male | 22 | 13 | (59.1) | 10 | (45.5) | 40.9 | (23.2-61.3) | 77.3 | (56.7-89.9) |
| Time to freezing | | p = 0.068 | | p = 0.017 | | p < 0.001 | | p = 0.023 | |
| >24 h | 18 | 6 | (33.3) | 11 | (57.9) | 5.6 | (1.0-25.7) | 50.0 | (29.0-71.0) |
| ≤24 h | 19 | 12 | (63.2) | 3 | (16.7) | 57.9 | (36.3-76.8) | 84.2 | (62.3-94.5) |
| Pre-freezing viability | | p = 0.41 | | p = 0.22 | | p = 0.03 | | p = 0.39 | |
| <97% | 7 | 2 | (28.6) | 1 | (14.3) | 0 | (−) | 57.1 | (25.0-84.1) |
| ≥97% | 30 | 16 | (53.3) | 13 | (43.3) | 40.0 | (24.7-57.6) | 70 | (52.1-83.4) |
| NRBC content | | p = 0.019 | | p = 0.007 | | p < 0.001 | | p = 0.06 | |
| ≤8 × 10⁷ cells | 28 | 17 | (60.7) | 14 | (50.0) | 42.9 | (26.5-61.1) | 75.0 | (56.6-87.3) |
| >8 × 10⁷ cells | 9 | 1 | (11.1) | 0 | (0.0) | 0 | (−) | 44.4 | (18.8-73.4) |
| TNC content | | p = 0.73 | | p = 0.27 | | p = 0.57 | | p = 0.20 | |
| ≤25% percentile | 26 | 12 | (46.2) | 8 | (30.8) | 26.9 | (13.7-46.1) | 61.5 | (42.6-77.5) |
| >25% percentile | 11 | 6 | (54.5) | 6 | (54.5) | 45.5 | (21.3-72.0) | 81.8 | (52.4-94.8) |
| Optimal CBU | | p = 0.008 | | p = 0.002 | | p < 0.001 | | p = 0.003 | |
| no | 21 | 6 | (28.6) | 3 | (14.3) | 4.8 | (0.9-22.4) | 47.6 | (28.3-67.6) |
| yes | 16 | 12 | (75.0) | 11 | (68.8) | 68.8 | (44.4-85.9) | 93.8 | (71.4-98.9) |

CAR NK Characteristics

| Transduction efficiency | | p = 0.71 | | p = 0.25 | | p = 0.17 | | p = 0.11 | |
|---|---|---|---|---|---|---|---|---|---|
| ≤25% percentile | 9 | 5 | (55.6) | 5 | (55.6) | 44.4 | (15.8-73.1) | 44.4 | (18.8-73.4) |
| >25% percentile | 28 | 13 | (46.4) | 9 | (32.1) | 28.6 | (15.3-47.0) | 75.0 | (56.6-87.3) |
| Fraction expansion | | p = 0.12 | | p = 0.43 | | p = 0.48 | | p = 0.62 | |
| ≤25% percentile | 9 | 2 | (22.2) | 2 | (22.2) | 22.2 | (6.3-54.8) | 61.5 | (35.5-82.3) |
| >25% percentile | 28 | 16 | (57.1) | 12 | (42.9) | 35.7 | (20.6-54.2) | 70.8 | (50.8-85.1) |
| Number of days in culture | | p = 0.75 | | p = 1.0 | | p = 0.28 | | p = 0.24 | |
| 15 days | 20 | 9 | (45.0) | 8 | (40.0) | 25.0 | (11.2-46.9) | 60.0 | (38.6-78.2) |
| 22 days | 17 | 9 | (52.9) | 6 | (35.3) | 41.2 | (21.7-64.0) | 76.5 | (52.7-90.5) |
| Dose level | | p = 0.43 | | p = 0.21 | | p = 0.39 | | p = 0.10 | |
| 1 × 10⁵ cells/kg | 3 | 2 | (66.7) | 2 | (66.7) | 66.7 | (20.8-93.9) | 66.7 | (20.8-93.9) |
| 1 × 10⁶ cells/kg | 4 | 3 | (75.0) | 3 | (75.0) | 25.0 | (4.5-70.0) | 100.0 | |
| 1 × 10⁷ cells/kg | 15 | 5 | (33.3) | 4 | (26.7) | 20.0 | (7.1-45.1) | 46.7 | (24.8-69.9) |
| 8 × 10⁸ cells | 15 | 8 | (53.3) | 5 | (33.3) | 40.0 | (19.9-64.2) | 80.0 | (54.9-92.9) |

flat dose

Notations:
*¹ Low grade lymphoma includes follicular lymphoma and marginal zone lymphoma; *² Number of HLA matches between the cord blood unit and the patient at HLA loci A, B and DRß1.
Abbreviations: OR = objective or overall response; CR = complete response; PFS = progression-free survival; OS = overall survival; CI = confidence interval; NHL = non-Hodgkin's lymphoma; CLL = chronic lymphocytic leukemia; CLL-RT = chronic lymphocytic leukemia with Richter transformation; DLBCL = diffuse large B cell lymphoma; LDH = lactate dehydrogenase; CBU = cord blood unit; NRBC = nucleated red blood cell; TNC = total nucleated cell.

TABLE 3
List of adverse reactions (noted are all side-effects from the time of infusion
until day +40, irrespective of their attribution to the CAR-NK cell therapy;
abnormalities due to the original disease were not captured).

| | Grade | | | | Grade | | |
|---|---|---|---|---|---|---|---|
| Adverse reactions | I-II | III | IV | Adverse reactions | I-II | III | IV |
| Cytokine Release Syndrome | 1 | 0 | 0 | Constitutional | | | |
| Graft vs Host Disease | 0 | 0 | 0 | Anorexia | 1 | 0 | 0 |
| Neurological Toxicity | 0 | 0 | 0 | Asthenia | 12 | 1 | 0 |
| Hematological Toxicity | | | | Fever/Chills^{†} | 2 | 0 | 0 |
| Neutropenia | 3 | 5 | 29 | Insomnia | 2 | 0 | 0 |
| Lymphopenia | 0 | 0 | 37 | Somnolence | 0 | 1 | 0 |
| Thrombocytopenia | 23 | 4 | 6 | Gastrointestinal | | | |
| Anemia | 19 | 18 | 0 | Diarrhea | 4 | 0 | 0 |
| Petechia/other bleeding | 1 | 0 | 0 | Hemorrhoids | 3 | 0 | 0 |
| Laboratory values | | | | Ileus | 0 | 2 | 0 |
| Elevated creatinine | 4 | 0 | 0 | Mucositis | 4 | 0 | 0 |
| Elevated LFTs | 9 | 1 | 0 | Nausea/Vomiting | 8 | 0 | 0 |
| Elevated CRP | 2 | 0 | 0 | Cardiovascular | | | |
| Elevated ferritin | 5 | 0 | 0 | Chest pain | 2 | 0 | 0 |
| Hyperglycemia | 1 | 0 | 0 | Dysrhythmia | 7 | 0 | 0 |
| Elevated LDH | 3 | 0 | 0 | Hypertension | 1 | 0 | 0 |
| Hypomagnesemia | 1 | 0 | 0 | Hypotension | 5 | 0 | 0 |
| Respiratory | | | | Edema/Fluid overload | 2 | 1 | 0 |
| Dyspnea | 1 | 0 | 0 | Miscellaneous | | | |
| Pleural effusion | 0 | 1 | 0 | Anxiety | 3 | 0 | 0 |
| Pneumonitis | 1 | 1 | 0 | Headache/Dizziness | 2 | 0 | 0 |
| Infection | | | | Muscle/bone pain | 4 | 0 | 0 |
| Bacterial infection | 3 | 1 | 0 | Paresthesia | 4 | 0 | 0 |
| Viral infection | 4 | 3 | 0 | Rash/Skin discoloration | 4 | 0 | 0 |
| Febrile neutropenia ^{††} | 0 | 4 | 0 | Other | 2 | 0 | 0 |

Abbreviations: LFTs = liver function tests; CRP = C-reactive protein; LDH = lactate dehydrogenase.
Notations:
^{†}1 patient had isolated chills, 1 patient had fever and was classified as CRS corresponding to the reported CRS case.
^{††} 1 patient had fever related to MRSA pneumonia; 1 patient had a fever that resolved within 24 hrs of starting antibiotics; 1 patient had two neutropenic fever episodes (the 1^{st} episode occurred before the cells were given and the 2^{nd} episode was a recurrence of this fever and did not fit criteria for CRS).

TABLE 15
Patient, disease, donor cord blood unit (CBU) and CAR-NK
characteristics in the dose escalation and dose expansion cohorts

| | n (%) | |
|---|---|---|
| | Dose escalation | Dose expansion |
| Variable | cohort (n = 11) | cohort (n = 26) |

Patient characteristics

| Age | | | | |
|---|---|---|---|---|
| Median (range) | 60.0 | (49-70) | 65.5 | (26-79) |
| <65 years | 9 | (81.8) | 10 | (38.5) |
| ≥65 years | 2 | (18.2) | 16 | (61.5) |
| Sex | | | | |
| Male | 7 | (63.6) | 17 | (65.4) |
| Female | 4 | (36.4) | 9 | (34.6) |
| Race | | | | |
| Caucasian | 8 | (72.7) | 23 | (88.5) |
| Hispanic | 0 | (0.0) | 0 | (0.0) |
| Black | 0 | (0.0) | 1 | (3.8) |
| Asian | 1 | (9.1) | 1 | (3.8) |
| Other | 2 | (18.2) | 1 | (3.8) |
| Weight | | | | |
| Median (kg/range) | 81.0 | (60-138) | 77.9 | (47-140) |
| Diagnosis | | | | |
| Indolent lymphoma *¹ | 1 | (9.1) | 5 | (19.2) |
| CLL | 4 | (36.4) | 2 | (7.7) |
| CLL-RT | 1 | (9.1) | 4 | (15.4) |
| DLBCL | 5 | (45.5) | 12 | (46.2) |
| Mantle cell lymphoma | 0 | (0.0) | 1 | (3.8) |
| Acute lymphoblastic leukemia | 0 | (0.0) | 1 | (3.8) |
| Lymphoplasmacytic lymphoma | 0 | (0.0) | 1 | (3.8) |
| Karnofsky (%) | 100 | (90-100) | 90 | (80-100) |
| Median (range) | | | | |
| LDH prior to | | | | |
| conditioning chemotherapy | | | | |
| Median (range) | 422 | (188-1715) | 241 | (141-1765) |
| LDH > ULN | 8 | (72.7) | 16 | (61.5) |
| Number of prior therapies | | | | |
| Median (range) | 4 | (3-10) | 4 | (2-9) |
| ≥3 prior lines of therapy | 11 | (100.0) | 20 | (76.9) |
| Prior stem cell transplant | | | | |
| Autologous | 4 | (36.4) | 5 | (19.2) |
| Allogeneic | 0 | (0.0) | 1 | (3.8) |
| Disease status | | | | |
| Relapsed | 7 | (63.6) | 4 | (15.4) |
| Refractory to most recent therapy | 2 | (18.2) | 14 | (53.8) |
| History of primary refractory disease | 2 | (18.2) | 8 | (30.8) |
| Disease stage *² | | | | |
| I or II | 2 | (33.3) | 6 | (37.5) |
| III or IV | 4 | (66.6) | 10 | (62.5) |

CAR-NK characteristics

| Dose level | | | | |
|---|---|---|---|---|
| 1 × 10⁵ cells/kg | 3 | (27.3) | 0 | |
| 1 × 10⁶ cells/kg | 4 | (36.4) | 0 | |
| 1 × 10⁷ cells/kg | 4 | (36.4) | 11 | (42.3) |
| 8 × 10⁸ cells | 0 | | 15 | (57.7) |
| flat dose | | | | |
| Mean fluorescence intensity | | | | |
| Median (range) | 10875 | (7510-36328) | 13278 | (4836-31618) |
| 25% percentile | 8203 | | 10798 | |
| Transduction efficiency (%) | | | | |
| Median (range) | 49.0 | (22.7-66.45) | 77.4 | (41.6-91.1) |
| 25% percentile | 38.6 | | 65.9 | |
| CD3+ T-cells infused/kg | | | | |
| Median (range) | 500 | (30-8000) | 3000 | (500-16000) |

CBU characteristics

| HLA allelic match | | | | |
|---|---|---|---|---|
| 0/6 | 0 | | 5 | (19.2) |
| 1/6 | 1 | (9.1) | 8 | (30.8) |
| 2/6 | 1 | (9.1) | 1 | (3.8) |
| 3/6 | 0 | | 2 | (7.7) |
| 4/6 | 9 | (81.8) | 10 | (38.5) |
| KIR ligand mismatch | | | | |
| No | 8 | (72.7) | 13 | (50) |
| Yes | 3 | (27.3) | 13 | (50) |
| Sex | | | | |
| Male | 6 | (54.5) | 16 | (61.5) |
| Female | 5 | (45.5) | 10 | (38.5) |
| CBU race | | | | |
| Asian | 1 | (9.1) | 0 | |
| Black | 1 | (9.1) | 0 | |
| Hispanic | 1 | (9.1) | 10 | (38.5) |
| Caucasian | 7 | (63.6) | 16 | (61.5) |
| Multiple | 1 | (9.1) | 0 | |
| Time from collection to freezing (hours) | | | | |
| Median (range) | 24.3 | (16.5-42.4) | 24.5 | (13.4-34.9) |
| Pre-freezing CBU viability (%) | | | | |
| Median (range) | 99.0 | (89-99) | 99.0 | (91-100) |
| Days in culture | | | | |
| Median (range) | 15 | (15-22) | 22 | (15-22) |
| TNC content (×10⁷ cells) | | | | |
| Median (range) | 124.8 | (87.3-248.8) | 184.5 | (124.1-241.3) |
| 25% percentile | 111.9 | | 162.5 | |
| NRBC content (×10⁷ cells) | | | | |
| Median (range) | 3.2 | (1.0-23.8) | 5.2 | (0.0-19.2) |

| | | |
|---|---|---|
| 75% percentile | 2.8 | 2.7 |

Abbreviations: CLL = chronic lymphocytic leukemia; CLL-RT = chronic lymphocytic leukemia with Richter's transformation; DLBCL = diffuse large B cell lymphoma; LDH = lactate dehydrogenase; ULN = upper limit of normal; CBU = cord blood unit; TNC = total nucleated cell; NRBC = nucleated red blood cell.
Notations:
*¹ four patients had follicular lymphoma and two patients had marginal zone lymphoma;
*² only for non-Hodgkin's lymphoma (NHL) patients.

The results provided herein confirmed that allogeneic CAR NK-cells had an excellent safety profile. No cases of graft-v-host disease (GVHD) or neurotoxicity were observed, and only one case of grade 1 CRS was observed. No patients required treatment with steroids, tocilizumab, or the safety switch rimiducid dimerizer. No patients required transfer to ICU for management of complications, and no patients died of treatment-related causes. These results compared favorably with reports of up to 5% non-relapse mortality with CAR T cells and ICU treatment in 30% of cases (see e.g., Neelapu, S. S., et al., (2017) and Nastoupil, L. J., et al., (2020)). In this study the levels of inflammatory cytokines such as IL-6, TNF-α, and IFN-γ remained normal at all times, further supporting the safety profile of the CAR NK cell products.

Together, these results showed that iC9/CD19-CAR/IL15 CB-NK have at least an on par efficacy profile when compared to autologous anti CD19 CAR T-cells. Furthermore, the results provided herein showed that the safety profile of iC9/CD19-CAR/IL15 CB-NK was significantly improved when compared to that of anti CD19 CAR T cells, as CD19 CAR-NK cells were not associated with significant CRS or neurotoxicity. Additionally, as described herein, these results can potentially be improved by thoughtful selection of donor NK cells with characteristics that were found herein to be correlated with positive patient responses.

### Example 3-Donor Cord Blood Characteristic were the Most Important Determinants of Patient Outcomes

Previous studies with CAR T cells have reported the importance of CAR T cell characteristics in defining outcome. Considering this, the inventors investigated whether CB unit characteristics could influence patient outcomes. As reported herein, the inventors have identified cord blood unit (CBU) characteristics that can be used to select optimal CBUs for the manufacture of cell therapy products (see e.g., Table 2).

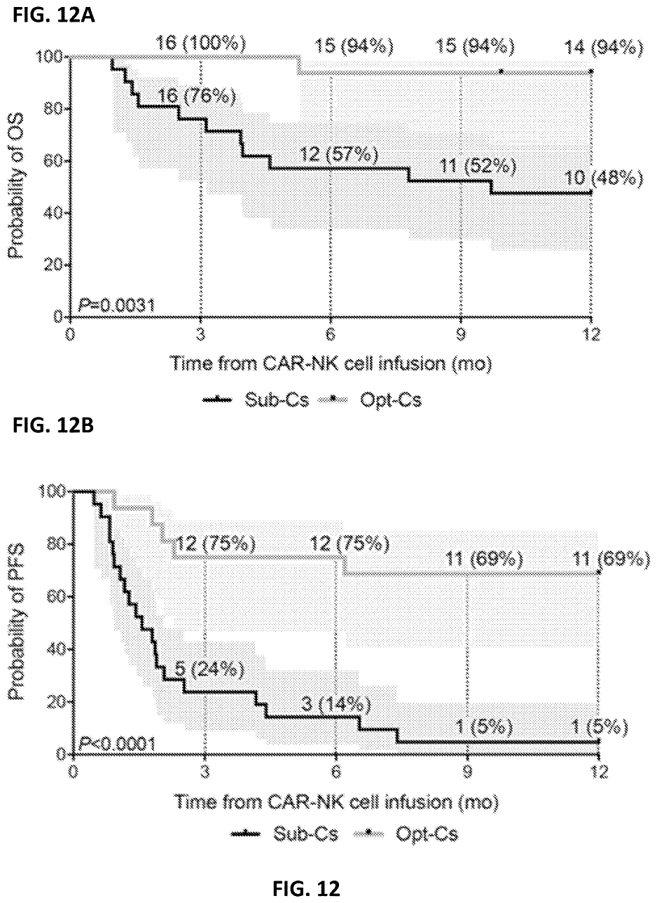

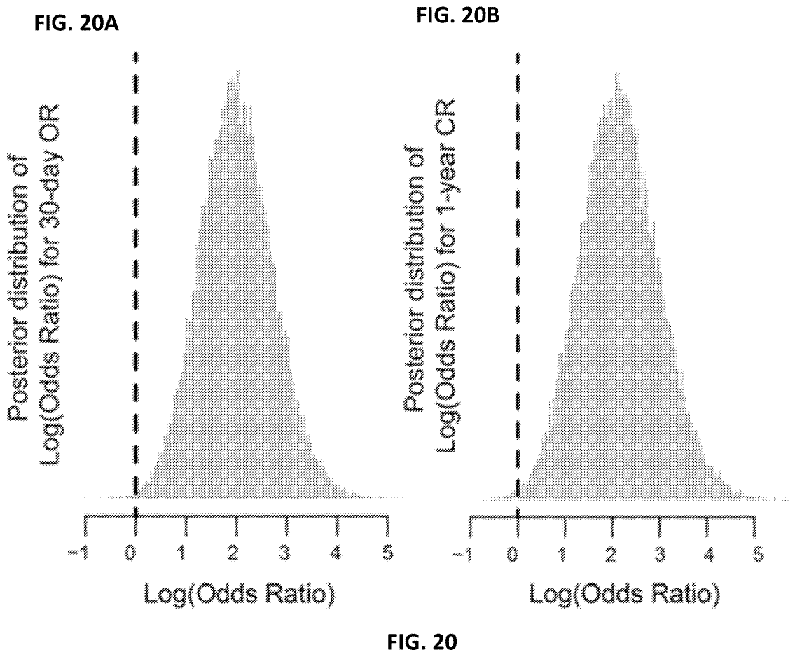

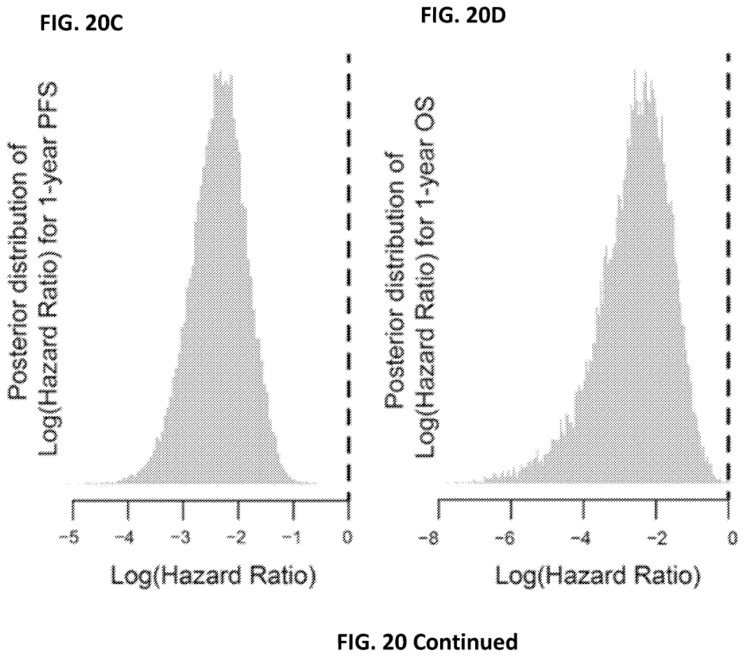

To account for the relatively small sample size provided herein, the inventors used Bayesian models to estimate the effect of a given covariate on a particular outcome. This effect was quantified by the probability of a beneficial effect (PBE), defined as the probability of a better outcome when the variable is present. A PBE near 0 implied a very harmful effect of the covariate, a value near 1 implied a very beneficial effect, and PBE close to 0.50 corresponded to no effect. Among the various CBU characteristics, time from birth to CBU processing (“collection-to-cryopreservation”) below or equal to 24 h (≤24 h) (Hazard Ratio [HR]-0.137, CI=0.103-0.572, p=0.001, PBE=1.00, 95% Credible Interval [95CrI] 0.054-0.317), and a nucleated red blood cell (NRBC) content of ≤8×10⁷ cells per CB unit (HR=0.173, CI=0.066-0.455, p<0.001; HR=0.119, 95CrI 0.052-0.4283, PBE=1.00) were found to be independent predictors for 1-year PFS. The 16 patients who received cell products derived from CBUs with both characteristics (e.g., collection-to-cryopreservation≤24 h and ≤8×10⁷ cells per CB unit, e.g., “optimal CBUs”; aka “Opt-Cs”) had a significantly superior day+30 OR (75.0% vs 28.6%, p=0.008; Odds Ratio=7.41, 95CrI 1.86-33.11, PBE=0.998), 1-year CR (68.8% vs 14.3%, p=0.002; Odds Ratio=12.40, 95CrI 2.78-65.04, PBE=1.00), 1-year PFS (68.8% vs 4.8%, p<0.001; HR=0.94, 95CrI 0.032-0.239, PBE=1.00), and 1-year OS (93.8% vs 47.6, p=0.003; HR=0.073, 95 CrI 0.01-0.384, PBE=1.00) when compared to the 21 patients who received products derived from CBUs with either a high NRBC content (a surrogate for fetal hypoxia and stress) ^{21,22} and/or that were cryopreserved greater than 24 h after birth (e.g., “sub-optimal CBUs”; aka “Sub-Cs”) (FIGS. 20A-20D, FIGS. 12A-12B, and Table 2). As previously reported, the degree of HLA match or the presence of KIR ligand mismatch between the donor and the patient did not impact the outcome (see e.g., Enli Liu et al., 2020; and Table 2).

The inventors also investigated patient, disease, and donor characteristics that could potentially influence treatment outcomes. Multivariate regression analyses were performed (as described in the Methods) to analyze optimal CBUs, the variables in Table 2, and those variables described below. Receipt of CAR-NK cells generated from an optimal CBU was strongly associated with higher probabilities of day 30+OR (Odds Ratio=13.05, 95CrI 1.50-137.4, PBE=0.991), 1-year CR (Odds Ratio=9.00, 95CrI 1.12-82.46, PBE=0.981), 1-year PFS (HR=0.041, 95CrI 0.012-0.134, PBE=1.000), and 1-year OS (HR=0.053, 95CrI 0.006-0.331, PBE=1.00). Karnofsky score >90% was also associated with a higher probability of day+30 OR (Odds Ratio=5.80, 95CrI 0.69-55.31, PBE=0.946) and probability of 1-year survival (HR=0.135, 95CrI 0.011-0.906, PBE=0.981).

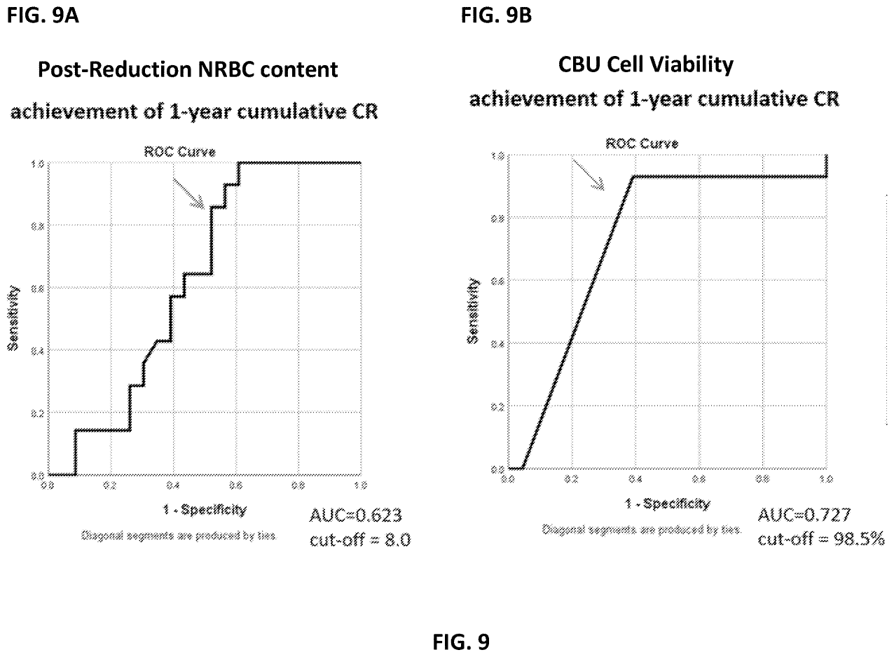

The inventors investigated whether pre-freezing cord blood unit (CBU) characteristics could be utilized to identify CBUs more likely to result in a clinically efficacious cell product. Receiver Operating Characteristic (ROC) curves were utilized to study the predictive value of the CBU characteristic of interest, and to identify the appropriate cut-off value that would facilitate classifying each individual CBU as likely (“good”) or unlikely (“bad”) to induce clinical response in patients. For example, as shown in FIG. 9A, the CBU post-reduction nucleated red blood cell (NRBC) content was analyzed. The blue arrow on the ROC curve indicated the value on the CBU post-reduction NRBC content that can be use to classify the CBU as “good or bad” with the best sensitivity and specificity (this is determined by the closets point to 100% sensitivity and 100% [1-specificity]). The curve plotted the sensitivity and the 1-specificity for each value of NRBC content. The arrow indicated the point of best sensitivity/specificity balance for that particular data. The best cut-off was the NRBC content that had that particular sensitivity and specificity. The arrow indicated the sensitivity and specificity of the best cut-off, not the cut-off value. In this case the value was 8.0×10⁷ cells. Then the response that patient had to CAR-NK cells was examined. Patients who received CAR-NK cells produced from CBU with a NRBC <8×10⁷ cells represented 50.08% of complete response, on the other hand patients who received CAR-NK cells manufactured from CBUs with a NRBC greater than 8×10⁷ cells had no responses. This result was statistically significant (Fisher exact test, p=0.007, see Table 4). Then a logistic regression model was utilized to verify that this result was independent of the clinical characteristics of the patient, such us remission status.

TABLE 4
Post-reduction CBU NRBC content response
crosstabulation (median 4.8 × 10⁷ (range 0.0 to 23.8),
AUC = 0.623, cut-off = 8.0 × 10⁷, p = 0.007)

| | | | Response | | |
|---|---|---|---|---|---|
| | | | No CR | CR | Total |
| NRBC | >8 × 10⁷ | Count | 9 | 0 | 9 |
| | cells | Expected Count | 5.6 | 3.4 | 9.0 |
| | | % within NRBC | 100.0% | 0.0% | 100.0% |
| | <8 × 10⁷ | Count | 14 | 14 | 28 |
| | cells | Expected Count | 17.4 | 10.6 | 28 |
| | | % within NRBC | 50.0% | 50.0% | 100.0% |
| Total | | Count | 23 | 14 | 37 |
| | | Expected Count | 23 | 14 | 37 |
| | | % within NRBC | 62.2% | 37.8% | 100.0% |

The inventors employed the same methodology described above with other CBU characteristics, for example the pre-process NRBC content of the CBU. Patients who received CAR-NK cells produced from CBU with a low cell content (less than or equal to 9.4×10⁷ NRBC) had 50.0% of complete response, on the other hand patients who received CAR-NK cells manufactured from CBUs with a higher NRBC content (greater than 9.4×10⁷) had only 15.4% response. This result was statistically significant (Pearson Chi-Square, p=0.038, see Table 5). Again, a multivariate logistic regression model was utilized to demonstrate that this effect was independent of clinical variables.

TABLE 5
Pre-reduction CBU NRBC content response crosstabulation
(median 6.9 × 10⁷ NRBC (range 1.0 to 39.2), AUC = 0.612,
cut-off ≤ 9.4 × 10⁷ cells, p = 0.038)

| | | | Response | | |
|---|---|---|---|---|---|
| | | | No CR | CR | Total |
| Pre-process | ≤9.4 × 10⁷ | Count | 12 | 12 | 24 |
| NRBC | cells | % within pre- | 50.0% | 50.0% | 100.0% |
| | | process NRBC | | | |
| | >9.4 × 10⁷ | Count | 11 | 2 | 13 |
| | cells | % within pre- | 84.6% | 15.4% | 100.0% |
| | | process NRBC | | | |
| Total | | Count | 23 | 14 | 37 |
| | | % within pre- | 62.2% | 37.8% | 100.0% |
| | | process NRBC | | | |

The inventors employed the same methodology described above with other CBU characteristics, for example the post-process NRBC percentage calculated as a percentage of total nucleated cells (TNC). Patients who received CAR-NK cells produced from CBU with a low NRBC % (less than or equal to 4%) had 52.2% of complete responses, on the other hand patients who received CAR-NK cells manufactured from CBUs with a higher NRBC percentage (greater than 4%) had only 14.3% of complete responses. This result was statistically significant (Fisher's Exact Test, p=0.023, see Table 6). Again, a multivariate logistic model was utilized to demonstrate that this effect was independent of clinical variables.

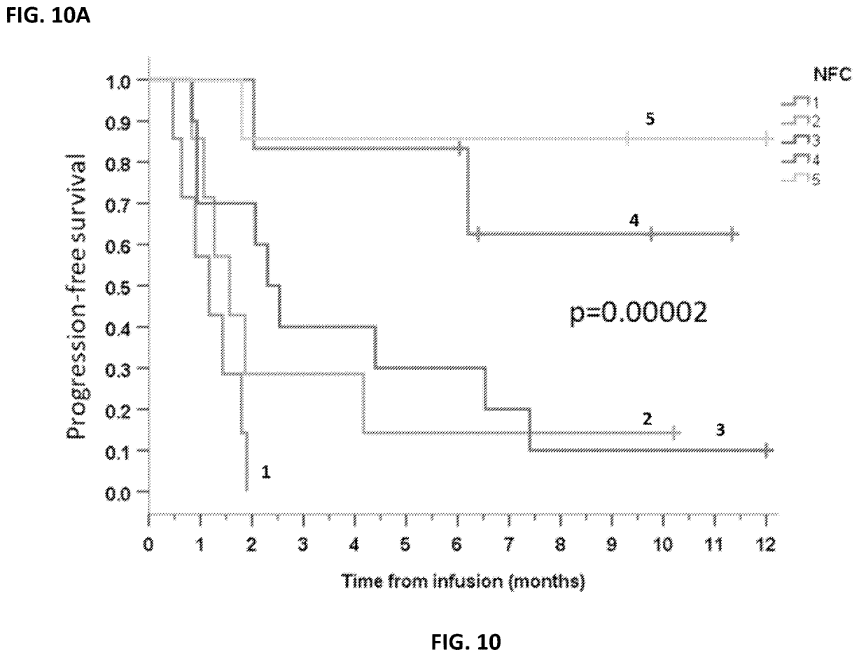

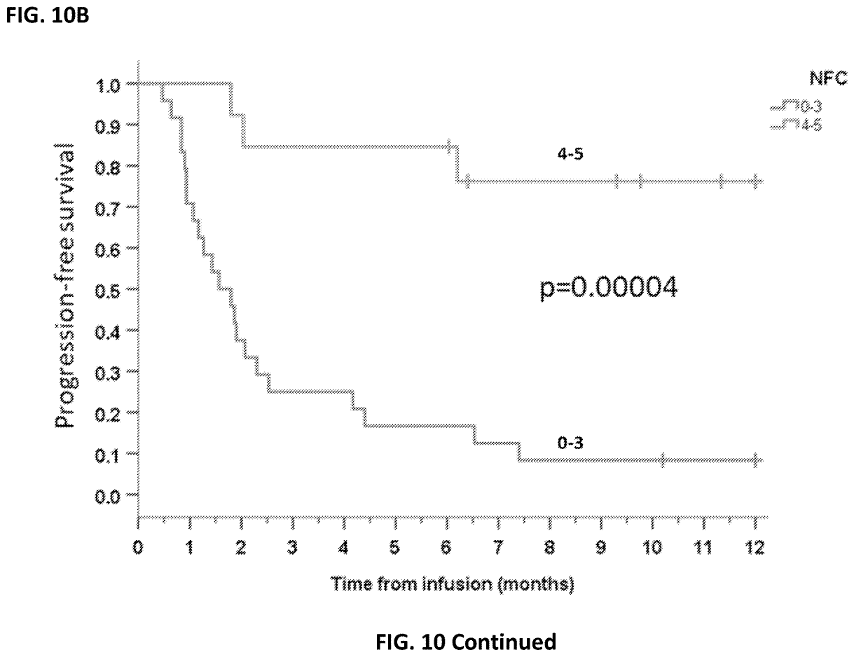

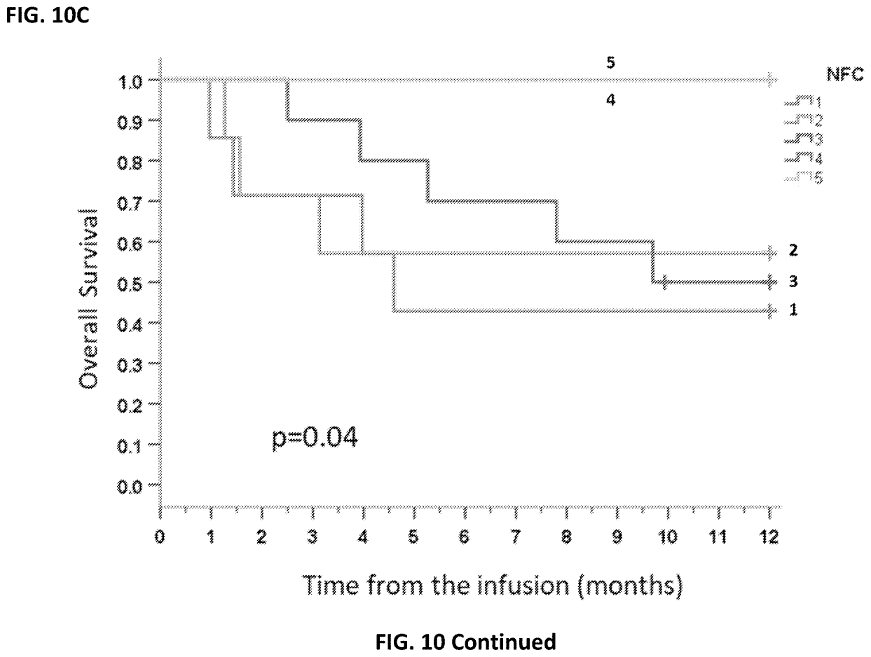

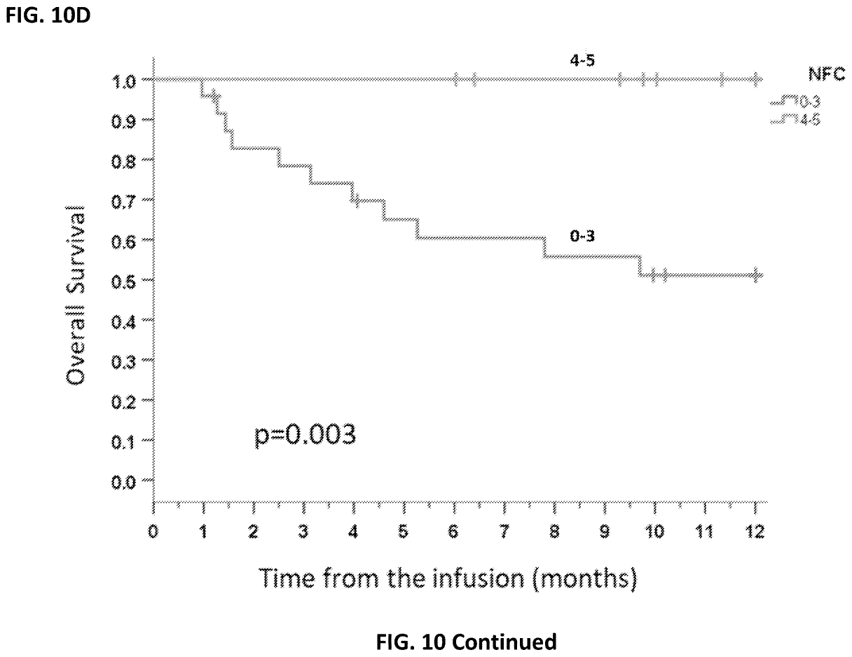

Because post-reduction NRBC content, pre-reduction NRBC and post-process NRBC % are closely related variables, only one of these variables were included in any given model. However, any one, two, or three of these variables could be utilized alone or together. For the purposes of additional exemplification, post-reduction NRBC content was utilized as the NRBC related variable when determining “optimal” CBUs (see e.g., FIG. 10 onwards).

TABLE 6
Post-process CBU NRBC content percentage of
TNC response crosstabulation (median 3.4% (range
0.0 to 12.6), AUC = 0.663, cut-off ≤ 4%, p = 0.023)

| | | | Response | | |
|---|---|---|---|---|---|
| | | | No CR | CR | Total |
| Post-process | ≤4% | Count | 11 | 12 | 23 |
| NRBC | | % within | 47.8% | 52.2% | 100.0% |
| | | NRBC% | | | |
| | >4% | Count % | 12 | 2 | 14 |
| | | within | 85.7% | 14.3% | 100.0% |
| | | NRBC | | | |
| Total | | % Count | 23 | 14 | 37 |
| | | % within | 62.2% | 37.8% | 100.0% |
| | | NRBC% | | | |

The inventors employed the same methodology described above with other CBU characteristics, for example the CBU pre-freezing cell viability. The blue arrow on the ROC curve (FIG. 9B) indicated a value on the CBU pre-freezing cell viability that can be used to classify the CBU as “good” or “bad” with the best sensitivity and specificity (this is determined by the closest point to 100% sensitivity and 100% [1-specificity]). In this case the value was 98.5% pre-freezing viability. Then the response that patients had to CAR-NK cells was investigated. Patients who received CAR-NK cells produced from CBU with a viability >98.5% had 59.1% response, on the other hand patients who received CAR-NK cells manufactured from CBUs with a viability of less than or equal to 98.5% had only 6.7% response. This result was statistically significant (Fisher exact test, p=0.002, see Table 7). A logistic regression model was used to verify that this result was independent of the clinical characteristics of the patient, such us remission status.

TABLE 7
Pre-freezing viability response crosstabulation
(median 97.9% (range 89.0 to 100.0),
AUC = 0.727, cut-off > 98.5%, p = 0.002)

| | | | Response | | |
|---|---|---|---|---|---|
| | | | No CR | CR | Total |
| Pre-freezing | ≤98.5% | Count | 14 | 1 | 15 |
| Viability | | % within Pre- | 93.3% | 6.7% | 100.0% |
| | | freezing | | | |
| | | viability | | | |
| | >98.5% | Count | 9 | 13 | 22 |
| | | % within Pre- | 40.9% | 59.1% | 100.0% |
| | | freezing | | | |
| | | viability | | | |
| Total | | Count | 23 | 14 | 37 |
| | | % within Pre- | 62.2% | 37.8% | 100.0% |
| | | freezing | | | |
| | | viability | | | |

The inventors employed the same methodology described above with other CBU characteristics, for example the CD34 positive cell percentage. Patients who received CAR-NK cells produced from CBU with a high CD34 positive percentage (>0.245%) had 35.7% of complete responses by day+30, on the other hand patients who received CAR-NK cells manufactured from CBUs with a lower CD34 positive % had no responses. This result was statistically significant (Pearson's Chi square, p=0.036, see Table 8). Analysis with a multivariate logistic model demonstrated that this effect was independent of clinical variables.

TABLE 8
CD34 positive percentage CR by day +30 response
crosstabulation (median 0.35% (range 0.1 to 0.91%),
AUC = 0.743, cut-off > 0.245%, p = 0.036)

| | | | CR by day +30 | | |
|---|---|---|---|---|---|
| | | | No CR | CR | Total |
| CD34 (%) | ≤0.245% | Count | 9 | 0 | 9 |
| | | % within | 100.0% | 0.0% | 100.0% |
| | | CD34 (%) | | | |
| | >0.245% | Count | 18 | 10 | 28 |
| | | % within | 64.3% | 35.7% | 100.0% |
| | | CD34 (%) | | | |
| Total | | Count | 27 | 10 | 37 |
| | | % within | 73.0% | 27.0% | 100.0% |
| | | CD34(%) | | | |

The inventors employed the same methodology described above with other CBU characteristics, for example the time from the baby birth until the CBU is cryopreserved (time measured in hours). Patients who received CAR-NK cells produced from CBU that was cryopreserved within 24 hours from birth had 61.1% of complete responses, on the other hand patients who received CAR-NK cells manufactured from CBUs that were cryopreserved after 24 hours from birth had only 15.8% response rate. This result was statistically significant (Fisher's Exact Test, p=0.007, see Table 9). Analysis with a multivariate logistic model demonstrated that this effect was independent of clinical variables.

TABLE 9
Time from birth to cryopreservation (hours (h)) response
crosstabulation (median 24.6 h (range 13.4 to 42.4),
AUC = 0.742, cut-off ≤ 24 h, p = 0.007)

| | | | Response | | |
|---|---|---|---|---|---|
| | | | No CR | CR | Total |
| Time from birth | >24 h | Count | 16 | 3 | 19 |
| to | | % within Time | 84.2% | 15.8% | 100.0% |
| cryopreservation | | from birth to | | | |
| | | cryopreservation | | | |
| | ≤24 h | Count | 7 | 11 | 18 |
| | | % within Time | 38.9% | 61.1% | 100.0% |
| | | from birth to | | | |
| | | cryopreservation | | | |
| Total | | Count | 23 | 14 | 37 |
| | | % within Time | 62.2% | 37.8% | 100.0% |
| | | from birth to | | | |
| | | cryopreservation | | | |

The inventors employed the same methodology described above with other CBU characteristics, for example the baby weight. Patients who received CAR-NK cells produced from CBU obtained from a baby who weighted >3650 grams (gr) represented 42.1% of complete responses by day 30, on the other hand patients who received CAR-NK cells manufactured from CBUs obtained from babies who weighted ≤3650 represented only 11.1% of the response rate. This result was statistically significant (Pearson Chi-Square, p=0.034, see Table 10). Analysis with a multivariate logistic model demonstrated that this effect was independent of clinical variables.

TABLE 10
Baby weight CR by day +30 response crosstabulation
(median 3658 grams (gr) (range 2656 to 4335),
AUC = 0.696, cut-off > 3650, p = 0.34)

| | | | CR by day +30 | | |
|---|---|---|---|---|---|
| | | | No CR | CR | Total |
| Weight | <3650 | Count | 16 | 2 | 18 |
| | | % within | 88.9% | 11.1% | 100.0% |
| | | Weight | | | |
| | >3650 | Count | 11 | 8 | 19 |
| | | % within | 57.9% | 42.1% | 100.0% |
| | | Weight | | | |
| Total | | Count | 27 | 10 | 37 |
| | | % within | 73.0% | 27.0% | 100.0% |
| | | Weight | | | |

The inventors employed the same methodology described above with other CBU characteristics, for example the mother's age. Patients who received CAR-NK cells produced from CBU obtained from a baby whose mother was 32 years or younger represented 48% of complete responses rate, on the other hand patients who received CAR-NK cells manufactured from CBUs obtained from babies whose mothers were older had only 16.7% response rate. (Fisher's Exact test, p=0.067, see Table 11). Analysis with a multivariate logistic model demonstrated that this effect was independent of clinical variables.

TABLE 11
Mother's age response
crosstabulation (median 29.9 years
(range 21.1 to 36.9), AUC = 0.537,
cut-off < 32, p = 0.067)

| | | | Response | | |
|---|---|---|---|---|---|
| | | | No CR | CR | Total |
| Mothers | <32 | Count | 13 | 12 | 25 |
| age | | % within | 52.0% | 48.0% | 100.0% |
| | | Mothers age | | | |
| | >32 | Count | 10 | 2 | 12 |
| | | % within | 83.3% | 16.7% | 100.0% |
| | | Mothers age | | | |
| Total | | Count | 23 | 14 | 37 |
| | | % within | 62.2% | 37.8% | 100.0% |
| | | Mothers age | | | |

The inventors employed the same methodology described above with other CBU characteristics, for example the race of the baby. Patients who received CAR-NK cells produced from CBU obtained from a baby with at least one Caucasian parent represented 39.1% of complete responses by day 30, on the other hand patients who received CAR-NK cells manufactured from CBUs obtained from babies with no Caucasian parents had a 7.1% response rate. This result was statistically significant (Pearson Chi-Square, p=0.034, see Table 12). Analysis with a multivariate logistic model demonstrated that this effect was independent of clinical variables.

TABLE 12
CBU Race CR by day +30 response crosstabulation (p = 0.34)

| | | | CR by day +30 | | |
|---|---|---|---|---|---|
| | | | No CR | CR | Total |
| CBU Race | Other | Count | 13 | 1 | 14 |
| | | % within | 92.9% | 7.1% | 100.0% |
| | | CBU Race | | | |
| | Caucasian | Count | 14 | 9 | 23 |
| | | % within | 60.9% | 39.1% | 100.0% |
| | | CBU Race | | | |
| Total | | Count | 27 | 10 | 37 |
| | | % within | 73.0% | 27.0% | 100.0% |
| | | CBU Race | | | |

The inventors then determined that the CBU characteristics described herein could be combined to further refine the selection process. For example, CBU characteristics of NRBC content (e.g., post-reduction CBU NRBC content), pre-freezing viability, time from birth to cryopreservation, baby race, and/or baby weight, were combined to provide predicted responses. In the cohort analyzed, all CBUs had at least one of the selected favorable characteristics. The probability of obtaining complete remission increased with the number of favorable characteristics in the CBU from which the CAR-NK were derived. Namely, the probability of CR increased from 0% in patients who received CAR NK derived from units with only one favorable characteristic, to 100% in patients who received a cell product derided from CBUs with all 5 of the selected characteristic. This results were statistically significant (Fisher's Exact Test, p<0.001, see Table 13). The results are depicted as survival curves in FIGS. 10A-10D.

TABLE 13
NFC response crosstabulation (p < 0.001)

| | | | Response | | |
|---|---|---|---|---|---|
| | | | No CR | CR | Total |
| NFC | 1 | Count | 7 | 0 | 7 |
| | | % within | 100.0% | 0.0% | 100.0% |
| | | NFCc ount | | | |
| | 2 | Count | 6 | 1 | 7 |
| | | % within | 85.7% | 14.3% | 100% |
| | | NFC count | | | |
| | 3 | Count | 7 | 3 | 10 |
| | | % within | 70.0% | 30.0% | 100.0% |
| | | NFC count | | | |
| | 4 | Count | 3 | 3 | 6 |
| | | % within | 50.0% | 50.0% | 100.0% |
| | | NFC count | | | |
| | 5 | Count | 0 | 7 | 7 |
| | | % within | 0.0% | 100.0% | 100.0% |
| | | NFC count | | | |
| Total | | Count | 23 | 14 | 37 |
| | | % within | 62.2% | 37.8% | 100.0% |
| | | NFC count | | | |

The inventors then validated these results in an independent sample of 19 patients treated with a different NK cell product. The results of the analysis were very similar. In this case, the day+30 CR rate was 0%, 33.3%, and 75% for patients who received cell products derived from CBUs that had 0-2, 3 or 4 characteristics respectively (p=0.029, see Table 14), where the selected characteristics were post-reduction NRBC content, pre-freeze viability, baby race, and baby weight, as an exemplary set of criteria.

TABLE 14
NFC validation response crosstabulation (p = 0.029)

| | | | Day 30 Response | | |
|---|---|---|---|---|---|
| | | | No CR | CR | Total |
| NFC | 0-2 | Count | 6 | 0 | 6 |
| | | Expected Count | 4.1 | 1.9 | 6 |
| | | % within | 100.0% | 0.0% | 100.0% |
| | | NFC count | | | |
| | 3 | Count | 6 | 3 | 9 |
| | | Expected Count | 6.2 | 2.8 | 9.0 |
| | | % within | 66.7% | 33.3% | 100.0% |
| | | NFC count | | | |
| | 4 | Count | 1 | 3 | 4 |
| | | Expected Count | 2.7 | 1.3 | 4.0 |
| | | % within | 25.0% | 75.0% | 100.0% |
| | | NFC count | | | |
| Total | | Count | 13 | 6 | 19 |
| | | Expected Count | 13.0 | 6.0 | 19.0 |
| | | % within | 68.4% | 31.6% | 100.0% |
| | | NFC count | | | |

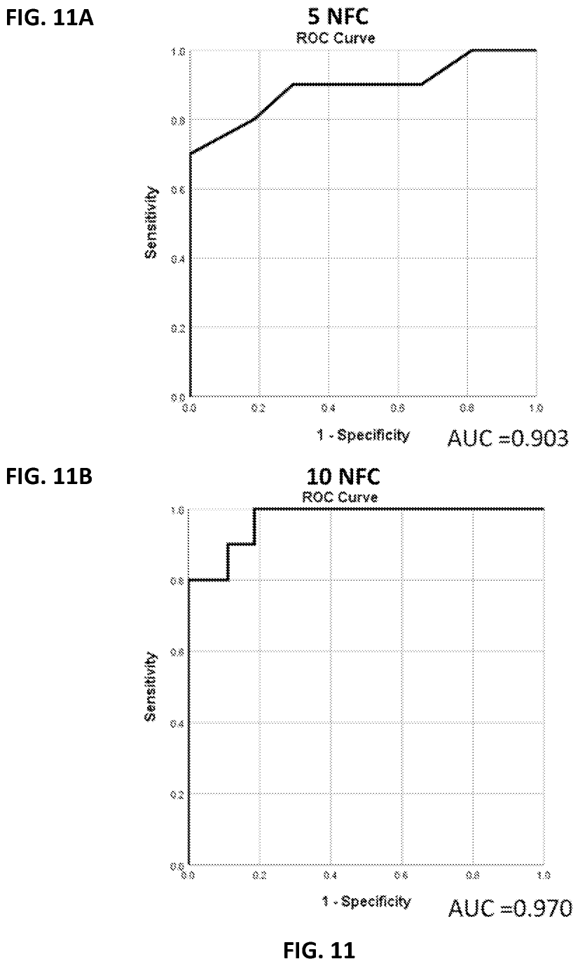

Additional favorable characteristic parameters could also be applied. For example, a) gestational age≤38 weeks, b) mothers age <32, c) intra utero collection method (e.g., blood collected from the placenta while the placenta is in the uterus), d) male baby, e) pre-processing volume≤120 ml, f) CD34%>0.245%, g) NK cell expansion between days 0 and 15 in culture ≥450 fold, and/or h) NK cell expansion between days 6 and 15 in culture ≥70 fold. An exemplary 5 NFC (viability, NRBC content, time to freezing, race, and weight) ROC curve is depicted in FIG. 11A with a predictive clinical response rate of 90.3%, while an exemplary 10 NFC (viability, NRBC content, time to freezing, race, weight, gestational age, gender, CD34%, pre-processing volume, and expansion rate) ROC curve is depicted in FIG. 11B with a predictive clinical response rate of 97.0%.

As shown in FIGS. 12A and 12B, Kaplan-Meier curves were determined for OS (FIG. 12A) and PFS (FIG. 12B) for the 37 patients enrolled in the clinical trial when the patients were categorized as receiving CBU's with positive characteristics (“Opt-Cs”) compared to those with negative characteristics (“Sub-Cs”), where Opt-Cs CBUs had a time to freezing≤24 h and a pre-reduction NRBC content of ≤8×10⁷, and Sub-Cs CBUs had a time to freezing >24 h and a pre-reduction NRBC content of >8×10⁷. CBU quality was the most significant predictor of response to CB-derived CD19 CAR-NK cell treatment, with both OS and PFS rates being significantly improved when NK cells were derived from CBUs that had both a time to freezing≤24 h and a pre-reduction NRBC content of ≤8×10⁷ (p=0.0031, and p<0.0001 respectively; referred to in these Figures and the associated Examples as “Opt-Cs” or “Opt-NK”).

### Example 4-Validation of Efficacious Cord Blood Characteristics In Vitro

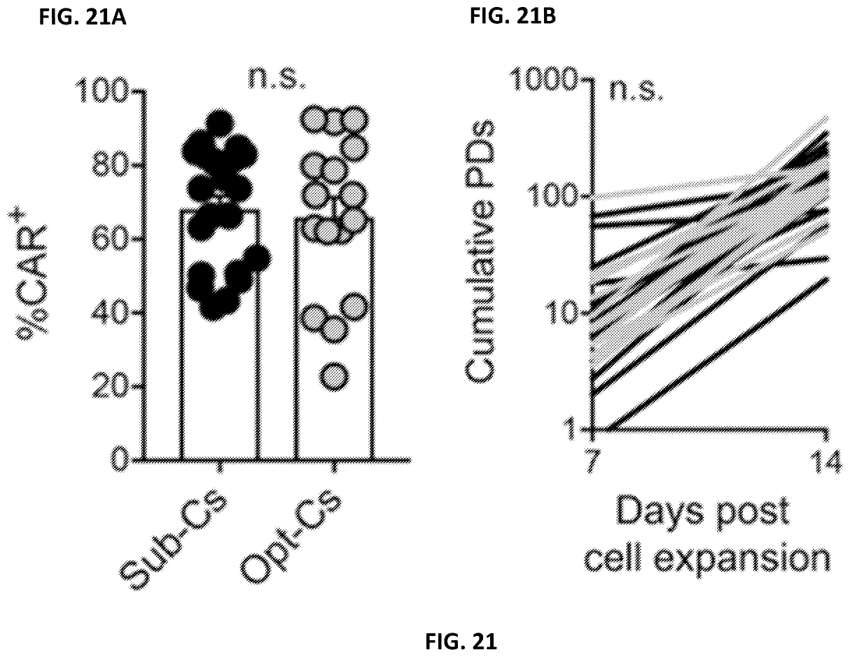

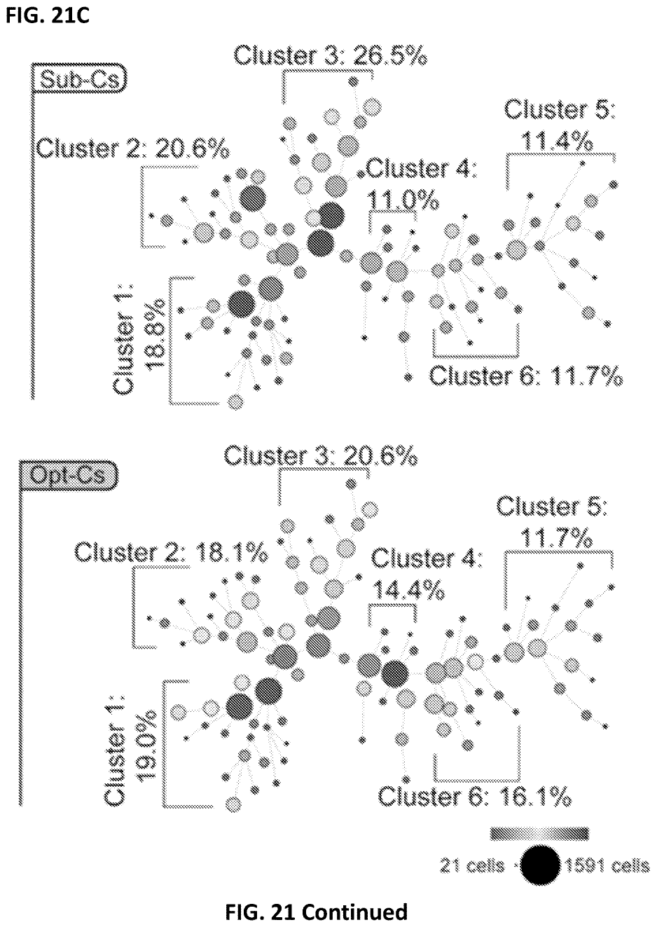

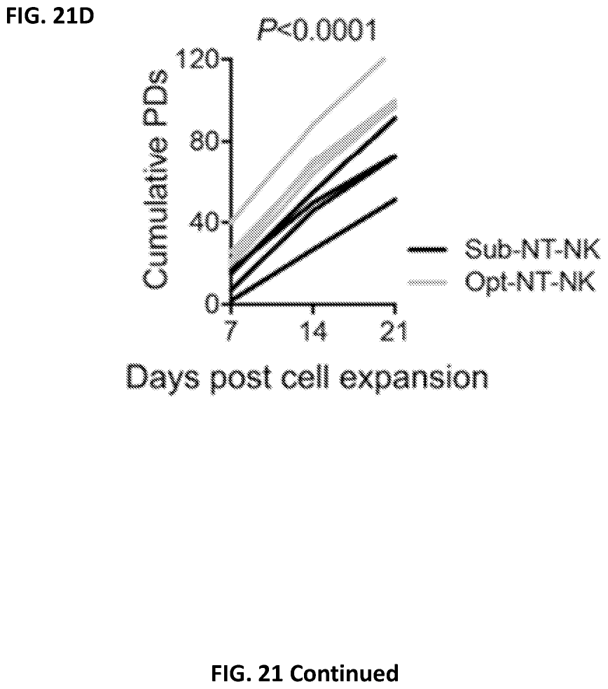

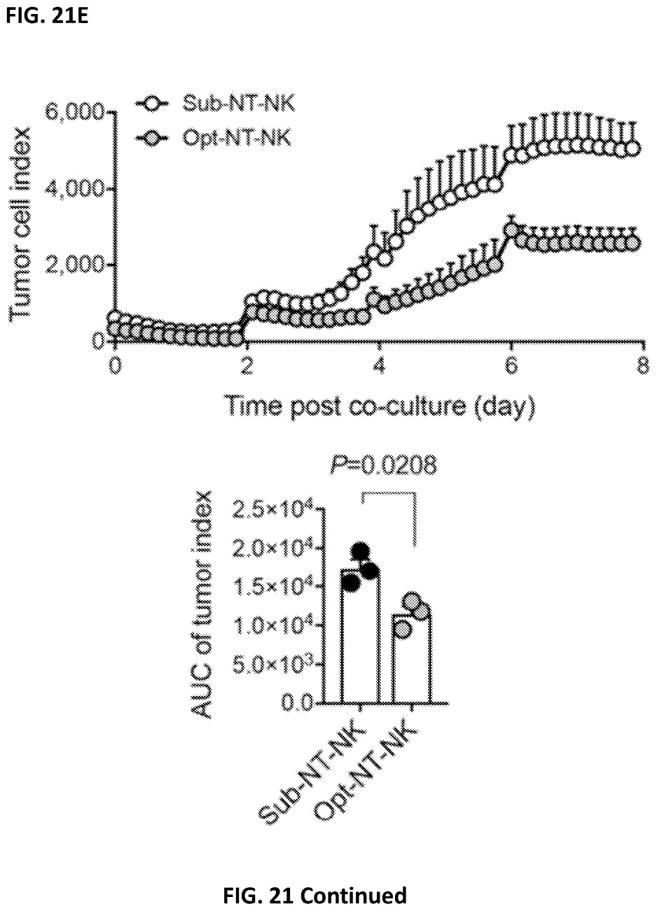

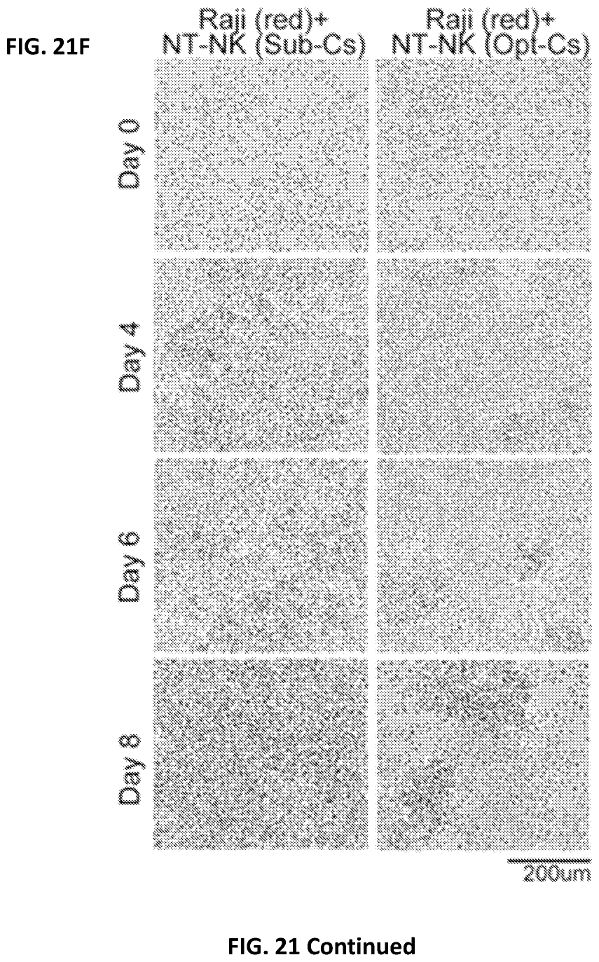

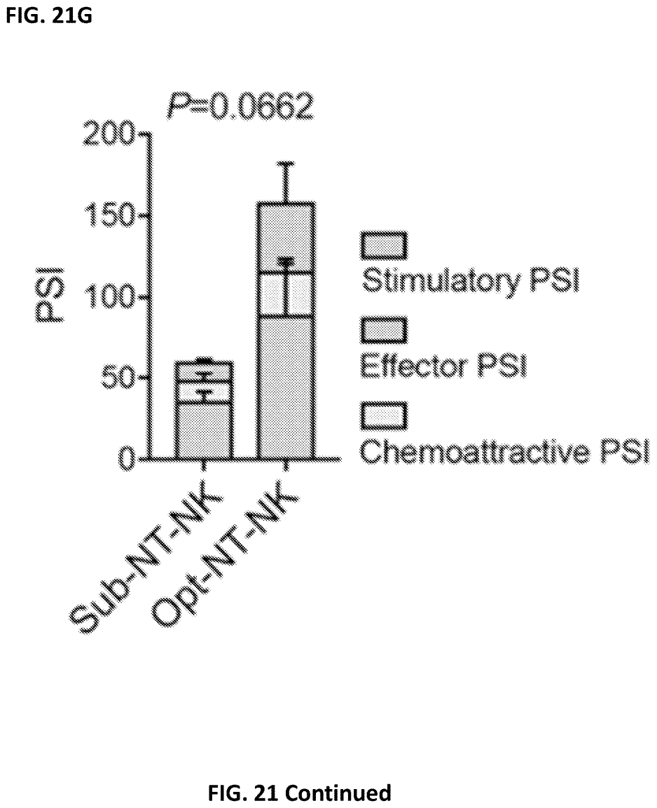

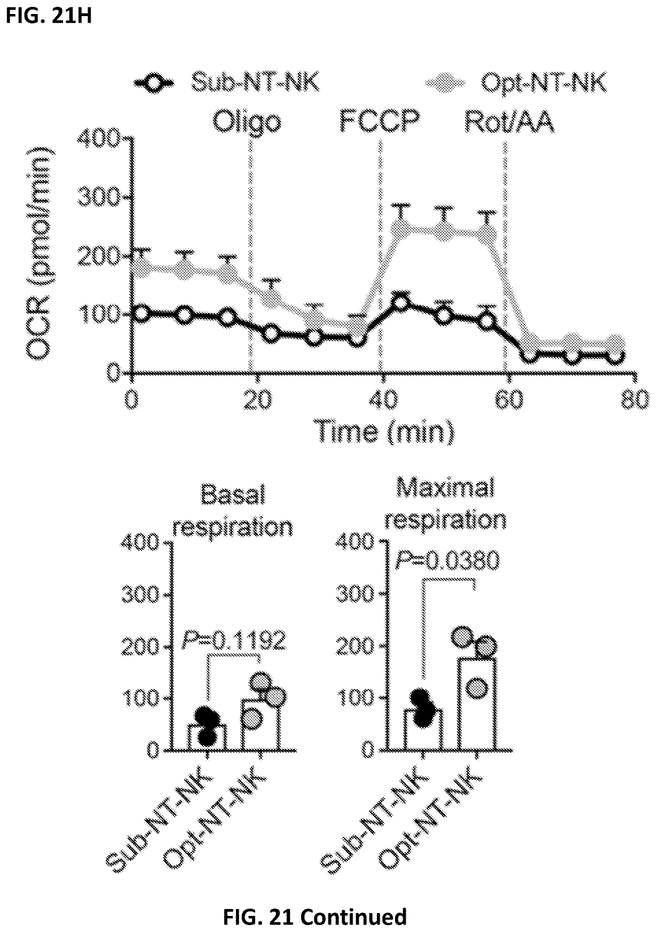

To facilitate elucidation of the underlying mechanisms for the differences in therapeutic efficacy based on CB quality, the inventors characterized CAR expression levels, in vitro proliferation, and the phenotypes of CAR19/IL-15 NK cells from the infused patients. These parameters were not significantly different between NK cells from Opt-Cs and Sub-Cs (see e.g., FIGS. 21A-21C). To assess the impact of cord quality on the proliferative capacity and cytotoxicity of NK cells against tumor targets, the inventors compared the cytotoxicity of clinical CAR NK cell products against Raji targets. There was no difference in CAR expression between the two groups of NK cells (Opt-Cs or Sub-Cs, where Opt-Cs were selected based upon time from birth to cryopreservation less than or equal to 24 h, and NRBC post-processing less than or equal to 8×10⁷). No differences were observed in the short-term cytotoxicity of CAR NK cells derived from optimal vs suboptimal CBUs when challenged with Raji cells at 1:1 effector: target ratios (E: T).

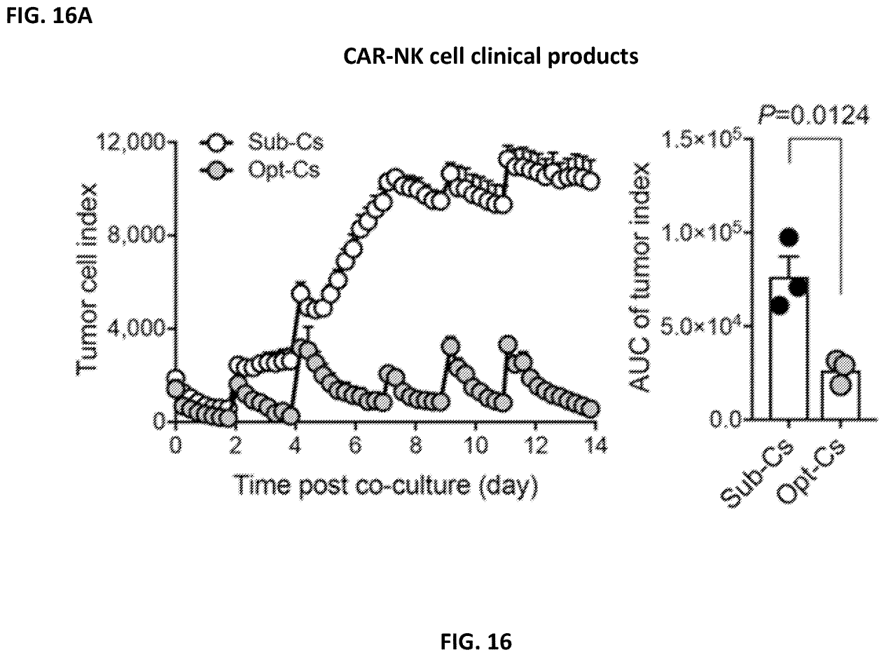

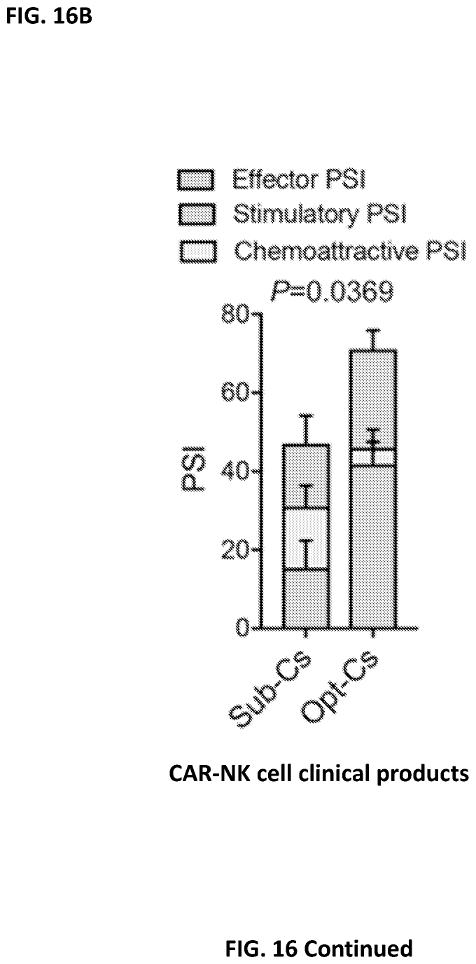

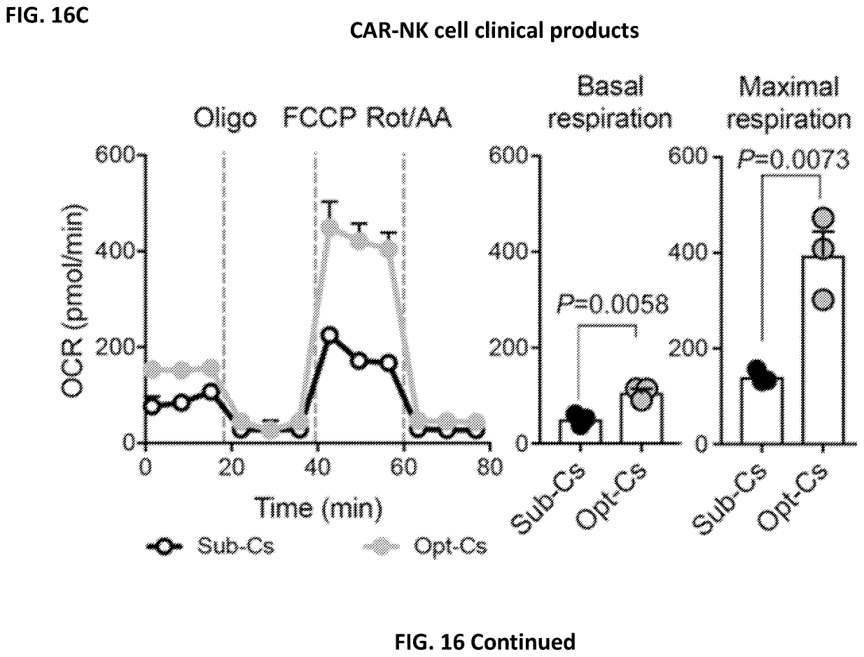

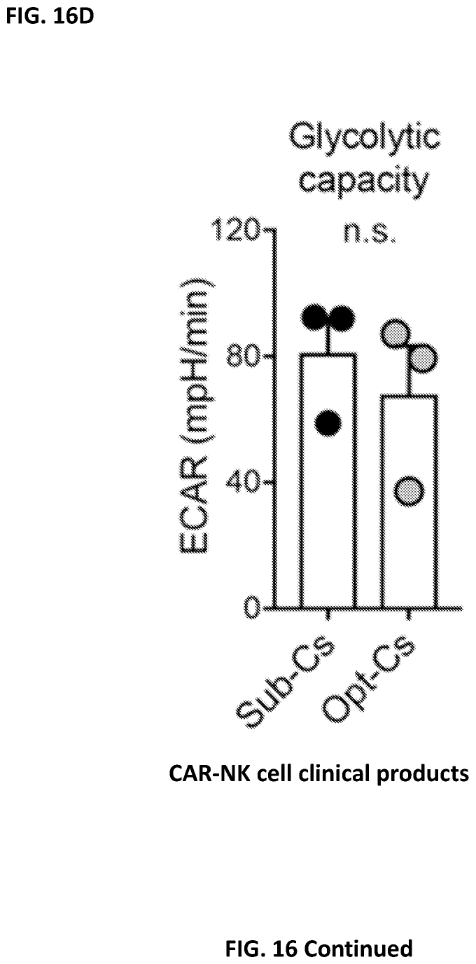

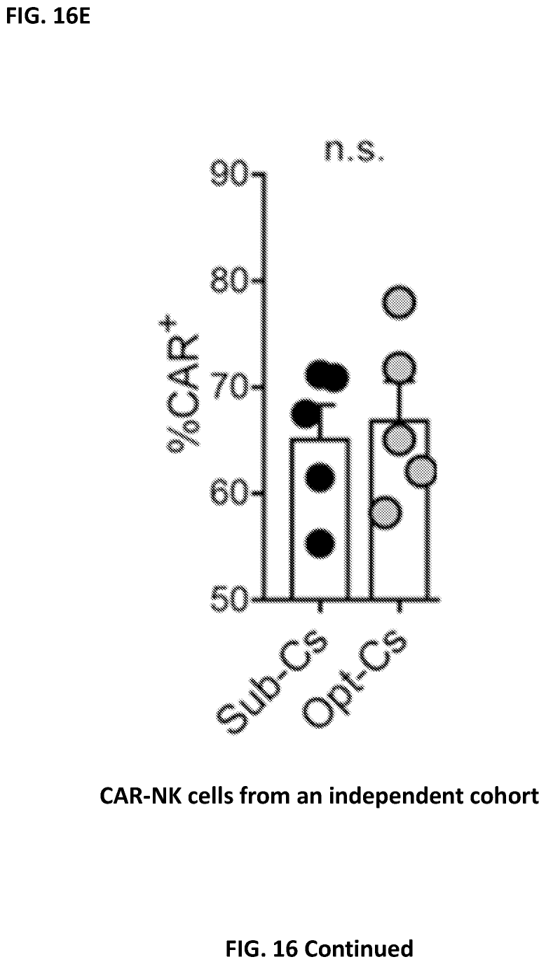

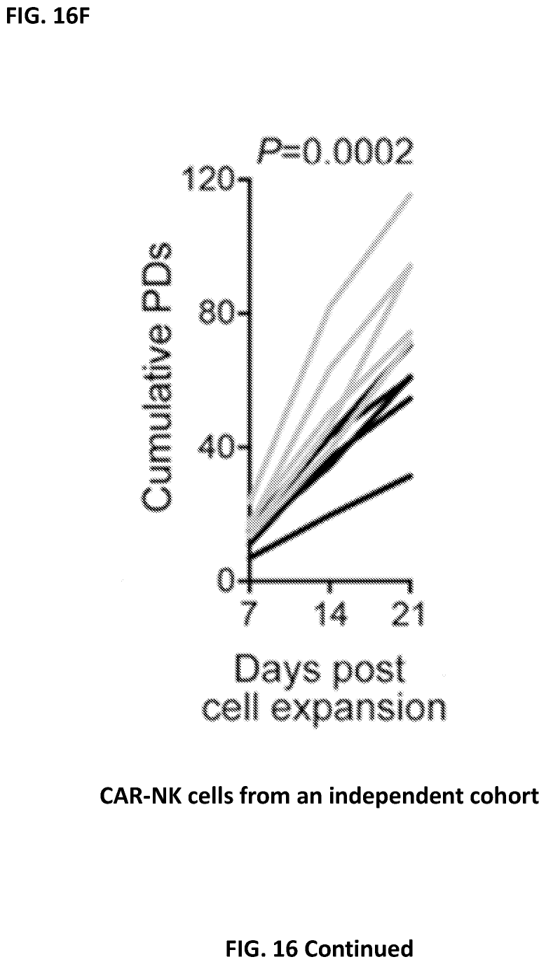

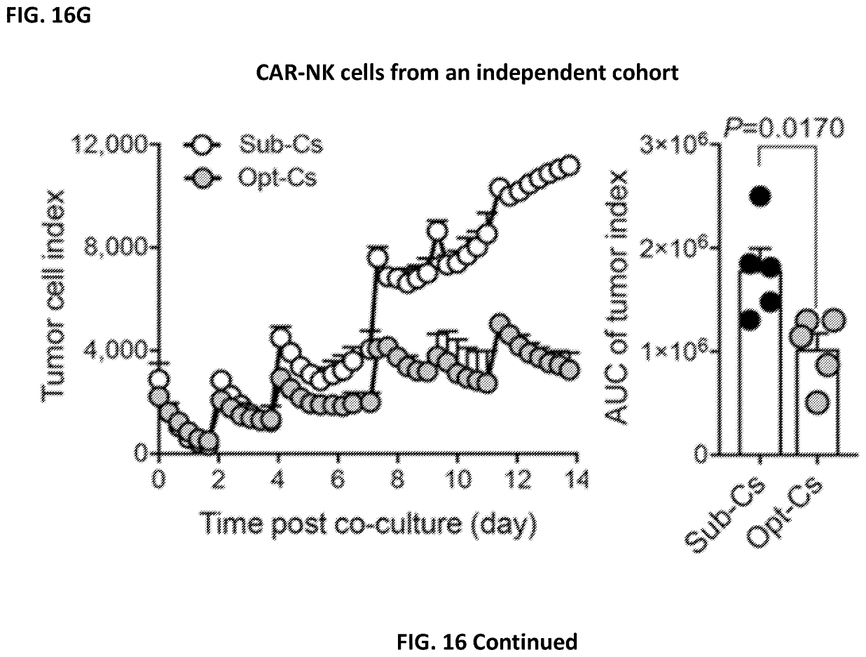

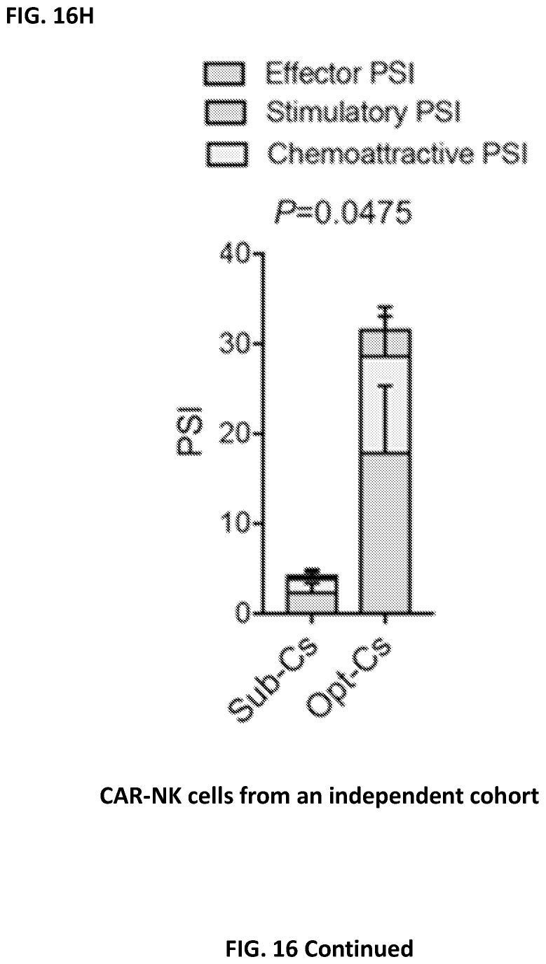

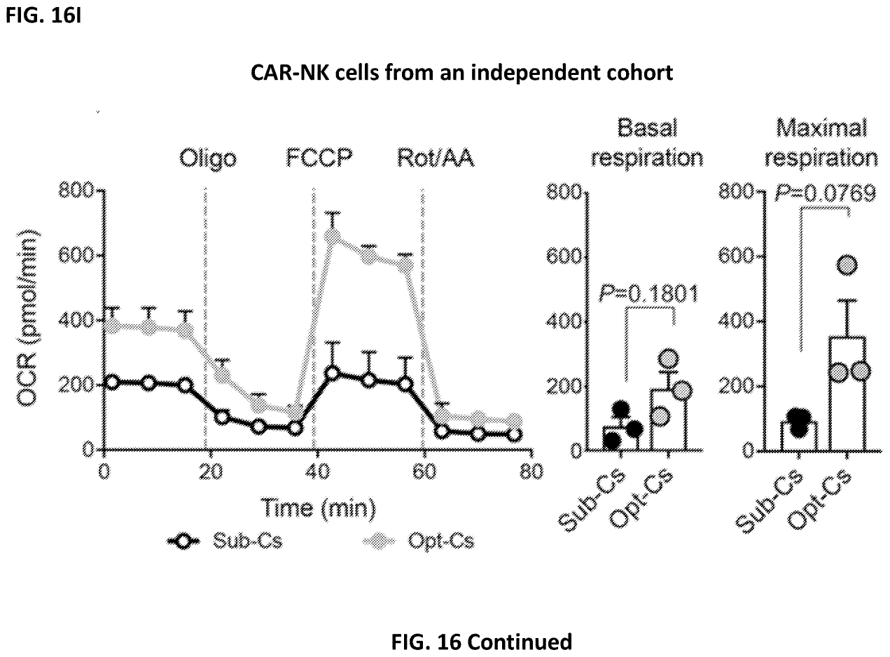

Polyfunctionality and metabolic fitness were considered important determinants of effective anti-tumor NK cell responses ^{28,29}. Single-cell IsoPlexis analysis showed that clinical CAR19/IL-15 NK cells from Opt-Cs had a significantly higher polyfunctional strength index (PSI) response to CD19 antigen stimulation compared to Sub-Cs (FIG. 16B). Analysis of mitochondrial metabolism and glycolytic activity showed higher oxidative phosphorylation in CAR19/IL-15 NK cells from Opt-Cs compared to Sub-Cs (FIG. 16C) with no difference in their glycolytic capacity (FIG. 16D), pointing to higher mitochondrial fitness.

The superior functional attirbutes of Opt-Cs was then validated in an independent cohort of CB units. The short- and long-term cytotoxicity of CD19-CAR/IL-15 NK cells generated from independent groups of Opt-Cs vs Sub-Cs was assessed against Raji targets. The inventors selected 12 additional CB units from the MDACC cord bank to generate CAR-NK cells. In vitro tumor rechallenge assays were conducted, in which the CAR NK cells were initially challenged at 1:1 E:T ratio by mCherry-transduced Raji cells, the CAR NK cells were then challenged with additional mCherry-transduced Raji cells (red) every 2-3 days for at least 6 tumor rechallenges. The results showed that CAR NK cells from both Opt-Cs and Sub-Cs cords were equally effective at eliminating Raji in the short-term at an Effector: Target ratio of 1:1. The results confirmed that CAR-NK and non-transduced (NT)-NK cells from Opt-Cs had superior long-term cytotoxicity against Raji tumor rechallenges, while those from Sub-Cs rapidly lost their ability to control respond to the Raji rechallenges despite excellent viability (FIGS. 16E-16G, and FIGS. 21D-21F).

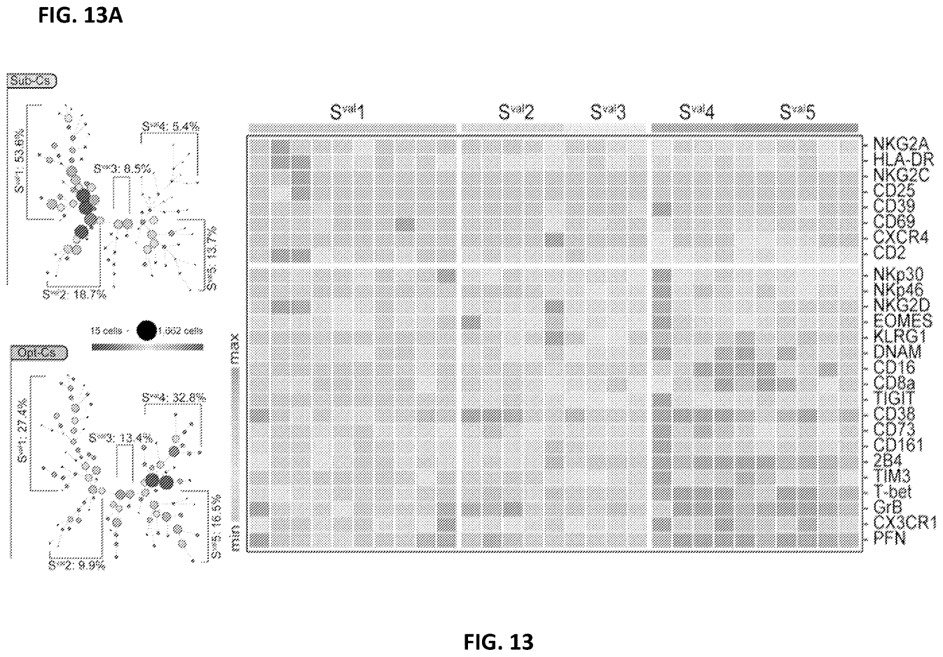

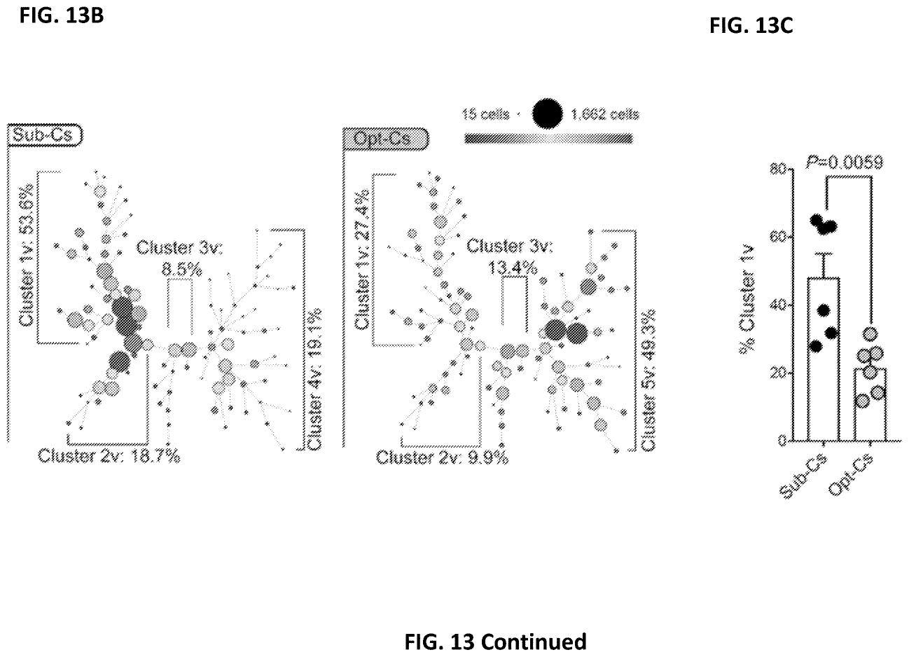

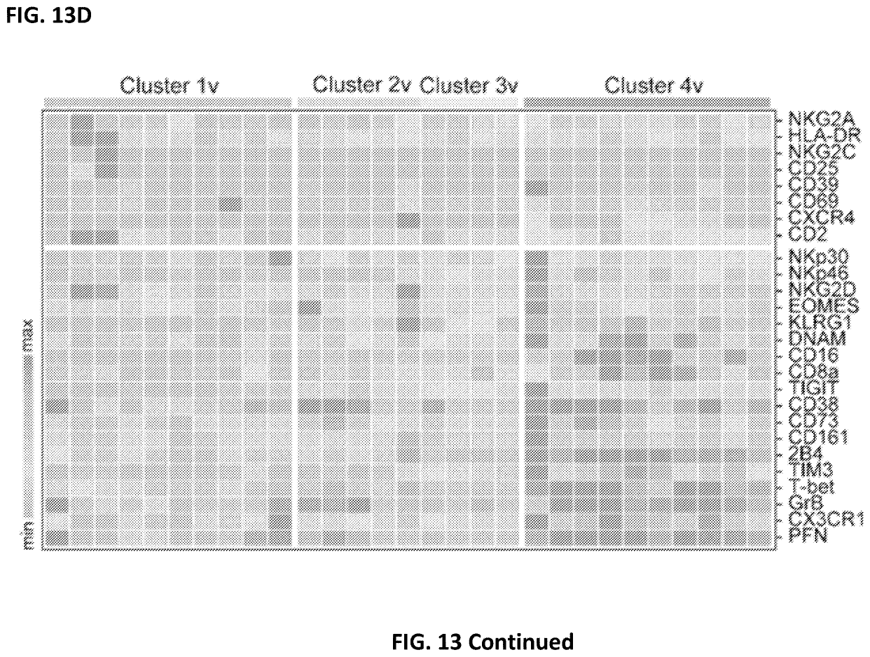

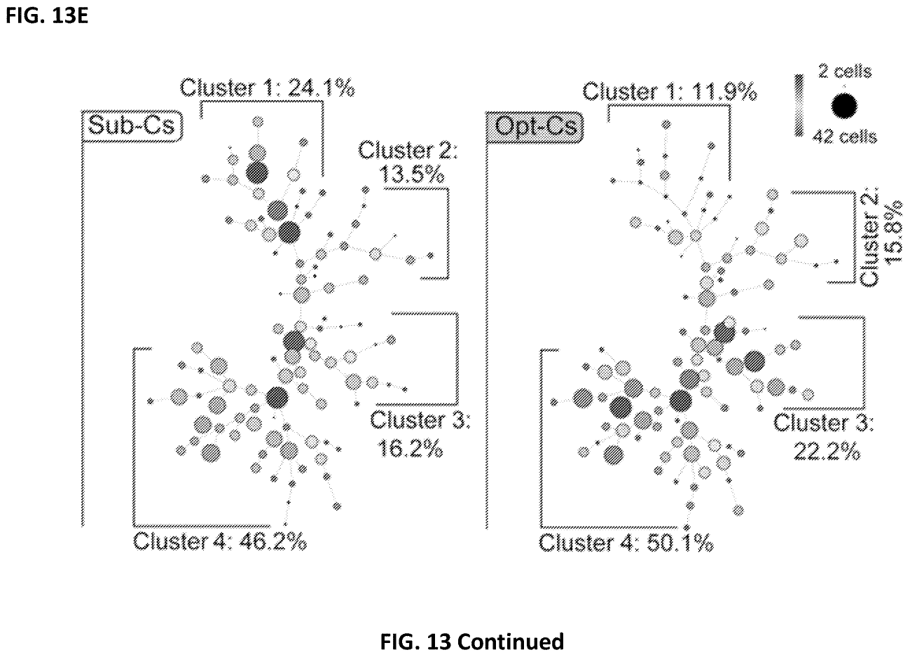

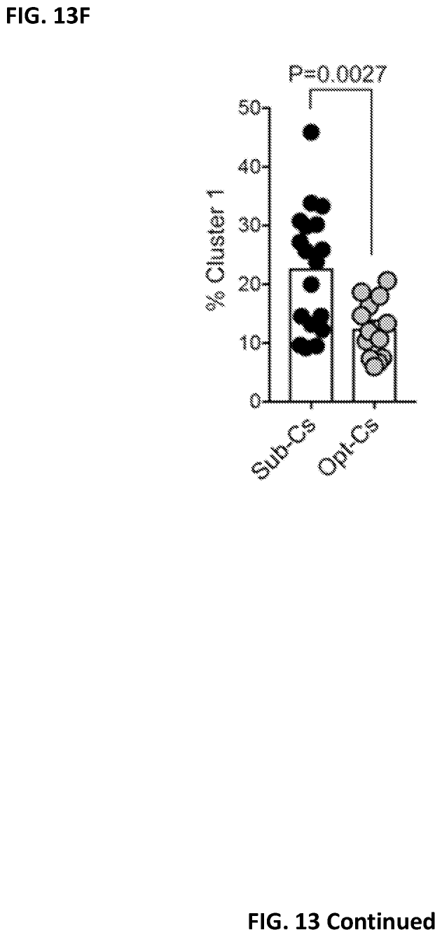

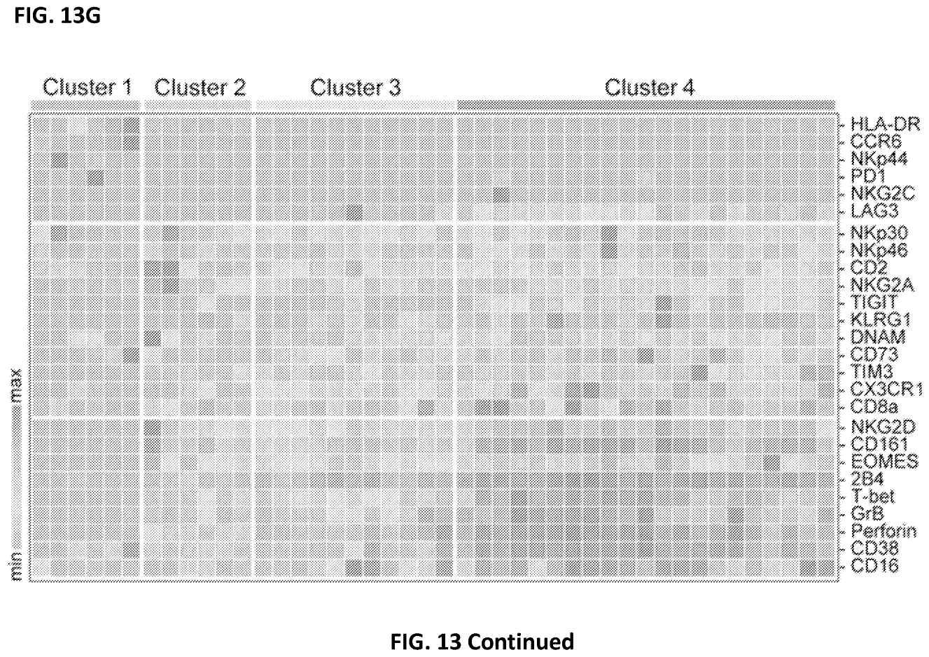

The phenotypic and transcriptional profiles of Sub-Cs and Opt-Cs derived NK cells were then analyzed. The phenotypes of NK cells generated from Sub-Cs and Opt-Cs CBUs were analyzed using multiparameter single cell analysis. CyTOF was utilized to phenotypically interrogate NK cells generated from the 16 Opt-Cs vs the 21 Sub-Cs clinical CBUs that were utilized to treat patients (e.g., the CBUs that were utilized to generate NK cells infused into patients; it was possible to produce many doses form a single CBU, thus on a number of occasions, 2 or 3 patients were treated with CAR-NK cells derived from a single CBU) (FIG. 13A). NK cells from Sub-Cs were present at higher frequencies in Cluster 1, while those from Opt-Cs were overrepresented in clusters 3 and 4 (FIG. 13A). Analysis of marker expression revealed clusters 3-4 to be enriched in NK cells with a highly functional phenotype, defined by the co-expression of multiple activating receptors (e.g., NKG2D, CD16, and 2B4), TFs important for NK cell activity (e.g., T-bet and EOMES), and cytotoxic granules (e.g., PFN, GZMA). In contrast, NK cells in cluster 1 did not express these functional/maturation markers (FIG. 13). The data confirmed a very similar signature of activation for NK cells expanded from Opt-Cs CBUs, even in the absence of CAR transduction, supporting the notion that the phenotypic differences in NK cells from Opt-Cs vs. Sub-Cs CBUs were NK cell-intrinsic and not driven by the CAR construct (see FIGS. 13B-13D). The superior PSI and mitochondrial fitness of Opt-Cs was also confirmed for both CAR-NK cells (FIGS. 16H-16I) and NT-NK cells (FIG. 21G-21H) from an independent cohort. Together, these data supported the notion that the superior effector function of NK cells from Opt-Cs was not induced or mediated by CAR19/IL-15 expression.

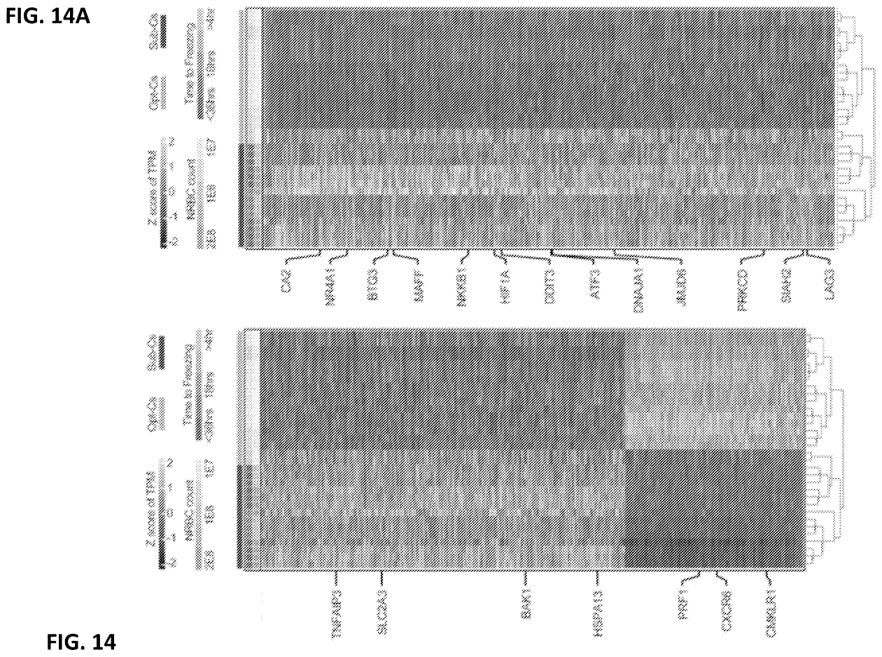

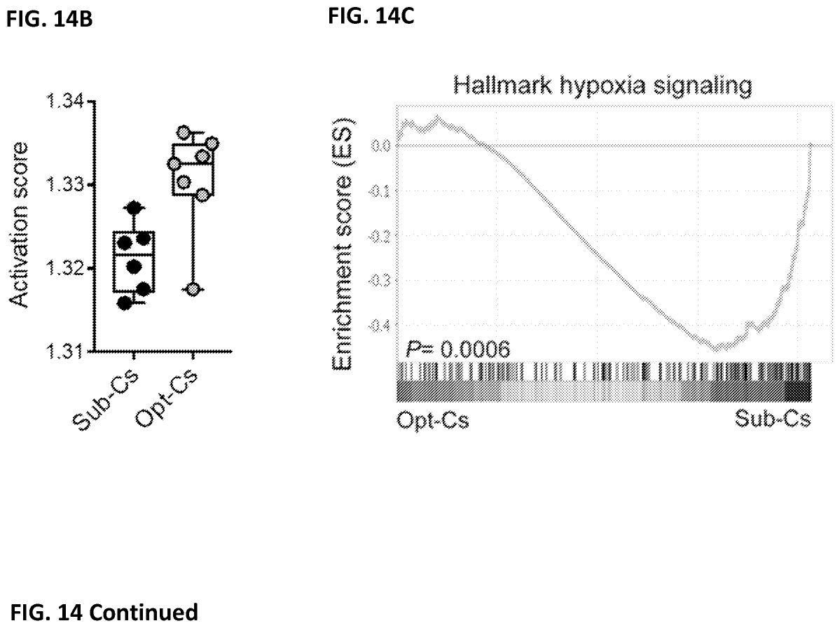

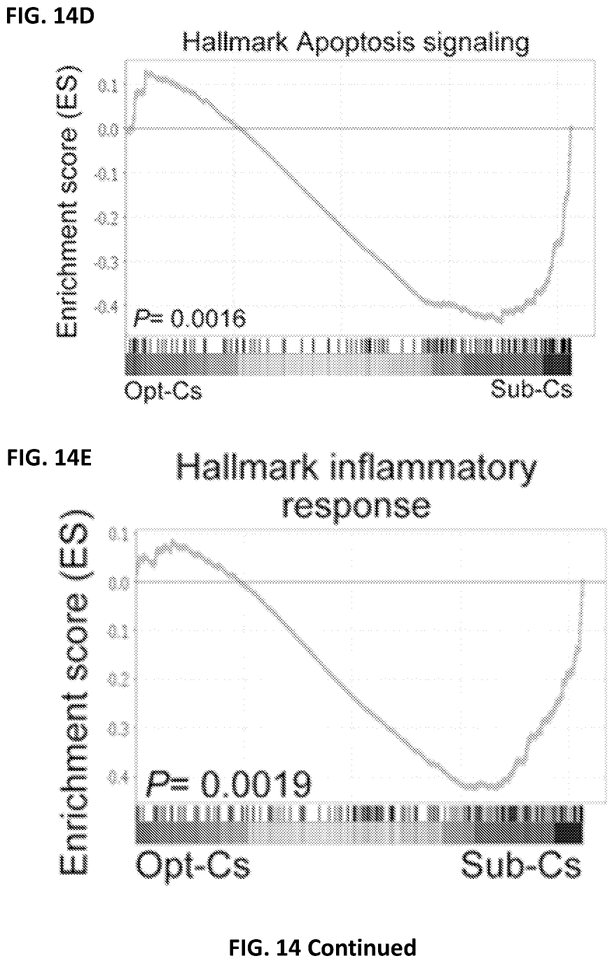

The transcriptomic profile of NK cells generated from Opt-Cs and Sub-Cs were investigated. Unmanipulated NK cells from Opt-Cs and Sub-Cs were found to have distinct phenotypic, transcriptomic, and epigenetic signatures. Sub-Cs were found to have a distinct transcriptomic profile (FIG. 14A) characterized by a hypoxia signature, and an apoptosis signature (FIG. 14C and FIG. 14D). Furthermore, Opt-Cs derived NK cells were found to have an increased activation score relative to Sub-Cs derived NK cells (FIG. 14B, activity of NK function signature (GZMA, PRF1, GZMB and CD247) was estimated in each sample using ssGSEA implemented in the R package GSVA. Difference between Opt-Cs and Sub-Cs was computed using two tailed Student's t-test.

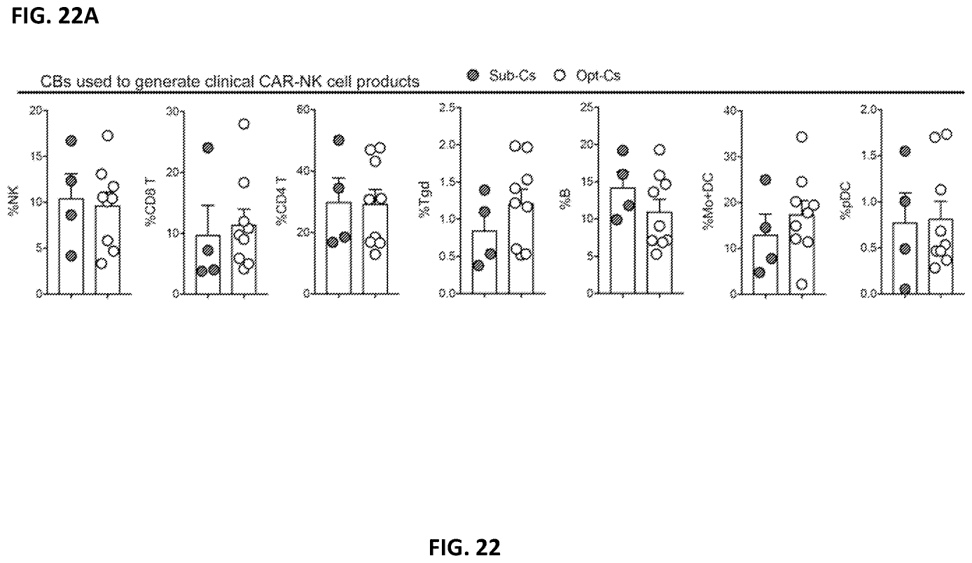

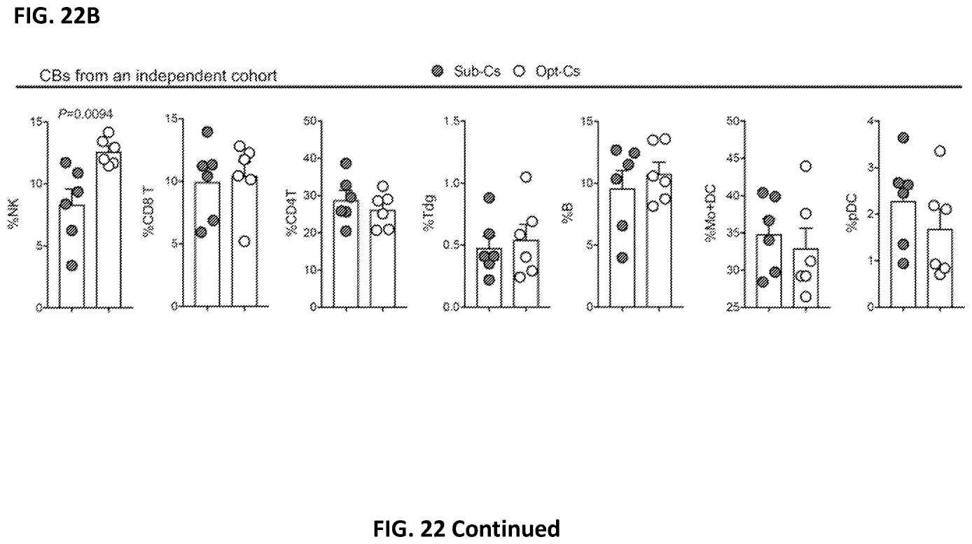

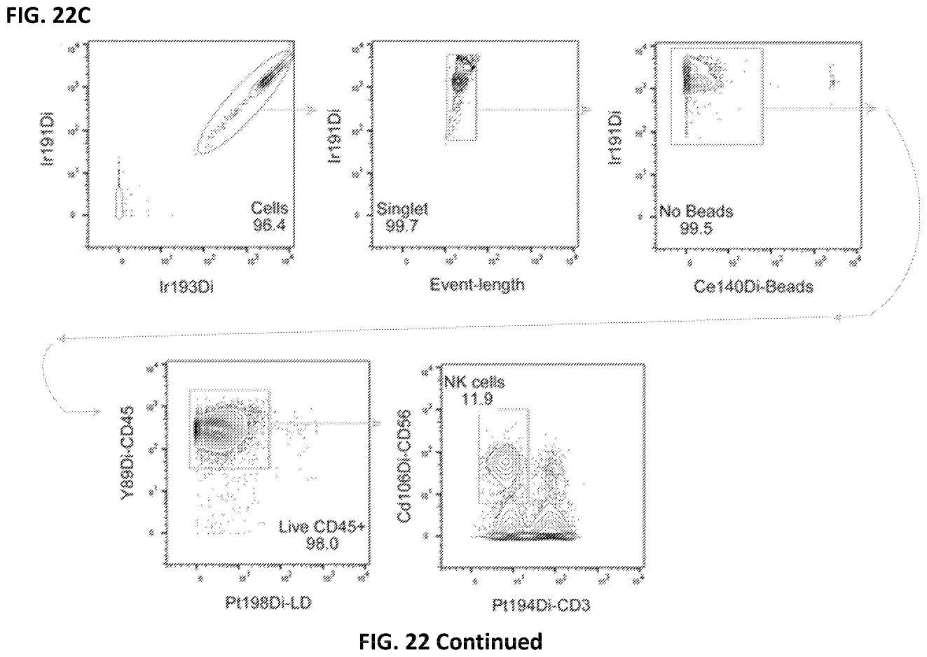

As shown in FIG. 21C, there were no significant phenotypic differences in the expanded CAR19/IL-15 NK cell products generated from Opt-Cs vs. Sub-Cs. As such, the inventors posited that ex vivo expansion could mask differences in the underlying phenotype of NK cells. Thus, the immune composition and the phenotype of unmanipulated NK cells in the cryopreserved CB mononuclear cells (CBMCs) stored in our cord bank from the cords used to manufacture the clinical CAR19/IL-15 NK cell products was examined. Strikingly, there were no significant differences in the frequencies of immune subsets in the CBMCs from Opt-Cs vs. Sub-Cs (FIGS. 22A-22B). Cytometry by time-of-flight (CyTOF) and built-in Spanning-tree Progression Analysis of Density-normalized Events (SPADE) analysis of CD45+CD56+CD3⁻ NK cells (gating strategy is shown in FIG. 22C) revealed 4 main clusters (Clusters 1-4; FIG. 13E). NK cells from Sub-Cs were present at higher frequencies in Cluster 1, while those from Opt-Cs were overrepresented in Clusters 3 and 4 (FIG. 13E-13F). Clusters 3 and 4 were enriched in NK cells with a highly functional phenotype, defined by the co-expression of multiple activating receptors (e.g., NKG2D, CD16 and 2B4), transcription factors (TFs) important for NK cell activity (e.g., T-bet and EOMES), and cytotoxic granules (e.g., PFN, GZMA), while NK cells in Cluster 1 did not express these functional/maturation markers (FIG. 13G). Differences in the phenotype of NK cells in CBMCs from Opt-Cs vs. Sub-Cs were validated in a second set of 12 CB units from MDACC's cord bank (FIG. 13B-13C).

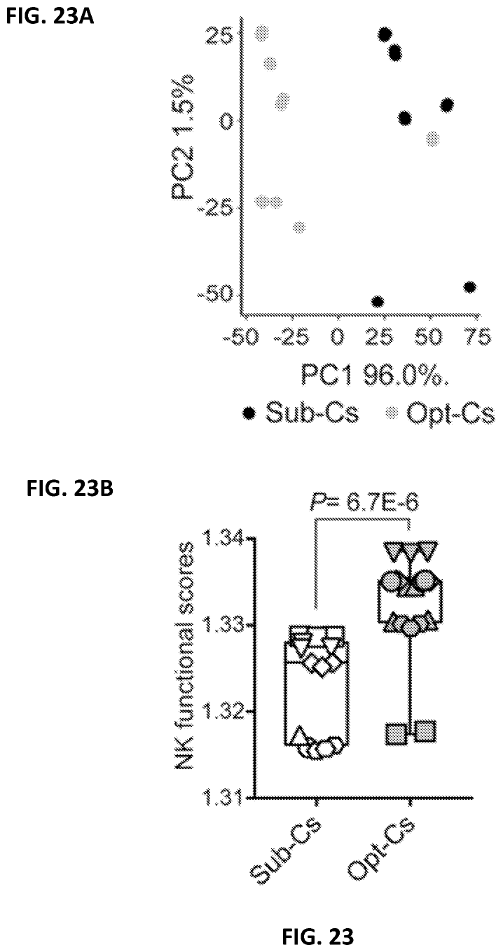

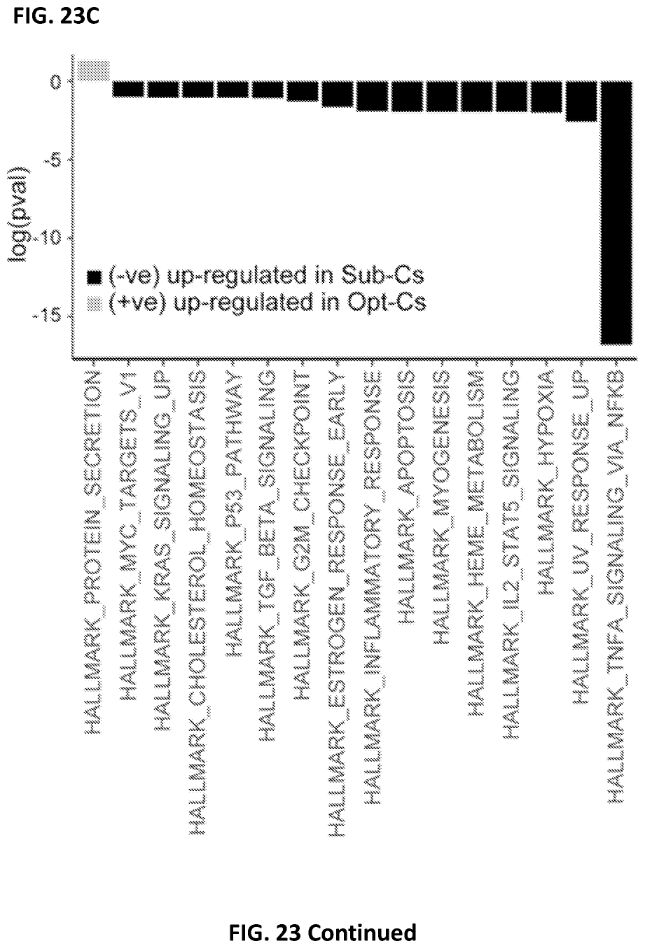

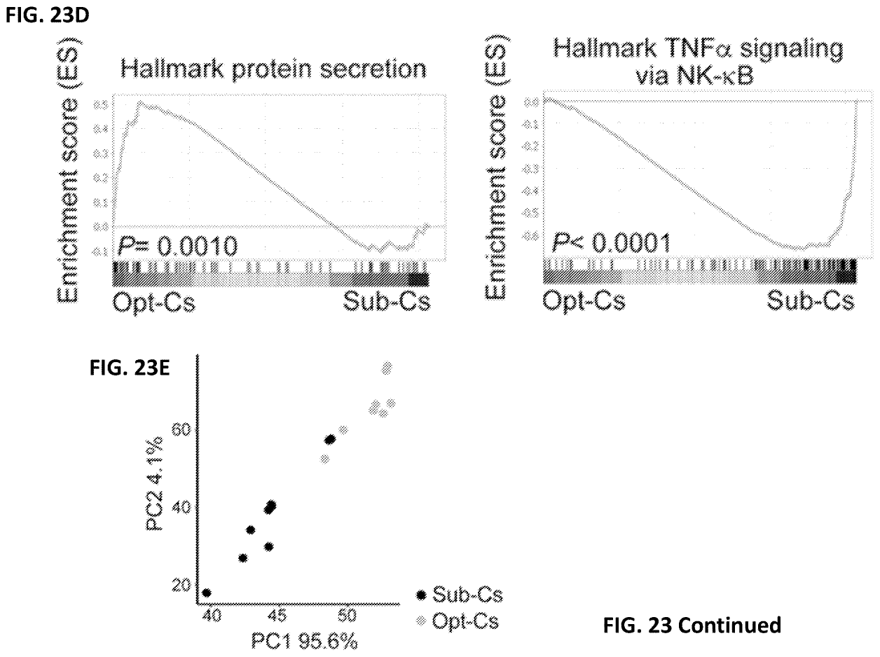

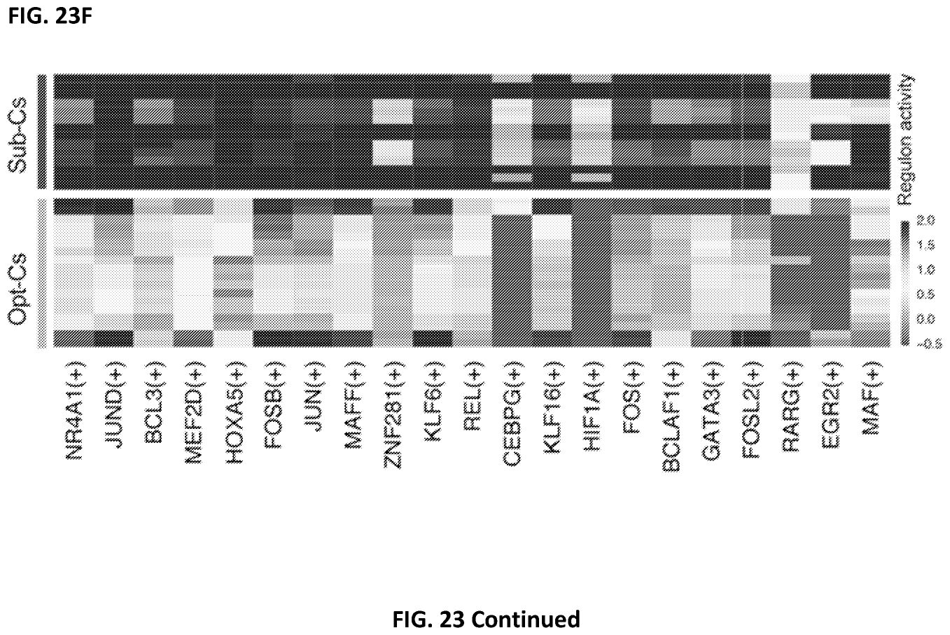

To investigate differences in NK cells from Opt-Cs and Sub-Cs at the transcriptomic level, bulk RNA sequencing (RNA-seq) was performed on unmanipulated NK cells purified from CBMCs of an independent set of nine CB units. Principal component analysis (PCA) resolved samples based on the optimal/suboptimal status (FIG. 23A), indicating their distinct transcriptomic landscapes. Analysis of differentially expressed genes (DEGs) in NK cells revealed important differences between the two groups (FIG. 14A). Opt-C NK cells were characterized by higher expression of effector genes like PRF1 and higher coordinated expression of NK functional genes (Methods; FIG. 23B), and chemokine signaling (e.g., CXCR6, CMKLR1), while Sub-Cs NK cells had upregulation of genes associated with hypoxia (e.g., HIF1A, MAFF, JMJD6, DDIT3, SIAH2), stress (e.g., NR4A1, DNAJA1, BAK1, ATF3, NFKB1) and immunosuppression (e.g., IL-10, LAG3; FIG. 3d). Notably, genes encoding stress-related heat shock proteins such as HASP90ABI, HSPA5, HSPA13, DNAJA1 were enriched in Sub-Cs compared to Opt-Cs. This pattern mirrored the stress response observed in T-cells in the context of immunotherapy resistance ³⁰ and the poor cytotoxicity seen in tumor-associated NK cells in a recent pan-cancer single cell atlas of human NK cells ²⁴.

Similarly, gene-set-enrichment analysis (GSEA) revealed activation of pathways related to protein secretion in Opt-Cs NK cells while pathways related to inflammation, hypoxia, apoptosis, TNF-α signaling via NF-κB response, and DNA damage were activated in Sub-Cs NK cells (FIGS. 14C-14E, and FIGS. 23C-23D). The distinctive hypoxia signature observed in NK cells from Sub-Cs was consistent with the higher NRBC content observed in these cords (e.g., potentially indicative of fetal hypoxia and stress) ^{21,22}.

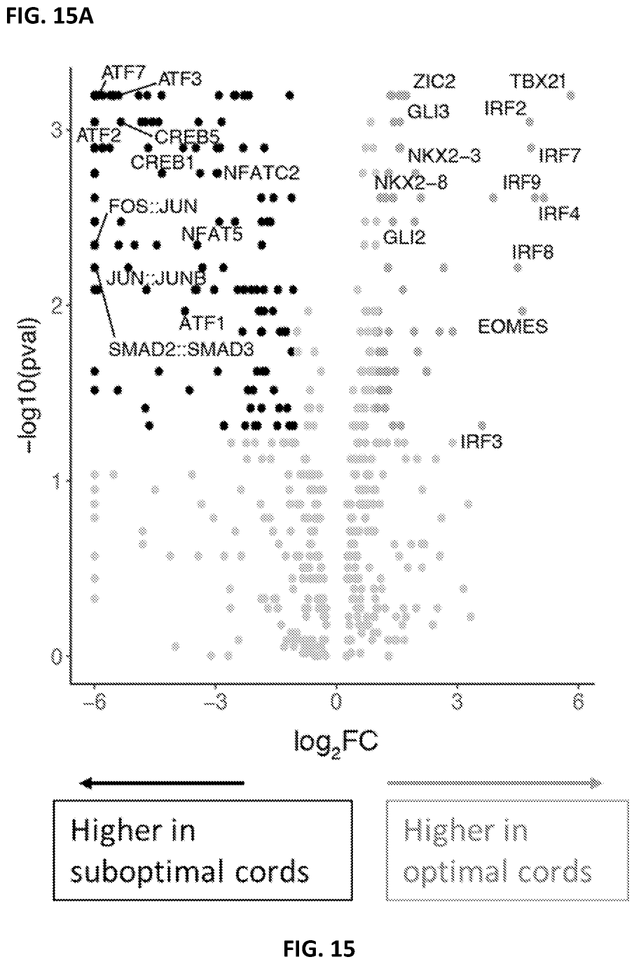

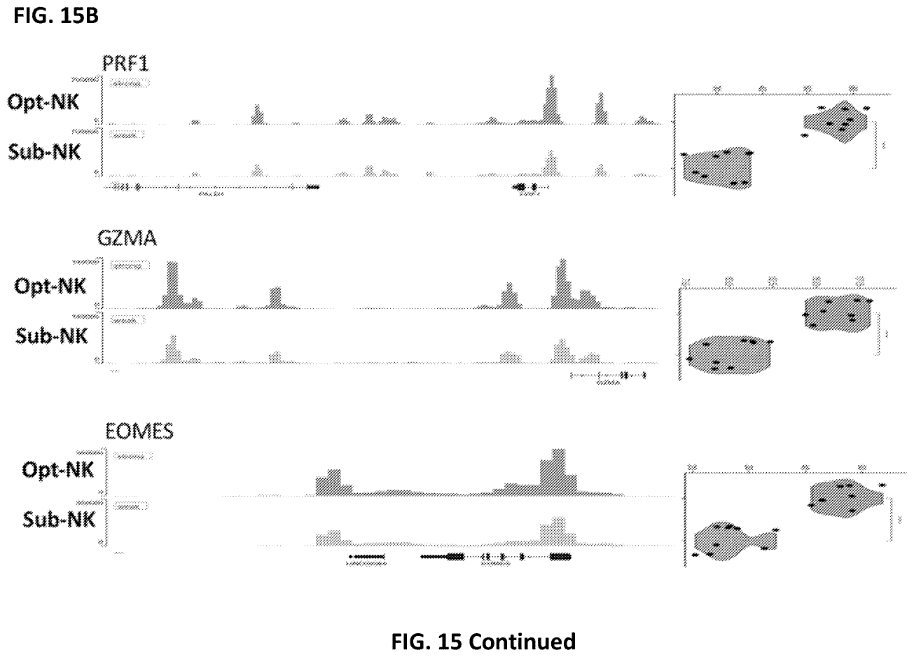

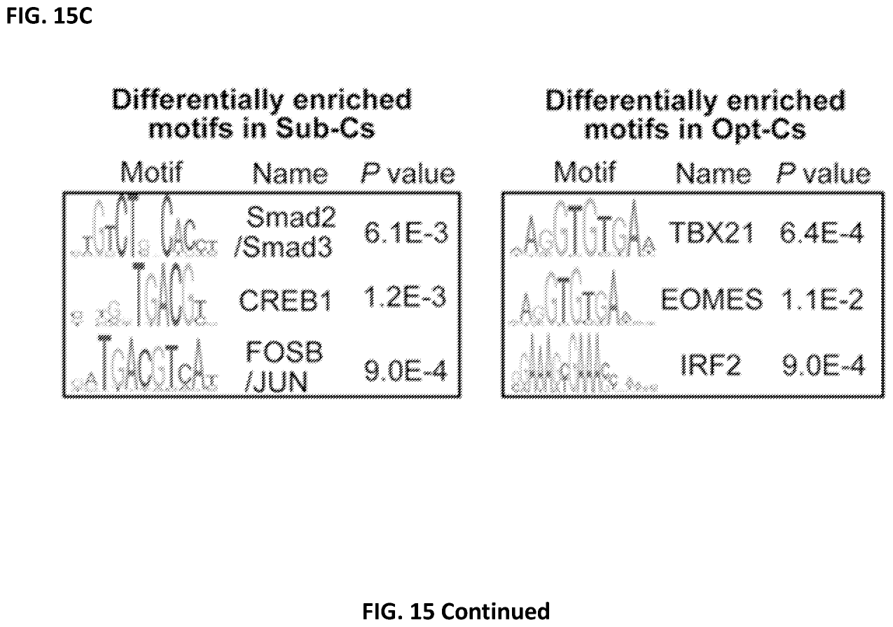

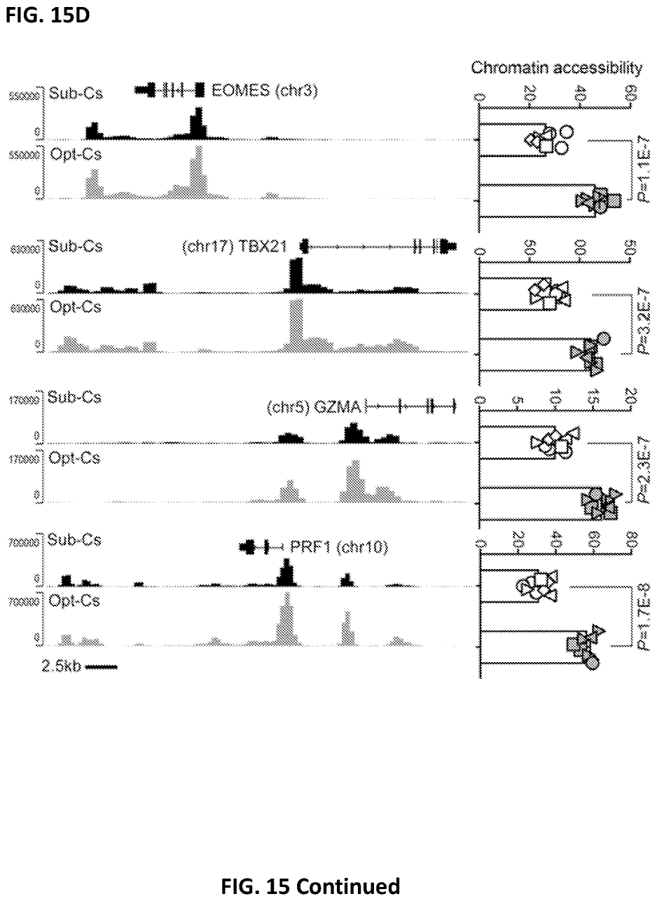

To understand differences in NK cells at the epigenetic level, the inventors performed ATAC-seq on ex vivo-purified unmanipulated NK cells derived from Opt-Cs and Sub-Cs. PCA of chromatin-accessible regions (ChARs) showed clear separation between the two groups (FIG. 23E). 13,729 differential ChARs were identified between the two groups (logFC>0.5, p<0.05). Differential motif enrichment analysis revealed NK cells from Opt-Cs had enrichment in motifs corresponding to TFs associated with NK effector function e.g., interferon regulatory factors (IRF) family (e.g., IRF4, IRF7, IRF8, IRF9, IRF2, IRF3), T-box (e.g., TBX21) and EOMES (FIG. 15A and FIG. 15C). Consistent with these findings and congruent with the activated state shown at the proteomic and transcriptomic levels, ATAC-seq track analysis revealed significantly greater accessibility at the transcription start sites and promoter regions of genes related to NK effector function such as PRF1, GZMA, EOMES, and TBX21 in Opt-Cs (FIG. 15D), supporting an epigenetic state poised towards increased effector function. In contrast, the motifs that were enriched in Sub-Cs NK cells corresponded to TFs that regulated cellular responses to stress and inflammation and that have been linked to immune dysfunction, such as the AP-1 complex family (e.g., FOS, JUN, JUNB, FOSL1) ^{31,32}.

To integrate and validate these ATAC-seq data findings with gene expression from RNA-seq data on the same samples, the pySCENIC workflow was utilized to predict key regulons, score their activities, and identify differentially active regulons in NK cells from Opt-Cs and Sub-Cs. Several consistent trends were observed between the RNA-seq and ATAC-seq analyses. Specifically, the activity of the HIF1A regulon, a hypoxia-induced master regulator of the cellular response to hypoxia, was significantly higher in NK cells from Sub-Cs than Opt-Cs (adjusted p-value<0.01), suggesting that these cells may have been exposed to hypoxic conditions (e.g., as also indicated by the higher NRBC content of the cords). In addition, several members of the AP-1 complex (e.g., JUND, FOSB, FOS, JUN, FOSL2) were significantly more active in Sub-Cs NK cells (adjusted p-value<0.01), consistent with the role of AP-1 in regulating cellular responses to stress and inflammation (FIG. 23F).

In summary, the results showed consistent biological differences at the proteomic, transcriptomic, and epigenetic levels in NK cells derived from the two CB groups. These traits may account for the observed superior clinical activity of CAR-NK cells generated from Opt-Cs relative to those generated from Sub-Cs.

### Example 5-Validation of Efficacious Cord Blood Characteristics In Vivo

To validate the in vivo antitumor function of CAR NK cells generated from Opt-Cs vs Sub-Cs CBUs, three different CAR constructs and three different preclinical models were utilized. Each experiment was performed with a different set of CB units that were distinct from those used in our clinical trial or in the validation studies described above. CAR-NK cells from Opt-Cs were found to exert superior anti-tumor activity in each of the independent preclinical models tested.

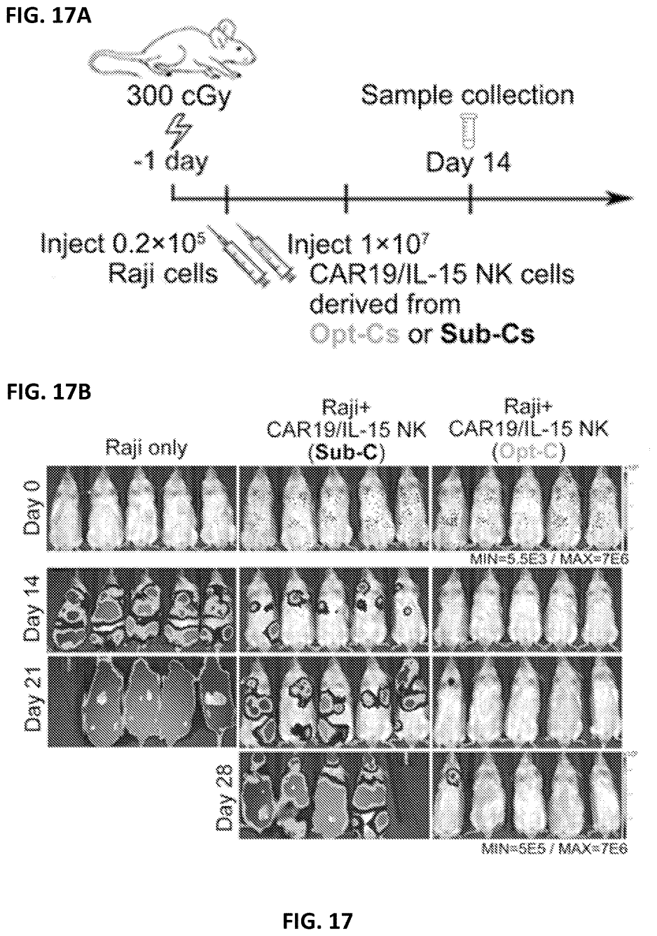

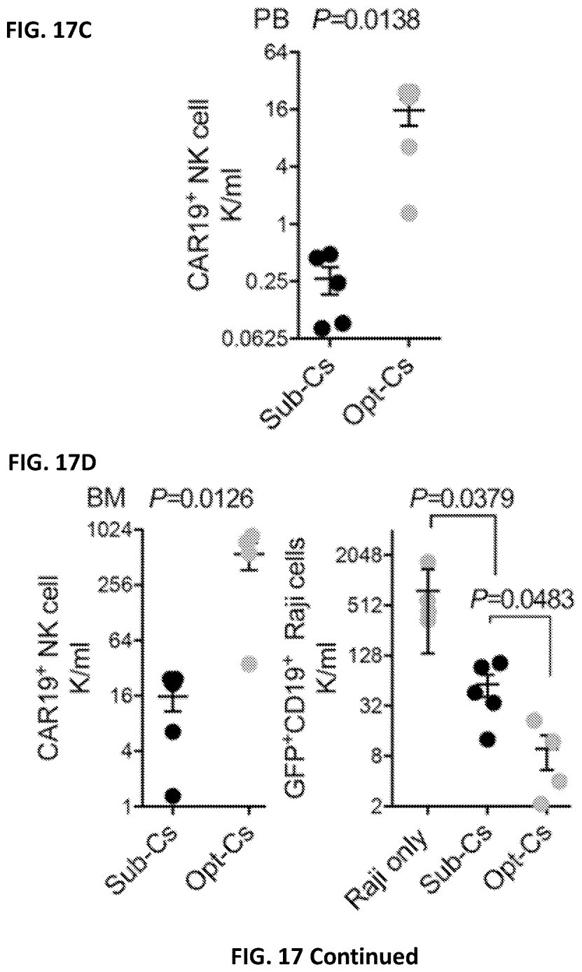

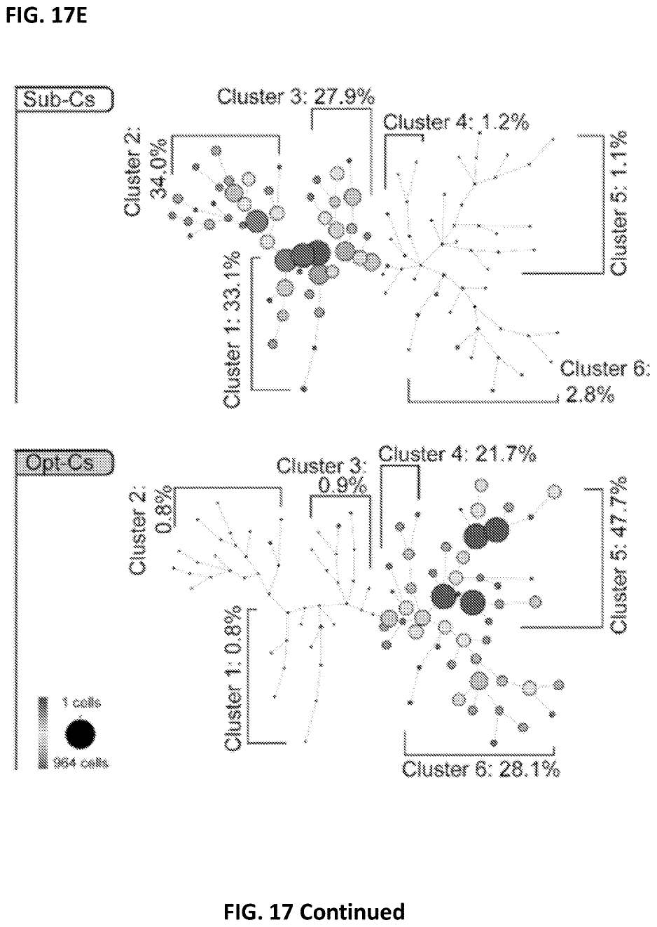

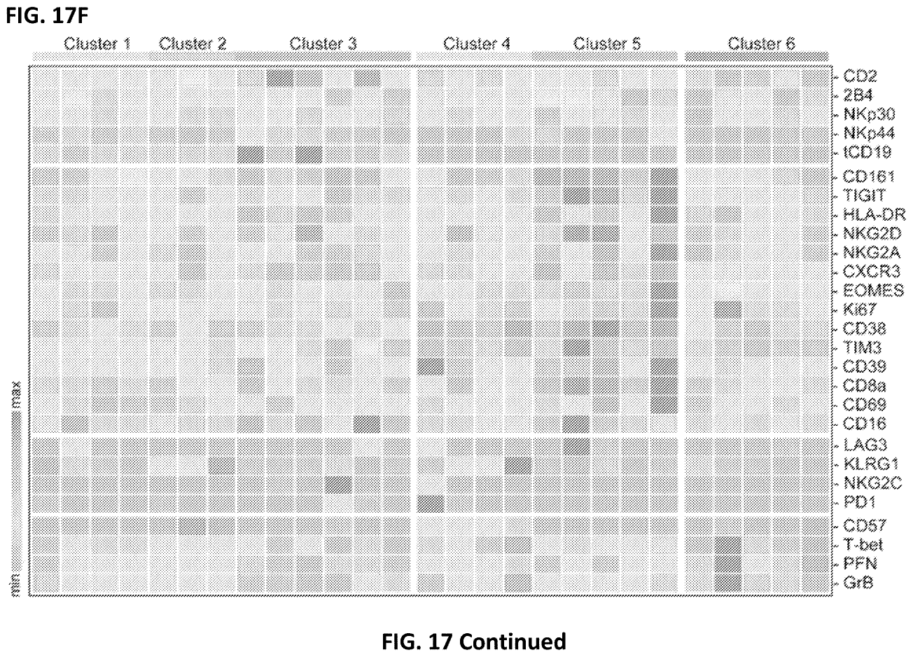

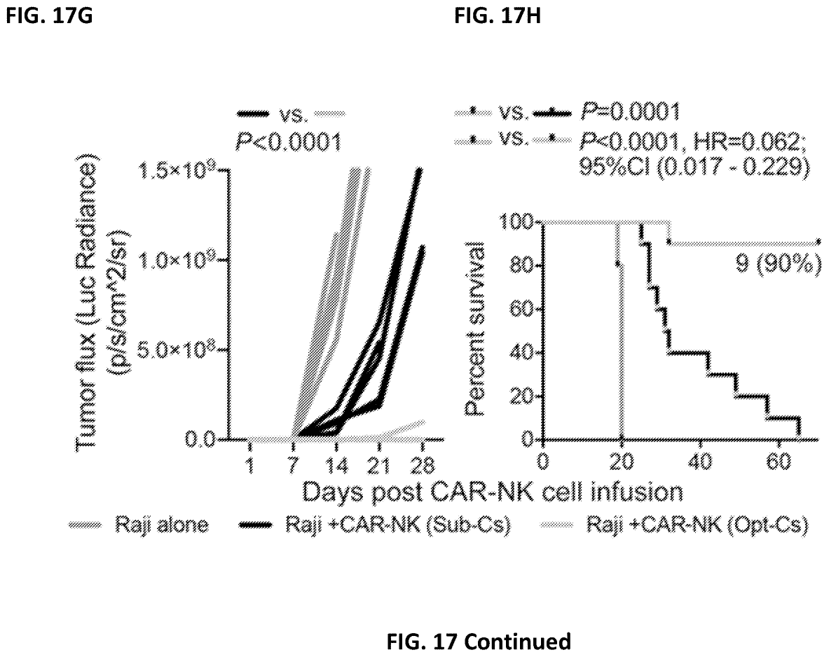

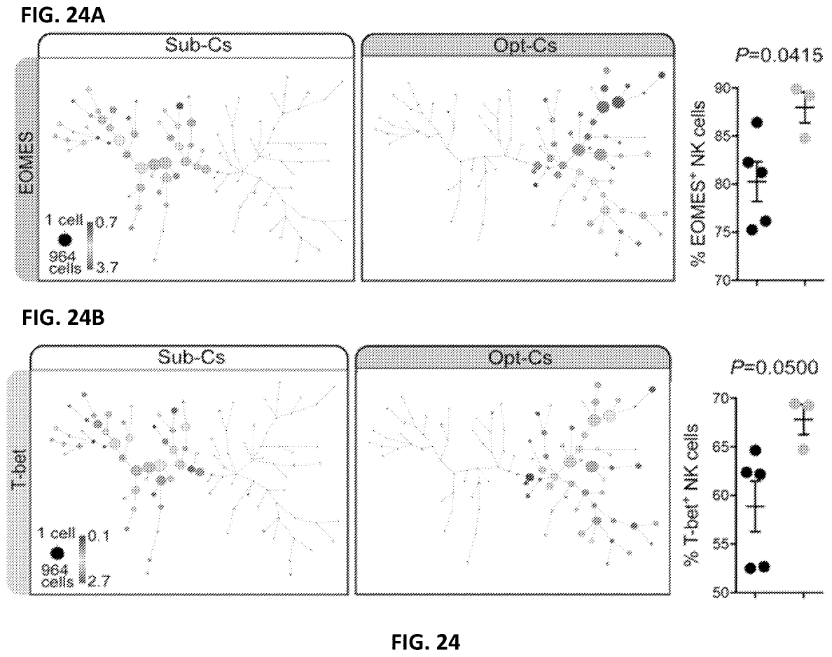

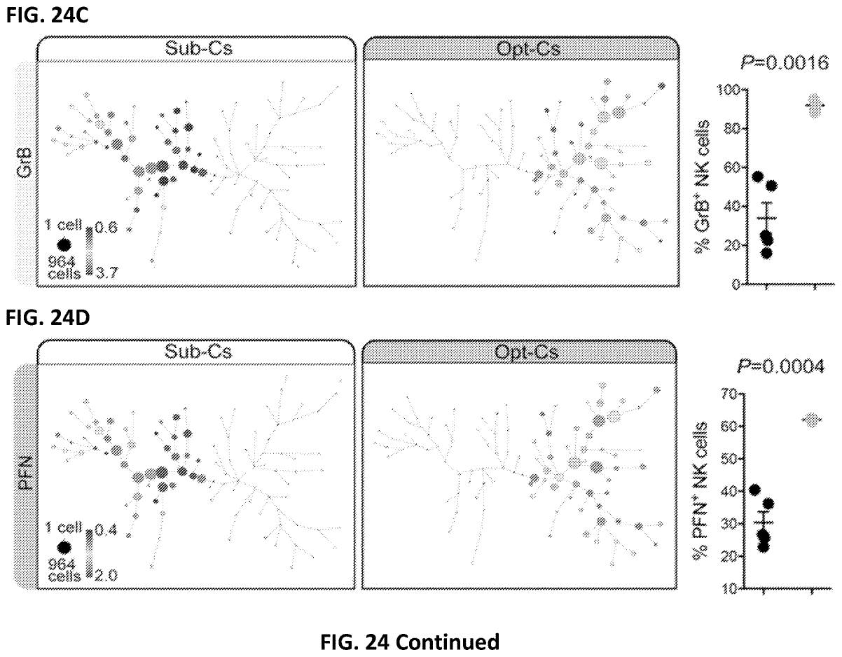

As shown in FIGS. 17A-17G, the in vivo kinetics of CAR-NK cell proliferation were analyzed in a mouse model of lymphoma (Raji cell). In one experimental cohort, mice were sacrificed at two weeks following CAR-NK cell infusion, with their blood and tissues harvested for comprehensive phenotypic analysis by CyTOF. The inventors observed a significantly higher frequency of CAR NK cells, which was associated with lower tumor burden, in animals treated with Opt-Cs derived CD19-CAR/IL-15 NK cells when compared to CD19-CAR/IL-15 cells derived from Sub-Cs. The phenotypic signatures of Opt-Cs vs. Sub-Cs derived CAR-NK cells in the bone marrow (BM) of mice at day 14 post infusion was performed using a Spanning-tree Progression Analysis of Density-normalized Events (SPADE). SPADE analysis segregated NK cells into six clusters, with CAR19/IL-15 NK cells from Opt-Cs dominating Clusters 4-6 and those from Sub-Cs preferentially located in Clusters 1-3 (FIG. 17E). In keeping with the earlier described in vitro data, CD19-CAR/IL-15 NK cells from optimal cords had higher expression of transcription factors such as EOMES and T-bet, cytolytic proteins (e.g., PFN and Granzyme B), cytokine receptors IL-2R (CD25), activating receptors (e.g., NKG2D), co-activating receptors, and chemokine receptors, and lower levels of trogocytosis (TROG)-antigen acquisition (tCD19) when compared with their Sub-Cs derived CD19-CAR/IL-15 NK cell counterparts (see FIGS. 17E-17F, and FIGS. 24A-24D). In an independent experimental cohort, the inventors confirmed that Raji-xenografted mice receiving CD19-CAR/IL-15 NK cells from Opt-Cs CBUs had superior tumor control and improved survival, with increased proliferation and longer persistence of CAR NK cells in vivo compared to animals treated with CD19-CAR/IL-15 cells NK cells from Sub-Cs CBUs (FIGS. 17B, and 17G-17H).

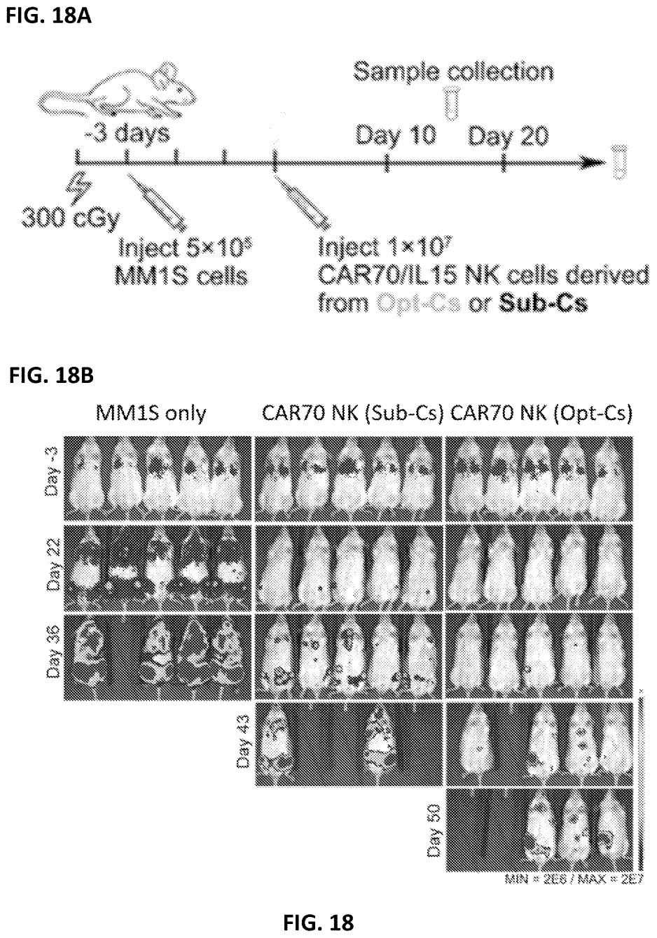

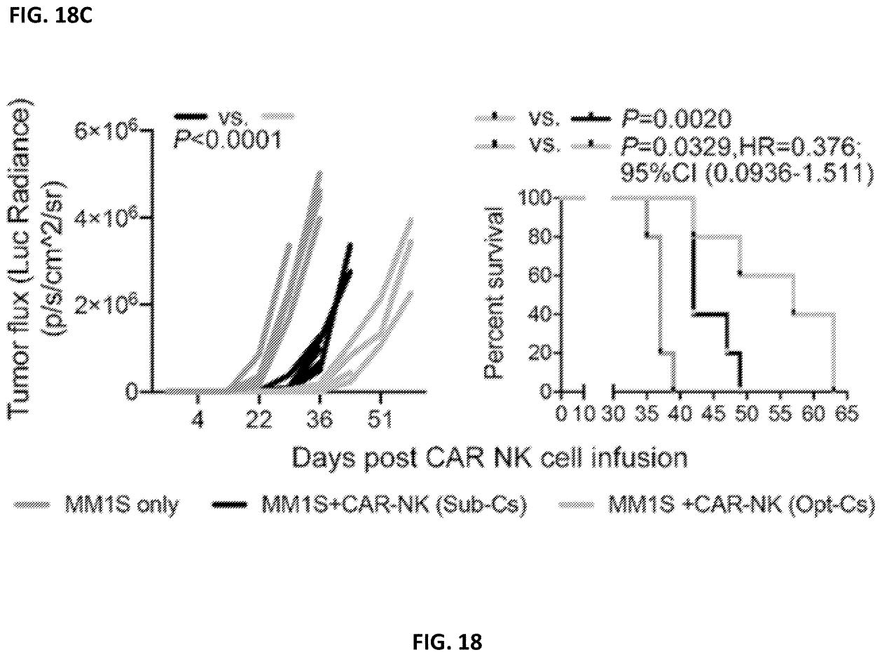

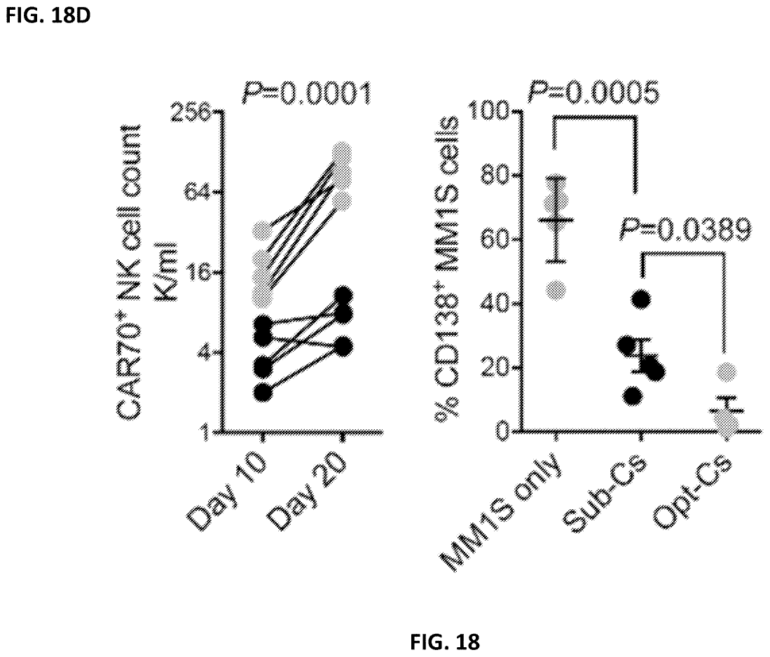

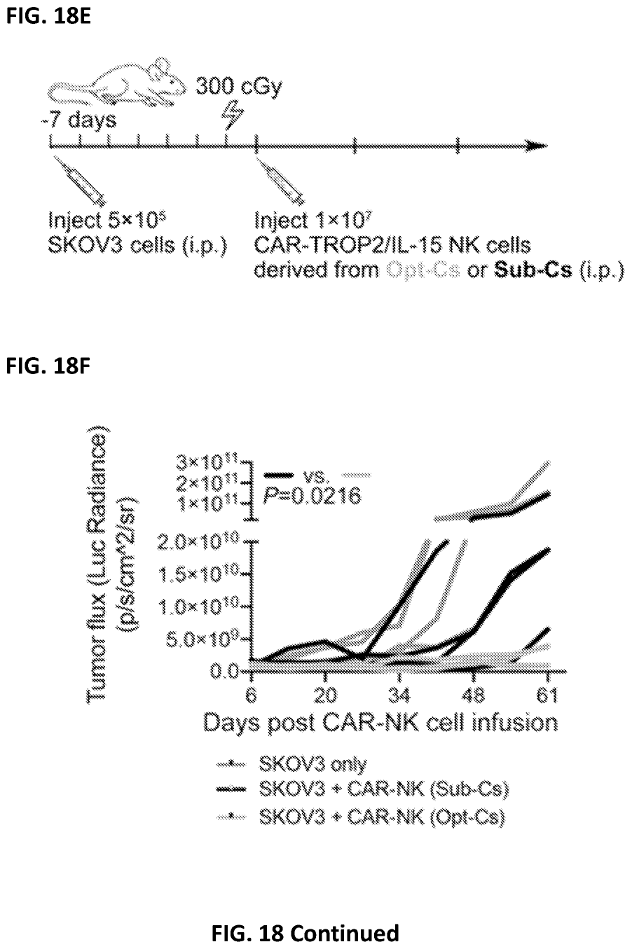

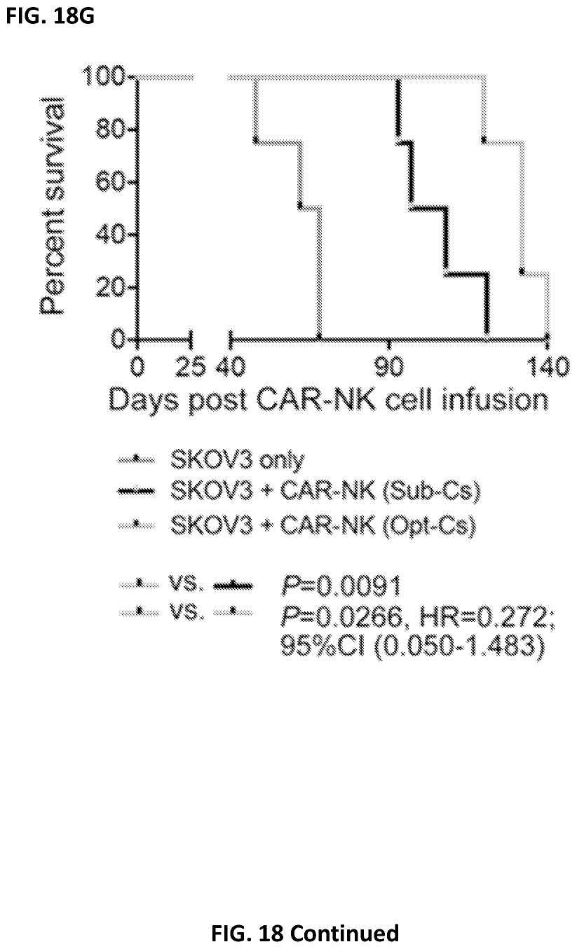

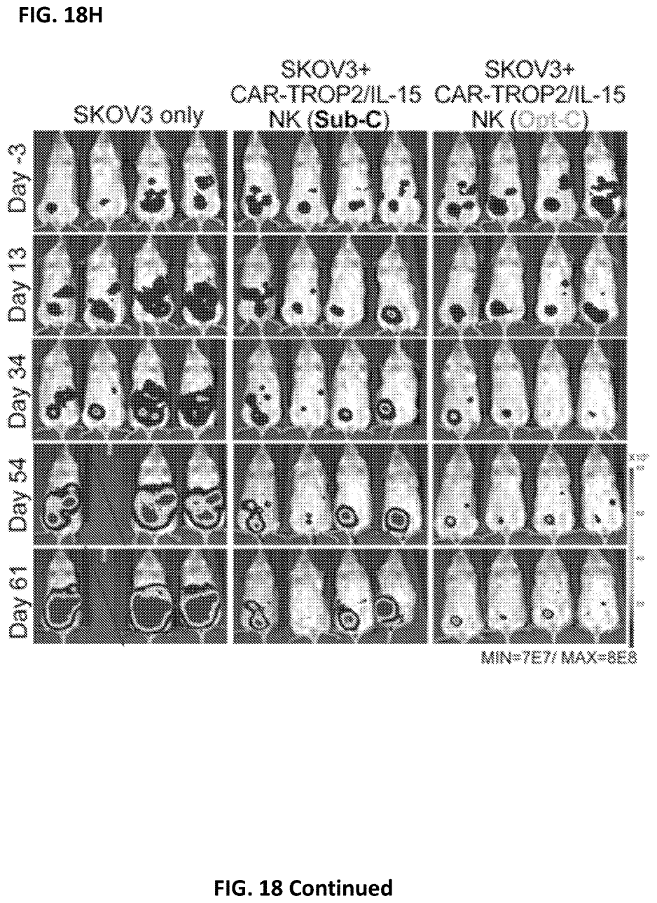

The inventors also verified the results described herein in a mouse mule of multiple myeloma. As shown in FIGS. 18A-18D, mice were engrafted with MM.1S tumor cells and treated intravenously (IV) with anti-CD70-CAR/IL-15 (CAR70/IL15) NK cells generated from Opt-Cs vs Sub-Cs CBUs. Significantly improved tumor control (FIGS. 18B-18D), in vivo proliferation of CAR NK cells (FIG. 18D), and superior survival in the animals (FIG. 18C) was observed for animals treated with Opt-Cs derived CAR/IL15 NK cells when compared to Sub-Cs derived CAR70/IL-15 NK cells. Finally, the inventors also verified the results in a solid tumor model of SKOV3 ovarian cancer treated with a single infusion of anti-TROP2-CAR/IL-15 NK cells (CAR-TROP2/IL-15). CAR-NK cells from Opt-Cs resulted in superior anti-tumor control and survival compared to those from Sub-Cs (FIGS. 18E-18H).

Together, these results provided compelling experimental evidence that immune effector cells (e.g., NK cells, e.g., CAR-NK cells), generated from Opt-Cs mediated a stronger anti-tumor response, associated with significantly better proliferation and persistence in vivo, and increased subject survival.

### Example 6-Discussion

Herein the inventors have presented the results of a first-in-human phase I-II study of CB-derived engineered NK cells expressing an anti-CD19 CAR, a cytokine (IL-15), and a safety switch based on inducible caspase-9. The clinical trial enrolled 37 heavily pretreated patients with multiple relapsed or refractory B-cell malignancies (see Table 1). Responses were rapid and observed at all dose levels, with 100% of patients with low grade NHL achieving an OR, 67% of patients with CLL without transformation achieving an OR, and 41% of patients with DLBCL achieving an OR. The majority of responses were CRs, with a 1-year CR rate of 83% 50% and 29%, respectively.

In the described clinical trial, CD19 CAR-NK cells were manufactured directly from banked CBUs, eliminating the need to perform a leukapheresis for each patient. This characteristic limited the direct comparison of the immediate results with those reported for similar patient populations treated with autologous anti-CD19 CAR T-cells, although indirect comparisons can be drawn. Additionally, most anti-CD19 CAR T-cell studies report analysis of outcomes only for patients who actually have received effector cells (a modified intention-to-treat analysis) and not from the initial screening visit (intention-to-treat analysis). The interval between diagnosis and treatment is known as an important prognostic factor for DLBCL (see e.g., Maurer, M. J., et al., Diagnosis-to-treatment interval is an important clinical factor in newly diagnosed diffuse large B-cell lymphoma and has implications for bias in clinical trials. J Clin Oncol 36, 1603-1610 (2018); which is incorporated herein by reference in its entirety for the purposes described herein), and those patients who can afford to wait for their therapy have naturally less aggressive disease with better prognosis. This is an important consideration when assessing the true efficacy of therapy as it creates an inherent selection bias and ‘filters out’ from the analysis those patients with aggressive disease who cannot wait for completion of cell manufacturing. For example, in the immediate study, the CR rate for patients with DLBCL was 29% which appears lower than that reported by others for autologous anti-CD19 CAR T-cell therapy (which report rates from 40 to 64%). However, when the data were analyzed on an intention to treat basis, the CR rate reported for DLBCL patients treated with autologous CAR-T cells is 34% (95% confidence interval of 27-42%), which is very similar to the immediate results when analyzed on an intention to treat basis, namely 27.8% (95% confidence interval of 10 to 53%) (see e.g., Schuster, S. J., et al., Tisagenlecleucel in adult relapsed or refractory diffuse large B-cell lymphoma. New England Journal of Medicine 380, 45-56 (2019); which is incorporated herein by reference in its entirety for the purposes described herein). In contrast, in the immediate study, results for patients with more indolent diseases such as low-grade lymphoma or CLL compared favorably with those results from patients treated with autologous CAR T-cells, where the literature reports CR rates of 71-73% for patients with indolent NHL ^{35,36}, and 28-45% of patients with CLL achieved CR ^{37,38}.

During the phase I portion of the immediate trial, two patients who had achieved a CR received consolidation with a stem cell transplant (SCT), while an additional two patients received additional targeted or immunomodulatory agents. In the phase 2 part of the immediate study, post-remission therapy was not administered to any patient. Responses were durable, with a 70% probability of remaining in CR at one year for those patients achieving early remission. Similar results have been reported for patients with lymphoid malignancies receiving autologous anti-CD19 CAR T-cells ^{34,39}, with achievement of an early CR being associated with sustained response.

The use of allogeneic immune cells from healthy donors can offer several advantages over autologous patient-derived cells, including but not limited to, generation of multiple therapeutic cell doses from a single donor that could be cryopreserved for off-the-shelf use, making the allogeneic products cost-effective, readily available, and with the potential for a consistent and high-quality treatment. The importance of the quality of the starting material for manufacturing has been described for autologous CAR T-cell therapies, where patient baseline T-cell characteristics such as polyfunctionality, increased stemness, and decreased exhaustion features were predictive for CAR T-cell proliferation, persistence, and therapeutic response ^{14,40-42}. However, it is important to note that even with healthy donors, there is heterogeneity with regards to their natural immunological host defenses. Indeed, in an 11-year follow-up study of >3,600 healthy donors, large variations in immune cytotoxic activity were observed among individuals. Notably, higher NK cell cytotoxicity was associated with reduced cancer risk while lower activity was associated with increased risk 43.

In the immediate study, donor-related factors such as the NRBC content and the time from collection-to-cryopreservation were discovered to be significant predictors for therapeutic outcome, defining the concept of the optimal CB unit. This discovery stresses the importance of identifying donor-specific predictors of response after allogeneic cell therapy, especially since one donor may be used to treat hundreds if not thousands of patients. Such biomarkers may be relevant to cell products beyond NK cells.

In the immediate study, the criteria for Opt-C selection was based on a limited sample size, a factor which may necessitate further validation in a larger clinical cohort. Nonetheless, the results provided herein have been extensively validated, with the selection criteria being applicable in multiple experimental models. First, the inventors measured the in vitro cytotoxicity of the CAR19/IL-15 NK cells infused to patients against CD19-expressing tumor cells, and showed that CAR-NK cells from Opt-Cs had greater long-term cytotoxicity against multiple tumor rechallenges, associated with greater metabolic fitness and polyfunctionality compared to Sub-Cs. Second, the inventors selected a different set of CB units from the MDACC cord bank and confirmed that NK cells derived Opt-Cs units had greater long-term cytotoxicity, greater metabolic fitness, and greater polyfunctionality. These findings were independent of whether the NK cells were transduced with CAR19/IL-15 or not, indicating that this was an NK cell intrinsic phenomenon and not driven by the CAR. Third, the inventors validated these findings in three different tumor mouse models: namely, Raji lymphoma treated with CAR19/IL-15 NK cells, MMIS multiple myeloma treated with CAR70/IL-15 NK cells, and an ovarian SKOV3 cancer model treated with CAR-TROP2/IL-15 NK cells. For each in vivo experiment, a new set of Opt-Cs and Sub-Cs was utilized for CAR-NK cell generation. In each disease model, CAR-NK cells generated from Opt-Cs had better in vivo proliferation and resulted in superior tumor control. These findings strongly supported the validity of the selection criteria utilized, irrespective of the CAR and/or the disease model being studied. Therefore, these criteria for donor selection are being implemented in ongoing and future clinical trials with CB-derived NK cells.

As reported herein, the biological mechanisms underlying the CB-derived NK cell variability were investigated. No significant phenotypic differences in the infused CAR-NK cells were observed; however, there were notable differences in the phenotype of the unmanipulated NK cells in the CBs prior to expansion. NK cells from Opt-Cs were enriched in a population of cells with a functional phenotype, characterized by expression of activating receptors, TFs such as EOMES and T-bet, and cytotoxic granules. At the transcriptomic level, and in keeping with the CyTOF analysis, NK cells from Opt-Cs had a higher functional score, while those from Sub-Cs had a signature of hypoxia, which may have been induced by fetal hypoxia as suggested by the higher NRBC ^{21,22,44}, and cellular stress possibly induced by longer time from collection-to-cryopreservation. Similarly, chromatin accessibility analysis by ATAC-seq revealed global differences between the two groups, with TFs associated with effector function and IRFs being more abundant in NK cells from Opt-Cs, while those associated with hypoxia (e.g., HIF1α) and cellular response to stress and inflammation ⁴⁵, such as members of the AP-1 complex, more abundant in Sub-Cs ^{31,32}. The data also suggested a degree of epigenetic scarring in NK cells from Sub-Cs, as their functional impairment was not reversible by ex vivo expansion and activation, despite recovery of their phenotype.

The adoptively-infused CAR NK cells were detectable in some cases for over a year following infusion, supporting the inventors previous reports of the persistence of these types of CAR NK cells, despite significant HLA-mismatch with the recipient in all patients. IL-15 expression in the construct may be what supports the continued in vivo proliferation and persistence of the engineered NK cells. As found in the immediate study, greater in vivo expansion of CAR NK cells was associated with clinical response, a similar finding was also previously reported after CAR T cell therapy.

The results provided herein have confirmed that allogeneic CAR NK-cells have an excellent safety profile. No cases of graft-versus-host disease (GVHD) or neurotoxicity were observed, and only one case of grade 1 CRS was observed. No patients required treatment with steroids, tocilizumab, or the safety switch rimiducid dimerizer. No patients required transfer to ICU for management of complications, and no patients died of treatment-related causes. These results compare favorably with reports of up to 5% non-relapse mortality with CAR T-cells and ICU treatment in 30% of cases (see e.g., Neelapu, S. S., et al., Axicabtagene ciloleucel CAR T-cell therapy in refractory large B-cell lymphoma. New England Journal of Medicine 377, 2531-2544 (2017); and Nastoupil, L. J., et al., Standard-of-care Axicabtagene Ciloleucel for relapsed or refractory large B-cell lymphoma: results from the US lymphoma CAR T Consortium. J. Clin Oncology. 2020 Sep. 20; 38 (27): 3119-3128 (Epub 2020 May 13); each of which are incorporated herein by reference in their entirety for the purposes described herein). Furthermore, in the immediate study, the levels of inflammatory cytokines such as IL-6, TNF-α and IFN-γ remained normal at all times, further supporting the safety profile of the CAR NK cell products.

In conclusion, the results provided herein showed that iC9/CD19-CAR/IL15 CB-NK cell therapy had a similar efficacy profile to that of autologous anti-CD19 CAR T-cell therapy. However, the safety profile was very different. CD19 CAR-NK cells were not associated with significant CRS or neurotoxicity. Additionally, through methods described herein (e.g., improved donor CBU selection methodologies, etc.) and/or continued developments in the field, these results can potentially be improved.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

The following references, and those cited elsewhere herein, to the extent that they provide exemplary procedural or other details supplementary to those set forth herein, are specifically incorporated herein by reference.
1. Abramson, J. S., et al. Lisocabtagene maraleucel for patients with relapsed or refractory large B-cell lymphomas (TRANSCEND NHL 001): a multicentre seamless design study. The Lancet 396, 839-852 (2020).
2. Neelapu, S. S., et al. Axicabtagene ciloleucel CAR T-cell therapy in refractory large B-cell lymphoma. New England Journal of Medicine 377, 2531-2544 (2017).
3. Schuster, S. J., et al. Tisagenlecleucel in adult relapsed or refractory diffuse large B-cell lymphoma. New England Journal of Medicine 380, 45-56 (2019).
4. Wang, M., et al. KTE-X19 CAR T-Cell Therapy in Relapsed or Refractory Mantle-Cell Lymphoma. N Engl J Med 382, 1331-1342 (2020).
5. Brudno, J. N. & Kochenderfer, J.N. Recent advances in CAR T-cell toxicity: Mechanisms, manifestations and management. Blood Rev 34, 45-55 (2019).
6. Laskowski, T. J., Biederstädt, A. & Rezvani, K. Natural killer cells in antitumour adoptive cell immunotherapy. Nat Rev Cancer 22, 557-575 (2022).
7. Cerwenka, A. & Lanier, L. L. Natural killer cell memory in infection, inflammation and cancer. Nature Reviews Immunology 16, 112-123 (2016).

8 Huntington, N. D., Cursons, J. & Rautela, J. The cancer-natural killer cell immunity cycle. Nature Reviews Cancer 20, 437-454 (2020).
9. Vivier, E., et al. Innate or adaptive immunity? The example of natural killer cells. Science 331, 44-49 (2011).
10. Daher, M. & Rezvani, K. Outlook for New CAR-Based Therapies with a Focus on CAR NK Cells: What Lies Beyond CAR-Engineered T Cells in the Race against Cancer. Cancer Discovery 11, 45-58 (2021).
11. Rafei, H., Daher, M. & Rezvani, K. Chimeric antigen receptor (CAR) natural killer (NK)-cell therapy: leveraging the power of innate immunity. British Journal of Haematology 193, 216-230 (2021).
12. Liu, E., et al. Cord blood NK cells engineered to express IL-15 and a CD19-targeted CAR show long-term persistence and potent antitumor activity. Leukemia 32, 520-531 (2018).
13. Liu, E., et al. Use of CAR-Transduced Natural Killer Cells in CD19-Positive Lymphoid Tumors. New England Journal of Medicine 382, 545-553 (2020).
14. Fraietta, J. A., et al. Determinants of response and resistance to CD19 chimeric antigen receptor (CAR) T cell therapy of chronic lymphocytic leukemia. Nature medicine 24, 563-571 (2018).
15. Burnham, R. E., et al. Characterization of Donor Variability for γδ T Cell ex vivo Expansion and Development of an Allogeneic γδ T Cell Immunotherapy. Front Med (Lausanne) 7, 588453 (2020).
16. Jonus, H. C., et al. Dissecting the cellular components of ex vivo γδ T cell expansions to optimize selection of potent cell therapy donors for neuroblastoma immunotherapy trials. Oncoimmunology 11, 2057012 (2022).
17. Longo, D. M., et al. Inter-donor variation in cell subset specific immune signaling responses in healthy individuals. Am J Clin Exp Immunol 1, 1-11 (2012).
18. Belderbos, M. E., et al. Donor-to-Donor Heterogeneity in the Clonal Dynamics of Transplanted Human Cord Blood Stem Cells in Murine Xenografts. Biol Blood Marrow Transplant 26, 16-25 (2020).
19. Hamieh, M., et al. CAR T cell trogocytosis and cooperative killing regulate tumour antigen escape. Nature 568, 112-116 (2019).
20. Li, Y., et al. KIR-based inhibitory CARs overcome CAR-NK cell trogocytosis-mediated fratricide and tumor escape. Nature Medicine 28, 2133-2144 (2022).
21. Goel, M., Dwivedi, R., Gohiya, P. & Hegde, D. Nucleated red blood cell in cord blood as a marker of perinatal asphyxia. J Clin Neonatol 2, 179-182 (2013).
22. Colaco, S. M., Ahmed, M., Kshirsagar, V. Y. & Bajpai, R. Study of Nucleated Red Blood Cell Counts in Asphyxiated Newborns and the Fetal Outcome, (2014).
23. Sarkar, S., et al. Hypoxia induced impairment of NK cell cytotoxicity against multiple myeloma can be overcome by IL-2 activation of the NK cells. PLOS One 8, e64835 (2013).
24. Tang, F., et al. A pan-cancer single-cell panorama of human natural killer cells. Cell (2023).
25. Shahbaz, S., et al. CD71+VISTA+erythroid cells promote the development and function of regulatory T cells through TGF-β. PLOS Biol 16, e2006649 (2018).
26. Kanemasa, H., et al. The immunoregulatory function of peripheral blood CD71 (+) erythroid cells in systemic-onset juvenile idiopathic arthritis. Sci Rep 11, 14396 (2021).
27. Yang, J., et al. Red Blood Cells in Type 2 Diabetes Impair Cardiac Post-Ischemic Recovery Through an Arginase-Dependent Modulation of Nitric Oxide Synthase and Reactive Oxygen Species. JACC: Basic to Translational Science 3, 450-463 (2018).
28. Paul, S. & Lal, G. The Molecular Mechanism of Natural Killer Cells Function and Its Importance in Cancer Immunotherapy. Frontiers in Immunology 8 (2017).
29. Choi, C. & Finlay, D.K. Optimising NK cell metabolism to increase the efficacy of cancer immunotherapy. Stem Cell Research & Therapy 12, 320 (2021).
30. Chu, Y., et al. Pan-cancer T cell atlas links a cellular stress response state to immunotherapy resistance. Nat Med (2023).
31. Selli, M. E., et al. Costimulatory domains direct distinct fates of CAR-driven T cell dysfunction. Blood (2023).
32. Belk, J. A., et al. Genome-wide CRISPR screens of T cell exhaustion identify chromatin remodeling factors that limit T cell persistence. Cancer Cell 40, 768-786.e767 (2022).
33. Maurer, M. J., et al. Diagnosis-to-Treatment Interval Is an Important Clinical Factor in Newly Diagnosed Diffuse Large B-Cell Lymphoma and Has Implication for Bias in Clinical Trials. J Clin Oncol 36, 1603-1610 (2018).
34. Cappell, K. M. & Kochenderfer, J.N. Long-term outcomes following CAR T cell therapy: what we know so far. Nature Reviews Clinical Oncology (2023).
35. Chong, E.A., Ruella, M. & Schuster, S. J. Five-Year Outcomes for Refractory B-Cell Lymphomas with CAR T-Cell Therapy. New England Journal of Medicine 384, 673-674 (2021).
36. Jacobson, C. A., et al. Axicabtagene ciloleucel in relapsed or refractory indolent non-Hodgkin lymphoma (ZUMA-5): a single-arm, multicentre, phase 2 trial. Lancet Oncol 23, 91-103 (2022).
37. Frey, N. V., et al. Long-term outcomes from a randomized dose optimization study of chimeric antigen receptor modified T cells in relapsed chronic lymphocytic leukemia. Journal of Clinical Oncology 38, 2862 (2020).
38. Siddiqi, T., et al. Phase 1 TRANSCEND CLL 004 study of lisocabtagene maraleucel in patients with relapsed/refractory CLL or SLL. Blood 139, 1794-1806 (2022).
39. Locke, F. L., et al. Long-term safety and activity of axicabtagene ciloleucel in refractory large B-cell lymphoma (ZUMA-1): a single-arm, multicentre, phase 1-2 trial. Lancet Oncol 20, 31-42 (2019).
40. Deng, Q., et al. Characteristics of anti-CD19 CAR T cell infusion products associated with efficacy and toxicity in patients with large B cell lymphomas. Nat Med 26, 1878-1887 (2020).
41. Locke, F. L., et al. Tumor burden, inflammation, and product attributes determine outcomes of axicabtagene ciloleucel in large B-cell lymphoma. Blood advances 4, 4898-4911 (2020).
42. Rossi, J., et al. Preinfusion polyfunctional anti-CD19 chimeric antigen receptor T cells are associated with clinical outcomes in NHL. Blood, The Journal of the American Society of Hematology 132, 804-814 (2018).
43. Imai, K., Matsuyama, S., Miyake, S., Suga, K. & Nakachi, K. Natural cytotoxic activity of peripheral-blood lymphocytes and cancer incidence: an 11-year follow-up study of a general population. Lancet 356, 1795-1799 (2000).
44. Bozorgmehr, N., et al. CD71 (+) erythroid cells suppress T-cell effector functions and predict immunotherapy outcomes in patients with virus-associated solid tumors. J Immunother Cancer 11 (2023).
45. Shanware, N. P., et al. Conserved and Distinct Modes of CREB/ATF Transcription Factor Regulation by PP2A/B56γ and Genotoxic Stress. PLOS ONE 5, e12173 (2010).
46. Thall, P. F., Simon, R. M. & Estey, E.H. Bayesian sequential monitoring designs for single-arm clinical trials with multiple outcomes. Stat Med 14, 357-379 (1995).
47. Cheson, B. D., et al. Recommendations for initial evaluation, staging, and response assessment of Hodgkin and non-Hodgkin lymphoma: the Lugano classification. Journal of clinical oncology 32, 3059 (2014).
48. Hallek, M., et al. iwCLL guidelines for diagnosis, indications for treatment, response assessment, and supportive management of CLL. Blood, The Journal of the American Society of Hematology 131, 2745-2760 (2018).
49. Hoyos, V., et al. Engineering CD19-specific T lymphocytes with interleukin-15 and a suicide gene to enhance their anti-lymphoma/leukemia effects and safety. Leukemia 24, 1160-1170 (2010).
50. Daher, M., et al. Targeting a cytokine checkpoint enhances the fitness of armored cord blood CAR-NK cells. Blood 137, 624-636 (2021).
51. Xue, Q., et al. Single-cell multiplexed cytokine profiling of CD19 CAR-T cells reveals a diverse landscape of polyfunctional antigen-specific response. J Immunother Cancer 5, 85 (2017).
52. Li, B. & Dewey, C. N. RSEM: accurate transcript quantification from RNA-Seq data with or without a reference genome. BMC Bioinformatics 12, 323 (2011).
53. Langmead, B. & Salzberg, S.L. Fast gapped-read alignment with Bowtie 2. Nat Methods 9, 357-359 (2012).
54. Love, M. I., Huber, W. & Anders, S. Moderated estimation of fold change and dispersion for RNA-seq data with DESeq2. Genome Biol 15, 550 (2014).
55. Soneson, C., Love, M. I. & Robinson, M.D. Differential analyses for RNA-seq: transcript-level estimates improve gene-level inferences. F1000Res 4, 1521 (2015).
56. Luo, W., Friedman, M.S., Shedden, K., Hankenson, K.D. & Woolf, P.J. GAGE: generally applicable gene set enrichment for pathway analysis. BMC Bioinformatics 10, 161 (2009).
57. Liberzon, A., et al. The Molecular Signatures Database (MSigDB) hallmark gene set collection. Cell Syst 1, 417-425 (2015).
58. Barbie, D. A., et al. Systematic RNA interference reveals that oncogenic KRAS-driven cancers require TBK1. Nature 462, 108-112 (2009).
59. Hänzelmann, S., Castelo, R. & Guinney, J. GSVA: gene set variation analysis for microarray and RNA-seq data. BMC Bioinformatics 14, 7 (2013).
60. Van de Sande, B., et al. A scalable SCENIC workflow for single-cell gene regulatory network analysis. Nature Protocols 15, 2247-2276 (2020).
61. Moerman, T., et al. GRNBoost2 and Arboreto: efficient and scalable inference of gene regulatory networks. Bioinformatics 35, 2159-2161 (2018).
62. Corces, M. R., et al. An improved ATAC-seq protocol reduces background and enables interrogation of frozen tissues. Nature Methods 14, 959-962 (2017).
63. Thiyagarajan, T., et al. Inhibiting androgen receptor splice variants with cysteine-selective irreversible covalent inhibitors to treat prostate cancer. Proceedings of the National Academy of Sciences 120, e2211832120 (2023).
64. Li, H. Aligning sequence reads, clone sequences and assembly contigs with BWA-MEM. arXiv: Genomics (2013).
65. Ramírez, F., et al. deepTools2: a next generation web server for deep-sequencing data analysis. Nucleic Acids Res 44, W160-165 (2016).
66. Zhang, Y., et al. Model-based Analysis of ChIP-Seq (MACS). Genome Biology 9, R137 (2008).
67. Stark, R. & Brown, G. DiffBind: Differential binding analysis of ChIP-Seq peak data. (2012).
68. Stuart, T., Srivastava, A., Madad, S., Lareau, C.A. & Satija, R. Single-cell chromatin state analysis with Signac. Nature Methods 18, 1333-1341 (2021).
69. Vera, J., et al. T lymphocytes redirected against the kappa light chain of human immunoglobulin efficiently kill mature B lymphocyte-derived malignant cells. Blood 108, 3890-3897 (2006).
70. Gelman A, C.J., Stern HS, Dunson DB, Vehtari A, Rubin D. Bayesian Data Analysis, (Chapman & Hall, CRC Texts in Statistical Science, 3rd Edition, Boca Raton, Florida, 2013).
71. Kruschke, J.K. Doing Bayesian Data Analysis: A Tutorial with R, JAGS, and Stan, (Academic Press, 2015).

What is claimed is:

1. A method of selecting cryopreserved cord blood units (CBUs) for manufacture of improved immune cells comprising, selecting CBUs based on the time from birth of a baby from which the CBU was derived and cryopreservation of the CBU.

2. The method of claim 1, further comprising selecting CBUs that were cryopreserved within about or exactly 32, 30, 28, 26, 24, 22, or 20 hours following birth of a baby from which the CBU was derived.

3. The method of claim 1, wherein the CBUs were cryopreserved within exactly or about 24 hours following birth of the baby.

4. The method of claim 1, further comprising selecting CBUs that comprise a nucleated red blood cell (NRBC) content that is:
a) less than or equal to, exactly or about 8.5×10⁷, 8.0×10⁷, or 7.5×10⁷ cells when measured post-reduction,
b) less than or equal to, exactly or about 9.9×10⁷, 9.4×10⁷, or 8.9×10⁷ cells when measured pre-reduction, and/or
c) less than or equal to, exactly or about 5%, 4%, or 3% of the total nucleated cells (TNC) when measured post-reduction.

5. The method of claim 4, wherein the NRBC content is less than or equal to, exactly or about 8.0×10⁷ cells when measured post-reduction, less than or equal to, exactly or about 9.4×10⁷ cells when measured pre-reduction, and/or less than or equal to, exactly or about 4% of the total nucleated cells (TNC) when measured post-reduction.

6. The method of claim 1, wherein the CBUs are not selected based on the relative levels of one or more immune cells.

7. The method of claim 6, wherein the CBUs are not selected based upon their percentage of NK cells, CD8+ T cells, CD4+ T cells, T regulatory cells, B cells, Monocyte-derived dendritic cells (Mo-DCS), and/or plasmacytoid dendritic cells (pDCs).

8. The method of claim 6, wherein the CBUs do not have significant differences in the percentages of NK cells, CD8+ T cells, CD4+ T cells, T regulatory cells, B cells, Monocyte-derived dendritic cells (Mo-DCS), and/or plasmacytoid dendritic cells (pDCs).

9. The method of claim 1, further comprising selecting CBUs based on:
a) total cell viability pre-cryopreservation,
b) total CD34 positive cell percentage,
c) weight of the baby,
d) race of the baby's parents,
e) baby's mothers age,
f) gestational age of the baby,
g) collection method of the cord blood,
h) sex of the baby, and/or
i) pre-process volume of the cord blood collected.

10. The method of claim 9, further comprising selecting CBUs based on:
a) the total cell viability pre-cryopreservation is greater than or equal to, exactly or about 95%,
b) the total CD34 positive cell percentage is greater than or equal to, exactly or about 0.2%,
c) the weight of baby is greater than or equal to, exactly or about 3,000 grams,
d) the baby has at least one Caucasian parents,
e) the mothers age is less than or equal to, exactly or about 34 years of age,
f) the gestational age of the baby is less than or equal to, exactly or about 40 weeks,
g) the cord blood was collected intra-utero and/or ex-utero,
h) the baby is male, and/or
i) the pre-process volume of the cord blood collected was less than or equal to, exactly or about 150 ml.

11. The method of claim 9, further comprising selecting CBUs based on:
a) the total cell viability pre-cryopreservation is equal to or greater than or equal to, exactly or about 98.5%,
b) the total CD34 positive cell percentage is greater than or equal to, exactly or about 0.245%,
c) the weight of baby is greater than or equal to, exactly or about 3,650 grams,
d) the baby has at least one Caucasian parents,
e) the mothers age is less than or equal to, exactly or about 32 years of age,
f) the gestational age of the baby is less than or equal to, exactly or about 38 weeks,
g) the cord blood was collected intra-utero,
h) the baby is male, and/or
i) the pre-process volume of the cord blood collected was less than or equal to, exactly or about 120 ml.

12. The method of claim 11, wherein at least 3 of the selection factors are utilized.

13. The method of claim 1, wherein the immune cells phenotypic, transcriptional, and/or epigenetic signatures are distinct from immune cells not selected based on the time from birth of a baby from which the CBU was derived and cryopreservation of the CBU.

14. The method of claim 13, wherein the immune cells have an increased polyfunctional strength index (PSI).

15. The method of claim 14, wherein the increased PSI comprises an increased effector PSI, increased stimulatory PSI, and/or increased chemoattractive PSI.

16. The method of claim 13, wherein the immune cells have increased chromatin accessibility and/or transcriptional levels of genes encoding ZIC2, GLI3, TBX21, IRF2, IRF3, IRF4, IRF7, IRF8, IRF9, NKX2-3, NKX2-8, GLI2, EOMES, GZMA, CXCR6, CMKLR1, NKG2D, CD16, 2B4, T-BET, PFN, GZMA, and/or PRF1.

17. The method of claim 13, wherein the immune cells have an increased population doubling rate and/or increased protein secretion rate.

18. The method of claim 13, wherein the immune cells have an increased basal respiration and/or maximal respiration rate.

19. The method of claim 13, wherein the immune cells have decreased chromatin accessibility and/or transcriptional levels of genes encoding ATF1, ATF2, ATF3, ATF7, CREB1, CREB5, NFAT2, NFATC2, FOX, JUN, JUNB, SMAD2, SMAD3, HIF1A, MAFF, JMJD6, DDIT3, SIAH2, NR4A1, DNAJA1, BAK1, NFKB1, IL-10, LAG3, HASP90AB1, HSPA5, and/or HSPA13.

20. The method of claim 13, wherein the immune cells have a decreased rate of trogocytosis and/or decreased transcriptional levels of hallmarks of TNFα signaling via NF-κβ, UV response, hypoxia, IL2 STAT5 signaling, Heme metabolism, apoptosis, inflammatory response, estrogen response early, G2M checkpoint, TGFβ signaling, p53 pathway, cholesterol homeostasis, KRAS signaling, and/or Myc targets V1.

21. The method of claim 13, wherein the immune cells have decreased NR4A1, JUND, BCL3, MEF2D, HOXA5, FOXB, JUN, MAFF, ZNF281, KLF6, REL, CEBPG, KLF16, HIF1A, FOS, BCLAF1, GATA3, FOSL2, RARG, EGR2, and/or MAF regulon activity.

22. The method of claim 1, wherein the immune cells are natural killer (NK) cells.

23. The method of claim 22, further comprising the step of expanding the NK cells.

24. The method of claim 23, wherein the CBUs are re-selected based on:
a) the NK cell expansion between days 0 and 15 of culture, and/or
b) the NK cell expansion between days 6 and 15 of culture.

25. The method of claim 24, wherein:
a) the NK cell expansion between days 0 and 15 of culture is greater than or equal to, exactly or about 350 fold, and/or
b) the NK cell expansion between days 6 and 15 of culture is greater than or equal to, exactly or about 50 fold.

26. The method of claim 24, wherein:
a) the NK cell expansion between days 0 and 15 of culture is greater than or equal to, exactly or about 450 fold, and/or
b) the NK cell expansion between days 6 and 15 of culture is greater than or equal to, exactly or about 70 fold.

27. The method of claim 22, further comprising the step of modifying the NK cells.

28. The method of claim 27, wherein the NK cells are modified to express one or more non-endogenous gene products.

29. The method of claim 28, wherein the non-endogenous gene product comprises an antigen receptor, a cytokine, a homing receptor, a chemokine receptor, and combinations thereof.

30. The method of claim 29, wherein the non-endogenous receptor is a chimeric receptor .

31. The method of claim 30, wherein the chimeric receptor is a chimeric antigen receptor (CAR).

32. The method of claim 31, wherein the CAR targets CD19, CD70, and/or TROP2.

33. The method of claim 29, wherein the non-endogenous receptor is a T-cell receptor (TCR).

34. The method of claim 28, further comprising expression of one or more non-endogenous cytokines.

35. The method of claim 34, wherein the cytokine is IL-15 and/or IL-21.

36. The method of claim 22, wherein the NK cells are pre-activated with one or more cytokines.

37. The method of claim 36, wherein the cytokines are IL-2, IL-7, IL-12, IL-15, IL-18, IL-21, or a combination thereof.

38. The method of claim 22, wherein the NK cell comprises one or more engineered mutations in an endogenous gene.

39. The method of claim 38, wherein the endogenous gene is GR, TGFBR2, CISH, and/or CD38.

40. A composition comprising a CBU identified by any one of the methods of claims 1 to 39.

41. The composition of claim 40, comprised in a pharmaceutically acceptable carrier.

42. The composition of claim 40, formulated with one or more cryoprotectants.

43. A composition comprising a population of immune cells derived from CBUs selected using the method of any one of claims 1 to 39.

44. A method of treating a subject with cancer comprising, administering the population of immune cells according to claim 43.

45. The method of claim 44, wherein the subject has increased rates of overall response (OR), complete response (CR), progression-free survival (PFS), and/or overall survival (OS) relative to a subject not treated with the population of immune cells.

46. A method for the manufacture of engineered immune cells, comprising: engineering an immune cell population to express one or more non-endogenous gene products, wherein the immune cells are derived from a population of cord blood cells from the birth of a baby, and wherein prior to cryopreservation the population of cord blood cells has the following characteristics:
(a) cord blood cells were cryopreserved within about or exactly 32, 30, 28, 26, 24, 22, or 20 hours following birth of a baby from which the cord blood cells were obtained; and
(b) comprise a nucleated red blood cell (NRBC) content that is:
i) less than or equal to, exactly or about 8.5×10⁷, 8.0×10⁷, or 7.5×10⁷ cells when measured post-reduction,
ii) less than or equal to, exactly or about 9.9×10⁷, 9.4×10⁷, or 8.9×10⁷ cells when measured pre-reduction, and/or
iii) less than or equal to, exactly or about 5%, 4%, or 3% of the total nucleated cells (TNC) when measured post-reduction;
and optionally,
(c) the total cell viability pre-cryopreservation is greater than or equal to, exactly or about 95%,
(d) the total CD34 positive cell percentage is greater than or equal to, exactly or about 0.2%,
(e) the weight of baby is greater than or equal to, exactly or about 3,000 grams,
(f) the baby has at least one Caucasian parents,
(g) the mothers age is less than or equal to, exactly or about 34 years of age,
(h) the gestational age of the baby is less than or equal to, exactly or about 40 weeks,
(i) the cord blood was collected intra-utero and/or ex-utero,
(j) the baby is male, and/or
(k) the pre-process volume of the cord blood collected was less than or equal to, exactly or about 150 ml.

47. The method of claim 46, wherein:
(a) cord blood cells were cryopreserved within exactly or about 24 hours following birth of the baby; and
(b) comprise a nucleated red blood cell (NRBC) content that is:
i) less than exactly or about 8.0×10⁷ cells when measured post-reduction,
ii) less than or equal to, exactly or about 9.4×10⁷ cells when measured pre-reduction, and/or
iii) less than or equal to, exactly or about 4% of the total nucleated cells (TNC) when measured post-reduction,
and optionally,
(c) the total cell viability pre-cryopreservation is equal to or greater than or equal to, exactly or about 98.5%,
(d) the total CD34 positive cell percentage is greater than or equal to, exactly or about 0.245%,
(e) the weight of baby is greater than or equal to, exactly or about 3,650 grams,
(f) the baby has at least one Caucasian parents,
(g) the mothers age is less than or equal to, exactly or about 32 years of age,
(h) the gestational age of the baby is less than or equal to, exactly or about 38 weeks,
(i) the cord blood was collected intra-utero,
(j) the baby is male, and/or
(k) the pre-process volume of the cord blood collected was less than or equal to, exactly or about 120 ml.

48. A method for the manufacture of a source material for the manufacture of a composition comprising immune cells, the method comprising, cryopreserving a cell population comprising immune cells derived from a population of cord blood cells from the birth of a baby, wherein prior to cryopreservation such population of cord blood cells has the following characteristics:
(a) cord blood cells were cryopreserved within about or exactly 32, 30, 28, 26, 24, 22, or 20 hours following birth of a baby from which the cord blood cells were obtained; and
(b) comprise a nucleated red blood cell (NRBC) content that is:
i) less than or equal to, exactly or about 8.5×10⁷, 8.0×10⁷, or 7.5×10⁷ cells when measured post-reduction,
ii) less than or equal to, exactly or about 9.9×10⁷, 9.4×10⁷, or 8.9×10⁷ cells when measured pre-reduction, and/or
iii) less than or equal to, exactly or about 5%, 4%, or 3% of the total nucleated cells (TNC) when measured post-reduction;
and optionally,
(c) the total cell viability pre-cryopreservation is greater than or equal to, exactly or about 95%,
(d) the total CD34 positive cell percentage is greater than or equal to, exactly or about 0.2%,
(e) the weight of baby is greater than or equal to, exactly or about 3,000 grams,
(f) the baby has at least one Caucasian parents,
(g) the mothers age is less than or equal to, exactly or about 34 years of age,
(h) the gestational age of the baby is less than or equal to, exactly or about 40 weeks,
(i) the cord blood was collected intra-utero and/or ex-utero,
(j) the baby is male, and/or
(k) the pre-process volume of the cord blood collected was less than or equal to, exactly or about 150 ml.

49. The method of claim 48, wherein:
(a) cord blood cells were cryopreserved within exactly or about 24 hours following birth of the baby; and
(b) comprise a nucleated red blood cell (NRBC) content that is:
i) less than exactly or about 8.0×10⁷ cells when measured post-reduction,
ii) less than or equal to, exactly or about 9.4×10⁷ cells when measured pre-reduction, and/or
iii) less than or equal to, exactly or about 4% of the total nucleated cells (TNC) when measured post-reduction,
and optionally,
(c) the total cell viability pre-cryopreservation is equal to or greater than or equal to, exactly or about 98.5%,
(d) the total CD34 positive cell percentage is greater than or equal to, exactly or about 0.245%,
(e) the weight of baby is greater than or equal to, exactly or about 3,650 grams,
(f) the baby has at least one Caucasian parents,
(g) the mothers age is less than or equal to, exactly or about 32 years of age,
(h) the gestational age of the baby is less than or equal to, exactly or about 38 weeks,
(i) the cord blood was collected intra-utero,
(j) the baby is male, and/or
(k) the pre-process volume of the cord blood collected was less than or equal to, exactly or about 120 ml.

50. A composition comprising an isolated population of cord-blood derived immune cells, wherein the immune cells are derived from one or more cord blood units from the birth of a baby that have the following characteristics:
(a) cord blood cells were cryopreserved within about or exactly 32, 30, 28, 26, 24, 22, or 20 hours following birth of a baby from which the cord blood cells were obtained; and
(b) comprise a nucleated red blood cell (NRBC) content that is:
i) less than or equal to, exactly or about 8.5×10⁷, 8.0×10⁷, or 7.5×10⁷ cells when measured post-reduction,
ii) less than or equal to, exactly or about 9.9×10⁷, 9.4×10⁷, or 8.9×10⁷ cells when measured pre-reduction, and/or
iii) less than or equal to, exactly or about 5%, 4%, or 3% of the total nucleated cells (TNC) when measured post-reduction;
and optionally,
(c) the total cell viability pre-cryopreservation is greater than or equal to, exactly or about 95%,
(d) the total CD34 positive cell percentage is greater than or equal to, exactly or about 0.2%,
(e) the weight of baby is greater than or equal to, exactly or about 3,000 grams,
(f) the baby has at least one Caucasian parents,
(g) the mothers age is less than or equal to, exactly or about 34 years of age,
(h) the gestational age of the baby is less than or equal to, exactly or about 40 weeks,
(i) the cord blood was collected intra-utero and/or ex-utero,
(j) the baby is male, and/or
(k) the pre-process volume of the cord blood collected was less than or equal to, exactly or about 150 ml.

51. The composition of claim 50, wherein:
(a) cord blood cells were cryopreserved within exactly or about 24 hours following birth of the baby; and
(b) comprise a nucleated red blood cell (NRBC) content that is:
i) less than exactly or about 8.0×10⁷ cells when measured post-reduction,
ii) less than or equal to, exactly or about 9.4×10⁷ cells when measured pre-reduction, and/or
iii) less than or equal to, exactly or about 4% of the total nucleated cells (TNC) when measured post-reduction,
and optionally,
(c) the total cell viability pre-cryopreservation is equal to or greater than or equal to, exactly or about 98.5%,
(d) the total CD34 positive cell percentage is greater than or equal to, exactly or about 0.245%,
(e) the weight of baby is greater than or equal to, exactly or about 3,650 grams,
(f) the baby has at least one Caucasian parents,
(g) the mothers age is less than or equal to, exactly or about 32 years of age,
(h) the gestational age of the baby is less than or equal to, exactly or about 38 weeks,
(i) the cord blood was collected intra-utero,
(j) the baby is male, and/or
(k) the pre-process volume of the cord blood collected was less than or equal to, exactly or about 120 ml.


SEQUENCE LISTING (6 sequences)

SEQ ID NO: 1 - DNA, 13 bp, Homo sapiens
  source 1..13: mol_type = genomic DNA
nwgbctvncwshh

SEQ ID NO: 2 - DNA, 12 bp, Homo sapiens
  source 1..12: mol_type = genomic DNA
bnbvdtkacghn

SEQ ID NO: 3 - DNA, 11 bp, Homo sapiens
  source 1..11: mol_type = genomic DNA
dntkwhrhhay

SEQ ID NO: 4-5 - skipped (2 entries)

SEQ ID NO: 6 - DNA, 18 bp, Homo sapiens
  source 1..18: mol_type = genomic DNA
brawarygaaashndnnn